(19) United States
(12) Patent Application Publication
(10) Pub. No.: US 2026/0085302 A1
(43) Pub. Date: Mar. 26, 2026

(54) COMPOSITIONS AND METHODS FOR CONTROLLING PLANT PESTS

(71) Applicant: Confluence Genetics, LLC, St. Louis, MO (US)

(72) Inventors: Matthew Brett Begemann, St. Louis, MO (US); Emma Elizabeth January, St. Louis, MO (US); Allison Jane Newton Antonakos, St. Louis, MO (US); Jason K. Bull, St. Louis, MO (US); Anna Singer, St. Louis, MO (US)

(73) Assignee: Confluence Genetics, LLC, St. Louis, MO (US)

(21) Appl. No.: 19/334,536

(22) Filed: Sep. 19, 2025

Related U.S. Application Data
(60) Provisional application No. 63/697,221, filed on Sep. 20, 2024.

(51) Int. Cl. C12N 9/22; A01N 63/50; A01N 63/60; A01P 1/00; A01P 3/00; A01P 5/00; A01P 7/04; A01P 17/00; C12N 15/113; C12N 15/52; C12N 15/82

(52) U.S. Cl. CPC C12N 9/226; A01N 63/50; A01N 63/60; A01P 1/00; A01P 3/00; A01P 5/00; A01P 7/04; A01P 17/00; C12N 15/113; C12N 15/52; C12N 15/8279; C12N 2310/20

(57) ABSTRACT

Compositions and methods for targeting pre-determined DNA or RNA sequences in plant pests are provided. The methods result in the targeted elimination of plant pests that comprise the pre-determined DNA or RNA sequence(s). Compositions comprise a Cas12a2 protein, or a polynucleotide encoding the same, and at least one guide polynucleotide, or a polynucleotide encoding the same, wherein each guide polynucleotide comprises a portion complementary to a pre-determined target sequence of a plant pest. Methods to use these compositions to selectively target and eliminate plant pests that harbor the targeted DNA or RNA sequence(s) are described herein.

### CROSS-REFERENCE TO RELATED APPLICATIONS

This application claims priority to U.S. Provisional Application No. 63/697,221, filed Sep. 20, 2024, which is incorporated by referenced herein in its entirety.

### REFERENCE TO A SEQUENCE LISTING SUBMITTED ELECTRONICALLY AS AN XML FILE

The instant application contains a Sequence Listing which has been submitted in xml format and is hereby incorporated by reference in its entirety. Said xml copy, created on Sep. 19, 2025, is named B88552_1670WO_00444_SL_v4, and is 447,756 bytes in size.

### FIELD OF THE INVENTION

The present invention relates to compositions and methods for controlling plant pests in a sequence-specific manner.

### BACKGROUND OF THE INVENTION

Stresses to plants may be caused by both abiotic and biotic agents. For example, abiotic stresses include, for example, excessive or insufficient available water, temperature extremes, and synthetic chemicals such as herbicides. Biotic causes of stress include infection with pathogen, infestation by pests, and parasitism by another plant.

These biotic stresses are financially costly, resulting in crop loss and/or in use of expensive pesticides to control pests. Synthetic chemical insecticidal compounds are used to control pests, but they can be environmentally unfriendly. Biotechnology in the last decades have presented new opportunities for pest control through genetic engineering. Advances in plant genetics coupled with the identification of factors deleterious to pests and naturally-occurring plant defensive compounds offer the opportunity to create transgenic crop plants capable of producing such defensive agents and thereby protect the plants against pest infestation.

Genetically engineered insect-resistant crop plants are now widely used in agriculture and have provided the farmer with an environmentally friendly and commercially attractive alternative to traditional insect control methods. While these genetically engineered crops have provided benefits, they may only provide resistance to particular pests (e.g., insect pests). In some cases, insects can develop resistance to the transgenically produced insecticidal compound, which highlights the need to identify alternative biological control agents for pest control. Accordingly, there remains a need for pest control agents effective against diverse pests and that may obviate any declining effectiveness of pest control due to developing resistance of the pests to defense compounds.

CRISPR systems have been proposed as a possible technology that may be adapted to selectively eliminate unwanted and/or harmful cells (Gomaa et al (2014) mBio e00928-13), with a focus on Type I CRISPR systems because these CRISPR systems have a processive DNase activity wherein the CRISPR nuclease hybridizes with the target sequence, then processively degrades DNA following this hybridization, sometimes resulting in the near complete elimination of the targeted DNA molecule (e.g., a targeted plasmid, viral DNA molecule, circular bacterial genome, or other DNA molecule).

While these properties of Type I CRISPR systems may be desirable in some applications, Type I CRISPR systems also have some drawbacks. For instance, Type I CRISPR systems are typically large, multi-component systems. Their size can make packaging of Type I CRISPR systems in commonly used plasmids, viral vectors, and other vectors difficult. Furthermore, Type I CRISPR systems may not show optimal activity in some cells that may be desirable to eliminate. While CRISPR systems show promise in their ability to target and eliminate undesirable cells, alternatives to Type I CRISPR systems would be valuable. Cas9-based CRISPR systems have been explored for their ability to selectively eliminate cells (Citorik et al (2014) Nat Biotechnol 32:1141-1145; Bikard et al (2014) Nat Biotechnol 32:1146-1150; U.S. patent application Ser. No. 14/475,785); however these systems may be hampered by the mechanism of Cas9 nucleases. Because Cas9 nucleases make a single DSB, repair of this DSB may result in survival of the unwanted or harmful cell.

Some Type V CRISPR enzymes have been shown to harbor a primary, sequence-specific, activity against a particular type of substrate; following this sequence-specific primary activity, the Type V enzyme is then able to access a secondary, collateral activity in a non-sequence-specific manner. As an example, Cpf1 (Cas12a) has been shown to harbor primary double-stranded break production activity against double-stranded DNA (dsDNA). After Cpf1 hybridizes with and cleaves its primary target, the protein is then capable of cleaving single-stranded DNA (ssDNA) in a non-sequence-specific manner (Chen et al (2018) Science 360:436-439). Other Type V CRISPR enzymes have been shown, for example, to harbor a primary activity against RNA, with secondary activities directed against RNA and ssDNA (Yan et al (2019) Science 363:88-91). The present invention describes the primary and secondary activities of certain Cas12a2 enzymes, a group of Type V CRISPR enzymes, whose secondary activities can result in the death of plant pests.

### SUMMARY OF THE INVENTION

Compositions and methods for selectively controlling plant pests using Cas12a2 CRISPR systems are provided. The methods result in cell death for those plant pests that harbor particular pre-determined and targeted DNA or RNA sequences leaving other cells that do not comprise the targeted DNA or RNA sequences unharmed. Compositions comprise a Cas12a2 polypeptide, or a polynucleotide encoding the Cas12a2 polypeptide, and at least one guide polynucleotide, or a polynucleotide encoding the same, that comprises a portion complementary to a target sequence of a plant pest and a portion that can interact with the Cas12a2 polypeptide and can guide Cas12a2 to bind to the target sequence. The Cas12a2 polypeptide can comprise one or more conserved amino acid motifs selected from: (a) a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I; (b) a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V; (c) a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V; (d) a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F; (e) a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid; (f) a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F; (g) a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K; (h) a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T; (i) a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K; (j) a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V; (k) a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A; (1) a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T; (m) a conserved motif 13 comprising the amino acid sequence set forth as X₁LX₃X₄X₅X₆X₇X₈KX₁₀GX₁₂X₁₃ANX₁₆X₁₇G X₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H; (n) a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I; (o) a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid; (p) a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid; and/or (q) a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N. In some embodiments, Cas12a2 does not require a tracrRNA for function. Methods to use these compositions to selectively target and eliminate plant pests, and thus increase the resistance or tolerance of a plant to such plant pests or control the plant pest in an area of cultivation, are described herein.

### BRIEF DESCRIPTION OF THE DRAWINGS

FIGS. 1A-1C show amino acid sequence alignments identifying conserved residues within three domains of the Sulf-type Cas12a2 proteins corresponding with SEQ ID NOs: 26-39, and 166. FIG. 1A shows alignment of amino acid residues 370 to 389. Unk106 and Unk107 amino acid residues (SEQ ID NO: 121) corresponding to amino acid residues 370 to 389 of SuCas12a2, Unk108 amino acid residues (SEQ ID NO: 122) corresponding to amino acid residues 370 to 389 of SuCas12a2, Unk89 amino acid residues (SEQ ID NO: 123) corresponding to amino acid residues 370 to 389 of SuCas12a2, Unk112 amino acid residues (SEQ ID NO: 124) corresponding to amino acid residues 370 to 389 of SuCas12a2, Unk113 amino acid residues (SEQ ID NO: 125) corresponding to amino acid residues 370 to 389 of SuCas12a2, Unk88 amino acid residues (SEQ ID NO: 126) corresponding to amino acid residues 370 to 389 of SuCas12a2, Unk119 and Unk120 amino acid residues (SEQ ID NO: 127) corresponding to amino acid residues 370 to 389 of SuCas12a2, Unk110 and Unk111 amino acid residues (SEQ ID NO: 128) corresponding to amino acid residues 370 to 389 of SuCas12a2, Unk115 amino acid residues (SEQ ID NO: 171) corresponding to amino acid residues 370 to 389 of SuCas12a2, SuCas12a2 amino acid residues 370 to 389 (SEQ ID NO: 129), Unk114 amino acid residues (SEQ ID NO: 130) corresponding to amino acid residues 370 to 389 of SuCas12a2, Unk109 amino acid residues (SEQ ID NO: 131) corresponding to amino acid residues 370 to 389 of SuCas12a2, and Unk97 amino acid residues (SEQ ID NO: 132) corresponding to amino acid residues 370 to 389 of SuCas12a2. FIG. 1B shows alignment of amino acid residues 896 to 919. Unk106 and Unk107 amino acid residues (SEQ ID NO: 133) corresponding to amino acid residues 896 to 919 of SuCas12a2, Unk108 amino acid residues (SEQ ID NO: 134) corresponding to amino acid residues 896 to 919 of SuCas12a2, Unk89 amino acid residues (SEQ ID NO: 135) corresponding to amino acid residues 896 to 919 of SuCas12a2, Unk112 amino acid residues (SEQ ID NO: 136) corresponding to amino acid residues 896 to 919 of SuCas12a2, Unk113 amino acid residues (SEQ ID NO: 137) corresponding to amino acid residues 896 to 919 of SuCas12a2, Unk88 amino acid residues (SEQ ID NO: 138) corresponding to amino acid residues 896 to 919 of SuCas12a2, Unk119 amino acid residues (SEQ ID NO: 139) corresponding to amino acid residues 896 to 919 of SuCas12a2, Unk120 amino acid residues (SEQ ID NO: 140) corresponding to amino acid residues 896 to 919 of SuCas12a2, Unk110 amino acid residues (SEQ ID NO: 141) corresponding to amino acid residues 896 to 919 of SuCas12a2, Unk111 amino acid residues (SEQ ID NO: 142) corresponding to amino acid residues 896 to 919 of SuCas12a2, SuCas12a2 amino acid residues 896 to 919 (SEQ ID NO: 143), Unk115 amino acid residues (SEQ ID NO: 172) corresponding to amino acid residues 896 to 919 of SuCas12a2, Unk114 amino acid residues (SEQ ID NO: 144) corresponding to amino acid residues 896 to 919 of SuCas12a2, Unk109 amino acid residues (SEQ ID NO: 145) corresponding to amino acid residues 896 to 919 of SuCas12a2, and Unk97 amino acid residues (SEQ ID NO: 146) corresponding to amino acid residues 896 to 919 of SuCas12a2. FIG. 1C shows alignment of amino acid residues 1028 to 1049. Unk106 and Unk107 amino acid residues (SEQ ID NO: 147) corresponding to amino acid residues 1028 to 1049 of SuCas12a2, Unk108 amino acid residues (SEQ ID NO: 148) corresponding to amino acid residues 1028 to 1049 of SuCas12a2, Unk89 amino acid residues (SEQ ID NO: 149) corresponding to amino acid residues 1028 to 1049 of SuCas12a2, Unk112 amino acid residues (SEQ ID NO: 150) corresponding to amino acid residues 1028 to 1049 of SuCas12a2, Unk113 and Unk88 amino acid residues (SEQ ID NO: 151) corresponding to amino acid residues 1028 to 1049 of SuCas12a2, Unk119 amino acid residues (SEQ ID NO: 152) corresponding to amino acid residues 1028 to 1049 of SuCas12a2, Unk120 amino acid residues (SEQ ID NO: 153) corresponding to amino acid residues 1028 to 1049 of SuCas12a2, Unk110 amino acid residues (SEQ ID NO: 154) corresponding to amino acid residues 1028 to 1049 of SuCas12a2, Unk111 amino acid residues (SEQ ID NO: 155) corresponding to amino acid residues 1028 to 1049 of SuCas12a2, SuCas12a2 amino acid residues 1028 to 1049 (SEQ ID NO: 156), Unk115 amino acid residues (SEQ ID NO: 173) corresponding to amino acid residues 1028 to 1049 of SuCas12a2, Unk114 amino acid residues (SEQ ID NO: 157) corresponding to amino acid residues 1028 to 1049 of SuCas12a2, Unk109 amino acid residues (SEQ ID NO: 158) corresponding to amino acid residues 1028 to 1049 of SuCas12a2, and Unk97 amino acid residues (SEQ ID NO: 159) corresponding to amino acid residues 1028 to 1049 of SuCas12a2. SuCas12a2 is also known as SuCms1 herein.

FIG. 2 graphically depicts the results of a toxicity assay in E. coli evaluating the toxicity of Unk97, Unk88, and Unk89 as compared to SuCas12a2 as a positive control.

FIGS. 3A and 3B show results of Pseudomonas syringae DC3000 induction experiments described in Example 8. Pseudomonas syringae DC3000 expressing Unk97 with HOPAA1-1 guide no longer cause robust infection in Arabidopsis (FIG. 3A) and show significantly less CFUs than Pseudomonas syringae DC3000 expressing the GFP control or wild type Pseudomonas syringae DC3000 (FIG. 3B).

### DETAILED DESCRIPTION OF THE INVENTION

Methods and compositions are provided herein for the selective targeting and elimination of plant pests that harbor certain pre-determined DNA or RNA sequences through the use of a CRISPR-Cas12a2 system and components thereof. The CRISPR enzymes of the invention are selected from a Cas12a2 enzyme, e.g. a Cas12a2 ortholog or a mutated Cas12a2 enzyme. Cas12a2 can be alternatively referred to as Cms1, which is an abbreviation for CRISPR from Microgenomates and Smithella, and is so named because some bacterial species in these groups encode Cms1 nucleases; the terms Cas12a2, Csm1, and Cms1 may be used interchangeably. The methods and compositions include nucleic acids to bind target DNA or RNA sequences. This is advantageous as nucleic acids are much easier and less expensive to produce than, for example, peptides, and the specificity can be varied according to the length of the stretch where homology is sought. Complex 3-D positioning of multiple fingers, for example is not required. In some preferred embodiments, the nucleic acids are guide polynucleotides such as guide RNAs (gRNAs; alternatively CRISPR RNAs or crRNAs) that are capable of interacting with a Cas12a2 enzyme and of hybridizing with a DNA or RNA sequence through base pairing. As used herein, guide RNAs that are capable of interacting or that are designed to interact with a Cas12a2 polypeptide can bind, associate with, or otherwise form a complex with the Cas12a2 polypeptide. Methods of measuring interaction of gRNAs with Cas12a2 polypeptide are well known in the art.

Also provided are nucleic acids encoding the Cas12a2 polypeptides, as well as methods of using Cas12a2 polypeptides to target specific DNA or RNA sequences of plant pests. The targeted DNA sequences may be present in genomic DNA, plasmid DNA, or other DNA elements harbored within the targeted cells. The targeted RNA sequences may be double-stranded RNA (dsRNA), single-stranded RNA (ssRNA), including mRNA, or other RNA elements harbored within the targeted cells. The Cas12a2 polypeptides interact with specific guide polynucleotides such as guide RNAs (gRNAs), which direct the Cas12a2 endonuclease to a specific target site. Cas12a2 activity can encompass primary activity that can result in an initial site-specific single or double-stranded cut to a polynucleotide followed by secondary activity that can result in a non-specific cleavage and/or degradation of polynucleotides in a cell. The primary activity can produce (i) a single-strand or double-strand break in dsDNA or dsRNA or (ii) a single-strand break in ssRNA or ssDNA. This site-specific primary activity occurs at a target sequence adjacent to a recognition sequence, which may be referred to as a protospacer-adjacent motif (PAM), a protospacer-flanking motif (PFM), or a protospacer-flanking sequence (PFS). While, in certain embodiments, the term PAM is used in the context of DNA targets and the terms PFM and PFS are used in the context of RNA targets, the terms PAM, PFM, and PFS may be used interchangeably in the context of DNA and RNA targets.

In certain embodiments, an RNA target sequence comprises the reverse complement of a corresponding DNA target sequence, such that the reverse complement of any DNA target sequence disclosed herein can function as an RNA target sequence. Moreover, DNA target sequences can be located 3′ from a PAM and, thus, an RNA target sequence can be located 5′ of a PFM, PFS, or PAM. As used herein, target sequences can refer to a DNA or RNA target sequence that results in site-specific cleavage of the polynucleotide and precedes non-specific cleavage and/or degradation of other DNA or RNA in the cell.

Without being limited by theory, the Cas12a2-gRNA complex hybridizes with the targeted DNA or RNA sequence (the “initial hybridization event”), at which site the Cas12a2 endonuclease introduces a break (e.g., single-stranded (SSB) or double-stranded break (DSB)) in a DNA or an RNA target polynucleotide (i.e. a primary activity of the Cas12a2 endonuclease). This process of hybridization and SSB or DSB production leads to a change in the structure of the Cas12a2 protein, resulting in a protein that is capable of degrading double-stranded DNA (dsDNA), single-stranded DNA (ssDNA), dsRNA, and/or ssRNA in a non-sequence-specific manner (i.e. a secondary activity of the Cas12a2 endonuclease), leading to cell death. In some embodiments, the Cas12a2-gRNA complex can hybridize to RNA, such as messenger RNA (mRNA), to initiate the structural change in the Cas12a2 protein that results in non-sequence specific cleavage of nucleic acids. Since the specificity of the initial hybridization event is provided by the guide RNA, the Cas12a2 polypeptide is universal and can be used with different guide RNAs to target different genomic sequences. Cas12a2-associated CRISPR arrays are processed into mature crRNAs without the requirement of an additional trans-activating crRNA (tracrRNA). Cas12a2 proteins can process crRNA arrays that include multiple spacer sequences; the compositions of the invention include, in some embodiments, crRNA arrays with multiple spacer sequences designed to target multiple different loci within the plant pest of interest or within multiple plant pests. In some embodiments, Cas12a2-gRNA systems can target DNA sequences adjacent to a variety of PAM (i.e. PFM, PFS) sequences, with the PAM (i.e. PFM, PFS) sequence located immediately 5′ of the DNA sequence targeted by Cas12a2. In some embodiments, Cas12a2-gRNA systems can target RNA sequences adjacent to a variety of PAM (i.e. PFM, PFS) sequences, with the PAM (i.e. PFM, PFS) sequence located immediately 3′ of the RNA sequence targeted by Cas12a2. The initial hybridization event is sequence-specific with limited off target effects, resulting in sequence-specific killing of cells of plant pests of interest without harming cells that do not harbor the target sequence(s) of interest.

### I. Cas12a2 Endonucleases and Guide Polynucleotides

Provided herein are Cas12a2 endonucleases, and no In general, Cas12a2 polypeptides comprise at least one RNA recognition and/or RNA binding domain. RNA recognition and/or RNA binding domains interact with guide RNAs. Typically, the guide RNA comprises a region with a stem-loop structure that interacts with the Cas12a2 polypeptide. This stem-loop often comprises the sequence UCUACN₃₋₅GUAGAU (SEQ ID NOs: 40-42, encoded by SEQ ID NOs: 43-45), with “UCUAC” and “GUAGA” base-pairing to form the stem of the stem-loop. N₃₋₅ denotes that any base may be present at this location, and 3, 4, or 5 nucleotides may be included at this location. Some CRISPR nucleases have been shown to function with guide polynucleotides in which some of the ribonucleotide residues have been replaced by deoxyribonucleotide residues (Yin et al (2018) Nat Chem Biol 14:311-316; U.S. Pat. No. 9,650,617); the present invention also encompasses embodiments in which the guide polynucleotide is a guide RNA, embodiments in which the guide polynucleotide is a guide DNA, and embodiments in which the guide polynucleotide comprises both DNA and RNA residues. In specific embodiments, a Cas12a2 polypeptide, or a polynucleotide encoding a Cas12a2 polypeptide, comprises: an RNA-binding portion that interacts with the guide RNA targeting a DNA or RNA sequence, and an activity portion that exhibits site-directed enzymatic activity, such as a RuvC endonuclease domain. Without being limited by theory, the RuvC endonuclease domain may also exhibit secondary, collateral activity directed against dsDNA, ssDNA, dsRNA, and/or ssRNA in a non-sequence-specific manner.

Cas12a2 polypeptides can be wild type Cas12a2 polypeptides, modified Cas12a2 polypeptides, or a fragment of a wild type or modified Cas12a2 polypeptide. The Cas12a2 polypeptide can be modified to increase nucleic acid binding affinity and/or specificity, alter an enzymatic activity, and/or change another property of the protein. For example, nuclease (i.e., DNase, RNase) domains of the Cas12a2 polypeptide can be modified, deleted, or inactivated. Alternatively, the Cas12a2 polypeptide can be modified or truncated to alter or remove domains that are not essential for the function of the protein.

In some embodiments, the Cas12a2 polypeptide can be derived from a wild type Cas12a2 polypeptide or fragment thereof. In other embodiments, the Cas12a2 polypeptide can be derived from a modified Cas12a2 polypeptide. For example, the amino acid sequence of the Cas12a2 polypeptide can be modified to alter one or more properties (e.g., nuclease activity, affinity, stability, solubility, etc.) of the protein.

Cas12a2 polypeptides of the disclosure can include Cas12a2 polypeptides disclosed in U.S. Pat. No. 9,896,696 and in International Publication No. WO 2019/030695, which are incorporated by reference herein in their entireties. In some embodiments, a Cas12a2 polypeptide of the disclosure include “Sulf-type” (set forth as any one of SEQ ID NOs: 1-39, and 166), as described in International Publication No. WO 2019/030695. In some embodiments, a Cms1 polypeptide of the disclosure includes Unk89 (SEQ ID NO: 26), Unk88 (SEQ ID NO: 27), Unk97 (SEQ ID NO: 28), Unk106 (SEQ ID NO: 29), Unk107 (SEQ ID NO: 30), Unk108 (SEQ ID NO: 31), Unk109 (SEQ ID NO: 32), Unk110 (SEQ ID NO: 33), Unk111 (SEQ ID NO: 34), Unk112 (SEQ ID NO: 35), Unk113 (SEQ ID NO: 36), Unk114 (SEQ ID NO: 37), Unk115 (SEQ ID NO: 166), Unk119 (SEQ ID NO: 38), and Unk120 (SEQ ID NO: 39).

In general, a CmsCas12a2 polypeptide comprises at least one nuclease domain, but need not contain an HNH domain such as the one found in Cas9 proteins. For example, a Cas12a2 polypeptide can comprise a RuvC or RuvC-like nuclease domain. Without being limited by theory, the RuvC or RuvC-like domain may comprise three catalytic residues that are typically aspartate, glutamate, and aspartate, respectively, and may be responsible for the Cas12a2 nuclease activity.

A Sulf-type Cas12a2 polypeptide of the disclosure can comprise three RuvC nuclease domains (RuvCI, RuvCII, and RuvCIII). A RuvCI domain of a Sulf-type Cas12a2 polypeptide can comprise a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 55 (e.g., wherein the Cas12a2 protein retains nuclease activity). A RuvCII domain of a Sulf-type Cas12a2 polypeptide can comprise a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I. In one embodiment, the Cas12a2 protein comprises a consensus motif having at least 90% identity (e.g., 90%, 91%, 92%, 93%, 94%, 95%, 96%, 97%, 98%, 99% or more identity) with the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I (e.g., and wherein the Cas12a2 protein retains nuclease activity). A RuvCIII domain of a Sulf-type Cas12a2 polypeptide can comprise a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 62 (e.g., wherein the Cas12a2 protein retains nuclease activity).

A Sulf-type Cas12a2 polypeptide of the disclosure can comprise a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 46 (e.g., wherein the Cas12a2 protein retains nuclease activity).

A Sulf-type Cas12a2 polypeptide of the disclosure can comprise a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V. In one embodiment, the Cas12a2 protein comprises a consensus motif having at least 90% identity (e.g., 90%, 91%, 92%, 93%, 94%, 95%, 96%, 97%, 98%, 99% or more identity) with the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V (e.g., and wherein the Cas12a2 protein retains nuclease activity).

A Sulf-type Cas12a2 polypeptide of the disclosure can comprise a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 48 (e.g., wherein the Cas12a2 protein retains nuclease activity).

A Sulf-type Cas12a2 polypeptide of the disclosure can comprise a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F. In one embodiment, the Cas12a2 protein comprises a consensus motif having at least 90% identity (e.g., 90%, 91%, 92%, 93%, 94%, 95%, 96%, 97%, 98%, 99% or more identity) with the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F (e.g., and wherein the Cas12a2 protein retains nuclease activity).

A Sulf-type Cas12a2 polypeptide of the disclosure can comprise a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 50 (e.g., wherein the Cas12a2 protein retains nuclease activity).

A Sulf-type Cas12a2 polypeptide of the disclosure can comprise a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F. In one embodiment, the Cas12a2 protein comprises a consensus motif having at least 90% identity (e.g., 90%, 91%, 92%, 93%, 94%, 95%, 96%, 97%, 98%, 99% or more identity) with the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F (e.g., and wherein the Cas12a2 protein retains nuclease activity).

A Sulf-type Cas12a2 polypeptide of the disclosure can comprise a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 52 (e.g., wherein the Cas12a2 protein retains nuclease activity).

A Sulf-type Cas12a2 polypeptide of the disclosure can comprise a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 53 (e.g., wherein the Cas12a2 protein retains nuclease activity).

A Sulf-type Cas12a2 polypeptide of the disclosure can comprise a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K. In one embodiment, the Cas12a2 protein comprises a consensus motif having at least 90% identity (e.g., 90%, 91%, 92%, 93%, 94%, 95%, 96%, 97%, 98%, 99% or more identity) with the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K (e.g., and wherein the Cas12a2 protein retains nuclease activity).

A Sulf-type Cas12a2 polypeptide of the disclosure can comprise a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 56 (e.g., wherein the Cas12a2 protein retains nuclease activity).

A Sulf-type Cas12a2 polypeptide of the disclosure can comprise a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T. In one embodiment, the Cas12a2 protein comprises a consensus motif having at least 90% identity (e.g., 90%, 91%, 92%, 93%, 94%, 95%, 96%, 97%, 98%, 99% or more identity) with the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T (e.g., and wherein the Cas12a2 protein retains nuclease activity).

A Sulf-type Cas12a2 polypeptide of the disclosure can comprise a conserved motif 13 comprising the amino acid sequence set forth as X₁LX₃X₄X₅X₆X₇X₈KX₁₀GX₁₂X₁₃ANX₁₆X₁₇GX₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 58 (e.g., wherein the Cas12a2 protein retains nuclease activity).

A Sulf-type Cas12a2 polypeptide of the disclosure can comprise a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 60 (e.g., wherein the Cas12a2 protein retains nuclease activity).

A Sulf-type Cas12a2 polypeptide of the disclosure can comprise a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid. In one embodiment, the Cas12a2 protein comprises a consensus motif having at least 90% identity (e.g., 90%, 91%, 92%, 93%, 94%, 95%, 96%, 97%, 98%, 99% or more identity) with SEQ ID NO: 61 (e.g., wherein the Cas12a2 protein retains nuclease activity).

In certain embodiments, the Cas12a2 polypeptide may be part of a protein-RNA complex comprising a guide polynucleotide. In some embodiments, the guide polynucleotide may be a guide RNA. The guide polynucleotide interacts with the Cas12a2 polypeptide to direct the Cas12a2 polypeptide to a specific target site in a plant pest, where the target site comprises: dsDNA that may be present in genomic DNA, plasmid DNA, other DNA components; or dsRNA, ssRNA (e.g., mRNA), or other RNA components in a cell of a plant pest of interest. In some embodiments, if a suitable protospacer-adjacent motif (PAM) sequence is present immediately 5′ of a dsDNA, ssDNA, dsRNA, or ssRNA target sequence, the Cas12a2-guide polynucleotide complex may hybridize with the dsDNA, ssDNA, dsRNA, or ssRNA target sequence. In some embodiments, if a PAM (i.e. PFM or PFS) is present immediately 3′ of a target dsRNA or ssRNA sequence, the Cas12a2-guide polynucleotide complex may hybridize with the dsRNA or ssRNA target sequence. Following this initial hybridization event, the Cas12a2 enzyme may cleave the target DNA or RNA (i.e. primary activity of a Cas12a2 polypeptide). Without being limited by theory, the Cas12a2 enzyme may then undergo a structural change that may allow the Cas12a2 enzyme to cleave dsDNA, ssDNA, dsRNA, and/or ssRNA in a non-sequence-specific manner (“secondary” or “collateral” activity of a Cas12a2 polypeptide). This secondary activity may result in cell death. As used herein, the term “DNA-targeting RNA” refers to a guide RNA that interacts with the Cas12a2 polypeptide and hybridizes to a DNA target site of interest in the genome of a cell of a plant pest. As used herein, the term “RNA-targeting RNA” refers to a guide RNA that interacts with the Cas12a2 polypeptide and hybridizes to an RNA target site of interest in a cell of a plant pest. A DNA-targeting or RNA-targeting RNA, or a DNA polynucleotide encoding a DNA-targeting or RNA-targeting RNA, can comprise: a first segment comprising a nucleotide sequence that is complementary to a sequence in the target DNA or RNA, and a second segment that interacts with a Cas12a2 polypeptide.

Cas12a2 polypeptides for use in the invention include, but are not limited to, Cas12a2 polypeptides that comprise at least one amino acid motif selected from the group consisting of SEQ ID NOs: 46-62. In certain preferred embodiments, a Cas12a2 polypeptide comprises more than one amino acid motif selected from the group consisting of SEQ ID NOs: 46-62. In some embodiments, a Cas12a2 polypeptide of the disclosure comprises the amino acid motif set forth as SEQ ID NO: 48. In some embodiments, a Cas12a2 polypeptide of the disclosure comprises the amino acid motif set forth as SEQ ID NO: 56. In some embodiments, a Cas12a2 polypeptide of the disclosure comprises the amino acid motif set forth as SEQ ID NO: 58.

A Cas12a2 polypeptide of the disclosure can comprise an amino acid sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of SEQ ID NOs: 1-39, and 166. In some embodiments, a Cas12a2 polypeptide of the disclosure comprises the amino acid sequence set forth as any one of SEQ ID NOs: 1-39, and 166.

A Cas12a2 polypeptide of the disclosure can comprise an amino acid sequence encoded by a polynucleotide comprising a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of SEQ ID NOs: 63-101, and 167. In some embodiments, a Cas12a2 polypeptide of the disclosure comprises an amino acid sequence encoded by a polynucleotide comprising a nucleotide sequence set forth as any one of SEQ ID NOs: 63-101, and 167.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a RuvCI domain comprising a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a RuvCI domain comprising a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a RuvCII domain comprising a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I.

In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a RuvCII domain comprising a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a RuvCIII domain comprising a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a RuvCIII domain comprising a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁=I, L, or V; X₁₂=I, L, or F. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 13 comprising the amino acid sequence set forth as X₁LX₃X₄X₅X₆X₇X₈KX₁₀GX₁₂X₁₃ANX₁₆X₁₇GX₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 13 comprising the amino acid sequence set forth as X₁LX₃X₄X₅X₆X₇X₈KX₁₀GX₁₂X₁₃ANX₁₆X₁₇GX₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid.

In some embodiments, a Cas12a2 polypeptide of the disclosure can comprise an amino acid sequence having at least 90% sequence identity to any one of SEQ ID NOs: 1-39, and 166, wherein the Cas12a2 polypeptide comprises a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid. In some embodiments, a Cas12a2 polypeptide of the disclosure can comprise an amino acid sequence having at least 95% sequence identity to any one of SEQ ID NOs: 1-39, and 166, wherein the Cas12a2 polypeptide comprises a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid.

Particular Cas12a2 polypeptide sequences are set forth in SEQ ID NOs: 26-39, and 166; particular Cas12a2 polypeptide-encoding polynucleotide sequences are set forth in SEQ ID NOs: 88-101, and 167. In certain embodiments, a Cas12a2 polypeptide has at least about 80% identity with a sequence selected from the group consisting of SEQ ID NOs: 26-39, and 166. In certain embodiments, Cas12a2 polypeptides for use in the invention include, but are not limited to, Cas12a2 polypeptides comprising at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 26-39, and 166, wherein said Cas12a2 polypeptides comprise at least one amino acid residue selected from any one of the following positions corresponding with the SuCas12a2 polypeptide (SEQ ID NO: 1): F370, Y375, P376, K378, A380, F381, W385, E386, A389, 1898, L899, D900, L901, L904, E907, L916, V917, D918, L1029, K1036, G1038, A1041, N1042, and G1045. In certain embodiments, a Cas12a2 polypeptide comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 26-39, and 166, and comprises the following amino acid residues at the positions corresponding with the SuCas12a2 polypeptide (SEQ ID NO: 1): F370, Y375, P376, K378, A380, F381, W385, E386, A389, 1898, L899, D900, L901, L904, E907, L916, V917, D918, L1029, K1036, G1038, A1041, N1042, and G1045. In certain embodiments, the Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 26-39, and 166, and comprises a Sulf-type Cas12a2 conserved motif selected from any one of SEQ ID NOs: 46-62 (e.g., wherein the Cas12a2 protein retains nuclease activity). In certain embodiments, the Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 26-39, and 166, and comprises a Sulf-type Cas12a2 conserved motif selected from any one of SEQ ID NOs: 48, 56, and 58 (e.g., wherein the Cas12a2 protein retains nuclease activity).

Particular Cas12a2 polypeptide sequences are set forth in SEQ ID NOs: 28, 31, 32, 33, 34, 36, 37, 38, and 166; particular Cas12a2 polypeptide-encoding polynucleotide sequences are set forth in SEQ ID NOs: 90, 93, 94, 95, 96, 98, 99, 100, and 167. In certain embodiments, a Cas12a2 polypeptide has at least about 80% identity with a sequence selected from the group consisting of SEQ ID NOs: 28, 31, 32, 33, 34, 36, 37, 38, and 166. In certain embodiments, Cas12a2 polypeptides for use in the invention include, but are not limited to, Cas12a2 polypeptides comprising at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 28, 31, 32, 33, 34, 36, 37, 38, and 166, wherein said Cas12a2 polypeptides comprise at least one amino acid residue selected from any one of the following positions corresponding with the SuCas12a2 polypeptide (SEQ ID NO: 1): F370, Y375, P376, K378, A380, F381, W385, E386, A389, 1898, L899, D900, L901, L904, E907, L916, V917, D918, L1029, K1036, G1038, A1041, N1042, and G1045. In certain embodiments, a Cas12a2 polypeptide comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 28, 31, 32, 33, 34, 36, 37, 38, and 166, and comprises the following amino acid residues at the positions corresponding with the SuCas12a2 polypeptide (SEQ ID NO: 1): F370, Y375, P376, K378, A380, F381, W385, E386, A389, 1898, L899, D900, L901, L904, E907, L916, V917, D918, L1029, K1036, G1038, A1041, N1042, and G1045. In certain embodiments, the Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 28, 31, 32, 33, 34, 36, 37, 38, and 166, and comprises a Sulf-type Cas12a2 conserved motif selected from any one of SEQ ID NOs: 46-62 (e.g., wherein the Cas12a2 protein retains nuclease activity). In certain embodiments, the Cas12a2 protein comprises at least about 80%, 85%, 86%, 87%, 88%, 89%, 90%, 91%, 92%, 93%, 94%, 95%, 96%, 97%, 98%, 99% or more identity with a sequence selected from the group consisting of SEQ ID NOs: 28, 31, 32, 33, 34, 36, 37, 38, and 166, and comprises a Sulf-type Cas12a2 conserved motif selected from any one of SEQ ID NOs: 48, 56, and 58 (e.g., wherein the Cas12a2 protein retains nuclease activity).

The polynucleotides encoding Cas12a2 polypeptides disclosed herein can be used to isolate corresponding sequences from other prokaryotic or eukaryotic organisms, or from metagenomically-derived sequences whose native host organism is unclear or unknown. In this manner, methods such as PCR, hybridization, and the like can be used to identify such sequences based on their sequence homology or identity to the sequences set forth herein. Sequences isolated based on their sequence identity to the entire Cas12a2 sequences set forth herein or to variants and fragments thereof are encompassed by the present invention. Such sequences include sequences that are orthologs of the disclosed Cas12a2 sequences. “Orthologs” is intended to mean genes derived from a common ancestral gene and which are found in different species as a result of speciation. Genes found in different species are considered orthologs when their nucleotide sequences and/or their encoded protein sequences share at least about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode polypeptides having Cas12a2 endonuclease activity and which share at least about 75% or more sequence identity to the sequences disclosed herein, are encompassed by the present invention.

Fragments and variants of the Cas12a2 polynucleotides and Cas12a2 amino acid sequences encoded thereby that retain Cas12a2 nuclease activity are encompassed herein. By “Cas12a2 nuclease activity” is intended the binding of and hybridization with a pre-determined DNA or RNA sequence (the “target sequence”) as mediated by a guide RNA. Cas12a2 nuclease activity can further comprise single or double-strand break production of the target sequence (“primary activity”), and can further comprise non-sequence-specific nuclease activity directed against dsDNA, ssDNA, dsRNA, and/or ssRNA (“secondary activity”) following the primary activity. By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein. Biologically active variants of a native polypeptide will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a protein of the invention may differ from that protein by as few as 1-15 amino acid residues, as few as 1-10, such as 6-10, as few as 5, as few as 4, 3, 2, or even 1 amino acid residue. Biologically active variants of a native polypeptide will have at least about 80%, 85%, 90%, 91%, 92%, 93%, 94%, 95%, 96%, 97%, 98%, 99% or more of the activity of the native polypeptide. Activity of Cas12a2 variant polypeptides can be measured by the ability of the polypeptide to bind and/or cleave a target site in the presence of the appropriate guide polynucleotide (e.g., guide RNA).

Variant sequences may also be identified by analysis of existing databases of sequenced genomes. In this manner, corresponding sequences can be identified and used in the methods of the invention. With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, California, USA). Alignments using these programs can be performed using the default parameters. The CLUSTAL program is well described by Higgins et al. (1988) Gene 73:237-244; Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The MUSCLE algorithm for multiple sequence alignment may be used for comparisons of multiple nucleic acid or protein sequences (Edgar (2004) Nucleic Acids Research 32:1792-1797). The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the website at www.ncbi.nlm.nih.gov. Alignment may also be performed manually by inspection.

The nucleic acid molecules encoding Cas12a2 polypeptides, or fragments or variants thereof, can be codon optimized for expression in an organism of interest (e.g., a cell of a plant pest or a plant cell). A “codon-optimized gene” is a gene having its frequency of codon usage designed to mimic the frequency of preferred codon usage of the host cell. Nucleic acid molecules can be codon optimized, either wholly or in part. Because any one amino acid (except for methionine and tryptophan) is encoded by a number of codons, the sequence of the nucleic acid molecule may be changed without changing the encoded amino acid. Codon optimization is when one or more codons are altered at the nucleic acid level such that the amino acids are not changed but expression in a particular host organism is increased. Those having ordinary skill in the art will recognize that codon tables and other references providing preference information for a wide range of organisms are available in the art (see, e.g., Zhang et al. (1991) Gene 105:61-72; Murray et al. (1989) Nucl. Acids Res. 17:477-508).

In some embodiments, DNA encoding the Cas12a2 polypeptides of the invention, and DNA encoding guide polynucleotide(s) of the invention, may be included as part of a bacteriophage or modified bacteriophage, or may be included as part of a plasmid (for example a conjugative plasmid), phagemid, cosmid, or other DNA molecule capable of replication in a cell of interest. The terms phage and bacteriophage may be used interchangeably. In some embodiments, a phage or a phagemid derived from M13, lambda, p22, T7, Mu, T4 phage, PBSX, P1Puna-like, P2, 13, Beep 1, Beep 43, Beep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage or 186 phage may be used to deliver a polynucleotide encoding a Cas12a2 polypeptide of the invention and/or one or more guide polynucleotide(s) of the invention, to the cell(s) of interest. Bacteriophage may be engineered, for example, to have a broad or narrow host range using methods known in the art (Yehl et al 2019 Cell 179: 459-469).

### II. Fusion Proteins

Fusion proteins are provided herein comprising a Cas12a2 polypeptide, or a fragment or variant thereof, and a heterologous polypeptide (also referred to as a “Cas12a2 fusion protein”). As used herein, “heterologous”, in reference to a polypeptide that is heterologous to another polypeptide (e.g., Cas12a2), is a polypeptide that is not operably fused to the presently described polypeptides, e.g., Cas12a2, in nature. The heterologous polypeptide can originate from a foreign species or from the same species. The heterologous polypeptide can be in its native form or is substantially modified from its native form in composition and/or genomic locus by deliberate human intervention. The heterologous polypeptide can be any polypeptide, including but not limited to a nuclear localization signal (NLSs), an export signal, a plastid signal peptide, a mitochondrial signal peptide, a signal peptide capable of trafficking proteins to one or more subcellular locations, a cell-penetrating domain (CPP), a translocation domain, and a marker domain (e.g., detectable label, a purification tag, an epitope tag).

Fusion proteins such as those of the present disclosure may be produced by means of recombinant protein technology known to persons skilled in the art. In general, a genetic construct comprising a polynucleotide encoding an operable fusion of polypeptides of interest, or functional variants thereof, can be generated such that the construct includes the in-frame fusion of the nucleic acid sequences encoding the polypeptides to be expressed as a fusion protein. Such a fusion protein can comprise or lack one or more peptide linkers. A fusion protein can refer to a polypeptide operably fused to a tag, including but not limited to, a purification tag (e.g., 6×His tag), a localization signal (e.g., PR1a, CPP described herein), and/or a detectable label (e.g., GFP). The genetic construct can be transformed or transfected into host cells for expression of the fusion protein. The nucleic acid sequences encoding the polypeptides in operable fusion may suitably be of genomic, cDNA, or synthetic origin. Alterations of the amino acid sequences of the polypeptides in operable fusion are accomplished by modification of the genetic code by well-known techniques.

A peptide linker that connects a Cas12a2 and a heterologous polypeptide in a fusion protein may predominantly include the following amino acid residues: glycine (Gly), serine (Ser), alanine (Ala), or threonine (Thr). The peptide linker should have a length that is adequate to link two polypeptides in such a way that they assume the correct conformation relative to one another so that they retain the desired activity. In some embodiments, the peptide linker is at least 1, 2, 3, 4, 5, 6, 7, 8, 9, 10, 15, 20, 25, 30, 40, or 50 amino acids in length. In some embodiments, the peptide linker is from about 1 to about 50 amino acids in length, from about 1 to about 30 amino acids in length, from about 1 to about 20 amino acids in length, from about 1 to about 10 amino acids in length, or from about 1 to about 5 amino acids in length. In some embodiments, the peptide linker is from about 10 to about 50 amino acids in length, from about 5 to about 20 amino acids in length, or from about 3 to about 20 amino acids in length. In some embodiments, the peptide linker is about 10, 11, 12, 13, 14, or 15 amino acids in length. In some embodiments, the peptide linker is 12 amino acids in length. Useful linkers include glycine-serine polymers, including for example (GS)n, (GGS)n, (GGGS)n (SEQ ID NO: 168), (GGGGS)n (SEQ ID NO: 169), and (GSGGS)n (SEQ ID NO: 170), where n is an integer of 1 or greater (e.g., 2, 3, 4, 5), glycine-alanine polymers, alanine-serine polymers, as well as any peptide sequence that allows for recombinant attachment of two polypeptides or domains with sufficient length and flexibility to allow each polypeptide or domain to retain its biological function. In some embodiments, the peptide linker is a 4× GGS linker set forth as SEQ ID NO: 161. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

In some embodiments, a Cas12a2 polypeptide of the disclosure is operably fused to at least one cell-penetrating domain (CPP), which facilitates cellular uptake of the protein. CPPs that can be used to facilitate cellular uptake of a Cas12a2 polypeptide include: transactivating transcriptional activator (TAT), transportan, and penetratin. CPPs can include sequences that are arginine-rich, for example, CPPs comprising at least 7 arginines (R), at least 8 R, at least 9 R, or at least 10 R. In some embodiments, a CPP comprises: an R9-TAT having an amino acid sequence set forth as SEQ ID NO: 116 from human immunodeficiency virus; a transportan having an amino acid sequence set forth as SEQ ID NO: 117 from galanin-mastoparan chimeric peptide; and penetratin having an amino acid sequence set forth as SEQ ID NO: 160.

In some embodiments, a Cas12a2 polypeptide of the disclosure is operably fused to at least one export signal. For example, the export signal can include a signal peptide of the tobacco pathogenesis-related protein 1a (PR1a). An export signal can direct a nascent polypeptide, as it is being synthesized, to enter the endoplasmic reticulum (ER). Once inside the ER, the protein moves through the secretory pathway, eventually being exported from the cell. An ER export signal can comprise a diacidic (DxE) amino acid motif, a dihydrophobic (LL) amino acid motif, or a diaromatic (FF, YY) amino acid motif. In some embodiments, these motifs are recognized by components of the COPII machinery, which can selectively package the polypeptide into transport vesicles destined for the Golgi apparatus, and optionally, further processing and sorting to its final destination. In some embodiments, an export signal comprises a PR1a having an amino acid sequence set forth as SEQ ID NO: 118 from Nicotiana tabacum.

In some embodiments, a Cas12a2 polypeptide of the disclosure is operably fused to at least one localization signal that can direct the expressed Cas12a2 polypeptide to a particular location or organelle in a cell or tissue. For example, the localization signal can be a nuclear localization signal (NLS). An NLS includes but is not limited to a SV40 peptide having an amino acid sequence set forth as SEQ ID NO: 119 from Simian vacuolating virus, and a nucleoplasmin peptide having an amino acid sequence set forth as SEQ ID NO: 120 from Xenopus.

In some embodiments, a Cas12a2 polypeptide of the disclosure is operably fused to an effector domain. The Cas12a2 polypeptide can be directed to a target site by a guide RNA, at which site the effector domain can modify or effect the targeted nucleic acid sequence. The effector domain can be a cleavage domain, an epigenetic modification domain, a transcriptional activation domain, a transcriptional repressor domain, or a deaminase domain. The fusion protein can further comprise at least one additional domain chosen from a nuclear localization signal, plastid signal peptide, mitochondrial signal peptide, signal peptide capable of protein trafficking to multiple subcellular locations, a cell-penetrating domain, or a marker domain. In some embodiments of an operable fusion of a Cas12a2 polypeptide and an effector domain, the Cas12a2 polypeptide can be modified as discussed herein such that its endonuclease activity is eliminated (e.g., when the effector domain is an epigenetic modification domain, a transcriptional activation domain, a transcriptional repressor domain, or a deaminase domain). For example, the Cas12a2 polypeptide can be modified by mutating the RuvC-like domain such that the polypeptide no longer possesses nuclease activity.

In some embodiments, the effector domain of the fusion protein is a cleavage domain. As used herein, a “cleavage domain” refers to a domain that cleaves DNA or RNA. The cleavage domain can be obtained from any endonuclease or exonuclease. Non-limiting examples of endonucleases from which a cleavage domain can be derived include, but are not limited to, restriction endonucleases and homing endonucleases. See, for example, New England Biolabs Catalog or Belfort et al. (1997) Nucleic Acids Res. 25:3379-3388. Additional enzymes that cleave DNA are known (e.g., S1 nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) can be used as a source of cleavage domains.

In some embodiments, the effector domain of a fusion protein is an epigenetic modification domain. In general, epigenetic modification domains alter histone structure and/or chromosomal structure without altering the DNA sequence. Changes in histone and/or chromatin structure can lead to changes in gene expression. Examples of epigenetic modification include, without limit, acetylation or methylation of lysine residues in histone proteins, and methylation of cytosine residues in DNA. Non-limiting examples of suitable epigenetic modification domains include histone acetyltansferase domains, histone deacetylase domains, histone methyltransferase domains, histone demethylase domains, DNA methyltransferase domains, and DNA demethylase domains.

In some embodiments, the effector domain of the fusion protein can be a transcriptional activation domain. In general, a transcriptional activation domain interacts with transcriptional control elements and/or transcriptional regulatory proteins (i.e., transcription factors, RNA polymerases, etc.) to increase and/or activate transcription of one or more genes. In some embodiments, the transcriptional activation domain can be, without limit, a herpes simplex virus VP16 activation domain, VP64 (which is a tetrameric derivative of VP16), a NFκB p65 activation domain, p53 activation domains 1 and 2, a CREB (cAMP response element binding protein) activation domain, an E2A activation domain, and an NFAT (nuclear factor of activated T-cells) activation domain. In other embodiments, the transcriptional activation domain can be Gal4, Gcn4, MLL, Rtg3, Gln3, Oaf1, Pip2, Pdr1, Pdr3, Pho4, and Leu3. The transcriptional activation domain may be wild type, or it may be a modified version of the original transcriptional activation domain. In some embodiments, the effector domain of the fusion protein is a VP16 or VP64 transcriptional activation domain.

In still other embodiments, the effector domain of the fusion protein can be a transcriptional repressor domain. In general, a transcriptional repressor domain interacts with transcriptional control elements and/or transcriptional regulatory proteins (i.e., transcription factors, RNA polymerases, etc.) to decrease and/or terminate transcription of one or more genes. Non-limiting examples of suitable transcriptional repressor domains include inducible cAMP early repressor (ICER) domains, Kruppel-associated box A (KRAB-A) repressor domains, YY1 glycine rich repressor domains, Sp1-like repressors, E(spl) repressors, I.kappa.B repressor, and MeCP2.

In yet other embodiments, the effector domain of the fusion protein can be a deaminase domain to generate a base editor. In some embodiments, the effector domain of the fusion protein is a cytosine deaminase to form a cytosine base editor (C-base editor or CBE) that deaminates cytosine into uracil, which is then subsequently converted to thymine through DNA replication or repair. In other embodiments, the effector domain of the fusion protein is an adenine deaminase to form an adenine base editor (A-base editor or ABE) that deaminates adenine into inosine that is subsequently recognized as a guanine by polymerases and allows for the incorporation of a cytosine on the complementary DNA strand across from the inosine, ultimately resulting in an A to G mutation.

In some embodiments, a Cas12a2 polypeptide of the disclosure is operably fused to at least one marker domain. Non-limiting examples of marker domains include detectable labels, purification tags, and epitope tags. In certain embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

A fusion protein comprising a heterologous polypeptide (e.g., a cell-penetrating domain, an export signal, a nuclear localization signal, a marker domain) operably fused to a Cas12a2 polypeptide of the disclosure can have the heterologous polypeptide located at the N-terminus, at the C-terminus, or at an internal location of the Cas12a2 polypeptide. A polypeptide (e.g., domain or peptide) located in or at an “internal location” of a Cas12a2 polypeptide refers to the operable fusion of the polypeptide (e.g., domain or peptide) at an internal amino acid position of the Cas12a2 polypeptide such that the polypeptide (e.g., domain or peptide) and the Cas12a2 polypeptide are in the same reading frame and allows translation of a functional fusion protein where function of each polypeptide is comparable to the function of each polypeptide alone. The Cas12a2 polypeptide can be directly fused to the heterologous polypeptide, or can be operably fused through a linker, such as a peptide linker.

In certain embodiments, any of the fusion proteins detailed above may be part of a protein-RNA complex comprising at least one guide RNA. A guide RNA interacts with the Cas12a2 polypeptide of the fusion protein to direct the fusion protein to a specific target site, wherein the 5′ end of the guide RNA base pairs with a specific protospacer sequence.

### III. Guide RNAs

A guide RNA of the disclosure interacts with the Cas12a2 polypeptide to direct the Cas12a2 polypeptide to a specific target site, at which site the guide RNA base pairs with a specific DNA or RNA sequence at the target site. Guide RNAs can comprise three regions: a first region that is complementary to the target DNA or RNA sequence at the target site, a second region that forms a stem loop structure, and a third region that remains essentially single-stranded. The first region of each guide RNA is different such that each guide RNA guides a Cas12a2 polypeptide to a specific target site. The second and third regions of each guide RNA can be the same in all guide RNAs.

The skilled artisan can identify a plant pest target site comprising one or more target DNA or RNA sequences. For example, such a plant pest target site can comprise a nucleic acid sequence that serves a direct or indirect role in such a pest's deleterious effects on a host plant. By way of example only, such a plant pest target site may be one that serves a role in pest growth, development, replication, reproduction, invasion, infection, or a combination thereof. The region of the guide RNA complementary to the target DNA or RNA sequence in the plant pest to be controlled is specific for the plant pest target DNA or RNA sequence such that secondary activity of a Cas12a2 polypeptide associated with a guide RNA would be triggered only when the plant pest target DNA or RNA sequence is present. In some embodiments, the guide RNA (in conjunction with a Cas12a2 polypeptide of the disclosure) used to control a plant pest has minimal or no complementarity to nucleic acid sequences in the plant itself or to nucleic acid sequences in organisms associated with the plant but not considered to be a pest of the plant (e.g., beneficial organisms that help the plant grow).

One region of the guide RNA is complementary to a target DNA or RNA sequence (i.e., a protospacer sequence of the guide RNA) at the target site such that the first region of the guide RNA can base pair with the target DNA or RNA sequence at the target site. The first region of the guide RNA that can base pair with a target DNA or RNA sequence at the target site is referred to as the “spacer”. In various embodiments, the first region (i.e. the spacer) of the guide RNA can comprise from about 8 nucleotides to more than about 30 nucleotides. For example, the region of base pairing between the first region of the guide RNA and the target DNA or RNA sequence can be about 8, about 9, about 10, about 11, about 12, about 13, about 14, about 15, about 16, about 17, about 18, about 19, about 20, about 22, about 23, about 24, about 25, about 27, about 30 or more than 30 nucleotides in length. In an exemplary embodiment, the first region (i.e. the spacer) of the guide RNA is about 20, 21, 22, 23, 24, or 25 nucleotides in length.

The presently disclosed guide RNAs comprise a spacer capable of targeting a Cas12a2 polypeptide to a target sequence of C. elegans. The target sequence of C. elegans can be within the AGE1-1 gene, wherein the target sequence has the nucleotide sequence set forth as SEQ ID NO: 103. In some embodiments, a spacer of the disclosure has a nucleotide sequence set forth as SEQ ID NO: 175 or a nucleotide sequence that differs in length and/or sequence from SEQ ID NO: 175 by 1 to 5 nucleotides. In some embodiments, the spacer has a nucleotide sequence that differs in length and/or sequence from from SEQ ID NO: 175 by 5 nucleotides. In some embodiments, the spacer has a nucleotide sequence that differs in length and/or sequence from from SEQ ID NO: 175 by 4 nucleotides. In some embodiments, the spacer has a nucleotide sequence that differs in length and/or sequence from from SEQ ID NO: 175 by 3 nucleotides. In some embodiments, the spacer has a nucleotide sequence that differs in length and/or sequence from from SEQ ID NO: 175 by 2 nucleotides. In some embodiments, the spacer has a nucleotide sequence that differs in length and/or sequence from from SEQ ID NO: 175 by 1 nucleotide.

The presently disclosed guide RNAs comprise a spacer capable of targeting a Cas12a2 polypeptide to a target sequence of C. elegans. The target sequence of C. elegans can be within the AGE1-2 gene, wherein the target sequence has the nucleotide sequence set forth as SEQ ID NO: 104. In some embodiments, a spacer of the disclosure has a nucleotide sequence set forth as SEQ ID NO: 176 or a nucleotide sequence that differs in length and/or sequence from SEQ ID NO: 176 by 1 to 5 nucleotides. In some embodiments, the spacer has a nucleotide sequence that differs in length and/or sequence from from SEQ ID NO: 176 by 5 nucleotides. In some embodiments, the spacer has a nucleotide sequence that differs in length and/or sequence from from SEQ ID NO: 176 by 4 nucleotides. In some embodiments, the spacer has a nucleotide sequence that differs in length and/or sequence from from SEQ ID NO: 176 by 3 nucleotides. In some embodiments, the spacer has a nucleotide sequence that differs in length and/or sequence from from SEQ ID NO: 176 by 2 nucleotides. In some embodiments, the spacer has a nucleotide sequence that differs in length and/or sequence from from SEQ ID NO: 176 by 1 nucleotide.

The presently disclosed guide RNAs comprise a spacer capable of targeting a Cas12a2 polypeptide to a target sequence of C. elegans. The target sequence of C. elegans can be within the AGE1-7 gene, wherein the target sequence has the nucleotide sequence set forth as SEQ ID NO: 105. In some embodiments, a spacer of the disclosure has a nucleotide sequence set forth as SEQ ID NO: 177 or a nucleotide sequence that differs in length and/or sequence from SEQ ID NO: 177 by 1 to 5 nucleotides. In some embodiments, the spacer has a nucleotide sequence that differs in length and/or sequence from from SEQ ID NO: 177 by 5 nucleotides. In some embodiments, the spacer has a nucleotide sequence that differs in length and/or sequence from from SEQ ID NO: 177 by 4 nucleotides. In some embodiments, the spacer has a nucleotide sequence that differs in length and/or sequence from from SEQ ID NO: 177 by 3 nucleotides. In some embodiments, the spacer has a nucleotide sequence that differs in length and/or sequence from from SEQ ID NO: 177 by 2 nucleotides. In some embodiments, the spacer has a nucleotide sequence that differs in length and/or sequence from from SEQ ID NO: 177 by 1 nucleotide.

The presently disclosed guide RNAs comprise a spacer capable of targeting a Cas12a2 polypeptide to a target sequence of C. elegans. The target sequence of C. elegans can be within the AKT1-2 gene, wherein the target sequence has the nucleotide sequence set forth as SEQ ID NO: 106. In some embodiments, a spacer of the disclosure has a nucleotide sequence set forth as SEQ ID NO: 178 or a nucleotide sequence that differs in length and/or sequence from SEQ ID NO: 178 by 1 to 5 nucleotides. In some embodiments, the spacer has a nucleotide sequence that differs in length and/or sequence from from SEQ ID NO: 178 by 5 nucleotides. In some embodiments, the spacer has a nucleotide sequence that differs in length and/or sequence from from SEQ ID NO: 178 by 4 nucleotides. In some embodiments, the spacer has a nucleotide sequence that differs in length and/or sequence from from SEQ ID NO: 178 by 3 nucleotides. In some embodiments, the spacer has a nucleotide sequence that differs in length and/or sequence from from SEQ ID NO: 178 by 2 nucleotides. In some embodiments, the spacer has a nucleotide sequence that differs in length and/or sequence from from SEQ ID NO: 178 by 1 nucleotide.

The presently disclosed guide RNAs comprise a spacer capable of targeting a Cas12a2 polypeptide to a target sequence of C. elegans. The target sequence of C. elegans can be within the AKT1-5 gene, wherein the target sequence has the nucleotide sequence set forth as SEQ ID NO: 107. In some embodiments, a spacer of the disclosure has a nucleotide sequence set forth as SEQ ID NO: 179 or a nucleotide sequence that differs in length and/or sequence from SEQ ID NO: 179 by 1 to 5 nucleotides. In some embodiments, the spacer has a nucleotide sequence that differs in length and/or sequence from from SEQ ID NO: 179 by 5 nucleotides. In some embodiments, the spacer has a nucleotide sequence that differs in length and/or sequence from from SEQ ID NO: 179 by 4 nucleotides. In some embodiments, the spacer has a nucleotide sequence that differs in length and/or sequence from from SEQ ID NO: 179 by 3 nucleotides. In some embodiments, the spacer has a nucleotide sequence that differs in length and/or sequence from from SEQ ID NO: 179 by 2 nucleotides. In some embodiments, the spacer has a nucleotide sequence that differs in length and/or sequence from from SEQ ID NO: 179 by 1 nucleotide.

The presently disclosed guide RNAs comprise a spacer capable of targeting a Cas12a2 polypeptide to a target sequence of C. elegans. The target sequence of C. elegans can be within the AKT1-8 gene, wherein the target sequence has the nucleotide sequence set forth as SEQ ID NO: 108. In some embodiments, a spacer of the disclosure has a nucleotide sequence set forth as SEQ ID NO: 180 or a nucleotide sequence that differs in length and/or sequence from SEQ ID NO: 180 by 1 to 5 nucleotides. In some embodiments, the spacer has a nucleotide sequence that differs in length and/or sequence from from SEQ ID NO: 180 by 5 nucleotides. In some embodiments, the spacer has a nucleotide sequence that differs in length and/or sequence from from SEQ ID NO: 180 by 4 nucleotides. In some embodiments, the spacer has a nucleotide sequence that differs in length and/or sequence from from SEQ ID NO: 180 by 3 nucleotides. In some embodiments, the spacer has a nucleotide sequence that differs in length and/or sequence from from SEQ ID NO: 180 by 2 nucleotides. In some embodiments, the spacer has a nucleotide sequence that differs in length and/or sequence from from SEQ ID NO: 180 by 1 nucleotide.

The presently disclosed guide RNAs comprise a spacer capable of targeting a Cas12a2 polypeptide to a target sequence of P. syringae. The target sequence of P. syringae can be within the AvrE1-2 gene, wherein the target sequence has the nucleotide sequence set forth as SEQ ID NO: 112. In some embodiments, a spacer of the disclosure has a nucleotide sequence set forth as SEQ ID NO: 184 or a nucleotide sequence that differs in length and/or sequence from SEQ ID NO: 184 by 1 to 5 nucleotides. In some embodiments, the spacer has a nucleotide sequence that differs in length and/or sequence from from SEQ ID NO: 184 by 5 nucleotides. In some embodiments, the spacer has a nucleotide sequence that differs in length and/or sequence from from SEQ ID NO: 184 by 4 nucleotides. In some embodiments, the spacer has a nucleotide sequence that differs in length and/or sequence from from SEQ ID NO: 184 by 3 nucleotides. In some embodiments, the spacer has a nucleotide sequence that differs in length and/or sequence from from SEQ ID NO: 184 by 2 nucleotides. In some embodiments, the spacer has a nucleotide sequence that differs in length and/or sequence from from SEQ ID NO: 184 by 1 nucleotide.

The presently disclosed guide RNAs comprise a spacer capable of targeting a Cas12a2 polypeptide to a target sequence of P. syringae. The target sequence of P. syringae can be within the AvrE1-4 gene, wherein the target sequence has the nucleotide sequence set forth as SEQ ID NO: 113. In some embodiments, a spacer of the disclosure has a nucleotide sequence set forth as SEQ ID NO: 185 or a nucleotide sequence that differs in length and/or sequence from SEQ ID NO: 185 by 1 to 5 nucleotides. In some embodiments, the spacer has a nucleotide sequence that differs in length and/or sequence from from SEQ ID NO: 185 by 5 nucleotides. In some embodiments, the spacer has a nucleotide sequence that differs in length and/or sequence from from SEQ ID NO: 185 by 4 nucleotides. In some embodiments, the spacer has a nucleotide sequence that differs in length and/or sequence from from SEQ ID NO: 185 by 3 nucleotides. In some embodiments, the spacer has a nucleotide sequence that differs in length and/or sequence from from SEQ ID NO: 185 by 2 nucleotides. In some embodiments, the spacer has a nucleotide sequence that differs in length and/or sequence from from SEQ ID NO: 185 by 1 nucleotide.

The presently disclosed guide RNAs comprise a spacer capable of targeting a Cas12a2 polypeptide to a target sequence of P. syringae. The target sequence of P. syringae can be within the HopAA1-1 gene, wherein the target sequence has the nucleotide sequence set forth as SEQ ID NO: 114. In some embodiments, a spacer of the disclosure has a nucleotide sequence set forth as SEQ ID NO: 186 or a nucleotide sequence that differs in length and/or sequence from SEQ ID NO: 186 by 1 to 5 nucleotides. In some embodiments, the spacer has a nucleotide sequence that differs in length and/or sequence from from SEQ ID NO: 186 by 5 nucleotides. In some embodiments, the spacer has a nucleotide sequence that differs in length and/or sequence from from SEQ ID NO: 186 by 4 nucleotides. In some embodiments, the spacer has a nucleotide sequence that differs in length and/or sequence from from SEQ ID NO: 186 by 3 nucleotides. In some embodiments, the spacer has a nucleotide sequence that differs in length and/or sequence from from SEQ ID NO: 186 by 2 nucleotides. In some embodiments, the spacer has a nucleotide sequence that differs in length and/or sequence from from SEQ ID NO: 186 by 1 nucleotide.

The presently disclosed guide RNAs comprise a spacer capable of targeting a Cas12a2 polypeptide to a target sequence of P. syringae. The target sequence of P. syringae can be within the HopAA1-3 gene, wherein the target sequence has the nucleotide sequence set forth as SEQ ID NO: 115. In some embodiments, a spacer of the disclosure has a nucleotide sequence set forth as SEQ ID NO: 187 or a nucleotide sequence that differs in length and/or sequence from SEQ ID NO: 187 by 1 to 5 nucleotides. In some embodiments, the spacer has a nucleotide sequence that differs in length and/or sequence from from SEQ ID NO: 187 by 5 nucleotides. In some embodiments, the spacer has a nucleotide sequence that differs in length and/or sequence from from SEQ ID NO: 187 by 4 nucleotides. In some embodiments, the spacer has a nucleotide sequence that differs in length and/or sequence from from SEQ ID NO: 187 by 3 nucleotides. In some embodiments, the spacer has a nucleotide sequence that differs in length and/or sequence from from SEQ ID NO: 187 by 2 nucleotides. In some embodiments, the spacer has a nucleotide sequence that differs in length and/or sequence from from SEQ ID NO: 187 by 1 nucleotide.

The presently disclosed guide RNAs comprise a spacer capable of targeting a Cas12a2 polypeptide to a target sequence of P. infestans. The target sequence of P. infestans can be within the Avrblb1-2 gene, wherein the target sequence has the nucleotide sequence set forth as SEQ ID NO: 162. In some embodiments, a spacer of the disclosure has a nucleotide sequence set forth as SEQ ID NO: 188 or a nucleotide sequence that differs in length and/or sequence from SEQ ID NO: 188 by 1 to 5 nucleotides. In some embodiments, the spacer has a nucleotide sequence that differs in length and/or sequence from from SEQ ID NO: 188 by 5 nucleotides. In some embodiments, the spacer has a nucleotide sequence that differs in length and/or sequence from from SEQ ID NO: 188 by 4 nucleotides. In some embodiments, the spacer has a nucleotide sequence that differs in length and/or sequence from from SEQ ID NO: 188 by 3 nucleotides. In some embodiments, the spacer has a nucleotide sequence that differs in length and/or sequence from from SEQ ID NO: 188 by 2 nucleotides. In some embodiments, the spacer has a nucleotide sequence that differs in length and/or sequence from from SEQ ID NO: 188 by 1 nucleotide.

The presently disclosed guide RNAs comprise a spacer capable of targeting a Cas12a2 polypeptide to a target sequence of P. infestans. The target sequence of P. infestans can be within the CRE8-1 gene, wherein the target sequence has the nucleotide sequence set forth as SEQ ID NO: 163. In some embodiments, a spacer of the disclosure has a nucleotide sequence set forth as SEQ ID NO: 189 or a nucleotide sequence that differs in length and/or sequence from SEQ ID NO: 189 by 1 to 5 nucleotides. In some embodiments, the spacer has a nucleotide sequence that differs in length and/or sequence from from SEQ ID NO: 189 by 5 nucleotides. In some embodiments, the spacer has a nucleotide sequence that differs in length and/or sequence from from SEQ ID NO: 189 by 4 nucleotides. In some embodiments, the spacer has a nucleotide sequence that differs in length and/or sequence from from SEQ ID NO: 189 by 3 nucleotides. In some embodiments, the spacer has a nucleotide sequence that differs in length and/or sequence from from SEQ ID NO: 189 by 2 nucleotides. In some embodiments, the spacer has a nucleotide sequence that differs in length and/or sequence from from SEQ ID NO: 189 by 1 nucleotide.

The presently disclosed guide RNAs comprise a spacer capable of targeting a Cas12a2 polypeptide to a target sequence of Leptinotarsa decemlineata. The target sequence of Leptinotarsa decemlineata can be within the LdNA_17212-1 gene, wherein the target sequence has the nucleotide sequence set forth as SEQ ID NO: 164. In some embodiments, a spacer of the disclosure has a nucleotide sequence set forth as SEQ ID NO: 190 or a nucleotide sequence that differs in length and/or sequence from SEQ ID NO: 190 by 1 to 5 nucleotides. In some embodiments, the spacer has a nucleotide sequence that differs in length and/or sequence from from SEQ ID NO: 190 by 5 nucleotides. In some embodiments, the spacer has a nucleotide sequence that differs in length and/or sequence from from SEQ ID NO: 190 by 4 nucleotides. In some embodiments, the spacer has a nucleotide sequence that differs in length and/or sequence from from SEQ ID NO: 190 by 3 nucleotides. In some embodiments, the spacer has a nucleotide sequence that differs in length and/or sequence from from SEQ ID NO: 190 by 2 nucleotides. In some embodiments, the spacer has a nucleotide sequence that differs in length and/or sequence from from SEQ ID NO: 190 by 1 nucleotide.

The presently disclosed guide RNAs comprise a spacer capable of targeting a Cas12a2 polypeptide to a target sequence of Leptinotarsa decemlineata. The target sequence of Leptinotarsa decemlineata can be within the LdNA_31771-2 gene, wherein the target sequence has the nucleotide sequence set forth as SEQ ID NO: 165. In some embodiments, a spacer of the disclosure has a nucleotide sequence set forth as SEQ ID NO: 191 or a nucleotide sequence that differs in length and/or sequence from SEQ ID NO: 191 by 1 to 5 nucleotides. In some embodiments, the spacer has a nucleotide sequence that differs in length and/or sequence from from SEQ ID NO: 191 by 5 nucleotides. In some embodiments, the spacer has a nucleotide sequence that differs in length and/or sequence from from SEQ ID NO: 191 by 4 nucleotides. In some embodiments, the spacer has a nucleotide sequence that differs in length and/or sequence from from SEQ ID NO: 191 by 3 nucleotides. In some embodiments, the spacer has a nucleotide sequence that differs in length and/or sequence from from SEQ ID NO: 191 by 2 nucleotides. In some embodiments, the spacer has a nucleotide sequence that differs in length and/or sequence from from SEQ ID NO: 191 by 1 nucleotide.

The guide RNA also can comprise a second region that forms a secondary structure. In some embodiments, the secondary structure comprises a stem or hairpin. The length of the stem can vary. For example, the stem can range from about 5, to about 6, to about 10, to about 15, to about 20, to about 25 base pairs in length. The stem can comprise one or more bulges of 1 to about 10 nucleotides. In some preferred embodiments, the hairpin structure comprises the sequence UCUACN₃₋₅GUAGAU (SEQ ID NOs: 40-42, encoded by SEQ ID NOs: 43-45), with “UCUAC” and “GUAGA” base-pairing to form the stem. “N₃₋₅” indicates 3, 4, or 5 nucleotides. Thus, the overall length of the second region can range from about 14 to about 25 nucleotides in length. In certain embodiments, the loop is about 3, 4, or 5 nucleotides in length and the stem comprises about 5, 6, 7, 8, 9, or 10 base pairs.

The guide RNA can also comprise a third region that remains essentially single-stranded. Thus, the third region has no complementarity to any nucleotide sequence in the cell of interest and has no complementarity to the rest of the guide RNA. The length of the third region can vary. In general, the third region is more than about 4 nucleotides in length. For example, the length of the third region can range from about 5 to about 60 nucleotides in length. The combined length of the second and third regions (also called the universal or scaffold region) of the guide RNA can range from about 30 to about 120 nucleotides in length. In one aspect, the combined length of the second and third regions of the guide RNA range from about 40 to about 45 nucleotides in length.

The scaffold region can comprise the nucleotide sequence of SEQ ID NO: 192, or an active variant or fragment thereof that when comprised within a guide RNA, is capable of directing the sequence-specific binding of an associated Cas12a2 polypeptide provided herein to a presently disclosed target DNA sequence within a target site specific to a plant pest. In some embodiments, an active scaffold region variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to a nucleotide sequence set forth as SEQ ID NO: 192. In some embodiments, an active scaffold region fragment comprises at least 5, 6, 7, 8, 9, 10, 11, 12, 13, 14, 15, 16, 17, 18, 19, 20, 21, 22, 23, 24, 25, 26, 27, 28, 29, or 30 contiguous nucleotides of a nucleotide sequence set forth as SEQ ID NO: 192.

In some embodiments, the scaffold region comprises a nucleotide sequence that differs in length and/or sequence from SEQ ID NO: 192 by 1 to 8 nucleotides. In some embodiments, the scaffold region comprises a nucleotide sequence that differs in length and/or sequence from SEQ ID NO: 192 by 8 nucleotides. In some embodiments, the scaffold region comprises a nucleotide sequence that differs in length and/or sequence from SEQ ID NO: 192 by 7 nucleotides. In some embodiments, the scaffold region comprises a nucleotide sequence that differs in length and/or sequence from SEQ ID NO: 192 by 6 nucleotides. In some embodiments, the scaffold region comprises a nucleotide sequence that differs in length and/or sequence from SEQ ID NO: 192 by 5 nucleotides. In some embodiments, the scaffold region comprises a nucleotide sequence that differs in length and/or sequence from SEQ ID NO: 192 by 4 nucleotides. In some embodiments, the scaffold region comprises a nucleotide sequence that differs in length and/or sequence from SEQ ID NO: 192 by 3 nucleotides. In some embodiments, the scaffold region comprises a nucleotide sequence that differs in length and/or sequence from SEQ ID NO: 192 by 2 nucleotides. In some embodiments, the scaffold region comprises a nucleotide sequence that differs in length and/or sequence from SEQ ID NO: 192 by 1 nucleotide. In some embodiments, the scaffold region comprises the nucleotide sequence set forth as SEQ ID NO: 192.

In a preferred embodiment, the guide RNA comprises a single molecule comprising all three regions. In other embodiments, the guide RNA can comprise two separate molecules. The first RNA molecule can comprise the first region (i.e. the spacer) of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 25 nucleotides) that base pairs to the other sequence to form a functional guide RNA. In specific embodiments, the guide RNA is a single molecule (i.e., crRNA) that interacts with the target site in the chromosome and the Cas12a2 polypeptide without the need for a second guide RNA (i.e., a tracrRNA).

### IV. Nucleic Acids Encoding Cas12a2 Polypeptides, Fusion Proteins, and Guide RNAs

Nucleic acids encoding any of the Cas12a2 polypeptides, fusion proteins, and/or guide RNAs described herein are provided. The nucleic acid can be RNA or DNA. Examples of polynucleotides that encode Cas12a2 polypeptides are set forth in the group consisting of SEQ ID NOs: 63-101, and 167. In some embodiments, the nucleic acid encoding the Cas12a2 polypeptide or fusion protein is mRNA. The mRNA can be 5′ capped and/or 3′ polyadenylated. In another embodiment, the nucleic acid encoding the Cas12a2 polypeptide or fusion protein is DNA. The DNA can be present in a phage, plasmid, or other vector.

Nucleic acids encoding the Cas12a2 polypeptide or fusion proteins can be codon optimized for efficient translation into protein in the cell of interest. Programs for codon optimization are available in the art (e.g., OPTIMIZER at genomes.urv.es/OPTIMIZER; OptimumGene™ from GenScript, World Wide Web at genscript.com/codon_opt.html).

In certain embodiments, DNA encoding the Cas12a2 polypeptide or fusion protein can be operably linked to at least one promoter sequence. The DNA coding sequence can be operably linked to a promoter control sequence for expression in a host cell of interest, for example a plant cell or cell from a plant pest. “Operably linked” is intended to mean a functional linkage between two or more elements. For example, an operable linkage between a promoter and a coding region of interest (e.g., region coding for a Cas12a2 polypeptide or guide RNA) is a functional link that allows for expression of the coding region of interest. Operably linked elements may be contiguous or non-contiguous. When used to refer to the joining of two protein coding regions (either at the N-terminus, at the C-terminus, or at an internal amino acid position), by “operably linked” or “operably fused” is intended that the coding regions are in the same reading frame, even if one protein is inserted into another. In some embodiments, polypeptides that are “operably linked” or “operably fused” means that the structure and/or biological activity of each individual polypeptide is also present in the fusion.

The promoter sequence can be derived from bacterial sequences, viral sequences, synthetically-designed sequences, or other sources. It is recognized that different applications can be enhanced by the use of different promoters in the nucleic acid molecules to modulate the timing, location and/or level of expression of the Cas12a2 polypeptide, fusion protein, and/or guide RNA. Such nucleic acid molecules may also contain, if desired, a promoter regulatory region (e.g., one conferring inducible, constitutive, or environmentally- or developmentally-regulated expression), a transcription initiation start site, a ribosome binding site, an RNA processing signal, a transcription termination site, and/or a polyadenylation signal. A promoter operably linked to a polynucleotide encoding a Cas12a2 polypeptide or fusion protein of the disclosure can include tissue-specific promoters, including but not limited to seed-specific, tuber-specific, stem-specific, pollen-specific, root-specific, leaf-specific, and green tissue-specific promoters. In some embodiments, a tissue-specific promoter operably linked to a polynucleotide encoding a Cas12a2 polypeptide of the disclosure comprises a promoter that targets any particular tissue of a plant.

The nucleic acid sequences encoding the Cas12a2 polypeptide or fusion protein can be operably linked to a promoter sequence that is recognized by a phage RNA polymerase for in vitro mRNA synthesis. In such embodiments, the in vitro-transcribed RNA can be purified for use in the methods of plant pest elimination described herein. For example, the promoter sequence can be a T7, T3, or SP6 promoter sequence or a variation of a T7, T3, or SP6 promoter sequence. In some embodiments, the sequence encoding the Cas12a2 polypeptide or fusion protein can be operably linked to a promoter sequence for in vitro expression of the Cas12a2 polypeptide or fusion protein. In such embodiments, the expressed protein and/or guide polynucleotide such as a guide RNA can be purified for use in the methods described herein.

The DNA encoding the Cas12a2 polypeptide, fusion protein, and/or guide RNA can be present in a vector. Suitable vectors include engineered bacteriophages, bacterial vectors, plasmid vectors (for example conjugative plasmid vectors), phagemids, cosmids, artificial/mini-chromosomes, transposons, and viral vectors (e.g., lentiviral vectors, adeno-associated viral vectors, etc.). In one embodiment, the DNA encoding the Cas12a2 polypeptide, fusion protein, and/or guide RNA is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001. In some embodiments, the DNA encoding the Cas12a2 polypeptide, fusion protein, and/or guide RNA is present in an engineered bacteriophage, where the native bacteriophage sequence is derived from a bacteriophage that is capable of infecting the bacterial cell(s) of interest.

In some embodiments, the expression vector comprising the sequence encoding the Cas12a2 polypeptide can further comprise a sequence encoding a guide polynucleotide (e.g., guide RNA). The sequence encoding the guide polynucleotide (e.g., guide RNA) can be operably linked to at least one transcriptional control sequence for expression of the guide RNA in the cell of interest. In some embodiments, the transcriptional control sequence can include tissue-specific promoters, including root-specific, leaf-specific, and green tissue-specific promoters.

### V. Methods for Targeting a Nucleotide Sequence in a Cell of a Plant Pest

Methods are provided herein for targeting a nucleotide sequence in a cell of a plant pest. The methods comprise introducing into a plant, plant part, or plant cell, one or more DNA-targeting or RNA-targeting polynucleotides such as, for example, a DNA-targeting or RNA-targeting RNA (“guide RNA,” “gRNA,” “CRISPR RNA,” or “crRNA”) or a DNA polynucleotide encoding a DNA-targeting or RNA-targeting RNA, wherein the DNA-targeting or RNA-targeting polynucleotide comprises: (a) a first segment comprising a nucleotide sequence that is complementary to a target DNA or RNA sequence of one or more plant pest; and (b) a second segment that interacts with a Cas12a2 polypeptide and also introducing to the plant, plant part, or plant cell, a Cas12a2 polypeptide, or a polynucleotide such as a DNA molecule or an RNA molecule encoding a Cas12a2 polypeptide, wherein the Cas12a2 polypeptide comprises: (a) a polynucleotide-binding portion that interacts with the gRNA or other DNA-targeting or RNA-targeting polynucleotide; and (b) an activity portion that may comprise a catalytic domain such as a RuvC domain that exhibits site-directed enzymatic activity.

The activity of Cas12a2 polypeptides used in the methods of the disclosure can encompass a primary activity that can result in an initial site-specific single or double-stranded cut to a polynucleotide followed by secondary activity that can result in a non-specific cleavage and/or degradation of polynucleotides in a cell. The primary activity can produce (i) a single-strand or double-strand break in dsDNA or dsRNA or (ii) a single-strand break in ssRNA or ssDNA. This site-specific primary activity occurs at a target sequence adjacent to a recognition sequence (i.e. a PAM, a PFM, or a PFS). In certain embodiments, an RNA target sequence comprises the reverse complement of a corresponding DNA target sequence, such that the reverse complement of any DNA target sequence disclosed herein can function as an RNA target sequence. Moreover, DNA target sequences can be located 3′ from a PAM and, thus, an RNA target sequence can be located 5′ of a PFM, PFS, or PAM. As used herein, target sequences can refer to a DNA or RNA target sequence that results in site-specific cleavage of the polynucleotide and precedes non-specific cleavage and/or degradation of other DNA or RNA in the cell.

A plant refers to a whole plant, any part thereof, or a cell or tissue culture derived from a plant, comprising any of: whole plants, plant components or organs (e.g., leaves, stems, roots, embryos, pollen, ovules, seeds, leaves, flowers, branches, fruit, pulp, juice, kernels, ears, cobs, husks, stalks, root tips, anthers, etc.), plant tissues, seeds, plant cells, protoplasts and/or progeny of the same. A plant cell is a biological cell of a plant, taken from a plant or derived through culture of a cell taken from a plant. Grain is intended to mean the mature seed produced by commercial growers for purposes other than growing or reproducing the species. Progeny, variants, and mutants of the regenerated plants are also included within the scope of the invention.

A plant, plant part, or plant cell of interest of the disclosure refers to a plant, plant part, or plant cell that is desirable to be modified by introduction of or contact with a composition comprising a Cas12a2 polypeptide (or a polynucleotide encoding a Cas12a2 polypeptide) and/or at least one guide polynucleotide (or at least one polynucleotide encoding the at least one guide polynucleotide) of the disclosure to achieve resistance or tolerance against one or more pest. A pest of interest of the disclosure refers to a pest that is desired to be controlled (e.g., killed, eliminated, reduced in viability or fitness) by a composition comprising a Cas12a2 polypeptide (or a polynucleotide encoding a Cas12a2 polypeptide) and/or at least one guide polynucleotide (or at least one polynucleotide encoding the at least one guide polynucleotide) of the disclosure.

Methods are provided herein for reducing an amount of a plant pest. An “amount” of a plant pest can refer to a number of the plant pest, a size of the plant pest, a weight of the plant pest, the fitness of the plant pest, or another characteristic of the plant pest that is a measure of how much of the plant pest is present and/or viable. An “amount” of a plant pest can include a population of a plant pest, one or more plant pest (e.g., 1, 2, 5, 10, 50, 100, 20, 500, 1000, 5000, 10,000, 50,000, 100,000, or more plant pests), one or more cells of a plant pest (e.g., 1, 2, 5, 10, 50, 100, 20, 500, 1000, 5000, 10,000, 50,000, 100,000, or more cells of a plant pest), or other property of a plant pest that can quantify the plant pest. A plant pest can be a microbe; thus, for example, an amount of the microbial plant pest can comprise an amount of cells (e.g., 1, 2, 5, 10, 50, 100, 20, 500, 1000, 5000, 10,000, 50,000, 100,000, or more cells of the microbial plant pest). A plant pest can be a virus; thus, for example, an amount of the viral plant pest can comprise a physical titer (e.g., viral particles per milliliter (vp/mL) or genomic copies per milliliter (GC/mL)) or an infectious titer (e.g., plaque-forming units per milliliter (PFU/mL), focus-forming units per milliliter (FFU/mL), 50% Tissue Culture Infectious Dose (TCID5/mL)).

Methods for reducing an amount of a plant pest comprise contacting the plant pest with a composition comprising: (a) a Cas12a2 polypeptide, or a polynucleotide encoding a Cas12a2 polypeptide, and (b) at least one guide polynucleotide, or a polynucleotide encoding at least one guide polynucleotide; wherein each guide polynucleotide is capable of binding the Cas12a2 polypeptide and hybridizing to a target sequence in one or more cells of the plant pest, wherein the target sequence is located immediately adjacent to a PAM sequence that is recognized by the Cas12a2 polypeptide. The at least one guide polynucleotide can be a DNA-targeting or RNA-targeting polynucleotide (e.g, RNA), wherein the DNA-targeting or RNA-targeting polynucleotide (e.g, RNA) comprises: (a) a first segment comprising a nucleotide sequence that is complementary to a target DNA or RNA sequence of the plant pest; and (b) a second segment that interacts with the Cas12a2 polypeptide. Methods for reducing an amount of a plant pest can further comprise selecting for individuals or cells of the plant pest that comprise (a) a Cas12a2 polypeptide, or a polynucleotide encoding a Cas12a2 polypeptide, and/or (b) at least one guide polynucleotide, or a polynucleotide encoding at least one guide polynucleotide. Selecting for individuals or cells of the plant pest can include using a selectable agent, such as an herbicide, an antibiotic, a carbohydrate, an amino acid, or a metabolite, as described herein, and culturing or growing the plant pest in media comprising the selectable agent.

The plant pest that has been contacted with a Cas12a2/guide composition of the disclosure can have reduced ability to infect or destroy a plant. Without being bound by any one hypothesis, when introducing or contacting a composition comprising a Cas12a2 polypeptide or Cas12a2 fusion protein (or a polynucleotide encoding such) and/or at least one guide polynucleotide (or at least one polynucleotide encoding the at least one guide polynucleotide) of the disclosure into a plant pest (e.g., by transfection, transformation, electroporation), a primary activity of the Cas12a2 polypeptide or fusion/guide polynucleotide complex results in an initial site-specific cleavage of the plant pest-specific target sequence. The primary activity is followed by a secondary activity that can result in non-specific cleavage and/or degradation of nucleic acid molecules in the plant pest, leading to a reduced ability of the plant pest to infect/infest or destroy a plant. For example, a reduced ability of the plant pest to infect/infest or destroy a plant or plant population may be due to killing of the plant pest, reduction in viability of the plant pest, reduction in fitness of the plant pest, and/or reduction in feeding of the plant pest on plants. Reducing an amount of a plant pest includes reducing a population of the plant pest. In some embodiments, a population of the plant pest is eliminated.

In some embodiments, these methods result in the partial or complete control (e.g., killing and/or elimination, or reduction in viability, fitness, feeding, or infestation) of the plant pest that feeds on or infests a plant, plant part, or plant cell into which the Cas12a2 or encoding polynucleotide and guide polynucleotide have been introduced. The disclosure also provides for control (e.g., killing and/or elimination, or reduction in viability, fitness, feeding, or infestation) of the plant pest due to contact of the plant pest with a plant, plant part, or plant cell that has been applied (e.g., sprayed) or contacted with a composition comprising (a) a Cas12a2 polypeptide, or a polynucleotide encoding a Cas12a2 polypeptide, and (b) at least one guide polynucleotide, or a polynucleotide encoding at least one guide polynucleotide, wherein each guide polynucleotide is capable of binding the Cas12a2 polypeptide and hybridizing to a target sequence in one or more cells of each corresponding plant pest. Without being bound by any one hypothesis, when introducing a composition comprising a Cas12a2 polypeptide (or a polynucleotide encoding a Cas12a2 polypeptide) and/or at least one guide polynucleotide (or at least one polynucleotide encoding the at least one guide polynucleotide), of the disclosure into a plant, plant part, or plant cell, the composition may be transferred to the plant pest (e.g., by feeding or infesting activity of the plant pest) such that the compositions end up in the plant pest and secondary activity of the Cas12a2 polypeptide in the plant pest leads to control (e.g., killing, elimination, reduction in viability, fitness, feeding, or infestation) of the plant pest.

The methods described herein can result in at least 1%, at least 2%, at least 3%, at least 4%, at least 5%, at least 10%, at least 15%, at least 20%, at least 25%, at least 30%, at least 50%, at least 75%, at least 80%, at least 85%, at least 90%, at least 95%, at least 96%, at least 97%, at least 98%, at least 99%, 100% or 5-20%, 25-50%, 50-60%, 60-75%, 50-80%, 80-90%, 80-95%, 80-99%, 90-95%, 90-99%, or more decrease in the viable pest population in which the Cas12a2 or encoding polynucleotide and guide polynucleotide have been introduced. For example, the viability of a cell of a plant pest can be measured by any method known in the art, including plate count (e.g., CFU, CFU/g, CFU/mL), turbidity measurement, cell lysis, viability stains, average life span analysis, cell morphology, host-specific phenotypes, or any other method known in the art. In specific embodiments, control of cells of a plant pest (e.g., killing, eliminating, reducing the number or size of cells of a plant pest) can refer to elimination of future growth or division of cells of the plant pest.

The methods described herein can result in at least 1%, at least 2%, at least 3%, at least 4%, at least 5%, at least 10%, at least 15%, at least 20%, at least 25%, at least 30%, at least 50%, at least 75%, at least 80%, at least 85%, at least 90%, at least 95%, at least 96%, at least 97%, at least 98%, at least 99%, 100% or 5-20%, 25-50%, 50-60%, 60-75%, 50-80%, 80-90%, 80-95%, 80-99%, 90-95%, 90-99%, or more decrease in the amount of nucleic acid molecules in cells of a plant pest that has been contacted with a composition comprising a Cas12a2 polypeptide or Cas12a2 fusion protein (or a polynucleotide encoding such) and/or at least one guide polynucleotide (or at least one polynucleotide encoding the at least one guide polynucleotide). The amount of nucleic acid molecules in cells, as a measure of degradation of the nucleic acid molecules due to secondary activity of the Cas12a2/guide complex, can be measured by an assay known to one of skill in the art, including spectrophotometry (e.g., absorbance at 260 nm), fluorometry (e.g., using dyes that bind to nucleic acid molecule), PCR, and electrophoresis. In some embodiments, the decrease in the amount of nucleic acid molecules in cells of a plant pest is as compared to the amount of nucleic acid molecules in cells of a control pest.

Compositions comprising Cas12a2 polypeptides (or polynucleotides encoding Cas12a2 polypeptides) and/or guide polynucleotides (or polynucleotides encoding guide polynucleotides) of the disclosure can have “pesticidal activity”, which refers to activity of the composition that can be measured by, but is not limited to, pest mortality, pest weight loss, pest repellency, pest growth stunting, pest feeding, pest infestation, and other behavioral and physical changes of a pest after feeding and exposure to the composition for an appropriate length of time. In this manner, pesticidal activity impacts at least one measurable parameter of pest fitness. Compositions comprising Cas12a2 polypeptides (or polynucleotides encoding Cas12a2 polypeptides) and/or guide polynucleotides (or polynucleotides encoding guide polynucleotides) of the disclosure that can “control a pest population” or “control a pest” refers to any effect on a pest that results in limiting the damage that the pest causes. Controlling a pest includes, but is not limited to, killing the pest, inhibiting development of the pest, altering fertility or growth of the pest in such a manner that the pest provides less damage to the plant, decreasing the number of offspring produced, producing less fit pests, producing pests more susceptible to predator attack, or deterring the pests from eating the plant.

The methods of the disclosure can produce a modified plant or modified plant population with increased resistance or tolerance to one or more plant pest. A plant or plant population exhibits “resistance” or “tolerance” to a pest when symptoms of infestation are reduced or not observed at all when the plant or plant population is exposed to the pest under conditions allowing infestation.

In some embodiments, resistance and tolerance can also be observed by looking at the performance of the pest on resistant or tolerant plants or plant populations compared to plants or plant populations with no resistance or tolerance. In some embodiments, a plant or plant population exhibits resistance or tolerance to one or more plant pest because the plant or plant population has been modified by introducing into the plant (or into a part or cell of the plant) or plant population a composition comprising: (a) a Cas12a2 polypeptide, or a polynucleotide encoding a Cas12a2 polypeptide, and (b) at least one guide polynucleotide, or a polynucleotide encoding at least one guide polynucleotide; and selecting for a modified plant, plant part, or plant cell that expresses the Cas12a2 polypeptide and the at least one guide polynucleotide, wherein each guide polynucleotide is capable of binding the Cas12a2 polypeptide and hybridizing to a target sequence in one or more cells of each corresponding plant pest. “Corresponding plant pest” refers to a plant pest to be controlled that has the target sequence capable of being hybridized by a guide polynucleotide recognizing that target sequence.

In some embodiments, a plant or plant population exhibits resistance or tolerance to one or more plant pest because a composition is applied (e.g., sprayed on) to the plant or plant population, wherein the composition comprises: (a) a Cas12a2 polypeptide, or a polynucleotide encoding a Cas12a2 polypeptide, and (b) at least one guide polynucleotide, or a polynucleotide encoding at least one guide polynucleotide, wherein each guide polynucleotide is capable of binding the Cas12a2 polypeptide and hybridizing to a target sequence in one or more cells of each corresponding plant pest, and secondary activity brought about by the composition the pest has come into contact with or been exposed controls the pest.

Effects of compositions comprising a Cas12a2 polypeptide, or a polynucleotide encoding the same and/or at least one guide polynucleotide, or at least one polynucleotide encoding the same, of the disclosure on a plant pest can be determined by measuring the amount, size, or weight of the pest and compared to a control pest or control pest population. In some embodiments, the control pest or control pest population is a corresponding (i.e. same type of pest) pest that has not been contacted or been exposed to the composition or a pest that does not have the target sequence. In some embodiments, the control pest or pest population is the pest or pest population prior to contact or exposure to the composition. In some embodiments, the amount, size, or weight of the pest is reduced by 5%, 10%, 15%, 20%, 25%, 30%, 50%, 75%, 80%, 85%, 90%, 95%, 96%, 97%, 98%, 99%, 100% or 5-20%, 25-50%, 50-60%, 60-75%, 50-80%, 80-90%, 80-95%, 80-99%, 90-95%, 90-99%, or more, as compared to the amount, size, or weight of a control pest or pest population.

A resistant or tolerant plant or plant population modified to comprise or contacted with a composition comprising a Cas12a2 polypeptide (or a polynucleotide encoding a Cas12a2 polypeptide) and/or at least one guide polynucleotide (or at least one polynucleotide encoding the at least one guide polynucleotide), of the disclosure can have improved agronomic traits. Agronomic traits include, but are not limited to: greenness, grain yield, growth rate, total biomass or rate of accumulation, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, tiller number, panicle size, early seedling vigor, and seedling emergence under low temperature stress. These agronomic traits are measurable parameters and can be measured by assays known to one of ordinary skill in the art.

An improved agronomic trait includes improved crop performance. Crop performance is used synonymously with plant performance and refers to of how well a plant grows under a set of environmental conditions and cultivation practices. Crop performance can be measured by any metric a user associates with a crop's productivity (e.g. yield), appearance and/or robustness (e.g. color, morphology, height, biomass, maturation rate), product quality (e.g., seed protein content, seed oil content, seed carbohydrate content, etc.), cost of goods sold (e.g. the cost of creating a seed, plant, or plant product in a commercial, research, or industrial setting) and/or a plant's tolerance to disease (e.g. a response associated with deliberate or spontaneous infection by a pathogen), pests, microbes, fungi, and/or environmental stress. Crop performance can also be measured by determining a crop's commercial value and/or by determining the likelihood that a particular inbred, hybrid, or variety will become a commercial product, and/or by determining the likelihood that the offspring of an inbred, hybrid, or variety will become a commercial product. Crop performance can be a quantity (e.g. the volume or weight of seed or other plant product measured in liters or grams) or some other metric assigned to some aspect of a plant that can be represented on a scale (i.e., assigning a 1 to 10 value to a plant based on its disease tolerance).

An improved or enhanced agronomic trait in a plant or plant population resistant or tolerant to one or more plant pest can have a decrease or an increase in a measurable parameter, as compared to a control plant or control plant population, depending upon the parameter. For example, improved yield can include an increase in seed yield in a plant or plant population resistant or tolerant to one or more plant pest, as compared to a control plant or control plant population. As another example, improved yield can include a decrease in seed yield loss in a plant or plant population resistant or tolerant to one or more plant pest, as compared to a control plant or control plant population. The terms “decreased,” “fewer”, or “slower” and “increased”, “greater”, or “faster” used herein can refer to a decrease or increase in a measurable parameter of an agronomic trait of a resistant or tolerant plant or plant population modified to comprise or contacted with a composition comprising a Cas12a2 polypeptide (or a polynucleotide encoding a Cas12a2 polypeptide) and/or at least one guide polynucleotide (or at least one polynucleotide encoding the at least one guide polynucleotide), of the disclosure, as compared to the same measurable parameter of the same agronomic trait of a control plant or control plant population. For example, a decrease in a measurable parameter of an agronomic trait of a resistant or tolerant plant or plant population modified to comprise or contacted with a composition of the disclosure may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least 60%, between 60% and 70%, between 70% and 80%, at least 75%, at least 80%, between 80% and 90%, at least 90%, between 90% and 100%, at least 100%, between 100% and 200%, at least 200%, at least 300%, at least 400%, or more lower, as compared to the same measurable parameter of the same agronomic trait of a control plant or control plant population. An increase in a measurable parameter of an agronomic trait of a resistant or tolerant plant or plant population modified to comprise or contacted with a composition of the disclosure may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least 60%, between 60% and 70%, between 70% and 80%, at least 75%, at least 80%, between 80% and 90%, at least 90%, between 90% and 100%, at least 100%, between 100% and 200%, at least 200%, at least 300%, at least 400%, or more higher, as compared to the same measurable parameter of the same agronomic trait of a control plant or control plant population.

In some embodiments, a control plant or control plant population is not exposed to the pest at all but grown under the same conditions as a plant or plant population infested with or exposed to the same pest. In some embodiments, a control plant or control plant population does not comprise or has not been contacted with a composition comprising a Cas12a2 polypeptide (or a polynucleotide encoding a Cas12a2 polypeptide) and/or at least one guide polynucleotide (or at least one polynucleotide encoding the at least one guide polynucleotide), of the disclosure but grown under the same conditions as a plant or plant population modified to comprise the composition or contacted with the composition.

Methods of the disclosure can use a Cas12a2 polypeptide comprising an amino acid sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of SEQ ID NOs: 1-39, and 166. In some embodiments, methods of the disclosure uses a Cas12a2 polypeptide comprising the amino acid sequence set forth as any one of SEQ ID NOs: 1-39, and 166.

Methods of the disclosure can use a Cas12a2 polypeptide comprising an amino acid sequence encoded by a polynucleotide comprising a nucleotide sequence having at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or more sequence identity to any one of SEQ ID NOs: 63-101, and 167. In some embodiments, methods of the disclosure uses a Cas12a2 polypeptide comprising an amino acid sequence encoded by a polynucleotide comprising the nucleotide sequence set forth as any one of SEQ ID NOs: 63-101, and 167.

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a RuvCI domain comprising a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a RuvCI domain comprising a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 55 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a RuvCII domain comprising a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a RuvCII domain comprising a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I (e.g., and wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a RuvCIII domain comprising a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a RuvCIII domain comprising a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 62 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 46 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁I, X₁₂, and X₁₃=any amino acid; X₁₄=L, I, or V; X₁₅=A or V. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V (e.g., and wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 48 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F (e.g., and wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 50 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F (e.g., and wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 52 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 53 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K (e.g., and wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 56 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T (e.g., and wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 13 comprising the amino acid sequence set forth as X₁LX₃X₄X₅X₆X₇X₈KX₁₀GX₁₂X₁₃ANX₁₆X₁₇GX₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 13 comprising the amino acid sequence set forth as X₁LX₃X₄X₅X₆X₇X₈KX₁₀GX₁₂X₁₃ANX₁₆X₁₇GX₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 58 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 60 (e.g., wherein the Cas12a2 protein retains nuclease activity).

Methods of the disclosure can use a Cas12a2 polypeptide comprising an amino acid sequence having at least 90% sequence identity to any one of SEQ ID NOs: 1-39, and 166, wherein the Cas12a2 polypeptide comprises a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid. In some embodiments, methods of the disclosure uses a Cas12a2 polypeptide comprising an amino acid sequence having at least 95% sequence identity to any one of SEQ ID NOs: 1-39, and 166, wherein the Cas12a2 polypeptide comprises a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid. In some embodiments, methods of the disclosure uses a Cas12a2 polypeptide comprises a consensus motif having at least 90% identity (e.g., 90%, 91%, 92%, 93%, 94%, 95%, 96%, 97%, 98%, 99% or more identity) with SEQ ID NO: 61 (e.g., wherein the Cas12a2 protein retains nuclease activity).

The methods disclosed herein comprise introducing into a plant, plant part, or plant cell at least one Cas12a2 polypeptide or a nucleic acid encoding at least one Cas12a2 polypeptide, as described herein. In some embodiments, the Cas12a2 polypeptide can be introduced into the plant, plant part, or plant cell as an isolated protein.

In some embodiments, the Cas12a2 polypeptide can be introduced into the plant, plant part, or plant cell as a nucleoprotein in complex with a guide polynucleotide (for instance, as a ribonucleoprotein in complex with a guide RNA). In other embodiments, the Cas12a2 polypeptide can be introduced into the genome host as an mRNA molecule that encodes the Cas12a2 polypeptide. In still other embodiments, the Cas12a2 polypeptide can be introduced into the plant, plant part, or plant cell as a DNA molecule comprising an open reading frame that encodes the Cas12a2 polypeptide. In general, DNA sequences encoding the Cas12a2 polypeptide or fusion protein described herein are operably linked to a promoter sequence that will function in the plant, plant part, or plant cell of interest. The DNA sequence can be linear, or the DNA sequence can be part of a vector. In still other embodiments, the Cas12a2 polypeptide can be introduced into the plant, plant part, or plant cell as an RNA-protein complex comprising the guide RNA. In certain embodiments, the Cas12a2 polypeptide, Cas12a2-gRNA ribonucleoprotein complex, and/or Cas12a2-encoding polynucleotide can be introduced into the plant, plant part, or plant cell of interest via methods including nanoparticle-aided transformation (Kumari et al 2017 FEMS Microbiol Lett 364:fnx081; French 2019 BioRxiv dx.doi.org/10.1101/559252), chemical transfection, transfection using liposomes, e.g., cationic liposomes, or transfection with cationic polymers, including DEAD-dextran or polyethylenimine, Agrobacterium-mediated transformation, particle bombardment, microinjection, bacterial vector or viral vector mediated transformation, microparticle-mediated gene transfer, and electroporation.

In certain embodiments, a nucleic acid molecule encoding the Cas12a2 polypeptide can further comprise a polynucleotide encoding one or more guide RNAs. In general, each of the sequences encoding the Cas12a2 polypeptide and the guide RNA(s) is operably linked to one or more appropriate promoter sequences that enable expression of the Cas12a2 polypeptide and the guide RNA(s), respectively, in the plant, plant part, or plant cellof interest. The nucleic acid molecule encoding the Cas12a2 polypeptide and the guide RNA(s) can further comprise additional expression control, regulatory, and/or processing sequence(s). The nucleic acid molecule encoding the Cas12a2 polypeptide and the guide RNA(s) can be linear or can be part of a vector.

Methods described herein further can also comprise introducing into a plant, plant part, or plant cell at least one guide RNA or polynucleotide encoding at least one guide RNA.

Guide RNAs used in the methods of the disclosure can comprise a spacer capable of targeting a Cas12a2 polypeptide to a target sequence of C. elegans. The target sequence of C. elegans can be within the AGE1-1 gene, wherein the target sequence has the nucleotide sequence set forth as SEQ ID NO: 103. In some embodiments, a spacer of the disclosure has a nucleotide sequence set forth as SEQ ID NO: 175 or a nucleotide sequence that differs in length and/or sequence from SEQ ID NO: 175 by 1 to 5 nucleotides. In some embodiments, the spacer has a nucleotide sequence that differs in length and/or sequence from from SEQ ID NO: 175 by 5 nucleotides. In some embodiments, the spacer has a nucleotide sequence that differs in length and/or sequence from from SEQ ID NO: 175 by 4 nucleotides. In some embodiments, the spacer has a nucleotide sequence that differs in length and/or sequence from from SEQ ID NO: 175 by 3 nucleotides. In some embodiments, the spacer has a nucleotide sequence that differs in length and/or sequence from from SEQ ID NO: 175 by 2 nucleotides. In some embodiments, the spacer has a nucleotide sequence that differs in length and/or sequence from from SEQ ID NO: 175 by 1 nucleotide.

The target sequence of C. elegans can be within the AGE1-2 gene, wherein the target sequence has the nucleotide sequence set forth as SEQ ID NO: 104. In some embodiments, a spacer of the disclosure has a nucleotide sequence set forth as SEQ ID NO: 176 or a nucleotide sequence that differs in length and/or sequence from SEQ ID NO: 176 by 1 to 5 nucleotides. In some embodiments, the spacer has a nucleotide sequence that differs in length and/or sequence from from SEQ ID NO: 176 by 5 nucleotides. In some embodiments, the spacer has a nucleotide sequence that differs in length and/or sequence from from SEQ ID NO: 176 by 4 nucleotides. In some embodiments, the spacer has a nucleotide sequence that differs in length and/or sequence from from SEQ ID NO: 176 by 3 nucleotides. In some embodiments, the spacer has a nucleotide sequence that differs in length and/or sequence from from SEQ ID NO: 176 by 2 nucleotides. In some embodiments, the spacer has a nucleotide sequence that differs in length and/or sequence from from SEQ ID NO: 176 by 1 nucleotide.

The target sequence of C. elegans can be within the AGE1-7 gene, wherein the target sequence has the nucleotide sequence set forth as SEQ ID NO: 105. In some embodiments, a spacer of the disclosure has a nucleotide sequence set forth as SEQ ID NO: 177 or a nucleotide sequence that differs in length and/or sequence from SEQ ID NO: 177 by 1 to 5 nucleotides. In some embodiments, the spacer has a nucleotide sequence that differs in length and/or sequence from from SEQ ID NO: 177 by 5 nucleotides. In some embodiments, the spacer has a nucleotide sequence that differs in length and/or sequence from from SEQ ID NO: 177 by 4 nucleotides. In some embodiments, the spacer has a nucleotide sequence that differs in length and/or sequence from from SEQ ID NO: 177 by 3 nucleotides. In some embodiments, the spacer has a nucleotide sequence that differs in length and/or sequence from from SEQ ID NO: 177 by 2 nucleotides. In some embodiments, the spacer has a nucleotide sequence that differs in length and/or sequence from from SEQ ID NO: 177 by 1 nucleotide.

The target sequence of C. elegans can be within the AKT1-2 gene, wherein the target sequence has the nucleotide sequence set forth as SEQ ID NO: 106. In some embodiments, a spacer of the disclosure has a nucleotide sequence set forth as SEQ ID NO: 178 or a nucleotide sequence that differs in length and/or sequence from SEQ ID NO: 178 by 1 to 5 nucleotides. In some embodiments, the spacer has a nucleotide sequence that differs in length and/or sequence from from SEQ ID NO: 178 by 5 nucleotides. In some embodiments, the spacer has a nucleotide sequence that differs in length and/or sequence from from SEQ ID NO: 178 by 4 nucleotides. In some embodiments, the spacer has a nucleotide sequence that differs in length and/or sequence from from SEQ ID NO: 178 by 3 nucleotides. In some embodiments, the spacer has a nucleotide sequence that differs in length and/or sequence from from SEQ ID NO: 178 by 2 nucleotides. In some embodiments, the spacer has a nucleotide sequence that differs in length and/or sequence from from SEQ ID NO: 178 by 1 nucleotide.

The target sequence of C. elegans can be within the AKT1-5 gene, wherein the target sequence has the nucleotide sequence set forth as SEQ ID NO: 107. In some embodiments, a spacer of the disclosure has a nucleotide sequence set forth as SEQ ID NO: 179 or a nucleotide sequence that differs in length and/or sequence from SEQ ID NO: 179 by 1 to 5 nucleotides. In some embodiments, the spacer has a nucleotide sequence that differs in length and/or sequence from from SEQ ID NO: 179 by 5 nucleotides. In some embodiments, the spacer has a nucleotide sequence that differs in length and/or sequence from from SEQ ID NO: 179 by 4 nucleotides. In some embodiments, the spacer has a nucleotide sequence that differs in length and/or sequence from from SEQ ID NO: 179 by 3 nucleotides. In some embodiments, the spacer has a nucleotide sequence that differs in length and/or sequence from from SEQ ID NO: 179 by 2 nucleotides. In some embodiments, the spacer has a nucleotide sequence that differs in length and/or sequence from from SEQ ID NO: 179 by 1 nucleotide.

The target sequence of C. elegans can be within the AKT1-8 gene, wherein the target sequence has the nucleotide sequence set forth as SEQ ID NO: 108. In some embodiments, a spacer of the disclosure has a nucleotide sequence set forth as SEQ ID NO: 180 or a nucleotide sequence that differs in length and/or sequence from SEQ ID NO: 180 by 1 to 5 nucleotides. In some embodiments, the spacer has a nucleotide sequence that differs in length and/or sequence from from SEQ ID NO: 180 by 5 nucleotides. In some embodiments, the spacer has a nucleotide sequence that differs in length and/or sequence from from SEQ ID NO: 180 by 4 nucleotides. In some embodiments, the spacer has a nucleotide sequence that differs in length and/or sequence from from SEQ ID NO: 180 by 3 nucleotides. In some embodiments, the spacer has a nucleotide sequence that differs in length and/or sequence from from SEQ ID NO: 180 by 2 nucleotides. In some embodiments, the spacer has a nucleotide sequence that differs in length and/or sequence from from SEQ ID NO: 180 by 1 nucleotide.

Guide RNAs used in the methods of the disclosure can comprise a spacer capable of targeting a Cas12a2 polypeptide to a target sequence of P. syringae. The target sequence of P. syringae can be within the AvrE1-2 gene, wherein the target sequence has the nucleotide sequence set forth as SEQ ID NO: 112. In some embodiments, a spacer of the disclosure has a nucleotide sequence set forth as SEQ ID NO: 184 or a nucleotide sequence that differs in length and/or sequence from SEQ ID NO: 184 by 1 to 5 nucleotides. In some embodiments, the spacer has a nucleotide sequence that differs in length and/or sequence from from SEQ ID NO: 184 by 5 nucleotides. In some embodiments, the spacer has a nucleotide sequence that differs in length and/or sequence from from SEQ ID NO: 184 by 4 nucleotides. In some embodiments, the spacer has a nucleotide sequence that differs in length and/or sequence from from SEQ ID NO: 184 by 3 nucleotides. In some embodiments, the spacer has a nucleotide sequence that differs in length and/or sequence from from SEQ ID NO: 184 by 2 nucleotides. In some embodiments, the spacer has a nucleotide sequence that differs in length and/or sequence from from SEQ ID NO: 184 by 1 nucleotide.

The target sequence of P. syringae can be within the AvrE1-4 gene, wherein the target sequence has the nucleotide sequence set forth as SEQ ID NO: 113. In some embodiments, a spacer of the disclosure has a nucleotide sequence set forth as SEQ ID NO: 185 or a nucleotide sequence that differs in length and/or sequence from SEQ ID NO: 185 by 1 to 5 nucleotides. In some embodiments, the spacer has a nucleotide sequence that differs in length and/or sequence from from SEQ ID NO: 185 by 5 nucleotides. In some embodiments, the spacer has a nucleotide sequence that differs in length and/or sequence from from SEQ ID NO: 185 by 4 nucleotides. In some embodiments, the spacer has a nucleotide sequence that differs in length and/or sequence from from SEQ ID NO: 185 by 3 nucleotides. In some embodiments, the spacer has a nucleotide sequence that differs in length and/or sequence from from SEQ ID NO: 185 by 2 nucleotides. In some embodiments, the spacer has a nucleotide sequence that differs in length and/or sequence from from SEQ ID NO: 185 by 1 nucleotide.

The target sequence of P. syringae can be within the HopAA1-1 gene, wherein the target sequence has the nucleotide sequence set forth as SEQ ID NO: 114. In some embodiments, a spacer of the disclosure has a nucleotide sequence set forth as SEQ ID NO: 186 or a nucleotide sequence that differs in length and/or sequence from SEQ ID NO: 186 by 1 to 5 nucleotides. In some embodiments, the spacer has a nucleotide sequence that differs in length and/or sequence from from SEQ ID NO: 186 by 5 nucleotides. In some embodiments, the spacer has a nucleotide sequence that differs in length and/or sequence from from SEQ ID NO: 186 by 4 nucleotides. In some embodiments, the spacer has a nucleotide sequence that differs in length and/or sequence from from SEQ ID NO: 186 by 3 nucleotides. In some embodiments, the spacer has a nucleotide sequence that differs in length and/or sequence from from SEQ ID NO: 186 by 2 nucleotides. In some embodiments, the spacer has a nucleotide sequence that differs in length and/or sequence from from SEQ ID NO: 186 by 1 nucleotide.

The target sequence of P. syringae can be within the HopAA1-3 gene, wherein the target sequence has the nucleotide sequence set forth as SEQ ID NO: 115. In some embodiments, a spacer of the disclosure has a nucleotide sequence set forth as SEQ ID NO: 187 or a nucleotide sequence that differs in length and/or sequence from SEQ ID NO: 187 by 1 to 5 nucleotides. In some embodiments, the spacer has a nucleotide sequence that differs in length and/or sequence from from SEQ ID NO: 187 by 5 nucleotides. In some embodiments, the spacer has a nucleotide sequence that differs in length and/or sequence from from SEQ ID NO: 187 by 4 nucleotides. In some embodiments, the spacer has a nucleotide sequence that differs in length and/or sequence from from SEQ ID NO: 187 by 3 nucleotides. In some embodiments, the spacer has a nucleotide sequence that differs in length and/or sequence from from SEQ ID NO: 187 by 2 nucleotides. In some embodiments, the spacer has a nucleotide sequence that differs in length and/or sequence from from SEQ ID NO: 187 by 1 nucleotide.

Guide RNAs used in the methods of the disclosure can comprise a spacer capable of targeting a Cas12a2 polypeptide to a target sequence of P. infestans. The target sequence of P. infestans can be within the Avrblb1-2 gene, wherein the target sequence has the nucleotide sequence set forth as SEQ ID NO: 162. In some embodiments, a spacer of the disclosure has a nucleotide sequence set forth as SEQ ID NO: 188 or a nucleotide sequence that differs in length and/or sequence from SEQ ID NO: 188 by 1 to 5 nucleotides. In some embodiments, the spacer has a nucleotide sequence that differs in length and/or sequence from from SEQ ID NO: 188 by 5 nucleotides. In some embodiments, the spacer has a nucleotide sequence that differs in length and/or sequence from from SEQ ID NO: 188 by 4 nucleotides. In some embodiments, the spacer has a nucleotide sequence that differs in length and/or sequence from from SEQ ID NO: 188 by 3 nucleotides. In some embodiments, the spacer has a nucleotide sequence that differs in length and/or sequence from from SEQ ID NO: 188 by 2 nucleotides. In some embodiments, the spacer has a nucleotide sequence that differs in length and/or sequence from from SEQ ID NO: 188 by 1 nucleotide.

The target sequence of P. infestans can be within the CRE8-1 gene, wherein the target sequence has the nucleotide sequence set forth as SEQ ID NO: 163. In some embodiments, a spacer of the disclosure has a nucleotide sequence set forth as SEQ ID NO: 189 or a nucleotide sequence that differs in length and/or sequence from SEQ ID NO: 189 by 1 to 5 nucleotides. In some embodiments, the spacer has a nucleotide sequence that differs in length and/or sequence from from SEQ ID NO: 189 by 5 nucleotides. In some embodiments, the spacer has a nucleotide sequence that differs in length and/or sequence from from SEQ ID NO: 189 by 4 nucleotides. In some embodiments, the spacer has a nucleotide sequence that differs in length and/or sequence from from SEQ ID NO: 189 by 3 nucleotides. In some embodiments, the spacer has a nucleotide sequence that differs in length and/or sequence from from SEQ ID NO: 189 by 2 nucleotides. In some embodiments, the spacer has a nucleotide sequence that differs in length and/or sequence from from SEQ ID NO: 189 by 1 nucleotide.

Guide RNAs used in the methods of the disclosure can comprise a spacer capable of targeting a Cas12a2 polypeptide to a target sequence of Leptinotarsa decemlineata. The target sequence of Leptinotarsa decemlineata can be within the LdNA_17212-1 gene, wherein the target sequence has the nucleotide sequence set forth as SEQ ID NO: 164. In some embodiments, a spacer of the disclosure has a nucleotide sequence set forth as SEQ ID NO: 190 or a nucleotide sequence that differs in length and/or sequence from SEQ ID NO: 190 by 1 to 5 nucleotides. In some embodiments, the spacer has a nucleotide sequence that differs in length and/or sequence from from SEQ ID NO: 190 by 5 nucleotides. In some embodiments, the spacer has a nucleotide sequence that differs in length and/or sequence from from SEQ ID NO: 190 by 4 nucleotides. In some embodiments, the spacer has a nucleotide sequence that differs in length and/or sequence from from SEQ ID NO: 190 by 3 nucleotides. In some embodiments, the spacer has a nucleotide sequence that differs in length and/or sequence from from SEQ ID NO: 190 by 2 nucleotides. In some embodiments, the spacer has a nucleotide sequence that differs in length and/or sequence from from SEQ ID NO: 190 by 1 nucleotide.

The target sequence of Leptinotarsa decemlineata can be within the LdNA_31771-2 gene, wherein the target sequence has the nucleotide sequence set forth as SEQ ID NO: 165. In some embodiments, a spacer of the disclosure has a nucleotide sequence set forth as SEQ ID NO: 191 or a nucleotide sequence that differs in length and/or sequence from SEQ ID NO: 191 by 1 to 5 nucleotides. In some embodiments, the spacer has a nucleotide sequence that differs in length and/or sequence from from SEQ ID NO: 191 by 5 nucleotides. In some embodiments, the spacer has a nucleotide sequence that differs in length and/or sequence from from SEQ ID NO: 191 by 4 nucleotides. In some embodiments, the spacer has a nucleotide sequence that differs in length and/or sequence from from SEQ ID NO: 191 by 3 nucleotides. In some embodiments, the spacer has a nucleotide sequence that differs in length and/or sequence from from SEQ ID NO: 191 by 2 nucleotides. In some embodiments, the spacer has a nucleotide sequence that differs in length and/or sequence from from SEQ ID NO: 191 by 1 nucleotide.

Guide RNAs used in the methods of the disclosure can comprise a scaffold region comprising the nucleotide sequence of SEQ ID NO: 192, or an active variant or fragment thereof. In some embodiments, an active scaffold region variant comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to a nucleotide sequence set forth as SEQ ID NO: 192. In some embodiments, an active scaffold region fragment comprises at least 5, 6, 7, 8, 9, 10, 11, 12, 13, 14, 15, 16, 17, 18, 19, 20, 21, 22, 23, 24, 25, 26, 27, 28, 29,or 30 contiguous nucleotides of a nucleotide sequence set forth as SEQ ID NO: 192.

In some embodiments, the scaffold region comprises a nucleotide sequence that differs in length and/or sequence from SEQ ID NO: 192 by 1 to 8 nucleotides. In some embodiments, the scaffold region comprises a nucleotide sequence that differs in length and/or sequence from SEQ ID NO: 192 by 8 nucleotides. In some embodiments, the scaffold region comprises a nucleotide sequence that differs in length and/or sequence from SEQ ID NO: 192 by 7 nucleotides. In some embodiments, the scaffold region comprises a nucleotide sequence that differs in length and/or sequence from SEQ ID NO: 192 by 6 nucleotides. In some embodiments, the scaffold region comprises a nucleotide sequence that differs in length and/or sequence from SEQ ID NO: 192 by 5 nucleotides. In some embodiments, the scaffold region comprises a nucleotide sequence that differs in length and/or sequence from SEQ ID NO: 192 by 4 nucleotides. In some embodiments, the scaffold region comprises a nucleotide sequence that differs in length and/or sequence from SEQ ID NO: 192 by 3 nucleotides. In some embodiments, the scaffold region comprises a nucleotide sequence that differs in length and/or sequence from SEQ ID NO: 192 by 2 nucleotides. In some embodiments, the scaffold region comprises a nucleotide sequence that differs in length and/or sequence from SEQ ID NO: 192 by 1 nucleotide. In some embodiments, the scaffold region comprises the nucleotide sequence set forth as SEQ ID NO: 192.

In certain embodiments, the guide RNA(s) can be introduced into the plant, plant part, or plant cellas an RNA molecule. The RNA molecule can be transcribed in vitro. Alternatively, the RNA molecule can be chemically synthesized. In other embodiments, the guide RNA can be introduced into the genome host as a DNA molecule that encodes the guide RNA. In such cases, the DNA encoding the guide RNA can be operably linked to one or more promoter sequences for expression of the guide RNA in the plant, plant part, or plant cellof interest.

In some embodiments, multiple guide RNAs may be designed to target multiple target sequences in the pest of interest or multiple pests of interest and may be introduced into a plant, plant part, or plant cell of interest in the form of a CRISPR array in the format direct repeat-spacer-direct repeat-spacer, etc., repeating for the number of desired spacers. In these CRISPR arrays, the direct repeat sequences represent the portion of the gRNA that is recognized by Cas12a2. The direct repeat is processed by Cas12a2 enzymes to generate mature crRNAs that associate with the Cas12a2 protein to form the ribonucleoprotein complex that hybridizes with the targeted sequences in the pest of interest. In some embodiments, multiple guide RNAs may be designed to target multiple target sequences in the pest of interest and may be introduced into a plant, plant part, or plant cellof interest in the form of a CRISPR array in which the mature gRNAs are processed by ribozymes or by tRNA processing pathways (WO 2019/138052; Port and Bullock (2016) BioRxiv dx.doi.org/10.1101/046417).

The nucleic acid molecule encoding the Cas12a2 enzyme, a Cas12a2 fusion protein, and/or the guide RNA(s) can be linear or circular. In some embodiments, the nucleic acid sequence encoding the Cas12a2 enzyme, a Cas12a2 fusion protein, and/or the guide RNA(s) can be part of a vector. Suitable vectors include plasmid vectors (for example conjugative plasmid vectors), phagemids, cosmids, artificial/mini-chromosomes, transposons, bacterial vectors, and viral vectors. In an exemplary embodiment, the DNA encoding the Cas12a2 enzyme, a Cas12a2 fusion protein, and/or the guide RNA(s) is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In another exemplary embodiment, the DNA encoding the Cas12a2 enzyme, a Cas12a2 fusion protein, and/or the guide RNA(s) can be part of a phagemid.

In embodiments in which both the Cas12a2 polypeptide and the guide RNA(s) are introduced into the genome host as nucleic acid molecules, each can be part of a separate molecule (e.g., one vector containing Cas12a2 polypeptide or fusion protein coding sequence and a second vector containing guide RNA coding sequence(s)) or both can be part of the same molecule (e.g., one vector containing coding (and regulatory) sequence for both the Cas12a2 polypeptide and the guide RNA(s)).

A Cas12a2 polypeptide (or Cas12a2 fusion protein) in conjunction with a guide RNA is directed to a target site (i.e., comprising a target DNA or RNA sequence or target sequence of a plant pest) in a pest, wherein the Cas12a2 polypeptide (or Cas12a2 fusion protein) hybridizes with the target DNA or RNA sequence (the “initial hybridization event”) and produces a single-stranded or double-stranded break (i.e., cleavage, primary activity of the Cas12a2 polypeptide) in the target DNA or RNA. The cleavage site can be located anywhere within the target DNA or RNA. Without being limited by theory, this initial hybridization event triggers a conformational change in the Cas12a2 polypeptide that allows the Cas12a2 polypeptide to degrade dsRNA, ssRNA, ssDNA, and/or dsDNA in a non-sequence-specific manner (i.e. secondary activity of the Cas12a2 polypeptide). The target site has no sequence limitation except that the sequence is immediately preceded (i.e. upstream or 5′ of the target sequence) by a consensus sequence. This consensus sequence is also known as a protospacer adjacent motif (PAM), a protospacer flanking motif (PFM), or a protospacer flanking sequence (PFS). Examples of PAM (i.e. PFM, PFS) sequences include, but are not limited to, TTTN, NTTN, TTTV, NTTV, TTNV, VTTV, and TCTV (wherein N is defined as any nucleotide and V is defined as A, G, or C). In some embodiments, a PAM (i.e. PFM, PFS) sequence recognized by a Cas12a2 polypeptide of the disclosure includes any one of TTAA, TTAC, TTAG, TTCA, TTCC, TTGG, TTGA, TTGC, TTGG, TTTA, TTTC, TTTG, ATTA, ATTC, ATTG, CTTA, CTTC, CTTG, GTTA, GTTC, GTTG, TCTA, TCTC, TCTG, NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN, where N is A, C, G, or T; V is A, C, or G; R is A or G; B is C, G, or T; Y is C or T; M is A or C; K is G or T; H is A, C, or T; S is C or G; and D is A, G, or T.

It is well-known in the art that a suitable PAM (i.e. PFM, PFS) sequence must be located at the correct location relative to the target DNA or RNA sequence to allow the Cas12a2 nuclease to produce the desired double-stranded break. For all Cas12a2 nucleases characterized to date, the PAM (i.e. PFM, PFS) sequence is located immediately 5′ of the target DNA sequence. Thus, in some embodiments, the target DNA sequence is immediately downstream (3′) of the PAM (i.e. PFM, PFS) sequence. In some embodiments, the PAM (i.e. PFM, PFS) sequence is located immediately 3′ of the target RNA sequence. Thus, in some embodiments, the target RNA sequence is immediately upstream (5′) of the PAM (i.e. PFM, PFS) sequence. In certain embodiments, the target DNA or RNA sequence is immediately adjacent to the PAM (i.e. PFM, PFS) sequence. ‘Immediately adjacent’ refers to the target DNA or RNA sequence being about 1 nucleotide to 50 nucleotides, about 5 nucleotides to 45 nucleotides, or about 7 nucleotides to 40 nucleotides either upstream (5′) or downstream (3′) of the PAM (i.e. PFM, PFS) sequence. In some embodiments, the target DNA or RNA sequence is immediately adjacent to the PAM (i.e. PFM, PFS) sequence when it is 1 nucleotide, 2 nucleotides, 3 nucleotides, 4 nucleotides, 5 nucleotides, 6 nucleotides, 7 nucleotides, 8 nucleotides, 9 nucleotides, 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides upstream (5′) or downstream (3′) of the PAM (i.e. PFM, PFS) sequence. The PAM (i.e. PFM, PFS) site requirements for a given Cas12a2 nuclease cannot at present be predicted computationally, and instead must be determined experimentally using methods available in the art (Zetsche et al. (2015) Cell 163:759-771; Marshall et al. (2018) Mol Cell 69:146-157). It is well-known in the art that PAM sequence specificity for a given nuclease enzyme is affected by enzyme concentration (Karvelis et al. (2015) Genome Biol 16:253). Thus, modulating the concentrations of Cas12a2 protein delivered to the cell or in vitro system of interest represents a way to alter the PAM (i.e. PFM, PFS) site requirements associated with that Cas12a2 enzyme. Modulating Cas12a2 protein concentration in the system of interest may be achieved, for instance, by altering the promoter used to express the Cas12a2-encoding gene, by altering the concentration of ribonucleoprotein delivered to the cell or in vitro system, or by adding or removing introns that may play a role in modulating gene expression levels. As detailed herein, the first region of the guide RNA is complementary to the protospacer of the target sequence. Typically, the first region of the guide RNA is about 19 to 25 nucleotides in length.

The target site comprising a target DNA or RNA sequence can be in a coding region (e.g., of a gene), in an intron (e.g., of a gene), in a control region (e.g., of a gene), in a non-coding region (e.g., between genes), etc. In embodiments where the target sequence is within a gene, the gene can be a protein coding gene or an RNA coding gene. The gene can be any gene of interest as described herein. Cas12a2 collateral (i.e. secondary) activity against dsRNA, ssRNA, ssDNA, and/or dsDNA may be activated through an in initial hybridization event with any target DNA or RNA sequence(s) in the pest of interest as long as a suitable PAM (i.e. PFM, PFS) site is located 5′ or 3′ of the target sequence(s). The target sequence can be an mRNA transcribed from any gene of interest or, in specific embodiments, from a gene that functions in the pathogenic mode of action of the pest.

In some embodiments, a plurality of cells (e.g., plant pest cells) are contacted by or exposed to a composition comprising the Cas12a2 protein, or Cas12a2 protein-encoding polynucleotide, and guide RNA(s), or polynucleotide(s) encoding the guide RNA(s), where the guide RNA(s) are designed to target sequences that are present only in a certain fraction of the cells (e.g., plant pest cells). In some embodiments, this will result in the elimination or reduction of those cells that comprise the target sequence(s) that the guide RNA(s) are designed to hybridize with.

By “predetermined” or “target sequence” is intended a nucleotide (e.g., DNA or RNA) sequence in the pest of interest that is unique to that pest. The predetermined or target sequence may be within genomic DNA, chromosomal DNA, plasmid or other extrachromosomal DNA, dsRNA, ssRNA (e.g., mRNA), or other RNA molecules present in the pest of interest, but not present in other DNA or RNA molecules that could be in the same environment as the pest. Thus a pest-specific target sequence is present in the polynucleotide of one or more pests, but not present non-pest polynucleotides in the same natural environment of the pest, or is present only in negligible amounts in the same natural environment of the pest. The target sequence may be RNA, such as mRNA, present in a cell of the pest of interest. Methods are available in the art to find unique sequences within genomes and include using a Pan-Core genome approach to find accessory genes of organisms. Additionally using a Best Bi-directional Blast analysis or using OrthoMCL etc, would identify accessory genes. Additionally, unique regions between a pair of genomes can be extracted from a pair-wise global alignment performed using any of the popular programs like Nucmer (MUMmer), Mauve, BLAST, and the like. The target gene of interest can be associated with a secretory pathway or toxin. In some embodiments, the target gene of interest is associated with pathogenicity, such that the gene might be present in one or more pests, but not present in a non-pest organism. In some embodiments, a target sequence of interest (e.g., plant pest target sequence) is a sequence that is part of an antibiotic resistance gene. In some embodiments, a target sequence (e.g., plant pest target sequence) includes the following genes in C. elegans: AGE1-1, AGE1-2, AGE1-7, AKT1-2, AKT1-5, AKT1-8, or a combination thereof. In some embodiments, C. elegans target sequences include sequences set forth as any one of SEQ ID NOs: 103-108. Target sequences (e.g., plant pest target sequences) in P. syringae include sequences of AvrE1-2, AvrE1-4, HopAA1-1, HopAA1-3 genes, or a combination thereof. In some embodiments, P. syringae target sequences include sequences set forth as any one of SEQ ID NOs: 112-115. Target sequences (e.g., plant pest target sequences) in Pseudomonas infestans include Avrblb1-2 and/or CRE8-1 genes. In some embodiments, P. infestans target sequences include sequences set forth as SEQ ID NO: 162 or 163. Target sequences (e.g., plant pest target sequences) in Leptinotarsa decemlineata include LdNA_17212-1 and/or LdNA_31771-2 genes. In some embodiments, L. decemlineata target sequences include sequences set forth as SEQ ID NO: 164 or 165.

The methods disclosed herein also include methods that comprise contacting a plant, plant part, or plant cell or applying to a plant, plant part, or plant cell at least one Cas12a2 polypeptide (or Cas12a2 fusion protein) and/or a guide polynucleotide that binds to the Cas12a2 polypeptide (or Cas12a2 fusion protein), or a ribonucleoprotein complex comprising a Cas12a2 polypeptide (or Cas12a2 fusion protein) and a guide polynucleotide, as described herein. In some embodiments, the Cas12a2 polypeptide (or Cas12a2 fusion protein) is an isolated protein. In other embodiments, the Cas12a2 polypeptide (or Cas12a2 fusion protein) is encoded by a polynucleotide. In some embodiments, the polynucleotide encoding the Cas12a2 polypeptide (or Cas12a2 fusion protein) is an mRNA molecule. In still other embodiments, the Cas12a2 polypeptide (or Cas12a2 fusion protein) is encoded by a DNA sequence in a DNA molecule. The DNA sequence encoding the Cas12a2 polypeptide (or Cas12a2 fusion protein) can be operably linked to a constitutive or inducible promoter. In specific embodiments, the nucleotide sequence encoding the Cas12a2 polypeptide (or Cas12a2 fusion protein) is operably linked to a constitutive promoter. The DNA molecule can be linear or can be part of a vector. In such embodiments, the Cas12a2 polypeptide is operably fused to at least one cell-penetrating domain (CPP), which facilitates cellular uptake of the protein. CPPs that can be used to facilitate cellular uptake of a Cas12a2 polypeptide include: transactivating transcriptional activator (TAT), transportan, and penetratin. CPPs can include sequences that are arginine-rich, for example, CPPs comprising at least 7 arginines (R), at least 8 R, at least 9 R, or at least 10 R. In some embodiments, a CPP comprises: an R9-TAT having an amino acid sequence set forth as SEQ ID NO: 116 from human immunodeficiency virus; a transportan having an amino acid sequence set forth as SEQ ID NO: 117 from galanin-mastoparan chimeric peptide; and penetratin having an amino acid sequence set forth as SEQ ID NO: 160.

In some embodiments, the Cas12a2 polypeptide is operably fused to at least one export signal. For example, the export signal can include a signal peptide of the tobacco pathogenesis-related protein 1a (PR1a). An export signal can direct a nascent polypeptide, as it is being synthesized, to enter the endoplasmic reticulum (ER). Once inside the ER, the protein moves through the secretory pathway, eventually being exported from the cell. An ER export signal can comprise a diacidic (DxE) amino acid motif, a dihydrophobic (LL) amino acid motif, or a diaromatic (FF, YY) amino acid motif. In some embodiments, an export signal comprises a PR1a having an amino acid sequence set forth as SEQ ID NO: 118 from Nicotiana tabacum.

In certain embodiments, the Cas12a2 polypeptide is operably fused to at least one localization signal that can direct the expressed Cas12a2 polypeptide to a particular location or organelle in a cell or tissue. For example, the localization signal can be a nuclear localization signal (NLS). An NLS includes but is not limited to a SV40 peptide having an amino acid sequence set forth as SEQ ID NO: 119 from Simian vacuolating virus, and a nucleoplasmin peptide having an amino acid sequence set forth as SEQ ID NO: 120 from Xenopus.

A composition comprising a Cas12a2 polypeptide (or Cas12a2 fusion protein) (or a polynucleotide encoding a Cas12a2 polypeptide or Cas12a2 fusion protein) and/or at least one guide polynucleotide (or at least one polynucleotide encoding the at least one guide polynucleotide), of the disclosure can be formulated with an acceptable carrier into a pesticidal or agricultural composition(s) that is, for example, a suspension, a solution, an emulsion, a dusting powder, a dispersible granule, a wettable powder, a dry flowable, a wettable granule, a spray dried cellular composition, an emulsifiable concentrate, an aerosol, an impregnated granule, an adjuvant, a coatable paste, and/or encapsulated in, for example, polymer substances. The agricultural composition may be applied to the environment of a plant or an area of cultivation (e.g., by spraying), or applied to the plant, plant part, plant cell, or seed (e.g., by spraying or coating).

Such agricultural compositions may further comprise the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors, or other preparations that influence pest feeding or infesting of a host plant. One or more agrochemicals including, but not limited to, herbicides, insecticides, fungicides, bactericides, nematicides, molluscicides, acaracides, plant growth regulators, harvest aids, and fertilizers, can be combined with carriers, surfactants or adjuvants customarily employed in the art of formulation or other components to facilitate product handling and application for particular target pests. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present disclosure (e.g., comprising a Cas12a2 polypeptide, or a polynucleotide encoding a Cas12a2 polypeptide, and/or at least one guide polynucleotide, or at least one polynucleotide encoding the at least one guide polynucleotide) are normally applied in the form of compositions and can be applied to the crop area, plant, or seed to be treated. For example, the compositions of the present disclosure may be applied to grain in preparation for or during storage in a grain bin or silo, etc. The compositions of the present disclosure may be applied simultaneously or in succession with other compounds. Methods of applying an active ingredient of the present disclosure (e.g., comprising a Cas12a2 polypeptide, or a polynucleotide encoding a Cas12a2 polypeptide, and/or at least one guide polynucleotide, or at least one polynucleotide encoding the at least one guide polynucleotide) include, but are not limited to, foliar application, seed coating, and soil application. The number of applications and the rate of application depend on the intensity of infestation by the corresponding pest.

Suitable surface-active agents include, but are not limited to, anionic compounds such as a carboxylate of, for example, a metal; a carboxylate of a long chain fatty acid; an N-acylsarcosinate; mono or di-esters of phosphoric acid with fatty alcohol ethoxylates or salts of such esters; fatty alcohol sulfates such as sodium dodecyl sulfate, sodium octadecyl sulfate or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate of dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g., polyoxyethylene sorbitar fatty acid esters, block copolymers of ethylene oxide and propylene oxide, acetylenic glycols such as 2,4,7,9-tetraethyl-5-decyn-4,7-diol, or ethoxylated acetylenic glycols. Examples of a cationic surface-active agent include, for instance, an aliphatic mono-, di-, or polyamine such as an acetate, naphthenate or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include but are not limited to inorganic minerals such as kaolin, phyllosilicates, carbonates, sulfates, phosphates, or botanical materials such as cork, powdered corncobs, peanut hulls, rice hulls, and walnut shells.

The compositions of the present disclosure can be in a suitable form for direct application or as a concentrate of primary composition that requires dilution with a suitable quantity of water or other diluent before application. The pesticidal concentration will vary depending upon the nature of the particular formulation, specifically, whether it is a concentrate or to be used directly. In some embodiments, the composition contains 1 to 98% of a solid or liquid inert carrier, and 0 to 50% or 0.1 to 50% of a surfactant. The compositions can be administered at the labeled rate for the commercial product, for example, about 0.01 lb-5.0 lb. per acre when in dry form and at about 0.01 pts.-10 pts. per acre when in liquid form.

In a further embodiment, the composition(s) provided herein can be treated prior to formulation to prolong the pesticidal activity when applied to the environment of a pest of interest as long as the pretreatment is not deleterious to the pesticidal activity. Such treatment can be by chemical and/or physical means as long as the treatment does not deleteriously affect the properties of the composition(s). Examples of chemical reagents include but are not limited to halogenating agents; aldehydes such as formaldehyde and glutaraldehyde; anti-infectives, such as zephiran chloride; alcohols, such as isopropanol and ethanol; and histological fixatives, such as Bouin's fixative and Helly's fixative (see, for example, Humason (1967) Animal Tissue Techniques (W.H. Freeman and Co.).

Pests may be killed or reduced in numbers in a given area by application of the compositions provided herein (e.g., comprising a Cas12a2 polypeptide or Cas12a2 fusion protein, or a polynucleotide encoding such, and/or at least one guide polynucleotide, or at least one polynucleotide encoding the at least one guide polynucleotide) to the area. Alternatively, the compositions may be prophylactically applied to an environmental area to prevent infestation by a susceptible pest. Preferably the pest ingests, or is contacted with, a pesticidally-effective amount of the compositions. By “pesticidally-effective amount” is intended an amount of the pesticide that is able to bring about death to at least one pest, or to noticeably reduce pest growth, feeding, or normal physiological development. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective compositions. The formulations or compositions may also vary with respect to climatic conditions, environmental considerations, and/or frequency of application and/or severity of pest infestation.

The active ingredients are normally applied in the form of compositions and can be applied to the crop area, plant, or seed to be treated. Methods are therefore provided for providing to a plant, plant cell, seed, plant part or an area of cultivation, an effective amount of the agricultural composition comprising a Cas12a2 polypeptide or Cas12a2 fusion protein, or a polynucleotide encoding such, and/or at least one guide polynucleotide, or at least one polynucleotide encoding the at least one guide polynucleotide). By “effective amount” is intended an amount of a composition of the disclosure having pesticidal activity that is sufficient to kill or control the pest or result in a noticeable reduction in pest growth, feeding, or normal physiological development. Such decreases in numbers, pest growth, feeding or normal development can comprise any statistically significant decrease, including, for example a decrease of about 5%, 10%, 15%, 20%, 25%, 30%, 35%, 40%, 45%, 50%, 55%, 60%, 65%, 70%, 75%, 85%, 90%, 95% or greater.

For example, the compositions may be applied to grain in preparation for or during storage in a grain bin or silo, etc. The compositions may be applied simultaneously or in succession with other compounds. Methods of applying an active ingredient or an agrochemical composition comprising at least one of the polypeptides, recombinogenic polypeptides or variants or fragments thereof as disclosed herein, include but are not limited to, foliar application, seed coating, and soil application.

Methods for increasing plant yield are provided. The methods comprise introducing into or applying to a plant a composition comprising: (a) a Cas12a2 polypeptide, or a polynucleotide encoding a Cas12a2 polypeptide, and (b) at least one guide polynucleotide, or a polynucleotide encoding at least one guide polynucleotide, wherein each guide polynucleotide is capable of binding the Cas12a2 polypeptide and hybridizing to a target sequence in one or more cells of a plant pest; and growing the plant or a seed thereof in a field infested with (or susceptible to infestation by) a pest against which said composition has pesticidal activity. In some embodiments, the composition has pesticidal activity against a lepidopteran, coleopteran, dipteran, hemipteran, or nematode pest, and said field is infested with a lepidopteran, hemipteran, coleopteran, dipteran, or nematode pest. As defined herein, the “yield” of the plant refers to the quality and/or quantity of biomass produced by the plant. By “biomass” is intended any measured plant product. An increase in biomass production is any improvement in the yield of the measured plant product. Increasing plant yield has several commercial applications. For example, increasing plant leaf biomass may increase the yield of leafy vegetables for human or animal consumption. Additionally, increasing leaf biomass can be used to increase production of plant-derived pharmaceutical or industrial products. An increase in yield can comprise any statistically significant increase including, but not limited to, at least a 1% increase, at least a 3% increase, at least a 5% increase, at least a 10% increase, at least a 20% increase, at least a 30%, at least a 50%, at least a 70%, at least a 100% or a greater increase in yield compared to a control plant. In specific embodiments, plant yield is increased as a result of improved pest resistance of a plant comprising or contacted with a composition comprising a Cas12a2 polypeptide or Cas12a2 fusion protein (or a polynucleotide encoding such) and/or at least one guide polynucleotide (or at least one polynucleotide encoding the at least one guide polynucleotide), of the disclosure.

### VI. Plants, Plant Parts, Population of Plants, and Plant Products Produced by Methods of the Disclosure

The present disclosure provides plants, plant parts, population of plants, and plant products produced according to the methods provided herein. In some embodiments, such plants, plant parts, population of plants, and plant products comprise a Cas12a2 polypeptide or Cas12a2 fusion protein (or a polynucleotide encoding such) and/or at least one guide polynucleotide (or at least one polynucleotide encoding the at least one guide polynucleotide), of the disclosure. In some embodiments, plants, plant parts, population of plants, and plant products produced according to the methods provided herein comprise increased resistance or tolerance to one or more pest, as compared to a control plant, plant part, population of plants, or plant product. In some embodiments, plant products include any composition derived from the plant or plant part, including plant extract, plant concentrate, plant powder, plant biomass, grains, plant protein composition, and food and beverage products.

Compositions comprising a Cas12a2 polypeptide or Cas12a2 fusion protein (or a polynucleotide encoding such) and/or at least one guide polynucleotide (or at least one polynucleotide encoding the at least one guide polynucleotide), of the disclosure may be introduced or applied to any plant species, e.g., both monocots and dicots. Plants or plant parts that can be modified for resistance or tolerance to one or more plant pest according to the methods disclosed herein can be a legume, i.e., a plant belonging to the family Fabaceae (or Leguminosae), or a part (e.g., fruit or seed) of such a plant. When used as a dry grain, the seed of a legume is also called a pulse. Examples of a legume include, without limitation, soybean (Glycine max), beans (Phaseolus spp.), common bean (Phaseolus vulgaris), fava bean (Vicia faba), mung bean (Vigna radiata), cowpea (Vigna unguiculata), adzuki bean (Vigna angularis), pea (Pisum sativum), chickpea (Cicer arietinum), peanut (Arachis hypogaea), lentils (Lens culinaris, Lens esculenta), lupins (Lupinus spp.), white lupin (Lupinus albus), mesquite (Prosopis spp.), carob (Ceratonia siliqua), tamarind (Tamarindus indica), alfalfa (Medicago sativa), barrel medic (Medicago truncatula), birdsfood trefoil (Lotus japonicus), licorice (Glycyrrhiza glabra), and clover (Trifolium spp.). Plants or plant parts that can be modified for resistance or tolerance to one or more plant pest can be a crop plant or part of a crop plant, including legumes. Examples of crop plants include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), camelina (Camelina sativa), millet (e.g., pearl millet (Pennisetum glaucum), proso millet (Panicum miliaceum), foxtail millet (Setaria italica), finger millet (Eleusine coracana)), sunflower (Helianthus annuus), quinoa (Chenopodium quinoa), chicory (Cichorium intybus), lettuce (Lactuca sativa), safflower (Carthamus tinctorius), wheat (Triticum aestivum), soybean (Glycine max), tobacco (Nicotiana spp., e.g., Nicotiana tabacum, Nicotiana sylvestris), potato (Solanum tuberosum), tomato (Solanum lycopersicum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), grapes (Vitis vinifera, Vitis riparia), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oil palm (Elaeis guineensis), poplar (Populus spp.), pea (Pisum sativum), eucalyptus (Eucalyptus spp.), oats (Avena sativa), barley (Hordeum vulgare), vegetables, ornamentals, flowers, and conifers. Additionally, plants or plant parts that can be modified for resistance or tolerance to one or more plant pest can be an oilseed plant (e.g., canola (Brassica napus), cotton (Gossypium sp.), camelina (Camelina sativa) and sunflower (Helianthus sp.)), or other species including wheat (Triticum sp., such as Triticum aestivum L. ssp. aestivum (common or bread wheat), other subspecies of Triticum aestivum, Triticum turgidum L. ssp. durum (durum wheat, also known as macaroni or hard wheat), Triticum monococcum L. ssp. monococcum (cultivated einkorn or small spelt), Triticum timopheevi ssp. timopheevi, Triticum turgigum L. ssp. dicoccon (cultivated emmer), and other subspecies of Triticum turgidum (Feldman)), barley (Hordeum vulgare), maize (Zea mays), oats (Avena sativa), and hemp (Cannabis sativa).

### VII. Pests of Interest

A pest is any organism that can affect the performance of a plant in an undesirable way. A pesticide is any substance that reduces the survivability and/or reproduction of a pest, e.g. fungicides, bactericides, insecticides, herbicides, and other toxins. Common pests include microbes, animals (e.g. insects and other herbivores), and/or plants (e.g. weeds). A microbe will be understood to be a microorganism, i.e. a microscopic organism, which can be single celled or multicellular. Microorganisms are very diverse and include all the bacteria, archaea, protozoa, fungi, and algae, especially cells of plant pathogens and/or plant symbionts. Certain animals are also considered microbes, e.g., rotifers. In various embodiments, a microbe can be any of several different microscopic stages of a plant or animal. Microbes also include viruses, viroids, and prions, especially those which are pathogens or symbionts to crop plants. Thus, a “pest” includes but is not limited to, insects, fungi, bacteria, viruses, nematodes, mites, ticks, mollusks, spiders, scorpions, caterpillars, animals, and the like.

Insect pests include insects selected from the orders Coleoptera, Diptera, Hymenoptera, Lepidoptera, Mallophaga, Homoptera, Hemiptera, Orthroptera, Thysanoptera, Dermaptera, Isoptera, Anoplura, Siphonaptera, Trichoptera, etc., particularly Lepidoptera and Coleoptera. In some embodiments, insect pests include corn insect pests, such as Western corn rootworm (Diabrotica virgifera virgifera), Northern corn rootworm (Diabrotica barberi), Southern corn rootworm (Diabrotica undecimpunctata howardi), fall armyworm (Spodoptera frugiperda), and corn earworm (Helicoverpa zea). In some embodiments, insect pests include soybean insect pests, such as aphids (Aphis glycines). In some embodiments, insect pests include cotton insect pests, such as boll weevils (Anthonomus grandis), flower thrips, onion thrips, western flower thrips, fall armyworm (Spodoptera frugiperda), beet armyworm (Spodoptera exigua), and bollworms (Helicoverpa armigera). In some embodiments, insect pests potato insect pests, such as Colorado potato beetle (Leptinotarsa decemlineata), leafhopper (Empoasca fabae), dryland wireworm (Ctenicera pruinina), Pacific coast wireworm or click beetle (Limonius canus), and sugarbeet wireworm (Limonius californicus).

Plant parasitic nematodes include Aphelenchoides spp. (foliar nematodes), Caenorhabditis spp., Ditylenchus spp., Globodera spp. (potato cyst and golden nematodes), Heterodera spp. (soybean cyst nematodes), Longidorus spp., Meloidogyne spp. (root-knot nematodes), Nacobbus spp., Pratylenchus spp. (lesion nematodes), Trichodorus spp., Xiphinema spp. (dagger nematodes), and Bursaphelenchus spp. (e.g, B. xylophilus; pine wood nematode). In some embodiments, plant parasitic nematodes include soybean cyst nematode (Heterodera glycines).

A fungus includes any cell or tissue derived from a fungus, for example whole fungus, fungus components, organs, spores, hyphae, mycelium, and/or progeny of the same. A fungus cell is a biological cell of a fungus, taken from a fungus or derived through culture of a cell taken from a fungus. Fungal pests include Phakopsora pachyrhizi (soybean rust), Fusarium solani (disease complex)(sudden death syndrome), Cercospora sojina (frog eye leaf spot), and Peronospora manshurica (downy mildew). In some embodiments, the pest is Botrytis cinerea, a necrotrophic pathogenic fungus with an exceptionally wide host range. The cultivated tomato (predominantly Lycopersicon esculentum) is also susceptible to infection by Botrytis and the fungus generally affects stem, leaves and fruit of the tomato plant. In some embodiments, fungal pests include corn fungal pests, such as Fusarium verticillioides and Fusarium graminearum. In some embodiments, fungal pests include soybean fungal pests, such as soybean rust (Phakopsora pachyrhizi). In some embodiments, fungal pests include potato fungal pests, such as late blight (Phytophthora infestans).

Bacterial species that grow on plants or plant material may be targeted and selectively eliminated by the compositions and methods of the present invention. Non-limiting examples of plant- or plant-material associated bacterial species of interest include Xanthomonas spp., Escherichia spp., Pseudomonas spp., Erwinia spp., Xylella spp., Clavibacter spp., Ralstonia spp., Pectobacterium spp., Streptomyces spp., Burkholderia spp., Phytoplasma spp., Acidovorax spp., Pantoea spp., Agrobacterium spp., Spiroplasma spp., Candidatus Liberibacter spp., Dickeya spp., Serratia spp., Sphingomonas spp., Rhizobacter spp., Rhizomonas spp., Xylophilus spp., Rickettsia spp., Bacillus spp., Clostridium spp., Arthrobacter spp., Curtobacterium spp., Leifsonia spp., Rhodococcus spp., and Phytoplasma spp. Plant-associated bacteria may include, for example, plant pathogens, nodulating bacteria, bacteria that grow on plants and may harm humans or other animals that consume the plant material, or other bacteria. In some embodiments, the plant-associated bacteria is Pseudomonas syringae pv. glycinea that causes leaf blight.

Pests include bacteria and fungi that cause disease in plants including, without limitation, the following diseases: anthracnose, armillaria, ascochyta, aspergillus, bacterial blight, bacterial canker, bacterial speck, bacterial spot, bacterial wilt, bitter rot, black leaf, blackleg, black rot, black spot, blast, blight, blue mold, botrytis, brown rot, brown spot, cercospora, charcoal rot, cladosporium, clubroot, covered smut, crater rot, crown rot, damping off, dollar spot, downy mildew, early blight, ergot, erwinia, false loose smut, fire blight, foot rot, fruit blotch, fusarium, gray leaf spot, gray mold, heart rot, late blight, leaf blight, leaf blotch, leaf curl, leaf mold, leaf rust, leaf spot, mildew, necrosis, peronospora, phoma, pink mold, powdery mildew, rhizopus, root canker, root rot, rust, scab, smut, southern blight, stem canker, stem rot, verticillium, white mold, wildfire and yellows.

In some embodiments, the pest is a plant virus. Exemplary of such plant viruses are soybean mosaic virus, bean pod mottle virus, tobacco ring spot virus, barley yellow dwarf virus, wheat spindle streak virus, soil born mosaic virus, wheat streak virus in maize, maize dwarf mosaic virus, maize chlorotic dwarf virus, cucumber mosaic virus, tobacco mosaic virus, alfalfa mosaic virus, potato virus X, potato virus Y, potato leaf roll virus and tomato golden mosaic virus.

The methods of the present invention may be applied pre-harvest (i.e., during plant growth) or post-harvest, or may be applied to seeds or isolated plant cells or cell cultures, plant parts, and may be applied, for example, to leaves, flowers, seeds, roots, stems, or other plant tissues. The compositions comprising a Cas12a2 polypeptide or Cas12a2 fusion protein (or a polynucleotide encoding such) and/or at least one guide polynucleotide (or at least one polynucleotide encoding the at least one guide polynucleotide) of the disclosure may be applied as a spray on a plant, where the spray comprises a phage comprising a polynucleotide encoding a Cas12a2 polypeptide and at least one polynucleotide encoding at least one guide polynucleotide (e.g., guide RNA). In some embodiments, the compositions and methods of the present invention may be used to reduce the number of cells of a given bacterial strain or species, or to eliminate all or nearly all of the cells of a given bacterial strain or species. In some embodiments, the compositions and methods of the present invention may be used to selectively target and eliminate (in whole or in part) only those cells of a given bacterial strain or species that harbor certain target sequences, whether those sequences are present in the bacterial chromosomal genome, in plasmids, in viruses or phages that have infected or are otherwise present in the bacteria, or in other DNA- or RNA-containing components found in those bacterial cells.

### VIII. Enrichment of Cell Types

The compositions and methods of the present invention may be used to reduce or eliminate the presence of cells that comprise undesirable DNA or RNA sequence(s). In some embodiments, the compositions and methods of the present invention may be used to enrich for cells, cell lines, cell types, or other groupings of cells that do not comprise undesirable DNA or RNA sequence(s). Enrichment of certain cell types may be desirable, for example, following genome editing experiments or other editing experiments designed to modify certain known regions of a genome, other DNA molecule, or an RNA molecule. In such embodiments, the genome editing experiment may be performed to produce a desired genomic or other nucleic acid modification, resulting in a pool of cells in which a portion of the cells remain wild-type while a portion of the cells comprises the desired DNA or RNA sequence modification(s). The compositions and methods of the present invention may be used to target, through the appropriate design of guide RNA(s) or other guide polynucleotides designed to hybridize with wild-type, but not with modified sequences. Introduction of a Cas12a2 polypeptide, or encoding polynucleotide, along with one or more appropriately designed guide RNA(s) or encoding DNA molecules, into the pool of cells (for example through the use of engineered phages or phagemids, or through the use of conjugative plasmids), results in an initial hybridization event in cells that retain the undesirable wild-type DNA or RNA sequence(s). This initial hybridization event triggers secondary, collateral activity of the Cas12a2 enzyme targeted against dsDNA, ssDNA, dsRNA, and/or ssRNA, resulting in cell death among those cells that comprise the undesirable wild-type DNA or RNA sequence(s). The result of the targeted elimination of wild-type cells is the enrichment of cells in the cell pool that comprise the desired DNA or RNA sequence(s). Such experiments may be used, for example, to increase the likelihood of identifying and recovering cells that comprise a desirable allele or other genetic sequence, particularly in cases when such a desirable allele or other genetic sequence is relatively rare among the cells in the cell pool prior to introduction of the Cas12a2 polypeptide and guide RNA(s) or guide polynucleotides.

The article “a” and “an” are used herein to refer to one or more than one (i.e., to at least one) of the grammatical object of the article. By way of example, “a polypeptide” means one or more polypeptides.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

Embodiments of the invention include:

1. A composition comprising:
(i) a Cas12a2 polypeptide, or a polynucleotide encoding a Cas12a2 polypeptide, and
(ii) at least one guide polynucleotide, or a polynucleotide encoding the at least one guide polynucleotide,
   wherein each guide polynucleotide comprises: (a) a portion complementary to a target sequence of a plant pest, and (b) a portion capable of binding the Cas12a2 polypeptide,
   wherein the target sequence is located immediately adjacent to a PAM sequence that is recognized by the Cas12a2 polypeptide.

2. The composition of embodiment 1, wherein the Cas12a2 polypeptide comprises one or more conserved amino acid motifs selected from:
(a) a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I;
(b) a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V;
(c) a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V;
(d) a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁=I, L, or V; X₁₂=I, L, or F;
(e) a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid;
(f) a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F;
(g) a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K;
(h) a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T;
(i) a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K;
(j) a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V;
(k) a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A;
(l) a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T;
(m) a conserved motif 13 comprising the amino acid sequence set forth as X₁LX₃X₄X₅X₆X₇X₈KX₁₀GX₁₂X₁₃ANX₁₆X₁₇G X₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=J or V; X₁₉=V or I; X₂₀=J or V; X₂₁=A, V, or N; X₂₂=Y, F, or H;
(n) a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I;
(o) a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid;
(p) a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid; and/or
(q) a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N, and wherein the Cas12a2 polypeptide does not require a tracrRNA for function.

3. The composition of embodiment 1 or 2, wherein the plant pest comprises a microbe, a plant parasitic nematode, an insect, a fungus, a virus, a mollusk, a spider, a scorpion, a caterpillar, an animal, a mite, a tick, or a combination thereof.

4. The composition of embodiment 3, wherein the plant parasitic nematode comprises Aphelenchoides spp. (foliar nematodes), Caenorhabditis spp., Ditylenchus spp., Globodera spp. (potato cyst and golden nematodes), Heterodera spp. (soybean cyst nematodes), Longidorus spp., Meloidogyne spp. (root-knot nematodes), Nacobbus spp., Pratylenchus spp. (lesion nematodes), Trichodorus spp., Xiphinema spp. (dagger nematodes), Bursaphelenchus spp. (pine wood nematode), or a combination thereof.

5. The composition of embodiment 3 or 4, wherein the parasitic nematode comprises Caenorhabditis elegans.

6. The composition of embodiment 3 or 4, wherein the parasitic nematode comprises Heterodera glycines.

7. The composition of embodiment 3, wherein the insect comprises Western corn rootworm (Diabrotica virgifera virgifera), Northern corn rootworm (Diabrotica barberi), Southern corn rootworm (Diabrotica undecimpunctata howardi), fall armyworm (Spodoptera frugiperda), corn earworm (Helicoverpa zea), aphids (Aphis glycines), boll weevils (Anthonomus grandis), flower thrips, onion thrips, western flower thrips, beet armyworm (Spodoptera exigua), bollworms (Helicoverpa armigera), Colorado potato beetle (Leptinotarsa decemlineata), leafhopper (Empoasca fabae), dryland wireworm (Ctenicera pruinina), Pacific coast wireworm (Limonius canus), or sugarbeet wireworm (Limonius californicus).

8. The composition of embodiment 3, wherein the fungus comprises Fusarium verticillioides, Fusarium graminearum, soybean rust (Phakopsora pachyrhizi), or late blight (Phytophthora infestans).

9. The composition of embodiment 3, wherein the virus comprises soybean mosaic virus.

10. The composition of any one of embodiments 1-9, wherein the Cas12a2 polypeptide comprises an amino acid sequence having at least 80%, at least 85%, at least 90%, at least 95%, or 100% sequence identity to a sequence selected from the group consisting of SEQ ID NOs: 1-39, and 166, or wherein the polynucleotide encoding the Cas12a2 polypeptide comprises a nucleotide sequence having at least 80%, at least 85%, at least 90%, at least 95%, or 100% sequence identity to a sequence selected from the group consisting of SEQ ID NOs: 63-101, and 167.

11. The composition of any one of embodiments 1-10, wherein the Cas12a2 polypeptide comprises an amino acid sequence having at least 80%, at least 85%, at least 90%, at least 95%, or 100% sequence identity to a sequence selected from the group consisting of SEQ ID NOs: 28, 31, 32, 33, 34, 36, 37, 38, and 166, or wherein the polynucleotide encoding the Cas12a2 polypeptide comprises a nucleotide sequence having at least 80%, at least 85%, at least 90%, at least 95%, or 100% sequence identity to a sequence selected from the group consisting of SEQ ID NOs: 90, 93, 94, 95, 96, 98, 99, 100, and 167.

12. The composition of any one of embodiments 1-11, wherein the Cas12a2 polypeptide is operably fused to a cell-penetrating domain (CPP).

13. The composition of embodiment 12, wherein the CPP comprises a transactivating transcriptional activator (TAT), a transportan, or a penetratin.

14. The composition of embodiment 12 or 13, wherein the CPP is set forth as SEQ ID NO: 116, 117, or 160.

15. The composition of any one of embodiments 1-14, wherein the Cas12a2 polypeptide is operably fused to an export signal.

16. The composition of embodiment 15, wherein the export signal comprises PR1a.

17. The composition of embodiment 15 or 16, wherein the export signal is set forth as SEQ ID NO: 118.

18. The composition of any one of embodiments 1-17, wherein the Cas12a2 polypeptide is operably fused to a nuclear localization signal (NLS).

19. The composition of embodiment 18, wherein the NLS comprises an SV40 peptide or a nucleoplasmin.

20. The composition of embodiment 18 or 19, wherein the NLS is set forth as SEQ ID NO: 119 or 120.

21. The composition of any one of embodiments 12-20, wherein the Cas12a2 polypeptide is operably fused to a CPP, an export signal, and/or an NLS through a peptide linker.

22. The composition of embodiment 21, wherein the peptide linker is a glycine-serine linker.

23. The composition of embodiment 21 or 22, wherein the peptide linker is set forth as any one of SEQ ID NOs: 161, 168, 169, and 170.

24. The composition of embodiment 23, wherein the peptide linker is set forth as SEQ ID NO: 161.

25. The composition of any one of embodiments 12-24, wherein the CPP, the export signal, and/or the NLS is operably fused at the N-terminus or at the C-terminus of the Cas12a2 polypeptide.

26. The composition of any one of embodiments 12-24, wherein the CPP, the export signal, and/or the NLS is operably fused at an internal location of the Cas12a2 polypeptide.

27. The composition of any one of embodiments 15-17, wherein the operable fusion of the Cas12a2 polypeptide and the export signal is direct and not through a peptide linker.

28. The composition of any one of embodiments 1-27, wherein the at least one guide polynucleotide is at least one guide RNA.

29. The composition of embodiment 28, wherein the at least one guide RNA comprises a spacer capable of hybridizing to a target sequence within a C. elegans AGE1-1 gene, and wherein the spacer comprises a nucleotide sequence that differs in length and/or sequence from SEQ ID NO: 175 by 1 to 5 nucleotides.

30. The composition of embodiment 28, wherein the at least one guide RNA comprises a spacer capable of hybridizing to a target sequence within a C. elegans AGE1-1 gene, and wherein the spacer comprises the nucleotide sequence set forth as SEQ ID NO: 175.

31. The composition of embodiment 28, wherein the at least one guide RNA comprises a spacer capable of hybridizing to a target sequence within a C. elegans AGE1-2 gene, and wherein the spacer comprises a nucleotide sequence that differs in length and/or sequence from SEQ ID NO: 176 by 1 to 5 nucleotides.

32. The composition of embodiment 28, wherein the at least one guide RNA comprises a spacer capable of hybridizing to a target sequence within a C. elegans AGE1-2 gene, and wherein the spacer comprises the nucleotide sequence set forth as SEQ ID NO: 176.

33. The composition of embodiment 28, wherein the at least one guide RNA comprises a spacer capable of hybridizing to a target sequence within a C. elegans AGE1-7 gene, and wherein the spacer comprises a nucleotide sequence that differs in length and/or sequence from SEQ ID NO: 177 by 1 to 5 nucleotides.

34. The composition of embodiment 28, wherein the at least one guide RNA comprises a spacer capable of hybridizing to a target sequence within a C. elegans AGE1-7 gene, and wherein the spacer comprises the nucleotide sequence set forth as SEQ ID NO: 177.

35. The composition of embodiment 28, wherein the at least one guide RNA comprises a spacer capable of hybridizing to a target sequence within a C. elegans AKT1-2 gene, and wherein the spacer comprises a nucleotide sequence that differs in length and/or sequence from SEQ ID NO: 178 by 1 to 5 nucleotides.

36. The composition of embodiment 28, wherein the at least one guide RNA comprises a spacer capable of hybridizing to a target sequence within a C. elegans AKT1-2 gene, and wherein the spacer comprises the nucleotide sequence set forth as SEQ ID NO: 178.

37. The composition of embodiment 28, wherein the at least one guide RNA comprises a spacer capable of hybridizing to a target sequence within a C. elegans AKT1-5 gene, and wherein the spacer comprises a nucleotide sequence that differs in length and/or sequence from SEQ ID NO: 179 by 1 to 5 nucleotides.

38. The composition of embodiment 28, wherein the at least one guide RNA comprises a spacer capable of hybridizing to a target sequence within a C. elegans AKT1-5 gene, and wherein the spacer comprises the nucleotide sequence set forth as SEQ ID NO: 179.

39. The composition of embodiment 28, wherein the at least one guide RNA comprises a spacer capable of hybridizing to a target sequence within a C. elegans AKT1-8 gene, and wherein the spacer comprises a nucleotide sequence that differs in length and/or sequence from SEQ ID NO: 180 by 1 to 5 nucleotides.

40. The composition of embodiment 28, wherein the at least one guide RNA comprises a spacer capable of hybridizing to a target sequence within a C. elegans AKT1-8 gene, and wherein the spacer comprises the nucleotide sequence set forth as SEQ ID NO: 180.

41. The composition of embodiment 28, wherein the at least one guide RNA comprises a spacer capable of hybridizing to a target sequence within a P. syringae AvrE1-2 gene, and wherein the spacer comprises a nucleotide sequence that differs in length and/or sequence from SEQ ID NO: 184 by 1 to 5 nucleotides.

42. The composition of embodiment 28, wherein the at least one guide RNA comprises a spacer capable of hybridizing to a target sequence within a P. syringae AvrE1-2 gene, and wherein the spacer comprises the nucleotide sequence set forth as SEQ ID NO: 184.

43. The composition of embodiment 28, wherein the at least one guide RNA comprises a spacer capable of hybridizing to a target sequence within a P. syringae AvrE1-4 gene, and wherein the spacer comprises a nucleotide sequence that differs in length and/or sequence from SEQ ID NO: 185 by 1 to 5 nucleotides.

44. The composition of embodiment 28, wherein the at least one guide RNA comprises a spacer capable of hybridizing to a target sequence within a P. syringae AvrE1-4 gene, and wherein the spacer comprises the nucleotide sequence set forth as SEQ ID NO: 185.

45. The composition of embodiment 28, wherein the at least one guide RNA comprises a spacer capable of hybridizing to a target sequence within a P. syringae HopAA1-1 gene, and wherein the spacer comprises a nucleotide sequence that differs in length and/or sequence from SEQ ID NO: 186 by 1 to 5 nucleotides.

46. The composition of embodiment 28, wherein the at least one guide RNA comprises a spacer capable of hybridizing to a target sequence within a P. syringae HopAA1-1 gene, and wherein the spacer comprises the nucleotide sequence set forth as SEQ ID NO: 186.

47. The composition of embodiment 28, wherein the at least one guide RNA comprises a spacer capable of hybridizing to a target sequence within a P. syringae HopAA1-3 gene, and wherein the spacer comprises a nucleotide sequence that differs in length and/or sequence from SEQ ID NO: 187 by 1 to 5 nucleotides.

48. The composition of embodiment 28, wherein the at least one guide RNA comprises a spacer capable of hybridizing to a target sequence within a P. syringae HopAA1-3 gene, and wherein the spacer comprises the nucleotide sequence set forth as SEQ ID NO: 187.

49. The composition of embodiment 28, wherein the at least one guide RNA comprises a spacer capable of hybridizing to a target sequence within a P. infestans Avrblb1-2 gene, and wherein the spacer comprises a nucleotide sequence that differs in length and/or sequence from SEQ ID NO: 188 by 1 to 5 nucleotides.

50. The composition of embodiment 28, wherein the at least one guide RNA comprises a spacer capable of hybridizing to a target sequence within a P. infestans Avrblb1-2 gene, and wherein the spacer comprises the nucleotide sequence set forth as SEQ ID NO: 188.

51. The composition of embodiment 28, wherein the at least one guide RNA comprises a spacer capable of hybridizing to a target sequence within a P. infestans CRE8-1 gene, and wherein the spacer comprises a nucleotide sequence that differs in length and/or sequence from SEQ ID NO: 189 by 1 to 5 nucleotides.

52. The composition of embodiment 28, wherein the at least one guide RNA comprises a spacer capable of hybridizing to a target sequence within a P. infestans CRE8-1 gene, and wherein the spacer comprises the nucleotide sequence set forth as SEQ ID NO: 189.

53. The composition of embodiment 28, wherein the at least one guide RNA comprises a spacer capable of hybridizing to a target sequence within a Leptinotarsa decemlineata LdNA_17212-1 gene, and wherein the spacer comprises a nucleotide sequence that differs in length and/or sequence from SEQ ID NO: 190 by 1 to 5 nucleotides.

54. The composition of embodiment 28, wherein the at least one guide RNA comprises a spacer capable of hybridizing to a target sequence within a Leptinotarsa decemlineata LdNA_17212-1 gene, and wherein the spacer comprises the nucleotide sequence set forth as SEQ ID NO: 190.

55. The composition of embodiment 28, wherein the at least one guide RNA comprises a spacer capable of hybridizing to a target sequence within a Leptinotarsa decemlineata LdNA_31771-2 gene, and wherein the spacer comprises a nucleotide sequence that differs in length and/or sequence from SEQ ID NO: 191 by 1 to 5 nucleotides.

56. The composition of embodiment 28, wherein the at least one guide RNA comprises a spacer capable of hybridizing to a target sequence within a Leptinotarsa decemlineata LdNA_31771-2 gene, and wherein the spacer comprises the nucleotide sequence set forth as SEQ ID NO: 191.

57. The composition of any one of embodiments 1-56, wherein the at least one guide polynucleotide comprises at least 2, at least 3, at least 4, at least 5, at least 6, at least 7, at least 8, at least 9, or at least 10 guide guide polynucleotides.

58. The composition of embodiment 57, wherein the guide polynucleotides are capable of hybridizing to target sequences of one plant pest.

59. The composition of embodiment 57, wherein the guide polynucleotides are capable of hybridizing to target sequences of more than one plant pest.

60. The composition of any one of embodiments 1-59, wherein the composition comprises the Cas12a2 polypeptide and the at least one guide polynucleotide as a ribonucleoprotein complex.

61. The composition of any one of embodiments 1-59, wherein the polynucleotide encoding the Cas12a2 polypeptide is an RNA polynucleotide.

62. The composition of embodiment 61, wherein the RNA polynucleotide is an mRNA.

63. The composition of any one of embodiments 1-59, wherein the polynucleotide encoding a Cas12a2 polypeptide and the polynucleotide encoding the at least one guide polynucleotide are each operably linked to a promoter functional in a plant.

64. The composition of embodiment 63, wherein the promoter is a tissue-specific promoter.

65. The composition of embodiment 64, wherein the tissue-specific promoter is a seed-specific, a tuber-specific, a stem-specific, a pollen-specific, a root-specific, a leaf-specific, or a green tissue-specific promoter.

66. The composition of any one of embodiments 1-65, wherein the polynucleotide encoding a Cas12a2 polypeptide and the polynucleotide encoding the at least one guide polynucleotide are part of a vector.

67. The composition of embodiment 66, wherein the vector is a viral vector or a bacterial vector.

68. The composition of embodiment 67, wherein the viral vector is within a virus or the bacterial vector is within a bacterium.

69. The composition of any one of embodiments 1-68, wherein the target sequence of the plant pest is associated with viability, growth, development, infectivity, pathogenicity, or a combination thereof, of the plant pest.

70. The composition of any one of embodiments 1-69, wherein the target sequence is within a gene of C. elegans, P. syringae, P. infestans, or L. decemlineata.

71. The composition of embodiment 70, wherein the C. elegans gene comprises AGE1-1, AGE1-2, AGE1-7, AKT1-2, AKT1-5, AKT1-8, or a combination thereof.

72. The composition of embodiment 71, wherein the C. elegans target sequence is set forth as any one of SEQ ID NOs: 103-108.

73. The composition of embodiment 70, wherein the P. syringae gene comprises AvrE1-2, AvrE1-4, HopAA1-1, HopAA1-3, or a combination thereof.

74. The composition of embodiment 73, wherein the P. syringae target sequence is set forth as any one of SEQ ID NOs: 112-115.

75. The composition of embodiment 70, wherein the P. infestans gene comprises Avrblb1-2 or CRE8-1.

76. The composition of embodiment 75, wherein the P. infestans target sequence is set forth as SEQ ID NO: 162 or 163.

77. The composition of embodiment 70, wherein the L. decemlineata gene comprises LdNA 17212-1 or LdNA 31771-2.

78. The composition of embodiment 77, wherein the L. decemlineata target sequence is set forth as SEQ ID NO: 164 or 165.

79. A plant, plant part, plant cell, or population of plants comprising the composition of any one of embodiments 1-78.

80. A method for increasing resistance or tolerance of a plant to one or more plant pest, the method comprising: introducing into a plant, plant part, or plant cell a composition comprising:
(a) a Cas12a2 polypeptide, or a polynucleotide encoding a Cas12a2 polypeptide, and
(b) at least one guide polynucleotide, or a polynucleotide encoding at least one guide polynucleotide,
   to produce a modified plant, plant part, or plant cell with increased resistance or tolerance to one or more plant pest, as compared to resistance or tolerance of a control plant to the one or more plant pest,
   wherein each guide polynucleotide is capable of binding the Cas12a2 polypeptide and hybridizing to a target sequence in one or more cells of each corresponding plant pest,
   wherein the target sequence is located immediately adjacent to a PAM sequence that is recognized by the Cas12a2 polypeptide,
   and wherein the Cas12a2 polypeptide comprises
(i) a primary activity of cleaving the target sequence;
(ii) a secondary activity of cleaving nucleic acid molecules in a non-sequence-specific manner in the one or more cells of the one or more plant pest.

81. The method of embodiment 80, wherein the Cas12a2 polypeptide comprises one or more conserved amino acid motifs selected from:
(a) a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I;
(b) a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V;
(c) a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V;
(d) a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F;
(e) a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid;
(f) a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F;
(g) a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K;
(h) a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T;
(i) a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K;
(j) a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V;
(k) a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A;
(l) a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T;
(m) a conserved motif 13 comprising the amino acid sequence set forth as X₁LX₃X₄X₅X₆X₇X₈KX₁₀GX₁₂X₁₃ANX₁₆X₁₇G X₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H;
(n) a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I;
(o) a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid;
(p) a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid; and/or
(q) a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N, and wherein the Cas12a2 polypeptide does not require a tracrRNA for function.

54. A method for producing a modified plant with increased resistance or tolerance to one or more plant pest, the method comprising: introducing into a plant, plant part, or plant cell a composition comprising:
(a) a Cas12a2 polypeptide, or a polynucleotide encoding a Cas12a2 polypeptide, and
(b) at least one guide polynucleotide, or a polynucleotide encoding at least one guide polynucleotide; and
   selecting for a modified plant, plant part, or plant cell that expresses the Cas12a2 polypeptide and the at least one guide polynucleotide,
   wherein each guide polynucleotide is capable of binding the Cas12a2 polypeptide and hybridizing to a target sequence in one or more cells of each corresponding plant pest,
   wherein the target sequence is located immediately adjacent to a PAM sequence that is recognized by the Cas12a2 polypeptide,
   and wherein the Cas12a2 polypeptide comprises
(i) a primary activity of cleaving the target sequence;
(ii) a secondary activity of cleaving nucleic acid molecules in a non-sequence-specific manner in the one or more cells of the one or more plant pest,
   thereby producing a modified plant with increased resistance or tolerance to the one or more plant pest, as compared to resistance or tolerance of a control plant to the one or more plant pest.

55. The method of embodiment 24, wherein the Cas12a2 polypeptide comprises one or more conserved amino acid motifs selected from:
(a) a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I;
(b) a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V;
(c) a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V;
(d) a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F;
(e) a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid;
(f) a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F;
(g) a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K;
(h) a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T;
(i) a conserved motif 9 comprising the amino acid sequence set forth as X₁RX₃X₄X₅X₆X₇X₈X₉X₁₀X₁₁X₁₂X₁₃X₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K;
j) a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V;
(k) a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A;
(l) a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T;
(m) a conserved motif 13 comprising the amino acid sequence set forth as X₁LX₃X₄X₅X₆X₇X₈KX₁₀GX₁₂X₁₃ANX₁₆X₁₇G X₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=J or V; X₁₉=V or I; X₂₀=J or V; X₂₁=A, V, or N; X₂₂=Y, F, or H;
(n) a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I;
(o) a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid;
(p) a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid; and/or
(q) a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N, and wherein the Cas12a2 polypeptide does not require a tracrRNA for function.

82. The method of embodiment 80 or 81, wherein the selecting comprises growing the plant, plant part, or plant cell in media comprising a selectable agent.

83. The method of embodiment 82, wherein the selectable agent is an herbicide, an antibiotic, a carbohydrate, an amino acid, or a metabolite.

84. The method of any one of embodiments 80-83, wherein the control plant is a corresponding plant or population of plants that does not comprise the composition.

85. The method of any one of embodiments 80-84, wherein the one or more plant pest comprises a microbe, a plant parasitic nematode, an insect, a fungus, a virus, a mollusk, a spider, a scorpion, a caterpillar, an animal, a mite, a tick, or a combination thereof.

86. The method of embodiment 85, wherein the plant parasitic nematode comprises Aphelenchoides spp. (foliar nematodes), Caenorhabditis spp., Ditylenchus spp., Globodera spp. (potato cyst and golden nematodes), Heterodera spp. (soybean cyst nematodes), Longidorus spp., Meloidogyne spp. (root-knot nematodes), Nacobbus spp., Pratylenchus spp. (lesion nematodes), Trichodorus spp., Xiphinema spp. (dagger nematodes), Bursaphelenchus spp. (pine wood nematode), or a combination thereof.

87. The method of embodiment 85 or 86, wherein the plant parasitic nematode comprises Caenorhabditis elegans.

88. The method of embodiment 85 or 86, wherein the parasitic nematode comprises Heterodera glycines.

89. The method of embodiment 85, wherein the insect comprises Western corn rootworm (Diabrotica virgifera virgifera), Northern corn rootworm (Diabrotica barberi), Southern corn rootworm (Diabrotica undecimpunctata howardi), fall armyworm (Spodoptera frugiperda), corn earworm (Helicoverpa zea), aphids (Aphis glycines), boll weevils (Anthonomus grandis), flower thrips, onion thrips, western flower thrips, beet armyworm (Spodoptera exigua), bollworms (Helicoverpa armigera), Colorado potato beetle (Leptinotarsa decemlineata), leafhopper (Empoasca fabae), dryland wireworm (Ctenicera pruinina), Pacific coast wireworm (Limonius canus), or sugarbeet wireworm (Limonius californicus).

90. The method of embodiment 85, wherein the fungus comprises Fusarium verticillioides, Fusarium graminearum, soybean rust (Phakopsora pachyrhizi), or late blight (Phytophthora infestans).

91. The method of embodiment 85, wherein the virus comprises soybean mosaic virus.

92. The method of any one of embodiments 80-91, wherein the modified plant comprises improved plant performance as compared to the control plant.

93. The method of embodiment 92, wherein the improved plant performance comprises increased biomass yield and/or seed yield.

94. The method of any one of embodiments 80-93, wherein the Cas12a2 polypeptide comprises an amino acid sequence having at least 80%, at least 85%, at least 90%, at least 95%, or 100% sequence identity to a sequence selected from the group consisting of SEQ ID NOs: 1-39, and 166, or wherein the polynucleotide encoding the Cas12a2 polypeptide comprises a nucleotide sequence having at least 80%, at least 85%, at least 90%, at least 95%, or 100% sequence identity to a sequence selected from the group consisting of SEQ ID NOs: 63-101, and 167.

95. The method of any one of embodiments 80-94, wherein the Cas12a2 polypeptide comprises an amino acid sequence having at least 80%, at least 85%, at least 90%, at least 95%, or 100% sequence identity to a sequence selected from the group consisting of SEQ ID NOs: 28, 31, 32, 33, 34, 36, 37, 38, and 166, or wherein the polynucleotide encoding the Cas12a2 polypeptide comprises a nucleotide sequence having at least 80%, at least 85%, at least 90%, at least 95%, or 100% sequence identity to a sequence selected from the group consisting of SEQ ID NOs: 90, 93, 94, 95, 96, 98, 99, 100, and 167.

96. The method of any one of embodiments 80-95, wherein the Cas12a2 polypeptide is operably fused to a cell-penetrating domain (CPP).

97. The method of embodiment 96, wherein the CPP comprises a transactivating transcriptional activator (TAT), a transportan, or a penetratin.

98. The method of embodiment 96 or 97, wherein the CPP is set forth as SEQ ID NO: 116, 117, or 160.

99. The method of any one of embodiments 80-98, wherein the Cas12a2 polypeptide is operably fused to an export signal.

100. The method of embodiment 99, wherein the export signal comprises PR1a.

101. The method of embodiment 99 or 100, wherein the export signal is set forth as SEQ ID NO: 118.

102. The method of any one of embodiments 80-101, wherein the Cas12a2 polypeptide is operably fused to a nuclear localization signal (NLS).

103. The method of embodiment 102, wherein the NLS comprises an SV40 peptide or a nucleoplasmin.

104. The method of embodiment 102 or 103, wherein the NLS is set forth as SEQ ID NO: 119 or 120.

105. The method of any one of embodiments 96-104, wherein the Cas12a2 polypeptide is operably fused to a CPP, an export signal, and/or an NLS through a peptide linker.

106. The method of embodiment 105, wherein the peptide linker is a glycine-serine linker.

107. The method of embodiment 105 or 106, wherein the peptide linker is set forth as any one of SEQ ID NOs: 161, 168, 169, and 170.

108. The method of embodiment 107, wherein the peptide linker is set forth as SEQ ID NO: 161.

109. The method of any one of embodiments 96-108, wherein the CPP, the export signal, and/or the NLS is operably fused at the N-terminus or at the C-terminus of the Cas12a2 polypeptide.

110. The method of any one of embodiments 96-108, wherein the CPP, the export signal, and/or the NLS is operably fused at an internal location of the Cas12a2 polypeptide.

111. The method of any one of embodiments 99-101, wherein the operable fusion of the Cas12a2 polypeptide and the export signal is direct and not through a peptide linker.

112. The method of any one of embodiments 80-111, wherein the at least one guide polynucleotide is at least one guide RNA.

113. The method of embodiment 112, wherein the at least one guide RNA comprises a spacer capable of hybridizing to a target sequence within a C. elegans AGE1-1 gene, and wherein the spacer comprises a nucleotide sequence that differs in length and/or sequence from SEQ ID NO: 175 by 1 to 5 nucleotides.

114. The method of embodiment 112, wherein the at least one guide RNA comprises a spacer capable of hybridizing to a target sequence within a C. elegans AGE1-1 gene, and wherein the spacer comprises the nucleotide sequence set forth as SEQ ID NO: 175.

115. The method of embodiment 112, wherein the at least one guide RNA comprises a spacer capable of hybridizing to a target sequence within a C. elegans AGE1-2 gene, and wherein the spacer comprises a nucleotide sequence that differs in length and/or sequence from SEQ ID NO: 176 by 1 to 5 nucleotides.

116. The method of embodiment 112, wherein the at least one guide RNA comprises a spacer capable of hybridizing to a target sequence within a C. elegans AGE1-2 gene, and wherein the spacer comprises the nucleotide sequence set forth as SEQ ID NO: 176.

117. The method of embodiment 112, wherein the at least one guide RNA comprises a spacer capable of hybridizing to a target sequence within a C. elegans AGE1-7 gene, and wherein the spacer comprises a nucleotide sequence that differs in length and/or sequence from SEQ ID NO: 177 by 1 to 5 nucleotides.

118. The method of embodiment 112, wherein the at least one guide RNA comprises a spacer capable of hybridizing to a target sequence within a C. elegans AGE1-7 gene, and wherein the spacer comprises the nucleotide sequence set forth as SEQ ID NO: 177.

119. The method of embodiment 112, wherein the at least one guide RNA comprises a spacer capable of hybridizing to a target sequence within a C. elegans AKT1-2 gene, and wherein the spacer comprises a nucleotide sequence that differs in length and/or sequence from SEQ ID NO: 178 by 1 to 5 nucleotides.

120. The method of embodiment 112, wherein the at least one guide RNA comprises a spacer capable of hybridizing to a target sequence within a C. elegans AKT1-2 gene, and wherein the spacer comprises the nucleotide sequence set forth as SEQ ID NO: 178.

121. The method of embodiment 112, wherein the at least one guide RNA comprises a spacer capable of hybridizing to a target sequence within a C. elegans AKT1-5 gene, and wherein the spacer comprises a nucleotide sequence that differs in length and/or sequence from SEQ ID NO: 179 by 1 to 5 nucleotides.

122. The method of embodiment 112, wherein the at least one guide RNA comprises a spacer capable of hybridizing to a target sequence within a C. elegans AKT1-5 gene, and wherein the spacer comprises the nucleotide sequence set forth as SEQ ID NO: 179.

123. The method of embodiment 112, wherein the at least one guide RNA comprises a spacer capable of hybridizing to a target sequence within a C. elegans AKT1-8 gene, and wherein the spacer comprises a nucleotide sequence that differs in length and/or sequence from SEQ ID NO: 180 by 1 to 5 nucleotides.

124. The method of embodiment 112, wherein the at least one guide RNA comprises a spacer capable of hybridizing to a target sequence within a C. elegans AKT1-8 gene, and wherein the spacer comprises the nucleotide sequence set forth as SEQ ID NO: 180.

125. The method of embodiment 112, wherein the at least one guide RNA comprises a spacer capable of hybridizing to a target sequence within a P. syringae AvrE1-2 gene, and wherein the spacer comprises a nucleotide sequence that differs in length and/or sequence from SEQ ID NO: 184 by 1 to 5 nucleotides.

126. The method of embodiment 112, wherein the at least one guide RNA comprises a spacer capable of hybridizing to a target sequence within a P. syringae AvrE1-2 gene, and wherein the spacer comprises the nucleotide sequence set forth as SEQ ID NO: 184.

127. The method of embodiment 112, wherein the at least one guide RNA comprises a spacer capable of hybridizing to a target sequence within a P. syringae AvrE1-4 gene, and wherein the spacer comprises a nucleotide sequence that differs in length and/or sequence from SEQ ID NO: 185 by 1 to 5 nucleotides.

128. The method of embodiment 112, wherein the at least one guide RNA comprises a spacer capable of hybridizing to a target sequence within a P. syringae AvrE1-4 gene, and wherein the spacer comprises the nucleotide sequence set forth as SEQ ID NO: 185.

129. The method of embodiment 112, wherein the at least one guide RNA comprises a spacer capable of hybridizing to a target sequence within a P. syringae HopAA1-1 gene, and wherein the spacer comprises a nucleotide sequence that differs in length and/or sequence from SEQ ID NO: 186 by 1 to 5 nucleotides.

130. The method of embodiment 112, wherein the at least one guide RNA comprises a spacer capable of hybridizing to a target sequence within a P. syringae HopAA1-1 gene, and wherein the spacer comprises the nucleotide sequence set forth as SEQ ID NO: 186.

131. The method of embodiment 112, wherein the at least one guide RNA comprises a spacer capable of hybridizing to a target sequence within a P. syringae HopAA1-3 gene, and wherein the spacer comprises a nucleotide sequence that differs in length and/or sequence from SEQ ID NO: 187 by 1 to 5 nucleotides.

132. The method of embodiment 112, wherein the at least one guide RNA comprises a spacer capable of hybridizing to a target sequence within a P. syringae HopAA1-3 gene, and wherein the spacer comprises the nucleotide sequence set forth as SEQ ID NO: 187.

133. The method of embodiment 112, wherein the at least one guide RNA comprises a spacer capable of hybridizing to a target sequence within a P. infestans Avrblbl-2 gene, and wherein the spacer comprises a nucleotide sequence that differs in length and/or sequence from SEQ ID NO: 188 by 1 to 5 nucleotides.

134. The method of embodiment 112, wherein the at least one guide RNA comprises a spacer capable of hybridizing to a target sequence within a P. infestans Avrblbl-2 gene, and wherein the spacer comprises the nucleotide sequence set forth as SEQ ID NO: 188.

135. The method of embodiment 112, wherein the at least one guide RNA comprises a spacer capable of hybridizing to a target sequence within a P. infestans CRE8-1 gene, and wherein the spacer comprises a nucleotide sequence that differs in length and/or sequence from SEQ ID NO: 189 by 1 to 5 nucleotides.

136. The method of embodiment 112, wherein the at least one guide RNA comprises a spacer capable of hybridizing to a target sequence within a P. infestans CRE8-1 gene, and wherein the spacer comprises the nucleotide sequence set forth as SEQ ID NO: 189.

137. The method of embodiment 112, wherein the at least one guide RNA comprises a spacer capable of hybridizing to a target sequence within a Leptinotarsa decemlineata LdNA_17212-1 gene, and wherein the spacer comprises a nucleotide sequence that differs in length and/or sequence from SEQ ID NO: 190 by 1 to 5 nucleotides.

138. The method of embodiment 112, wherein the at least one guide RNA comprises a spacer capable of hybridizing to a target sequence within a Leptinotarsa decemlineata LdNA_17212-1 gene, and wherein the spacer comprises the nucleotide sequence set forth as SEQ ID NO: 190.

139. The method of embodiment 112, wherein the at least one guide RNA comprises a spacer capable of hybridizing to a target sequence within a Leptinotarsa decemlineata LdNA_31771-2 gene, and wherein the spacer comprises a nucleotide sequence that differs in length and/or sequence from SEQ ID NO: 191 by 1 to 5 nucleotides.

140. The method of embodiment 112, wherein the at least one guide RNA comprises a spacer capable of hybridizing to a target sequence within a Leptinotarsa decemlineata LdNA_31771-2 gene, and wherein the spacer comprises the nucleotide sequence set forth as SEQ ID NO: 191.

141. The method of any one of embodiments 80-140, wherein the at least one guide polynucleotide comprises at least 2, at least 3, at least 4, at least 5, at least 6, at least 7, at least 8, at least 9, or at least 10 guide polynucleotides.

142. The method of embodiment 141, wherein the guide polynucleotides are capable of hybridizing to target sequences of one plant pest.

143. The method of embodiment 141, wherein the guide polynucleotides are capable of hybridizing to target sequences of more than one plant pest.

144. The method of any one of embodiments 80-143, wherein the composition comprises the Cas12a2 polypeptide and the at least one guide polynucleotide as a ribonucleoprotein complex.

145. The method of any one of embodiments 80-143, wherein the polynucleotide encoding the Cas12a2 polypeptide is an RNA polynucleotide.

146. The method of embodiment 145, wherein the RNA polynucleotide is an mRNA.

147. The method of any one of embodiments 80-146, wherein the introducing comprises introducing into the plant cell, and culturing the plant cell to regenerate a plant or plant part comprising the composition.

148. The method of any one of embodiments 80-147, wherein the introducing comprises contacting with a virus or viral nucleic acid molecule comprising the composition, microinjection, electroporation, Agrobacterium-mediated transformation, direct gene transfer, particle mediated delivery, topical application, silicon carbide fiber mediated delivery, delivery via cell-penetrating peptides, or a combination thereof.

149. The method of any one of embodiments 80-147, wherein the polynucleotide encoding a Cas12a2 polypeptide and the polynucleotide encoding the at least one guide polynucleotide are part of a vector.

150. The method of embodiment 149, wherein the vector is a viral vector or a bacterial vector.

151. The method of embodiment 150, wherein the viral vector is within a virus or the bacterial vector is within a bacterium.

152. The method of any one of embodiments 80-151, wherein the PAM sequence that is recognized by the Cas12a2 polypeptide is selected from the group consisting of TTAA, TTAC, TTAG, TTCA, TTCC, TTGG, TTGA, TTGC, TTGG, TTTA, TTTC, TTTG, ATTA, ATTC, ATTG, CTTA, CTTC, CTTG, GTTA, GTTC, GTTG, TCTA, TCTC, TCTG, NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.

153. The method of any one of embodiments 80-152, wherein each target sequence is associated with viability, growth, development, infectivity, pathogenicity, or a combination thereof, of the corresponding plant pest.

154. The method of embodiment 153, wherein the target sequence is within a gene of C. elegans, P. syringae, P. infestans, or L. decemlineata.

155. The method of embodiment 154, wherein the C. elegans gene comprises AGE1-1, AGE1-2, AGE1-7, AKT1-2, AKT1-5, AKT1-8, or a combination thereof.

156. The method of embodiment 155, wherein the C. elegans target sequence is set forth as any one of SEQ ID NOs: 103-108.

157. The method of embodiment 153, wherein the P. syringae gene comprises AvrE1-2, AvrE1-4, HopAA1-1, HopAA1-3, or a combination thereof.

158. The method of embodiment 157, wherein the P. syringae target sequence is set forth as any one of SEQ ID NOs: 112-115.

159. The method of embodiment 153, wherein the P. infestans gene comprises Avrblb1-2 or CRE8-1.

160. The method of embodiment 159, wherein the P. infestans target sequence is set forth as SEQ ID NO: 162 or 163.

161. The method of embodiment 153, wherein the L. decemlineata gene comprises LdNA_17212-1 or LdNA_31771-2.

162. The method of embodiment 161, wherein the L. decemlineata target sequence is set forth as SEQ ID NO: 164 or 165.

163. The method of any one of embodiments 80-162, wherein the plant, plant part, or plant cell is soybean (Glycine max), corn (Zea mays), Brassica species, Brassica napus, Brassica rapa, Brassica juncea, rice (Oryza sativa), rye (Secale cereale), sorghum (Sorghum bicolor, Sorghum vulgare), millet, pearl millet (Pennisetum glaucum), proso millet (Panicum miliaceum), foxtail millet (Setaria italica), finger millet (Eleusine coracana), sunflower (Helianthus annuus), safflower (Carthamus tinctorius), wheat (Triticum aestivum), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, flowers, and conifers.

164. A modified plant produced by the method of any one of embodiments 80-163.

165. A method of reducing an amount of a plant pest, the method comprising: contacting the plant pest with a composition comprising:
(a) a Cas12a2 polypeptide, or a polynucleotide encoding a Cas12a2 polypeptide, and
(b) at least one guide polynucleotide, or a polynucleotide encoding at least one guide polynucleotide;
   wherein each guide polynucleotide is capable of binding the Cas12a2 polypeptide and hybridizing to a target sequence in one or more cells of the plant pest,
   wherein the target sequence is located immediately adjacent to a PAM sequence that is recognized by the Cas12a2 polypeptide,
   and wherein the Cas12a2 polypeptide comprises
(i) a primary activity of cleaving the target sequence;
(ii) a secondary activity of cleaving nucleic acid molecules in a non-sequence-specific manner in the one or more cells of the plant pest, thereby reducing the amount of the plant pest.

166. The method of embodiment 165, wherein a population of the plant pest is reduced as compared to a corresponding population of the plant pest that has not been contacted with the composition.

167. The method of embodiment 166, wherein the reduction in population of the plant pest is a reduction in colony forming units of the plant pest.

168. The method of embodiment 165, wherein a population of the plant pest is eliminated.

169. The method of any one of embodiments 165-168, wherein the method further comprises selecting for individuals or cells of the plant pest that comprise (a) and/or (b).

170. The method of embodiment 169, wherein the selecting comprises culturing or growing the plant pest in media comprising an herbicide, an antibiotic, a carbohydrate, an amino acid, or a metabolite.

171. The method of any one of embodiments 165-170, wherein the plant pest that has been contacted with the composition has reduced ability to infect or destroy a plant.

172. The method of any one of embodiments 165-171, wherein the Cas12a2 polypeptide comprises one or more conserved amino acid motifs selected from:
(a) a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I;
(b) a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V;
(c) a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V;
(d) a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F;
(e) a conserved motif 5 comprising the amino acid sequence set forth as X₁X₂X₃X₄SX₆TSX₉X₁₀X₁₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid;
(f) a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F;
(g) a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K;
(h) a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T;
(i) a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K;
(j) a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V;
(k) a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A;
(l) a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T;
(m) a conserved motif 13 comprising the amino acid sequence set forth as X₁LX₃X₄X₅X₆X₇X₈KX₁₀GX₁₂X₁₃ANX₁₆X₁₇G X₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H;
(n) a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I;
(o) a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid;
(p) a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid; and/or
(q) a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N,
and wherein the Cas12a2 polypeptide does not require a tracrRNA for function.

173. The method any one of embodiments 165-172, wherein the plant pest comprises a microbe, a plant parasitic nematode, an insect, a fungus, a virus, a mollusk, a spider, a scorpion, a caterpillar, an animal, a mite, a tick, or a combination thereof.

174. The method of embodiment 173, wherein the plant parasitic nematode comprises Aphelenchoides spp. (foliar nematodes), Caenorhabditis spp., Ditylenchus spp., Globodera spp. (potato cyst and golden nematodes), Heterodera spp. (soybean cyst nematodes), Longidorus spp., Meloidogyne (root-knot nematodes) spp., Nacobbus spp., Pratylenchus spp. (lesion nematodes), Trichodorus spp., Xiphinema spp. (dagger nematodes), Bursaphelenchus spp. (pine wood nematode), or a combination thereof.

175. The method of embodiment 173 or 174, wherein the parasitic nematode comprises Caenorhabditis elegans or Heterodera glycines.

176. The method of embodiment 173, wherein the insect comprises Western corn rootworm (Diabrotica virgifera virgifera), Northern corn rootworm (Diabrotica barberi), Southern corn rootworm (Diabrotica undecimpunctata howardi), fall armyworm (Spodoptera frugiperda), corn earworm (Helicoverpa zea), aphids (Aphis glycines), boll weevils (Anthonomus grandis), flower thrips, onion thrips, western flower thrips, beet armyworm (Spodoptera exigua), bollworms (Helicoverpa armigera), Colorado potato beetle (Leptinotarsa decemlineata), leafhopper (Empoasca fabae), dryland wireworm (Ctenicera pruinina), Pacific coast wireworm (Limonius canus), or sugarbeet wireworm (Limonius californicus).

177. The method of embodiment 173, wherein the fungus comprises Fusarium verticillioides, Fusarium graminearum, soybean rust (Phakopsora pachyrhizi), or late blight (Phytophthora infestans).

178. The method of embodiment 173, wherein the virus comprises soybean mosaic virus.

179. The method of any one of embodiments 165-178, wherein the Cas12a2 polypeptide comprises an amino acid sequence having at least 80%, at least 85%, at least 90%, at least 95%, or 100% sequence identity to a sequence selected from the group consisting of SEQ ID NOs: 1-39, and 166, or wherein the polynucleotide encoding the Cas12a2 polypeptide comprises a nucleotide sequence having at least 80%, at least 85%, at least 90%, at least 95%, or 100% sequence identity to a sequence selected from the group consisting of SEQ ID NOs: 63-101, and 167.

180. The method of any one of embodiments 165-179, wherein the Cas12a2 polypeptide comprises an amino acid sequence having at least 80%, at least 85%, at least 90%, at least 95%, or 100% sequence identity to a sequence selected from the group consisting of SEQ ID NOs: 28, 31, 32, 33, 34, 36, 37, 38, and 166, or wherein the polynucleotide encoding the Cas12a2 polypeptide comprises a nucleotide sequence having at least 80%, at least 85%, at least 90%, at least 95%, or 100% sequence identity to a sequence selected from the group consisting of SEQ ID NOs: 90, 93, 94, 95, 96, 98, 99, 100, and 167.

181. The method of any one of embodiments 165-180, wherein the PAM sequence that is recognized by the Cas12a2 polypeptide is selected from the group consisting of TTAA, TTAC, TTAG, TTCA, TTCC, TTGG, TTGA, TTGC, TTGG, TTTA, TTTC, TTTG, ATTA, ATTC, ATTG, CTTA, CTTC, CTTG, GTTA, GTTC, GTTG, TCTA, TCTC, TCTG, NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.

182. The method of any one of embodiments 165-181, wherein the target sequence is associated with viability, growth, development, infectivity, pathogenicity, or a combination thereof, of the plant pest.

183. The method of embodiment 182, wherein the target sequence is within a gene of C. elegans, P. syringae, P. infestans, or L. decemlineata.

184. The method of embodiment 183, wherein the C. elegans gene comprises AGE1-1, AGE1-2, AGE1-7, AKT1-2, AKT1-5, AKT1-8, or a combination thereof.

185. The method of embodiment 184, wherein the C. elegans target sequence is set forth as any one of SEQ ID NOs: 103-108.

186. The method of embodiment 183, wherein the P. syringae gene comprises AvrE1-2, AvrE1-4, HopAA1-1, HopAA1-3, or a combination thereof.

187. The method of embodiment 186, wherein the P. syringae target sequence is set forth as any one of SEQ ID NOs: 112-115.

188. The method of embodiment 183, wherein the P. infestans gene comprises Avrblb1-2 or CRE8-1.

189. The method of embodiment 188, wherein the P. infestans target sequence is set forth as SEQ ID NO: 162 or 163.

190. The method of embodiment 129, wherein the L. decemlineata gene comprises LdNA_17212-1 or LdNA_31771-2.

191. The method of embodiment 136, wherein the L. decemlineata target sequence is set forth as SEQ ID NO: 164 or 165.

192. The method of any one of embodiments 165-191, wherein the at least one guide polynucleotide is at least one guide RNA.

193. The method of embodiment 192, wherein the at least one guide RNA comprises a spacer capable of hybridizing to a target sequence within a C. elegans AGE1-1 gene, and wherein the spacer comprises a nucleotide sequence that differs in length and/or sequence from SEQ ID NO: 175 by 1 to 5 nucleotides.

194. The method of embodiment 192, wherein the at least one guide RNA comprises a spacer capable of hybridizing to a target sequence within a C. elegans AGE1-1 gene, and wherein the spacer comprises the nucleotide sequence set forth as SEQ ID NO: 175.

195. The method of embodiment 192, wherein the at least one guide RNA comprises a spacer capable of hybridizing to a target sequence within a C. elegans AGE1-2 gene, and wherein the spacer comprises a nucleotide sequence that differs in length and/or sequence from SEQ ID NO: 176 by 1 to 5 nucleotides.

196. The method of embodiment 192, wherein the at least one guide RNA comprises a spacer capable of hybridizing to a target sequence within a C. elegans AGE1-2 gene, and wherein the spacer comprises the nucleotide sequence set forth as SEQ ID NO: 176.

197. The method of embodiment 192, wherein the at least one guide RNA comprises a spacer capable of hybridizing to a target sequence within a C. elegans AGE1-7 gene, and wherein the spacer comprises a nucleotide sequence that differs in length and/or sequence from SEQ ID NO: 177 by 1 to 5 nucleotides.

198. The method of embodiment 192, wherein the at least one guide RNA comprises a spacer capable of hybridizing to a target sequence within a C. elegans AGE1-7 gene, and wherein the spacer comprises the nucleotide sequence set forth as SEQ ID NO: 177.

199. The method of embodiment 192, wherein the at least one guide RNA comprises a spacer capable of hybridizing to a target sequence within a C. elegans AKT1-2 gene, and wherein the spacer comprises a nucleotide sequence that differs in length and/or sequence from SEQ ID NO: 178 by 1 to 5 nucleotides.

200. The method of embodiment 192, wherein the at least one guide RNA comprises a spacer capable of hybridizing to a target sequence within a C. elegans AKT1-2 gene, and wherein the spacer comprises the nucleotide sequence set forth as SEQ ID NO: 178.

201. The method of embodiment 192, wherein the at least one guide RNA comprises a spacer capable of hybridizing to a target sequence within a C. elegans AKT1-5 gene, and wherein the spacer comprises a nucleotide sequence that differs in length and/or sequence from SEQ ID NO: 179 by 1 to 5 nucleotides.

202. The method of embodiment 192, wherein the at least one guide RNA comprises a spacer capable of hybridizing to a target sequence within a C. elegans AKT1-5 gene, and wherein the spacer comprises the nucleotide sequence set forth as SEQ ID NO: 179.

203. The method of embodiment 192, wherein the at least one guide RNA comprises a spacer capable of hybridizing to a target sequence within a C. elegans AKT1-8 gene, and wherein the spacer comprises a nucleotide sequence that differs in length and/or sequence from SEQ ID NO: 180 by 1 to 5 nucleotides.

204. The method of embodiment 192, wherein the at least one guide RNA comprises a spacer capable of hybridizing to a target sequence within a C. elegans AKT1-8 gene, and wherein the spacer comprises the nucleotide sequence set forth as SEQ ID NO: 180.

205. The method of embodiment 192, wherein the at least one guide RNA comprises a spacer capable of hybridizing to a target sequence within a P. syringae AvrE1-2 gene, and wherein the spacer comprises a nucleotide sequence that differs in length and/or sequence from SEQ ID NO: 184 by 1 to 5 nucleotides.

206. The method of embodiment 192, wherein the at least one guide RNA comprises a spacer capable of hybridizing to a target sequence within a P. syringae AvrE1-2 gene, and wherein the spacer comprises the nucleotide sequence set forth as SEQ ID NO: 184.

207. The method of embodiment 192, wherein the at least one guide RNA comprises a spacer capable of hybridizing to a target sequence within a P. syringae AvrE1-4 gene, and wherein the spacer comprises a nucleotide sequence that differs in length and/or sequence from SEQ ID NO: 185 by 1 to 5 nucleotides.

208. The method of embodiment 192, wherein the at least one guide RNA comprises a spacer capable of hybridizing to a target sequence within a P. syringae AvrE1-4 gene, and wherein the spacer comprises the nucleotide sequence set forth as SEQ ID NO: 185.

209. The method of embodiment 192, wherein the at least one guide RNA comprises a spacer capable of hybridizing to a target sequence within a P. syringae HopAA1-1 gene, and wherein the spacer comprises a nucleotide sequence that differs in length and/or sequence from SEQ ID NO: 186 by 1 to 5 nucleotides.

210. The method of embodiment 192, wherein the at least one guide RNA comprises a spacer capable of hybridizing to a target sequence within a P. syringae HopAA1-1 gene, and wherein the spacer comprises the nucleotide sequence set forth as SEQ ID NO: 186.

211. The method of embodiment 192, wherein the at least one guide RNA comprises a spacer capable of hybridizing to a target sequence within a P. syringae HopAA1-3 gene, and wherein the spacer comprises a nucleotide sequence that differs in length and/or sequence from SEQ ID NO: 187 by 1 to 5 nucleotides.

212. The method of embodiment 192, wherein the at least one guide RNA comprises a spacer capable of hybridizing to a target sequence within a P. syringae HopAA1-3 gene, and wherein the spacer comprises the nucleotide sequence set forth as SEQ ID NO: 187.

213. The method of embodiment 192, wherein the at least one guide RNA comprises a spacer capable of hybridizing to a target sequence within a P. infestans Avrblb1-2 gene, and wherein the spacer comprises a nucleotide sequence that differs in length and/or sequence from SEQ ID NO: 188 by 1 to 5 nucleotides.

214. The method of embodiment 192, wherein the at least one guide RNA comprises a spacer capable of hybridizing to a target sequence within a P. infestans Avrblb1-2 gene, and wherein the spacer comprises the nucleotide sequence set forth as SEQ ID NO: 188.

215. The method of embodiment 192, wherein the at least one guide RNA comprises a spacer capable of hybridizing to a target sequence within a P. infestans CRE8-1 gene, and wherein the spacer comprises a nucleotide sequence that differs in length and/or sequence from SEQ ID NO: 189 by 1 to 5 nucleotides.

216. The method of embodiment 192, wherein the at least one guide RNA comprises a spacer capable of hybridizing to a target sequence within a P. infestans CRE8-1 gene, and wherein the spacer comprises the nucleotide sequence set forth as SEQ ID NO: 189.

217. The method of embodiment 192, wherein the at least one guide RNA comprises a spacer capable of hybridizing to a target sequence within a Leptinotarsa decemlineata LdNA_17212-1 gene, and wherein the spacer comprises a nucleotide sequence that differs in length and/or sequence from SEQ ID NO: 190 by 1 to 5 nucleotides.

218. The method of embodiment 192, wherein the at least one guide RNA comprises a spacer capable of hybridizing to a target sequence within a Leptinotarsa decemlineata LdNA_17212-1 gene, and wherein the spacer comprises the nucleotide sequence set forth as SEQ ID NO: 190.

219. The method of embodiment 192, wherein the at least one guide RNA comprises a spacer capable of hybridizing to a target sequence within a Leptinotarsa decemlineata LdNA_31771-2 gene, and wherein the spacer comprises a nucleotide sequence that differs in length and/or sequence from SEQ ID NO: 191 by 1 to 5 nucleotides.

220. The method of embodiment 192, wherein the at least one guide RNA comprises a spacer capable of hybridizing to a target sequence within a Leptinotarsa decemlineata LdNA_31771-2 gene, and wherein the spacer comprises the nucleotide sequence set forth as SEQ ID NO: 191.

221. A method of controlling one or more plant pest in an area of cultivation, where said method comprises:
a) applying the composition of any one of embodiments 1-78 to said area of cultivation or to a plant or part thereof growing in said area of cultivation; or
b) planting a seed or a plant comprising the composition of any one of embodiments 1-78 in said area of cultivation.

222. A method of improving performance of a plant or plant population comprising applying the composition of any one of embodiments 1-78 to said plant or plant population.

223. The method of embodiment 221 or 222, wherein the applying comprises spraying.

The following examples are offered by way of illustration and not by way of limitation.

### EXPERIMENTAL

### Example 1—Sequence Analyses of Cas12a2 Nucleases

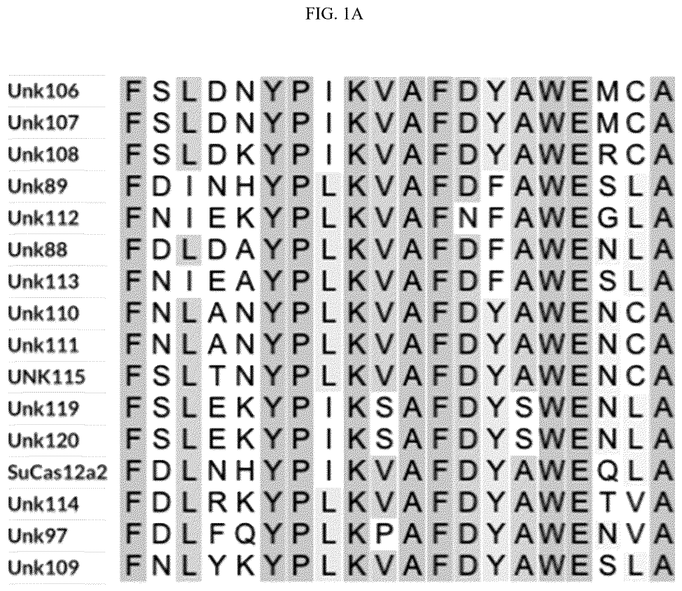

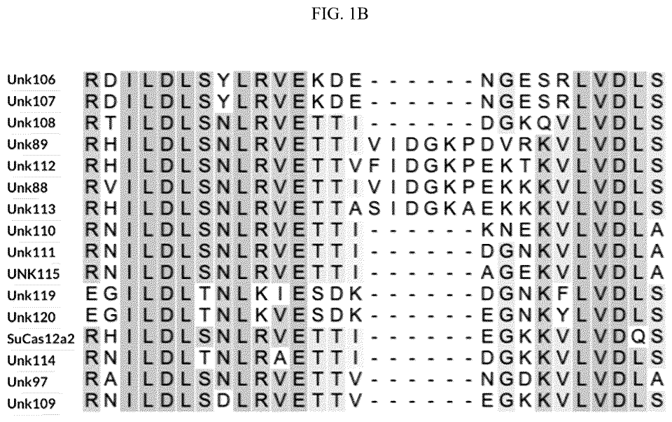

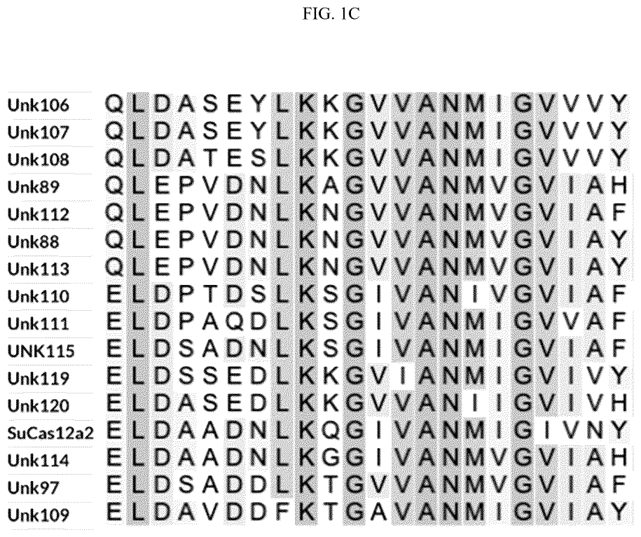

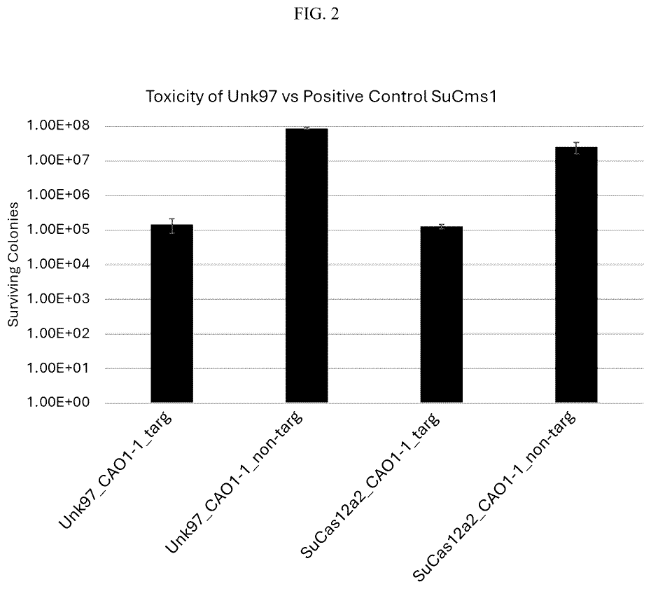

Cas12a2 nuclease amino acid sequence alignments were examined to identify amino acid residues within the protein sequences that are well-conserved among these nucleases. SulfCas12a2 (SuCas12a2; SEQ ID NO: 1) and SulfCas12a2 variant nucleases, including those proteins in the group consisting of SEQ ID NOs: 2-39, and 166, were aligned to identify partially and/or completely conserved amino acid residues among these nucleases. FIGS. 1A-1C show an alignment of three domains (FIG. 1A, residues 370 to 389; FIG. 1B, residues 896 to 921; and FIG. 1C, residues 1028 to 1049) within the SulfCas12a2-like nucleases. Within these three domains, individual amino acid residues were identified as being partially (lighter shading) or completely (darker shading) conserved between the SulfCas12a2-like nucleases.

### Example 2—Functionality of Unk97 Cas12a2 in a Cell Toxicity Assay in E. coli

The toxic activity of Unk97 Cas12a2 (amino acid sequence set forth as SEQ ID NO: 28) was validated in E. coli by conducting a toxicity assay. Two plasmids were transformed into BL21-AI E. coli cells and maintained through selection. Plasmid A, conferring Chloramphenicol-34 resistance, contained arabinose+IPTG inducible expression cassettes for Cas12a2 and a guide RNA corresponding to a target of interest. Plasmid B, conferring Kanamycin resistance, contained a fragment of the target gene of interest or a non-target gene fragment. For the purposes of this experiment, the target DNA sequence was in CAO1-1 from O. sativa. The two plasmid strains were grown overnight, fresh cultures were inoculated, and expression was induced for two hours before plating a 10-fold dilution series of each Cas12a2 and guide with its target and non-target control. Treatments were plated without selection for plasmid B. Colonies were counted the following day and the percent reduction of surviving colonies were recorded.

Table 1 shows the toxicity of Unk97 Cas12a2 compared to positive control SuCas12a2 and other Cas12a2 polypeptides.

TABLE 1
Results of Unk97 Cas12a2 toxicity assay in E. coli

| Target | | | | % Reduction |
|---|---|---|---|---|
| Species | Nuclease | Guide RNA | Targeted Test Sequence | vs non-target |
| O. sativa | SuCas12a2 | CAO1-1 | tggagcaacacctgaaggaaggct | 99.70% |
| | | | (SEQ ID NO: 102) | |
| O. sativa | Unk97 | CAO1-1 | tggagcaacacctgaaggaaggct | 99.80% |
| | | | (SEQ ID NO: 102) | |
| O. sativa | Unk88 | CAO1-1 | tggagcaacacctgaaggaaggct | 20% |
| | | | (SEQ ID NO: 102) | |
| O. sativa | Unk89 | CAO1-1 | tggagcaacacctgaaggaaggct | 0% |
| | | | (SEQ ID NO: 102) | |

### Example 3—Functionality of SuCas12a2 Against Diverse Target Sequences in a Cell Toxicity Assay in E. coli

Guide RNAs were validated in E. coli by conducting a toxicity assay, wherein two plasmids were transformed into BL21-AI cells and maintained through selection. Plasmid A, conferring Chloramphenicol-34 resistance, contained arabinose+IPTG-inducible expression cassettes for SuCas12a2 (SEQ ID NO: 1) and a guide RNA corresponding to a target of interest. Plasmid B, conferring Kanamycin resistance, contained a fragment of the target gene of interest or a non-target gene fragment. The two plasmid strains were grown overnight, fresh cultures were inoculated, and expression was induced for two hours before plating a 10-fold dilution series of each guide with its target and non-target control. Treatments were plated without selection for plasmid B. Colonies were counted the following day and % Reduction of surviving colonies were recorded. Results in Table 2 show that SuCas12a2 was effective at killing cells when tested against diverse target sequences, including from rice, worm, mammalian cells, and bacteria.

TABLE 2
Results of SuCas12a2 activity as measured by cell killing assay in E. coli

| Target | | | | % Reduction vs |
|---|---|---|---|---|
| Species | Nuclease | Guide | Sequence (SEQ ID NO) | non-target |
| O. sativa | PbCpf1 | CAO1-1 | tggagcaacacctgaaggaaggct | 0 |
| | | | (SEQ ID NO: 102) | |
| O. sativa | SuCas12a2 | CAO1-1 | tggagcaacacctgaaggaaggct | 99.7% |
| | | | (SEQ ID NO: 102) | |
| C. elegans | SuCas12a2 | AGE1-1 | CGGAATATGTTCCCCAC | 96.4% |
| | | | TTCATCG (SEQ ID NO: | |
| | | | 103) | |
| C. elegans | SuCas12a2 | AGE1-2 | GCCGAAATTATTCAACT | 98.8% |
| | | | GTCTGAA (SEQ ID NO: | |
| | | | 104) | |
| C. elegans | SuCas12a2 | AGE1-7 | TCCAGTGAGTATCCTAG | 99.0% |
| | | | ACAATGA (SEQ ID NO: | |
| | | | 105) | |
| C. elegans | SuCas12a2 | AKT1-2 | CATTAATTCCTCTTGTG | 98.3% |
| | | | GATTTGC (SEQ ID NO: | |
| | | | 106) | |
| C. elegans | SuCas12a2 | AKT1-5 | GCTGCTGATTctaaaagaatttt | 98.2% |
| | | | t (SEQ ID NO: 107) | |
| C. elegans | SuCas12a2 | AKT1-8 | GCAGCTTCGCTGGTGTC | 94.7% |
| | | | AGCAATA (SEQ ID NO: | |
| | | | 108) | |
| HEK293T | SuCas12a2 | PIK3CA-2 | GTAGTAAACATTCTACT | 99.7% |
| | | | AGGATTC (SEQ ID NO: | |
| | | | 109) | |
| HEK293T | SuCas12a2 | PIK3CA-7 | AAAAGGGTTGAAAAAG | 99.3% |
| | | | CCGAAGGT (SEQ ID NO: | |
| | | | 110) | |
| HEK293T | SuCas12a2 | PIK3CA-8 | TGGTTATTAATGTAGCC | (retest pending) |
| | | | TCACGGA (SEQ ID NO: | |
| | | | 111) | |
| P. syringae | SuCas12a2 | AvrE1-2 | ATGCGTCTGCGCGCCCG | 97.9% |
| | | | AGCTGCT (SEQ ID NO: | |
| | | | 112) | |
| P. syringae | SuCas12a2 | AvrE1-4 | ATCACTCGGTCGTCGAG | 99.7% |
| | | | GCCTTCG (SEQ ID NO: | |
| | | | 113) | |
| P. syringae | SuCas12a2 | HopAA1-1 | TCCTTCAAACCGAGCAC | 99.8% |
| | | | TAATGCA (SEQ ID NO: | |
| | | | 114) | |
| P. syringae | SuCas12a2 | HopAA1-3 | ACCGCGGGATCGGTTGT | 99.7% |
| | | | CAGCGCG (SEQ ID NO: | |
| | | | 115) | |

### Example 4—Functionality of Cas12a2 Against Diverse Target Sequences in a Cell Toxicity Assay in Pseudomonas syringae

Guide RNAs were validated in Pseudomonas syringae by conducting a toxicity assay. In this assay, one plasmid is transformed into Pseudomonas cells and maintained through selection. The plasmid has a Chloramphenicol-34 resistance gene, along with an IPTG-inducible expression cassette comprising polynucleotide sequences encoding a Cas12a2 and a guide RNA targeting a target sequence of interest. The target of interest in this assay is in the Pseudomonas genome. The plasmid was transformed into Pseudomonas by electroporation, and transformed cells were selected on chloramphenicol. Colonies containing the plasmid were grown overnight, fresh cultures were inoculated, and expression was induced for four hours before plating a 10-fold dilution series of each guide. Colonies were counted after 48 hrs and % reduction of surviving colonies were recorded. Results in Table 3 show that Unk97 Cas12a2 is effective at killing Pseudomonas syringae cells when tested against multiple P. syringae target sequences.

TABLE 3
Results of Cas12a2 activity as measured by cell killing assay in Pseudomonas syringae

| Target | | | | | |
|---|---|---|---|---|---|
| Species | Nuclease | Guide | Sequence | % Reduction | p-value |
| P. syringae | SuCas12a2 | HopAA1-1 | TCCTTCAAACCGAGCACTA | **89% | 0.001 |
| | | | ATGCA (SEQ ID NO: 114) | | |
| P. syringae | Mc.46 | HopAA1-1 | TCCTTCAAACCGAGCACTA | 23% | 0.700 |
| | | | ATGCA (SEQ ID NO: 114) | | |
| P. syringae | Unk97 | HopAA1-1 | TCCTTCAAACCGAGCACTA | *95% | 0.068 |
| | | | ATGCA (SEQ ID NO: 114) | | |
| P. syringae | Unk97 | HopAA1-3 | ACCGCGGGATCGGTTGTCA | 94% | low reps |
| | | | GCGCG (SEQ ID NO: 115) | | |
| P. syringae | Unk97 | AvrE1-2 | ATGCGTCTGCGCGCCCGAG | *99% | 0.076 |
| | | | CTGCT (SEQ ID NO: 112) | | |
| P. syringae | Unk97 | AvrE1-4 | ATCACTCGGTCGTCGAGGC | 99% | low reps |
| | | | CTTCG (SEQ ID NO: 113) | | |

*=p < 0.1, **=p < 0.05

### Example 5—Optimizing Peptide Fusions for Delivery of Cas12a2 Across Membranes

Pathogenesis-related protein 1a (PR1a) signaling peptide and cell penetrating peptides (CPPs) may be operably fused to Cas12a2 as a strategy for delivery of the nuclease across membranes, such as delivery into a bacterial pest (e.g., Pseudomonas) or delivery to the apoplast of a plant cell. Unk97 Cas12a2 operably fused to various peptides and N-terminal vs. C-terminal fusions were tested. Functionality of the tagged Unk97 Cas12a2 was assessed by the cell toxicity assay in E. coli as described in Examples 2 and 3, with rice CAO1-1 as the target sequence. The indicated peptide(s) were operably fused at the N-terminus or at the C-terminus of Unk97 Cas12a2 via a 4× GGS (glycine-glycine-serine) linker (Table 4; the 4× GGS linker is set forth as SEQ ID NO: 161). Results in Table 4 show that Unk97 Cas12a2 having operable fusion of peptide(s) at its C-terminus is effective at killing cells.

The operable fusion of a tag at the N-terminus of Cas12a2 was further explored. A number of protein tags aimed at exporting Cas12a2 to the apoplast of the plant cell (PR1a, AtChitinase, XBiP, and SlPR1a (Solanum lycopersicum PR1a)) were tested as operable fusions at the N-terminus of the Cas12a2. All tested Cas12a2 proteins had an operable fusion of R9-TAT CPP at their C-termini via a 4× GGS linker. Table 5 shows that a 4× GGS linker does not help stabilize an N-terminal fusion of PR1a to Unk97 Cas12a2 and that operable fusion of PR1a at the N-terminus for Unk97, Unk109, Unk110, and Unk114 without a linker allows robust function for these Cas12a2 nucleases.

As discussed above, Table 4 shows that peptide(s) operably fused to the C-terminus of Unk97 Cas12a2 via a 4× GGS linker were effective at killing cells. Additional CPPs were tested to see which ones would allow function of Cas12a2 when in operable fusion with Cas12a2 at its C-terminus via a 4× GGS linker. Table 6 shows that two other CPPs: penetratin (SEQ ID NO: 160) and transportan (SEQ ID NO: 117) allow function for Cas12a2.

TABLE 4
Unk97 Cas12a2 activity with different peptides
and peptide configurations as measured
by cell killing assay in E. coli

| | Terminus of | |
|---|---|---|
| | Cas12a2 at which | |
| | peptide is | |
| Peptide | operably fused† | % Reduction |
| R9-TAT | N-terminus | 46% |
| PR1a | N-terminus | 76% |
| R9-TAT_PR1a | N-terminus | NS\hat{ } |
| R9-TAT | C-terminus | 99% |
| PR1a | C-terminus | 97% |
| R9-TAT_PR1a | C-terminus | 98% |

†= the peptide and Cas12a2 are operably fused via a 4x GGS linker
\hat{ }= not significant

TABLE 5
Further testing of signal peptides at the N-terminus of Cas12a2
for function as measured by cell killing assay in E. coli

| | N-terminal peptide§ | | | | |
|---|---|---|---|---|---|
| Nuclease* | PR1a + | | | | |
| Cas12a2 | Linker | PR1a | AtChitinase | XBiP | SIPR1a |
| Su | 99.7% | >99% | 60% | NT† | NT |
| Unk97 | NS\hat{ } | 99% | NS | 86% | NS |
| Unk109 | NT | 91% | 81% | NT | NT |
| Unk110 | NT | >99% | 65% | NT | NT |
| Unk114 | NT | >99% | 92% | NT | NT |

§= the N-terminal peptide is operably fused to the Cas12a2 directly without a linker unless a linker is indicated
*= each Cas12a2 has an operable fusion of R9-TAT CPP at its C-terminus via a 4x GGS linker
\hat{ }= not significant
†= not tested

TABLE 6
Unk97 Cas12a2 is functional with various CPPs at its C-
terminus as measured by cell killing assay in E. coli

| Nuclease* | C-terminal CPP | % Reduction\hat{ } |
|---|---|---|
| Unk97 | Penetratin | 99% |
| Unk97 | TAT | 97% |
| Unk97 | Transportan | 98% |

*= Unk97 Cas12a2 is operably fused to the indicated CPP at the C-terminus via a 4x GGS linker
\hat{ }= % Reduction is percent reduction of surviving colonies as measured for a target gene as compared to a non-target control

### Example 6—Functionality of Unk97 Cas12a2 Against Phytophthora infestans (Late Blight) and Leptinotarsa decemlineata (Colorado Potato Beetle, CPB) Target Sequences in a Cell Toxicity Assay in E. coli

Two of the most detrimental pests for potato are the fungus Phytophthora infestans (late blight) and the beetle Leptinotarsa decemlineata (Colorado potato beetle, CPB). About 75% of potato acreage receive 4 or more fungicide applications every 7-10 days each year to limit growth of P. infestans. The larvae of L. decemlineata are most detrimental to potato foliage. CPB, considered a superpest, has developed resistance to 56 different compounds and is responsible for about 40% yield loss when not controlled.

Unk97 Cas12a2, along with an appropriate guide RNA targeting a P. infestans or L. decemlineata target sequence, was assessed for functionality by the cell toxicity assay in E. coli as described in Examples 2 and 3 but with the plasmids designed to contain nucleotide sequences encoding guide RNAs and target sequences specific to the target species in Table 7. Results in Table 7 show that Unk97 Cas12a2 and its appropriate guide RNA was effective at killing cells when tested against two distinct target sequences of P. infestans and L. decemlineata.

TABLE 7
Unk97 Cas12a2 activity against P. infestans and L. decemlineata as measured by cell
killing assay in E. coli

| Target | | | | % | |
|---|---|---|---|---|---|
| Species | Nuclease | Guide | Sequence | Reduction | p-value |
| P. infestans | Unk97 | Avrblb1-2 | AGATAGAAAACGCCCGC | **99.86% | 0.031 |
| | | | TCTTCGT (SEQ ID NO: | | |
| | | | 162) | | |
| P. Infestans | Unk97 | CRE8-1 | CTGCCGCCTCCTTTTGAG | **99.88% | 0.018 |
| | | | CATTCA (SEQ ID NO: 163) | | |
| L. | Unk97 | LdNA | TTGTGAATAGCGTTTTGA | **98.63% | 0.013 |
| decemlineata | | 17212-1 | CCTTCC (SEQ ID NO: 164) | | |
| L. | Unk97 | LdNA | GCATCGTATTGGCACGGG | **98.47% | 0.011 |
| decemlineata | | 31771-2 | GTATCT (SEQ ID NO: 165) | | |

*=p < 0.1, **=p < 0.05

### Example 7—Transgenic Plant Challenge Experiments to Assess Functionality of Unk97 Cas12a2 to Reduce or Eliminate Pseudomonas syringae Infection of a Plant

T-DNA constructs comprising polynucleotide sequences encoding Unk97 Cas12a2 and Pseudomonas syringae-specific HOPAA1-1 guide RNA as well as Basta resistance gene were transformed into Agrobacterium strain GV3101 using electroporation. GV3101 was used to transform Arabidopsis using floral dip following standard protocols.

T1 seed from two independent events were planted on soil and maintenance of T-DNA was confirmed by Basta selection after 1 week.

Developmental leaf 5 and 6 of 5-week-old transgenic T1s and Columbia-0 (WT) plants were infiltrated with Pseudomonas syringae DC3000 strain at an OD of 0.0002 in 10 mM MgCl₂. Plants were incubated under normal growth conditions for 5 days post infiltration.

On Day 5 post infiltration a 6.0 mm punch was taken from each infiltrated leaf and combined into a single 1.7 ml tube. Tissue was ground in 500 μl MgCl₂ and serial diluted up to 10⁷. All dilutions were plated on King's Media and allowed to grow at 28° C. before calculated final colony forming units (CFUs) per leaf disc.

Data was collected for 6 replicates of each T1 event and Col-0 control.

It is expected that T1 transgenic plants show significantly less CFUs then Columbia-0 control plants.

### Example 8—Pseudomonas syringae DC3000 Induction Experiments to Assess Functionality of Unk97 Cas12a2 to Reduce or Eliminate Pseudomonas syringae Infection of a Plant

Pseudomonas syringae strain DC3000 was transformed via electroporation with a plasmid having polynucleotide sequences encoding Unk97 Cas12a2 and Pseudomonas syringae-specific HOPAA1-1 guide RNA or a plasmid having a polynucleotide sequence encoding GFP, each under TAC inducible expression. Single colonies were isolated and confirmed to harbor the correct plasmid using PCR.

Strains for Unk97 with HOPAA1-1 guide or GFP were grown up in Kings Media with antibiotics until an OD of between 0.2-0.4 was reached. Once optimal OD was reached the cultures were induced using 5 mM IPTG to turn on expression of plasmids for 4 hrs.

After induction, cultures were diluted in MgCl₂ to 0.0002 OD using the GFP culture's OD as normalizer.

Developmental leaf 5 and 6 from 5-week-old Columbia-0 Arabidopsis plants were infiltrated. Plants were incubated under normal growth conditions for 5 days post infiltration.

On Day 5 post infiltration a 6.0 mm punch was taken from each infiltrated leaf and combined into a single 1.7 ml tube. Tissue was ground in 500 μl MgCl₂ and serial diluted up to 10⁷. All dilutions were plated on King's Media and allowed to grow at 28° C. before calculated final colony forming units (CFUs) per leaf disc.

Data was collected for 6 replicates of Col-0 for either Unk97 with HOPAA1-1 guide or GFP.

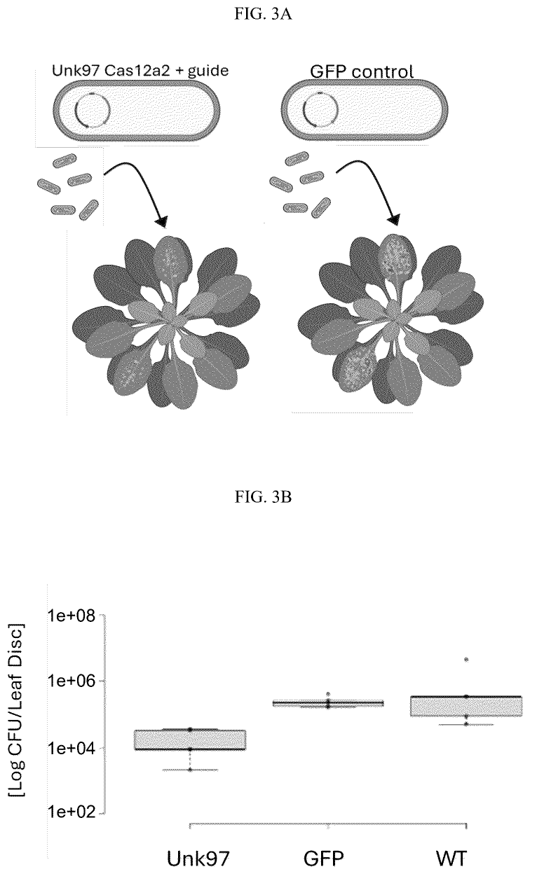

Pseudomonas syringae DC3000 expressing Unk97 with HOPAA1-1 guide no longer caused robust infection in Arabidopsis (FIG. 3A) and showed significantly less colony forming units (CFUs) than Pseudomonas syringae DC3000 expressing the GFP control or wild type (WT) Pseudomonas syringae DC3000 (FIG. 3B). Table 8 shows the percent reduction in CFUs of Pseudomonas syringae DC3000 expressing Unk97 Cas12a2 and HOPAA1-1 guide RNA as compared to two different negative controls, Pseudomonas syringae DC3000 expressing GFP or WT Pseudomonas syringae DC3000. The first column is labeled as Unk97+HOPAA1-1 and is the experimental treatment.

TABLE 8
Unk97 Cas12a2 activity against Pseudomonas syringae\hat{ }

| | | | % Reduction | |
|---|---|---|---|---|
| | % Reduction (as | | (as compared | |
| | compared to GFP | | to WT | |
| Construct | control) | P-value | control) | P-value |
| Unk97 + | 92.8% | 0.01 | 98.3% | 0.17 |
| HOPAA1-1 | | | | |

\hat{ }as measured by % reduction in CFUs from plant leaf infiltrated with P. syringae expressing Unk97 Cas12a2 and HOPAA1-1 guide RNA as compared to indicated control

### Example 9—C. Elegans Toxicity Experiment

Transgenic C. elegans nematodes are created either: having constructs comprising polynucleotide sequences encoding SuCas12a2 and two targeting guides (AGE-1 and AKT-1); or having a construct comprising a polynucleotide sequence encoding SuCas12a2 and lacking polynucleotide sequences encoding targeting guides; where expression of the SuCas12a2 and guide RNAs (if encoded) are heat shock inducible. The genetic constructs are introduced into C. elegans using a standard micro-injection protocol. Each transgenic strain is backcrossed 4× to remove any residual background mutation acquired during transformation.

### Survival Assay

Synchronized eggs are collected from transgenic strains for 1 hour and cultured at 16° C. until they reach L4 stage. At L4 stage, worms are heat shocked at 33° C. for 1 hour then placed at 20° C. for the remainder of the experiment. Worm death rate is recorded daily until all worms are deceased. For each condition 2 replicates are completed with a minimum of 60 worms counted in each replicate.

It is expected that there is lower survivability (or increased rate of death) for C. elegans expressing SuCas12a2 and two targeting guides compared to C. elegans expressing SuCas12a2 and no targeting guides.

TABLE 9
Description of Sequences of the Application

| SEQ ID | |
|---|---|
| NO: | Description |
| 1 | Sulfuricurvum sp. PC08-66 |
| | Cas12a2 |
| 2 | AuxCas12a2 |
| 3 | LAHSCas12a2 |
| 4 | Unk2 Cas12a2 |
| 5 | Unk6 Cas12a2 |
| 6 | Unk7 Cas12a2 |
| 7 | Unk11 Cas12a2 |
| 8 | Unk14 Cas12a2 |
| 9 | Unk17 Cas12a2 |
| 10 | Unk33 Cas12a2 |
| 11 | Unk34 Cas12a2 |
| 12 | Unk35 Cas12a2 |
| 13 | Unk36 Cas12a2 |
| 14 | Unk37 Cas12a2 |
| 15 | Unk38 Cas12a2 |
| 16 | Unk41 Cas12a2 |
| 17 | Unk51 Cas12a2 |
| 18 | Unk55 Cas12a2 |
| 19 | Unk56 Cas12a2 |
| 20 | Unk59 Cas12a2 |
| 21 | Unk63 Cas12a2 |
| 22 | Unk64 Cas12a2 |
| 23 | Unk66 Cas12a2 |
| 24 | Unk68 Cas12a2 |
| 25 | Unk71 Cas12a2 |
| 26 | Unk89 Cas12a2 |
| 27 | Unk88 Cas12a2 |
| 28 | Unk97 Cas12a2 |
| 29 | Unk106 Cas12a2 |
| 30 | Unk107 Cas12a2 |
| 31 | Unk108 Cas12a2 |
| 32 | Unk109 Cas12a2 |
| 33 | Unk110 Cas12a2 |
| 34 | Unk111 Cas12a2 |
| 35 | Unk112 Cas12a2 |
| 36 | Unk113 Cas12a2 |
| 37 | Unk114 Cas12a2 |
| 38 | Unk119 Cas12a2 |
| 39 | Unk120 Cas12a2 |
| 40 | gRNA Stem loop 1 (N3) |
| 41 | gRNA Stem loop 2 (N4) |
| 42 | gRNA Stem loop 3 (N5) |
| 43 | DNA Sequence encoding |
| | gRNA stem loop 1 |
| 44 | DNA Sequence encoding |
| | gRNA stem loop 2 |
| 45 | DNA Sequence encoding |
| | gRNA stem loop 2 |
| 46 | Sulf-type Cas12a2 conserved |
| | motif 1 |
| 47 | Sulf-type Cas12a2 conserved |
| | motif 2 |
| 48 | Sulf-type Cas12a2 conserved |
| | motif 3 |
| 49 | Sulf-type Cas12a2 conserved |
| | motif 4 |
| 50 | Sulf-type Cas12a2 conserved |
| | motif 5 |
| 51 | Sulf-type Cas12a2 conserved |
| | motif 6 |
| 52 | Sulf-type Cas12a2 conserved |
| | motif 7 |
| 53 | Sulf-type Cas12a2 conserved |
| | motif 8 |
| 54 | Sulf-type Cas12a2 conserved |
| | motif 9 |
| 55 | Sulf-type Cas12a2 conserved |
| | motif 10 |
| 56 | Sulf-type Cas12a2 conserved |
| | motif 11 |
| 57 | Sulf-type Cas12a2 conserved |
| | motif 12 |
| 58 | Sulf-type Cas12a2 conserved |
| | motif 13 |
| 59 | Sulf-type Cas12a2 conserved |
| | motif 14 |
| 60 | Sulf-type Cas12a2 conserved |
| | motif 15 |
| 61 | Sulf-type Cas12a2 conserved |
| | motif 16 |
| 62 | Sulf-type Cas12a2 conserved |
| | motif 17 |
| 63 | Sulfuricurvum sp. PC08-66 |
| | Cas12a2 nucleotide coding |
| | sequence |
| 64 | AuxCas12a2 nucleotide coding |
| | sequence |
| 65 | LAHSCas12a2 nucleotide |
| | coding sequence |
| 66 | Unk2 nucleotide coding |
| | sequence |
| 67 | Unk6 nucleotide coding |
| | sequence |
| 68 | Unk7 nucleotide coding |
| | sequence |
| 69 | Unk11 nucleotide coding |
| | sequence |
| 70 | Unk14 nucleotide coding |
| | sequence |
| 71 | Unk17 nucleotide coding |
| | sequence |
| 72 | Unk33 nucleotide coding |
| | sequence |
| 73 | Unk34 nucleotide coding |
| | sequence |
| 74 | Unk35 nucleotide coding |
| | sequence |
| 75 | Unk36 nucleotide coding |
| | sequence |
| 76 | Unk37 nucleotide coding |
| | sequence |
| 77 | Unk38 nucleotide coding |
| | sequence |
| 78 | Unk41 nucleotide coding |
| | sequence |
| 79 | Unk51 nucleotide coding |
| | sequence |
| 80 | Unk55 nucleotide coding |
| | sequence |
| 81 | Unk56 nucleotide coding |
| | sequence |
| 82 | Unk59 nucleotide coding |
| | sequence |
| 83 | Unk63 nucleotide coding |
| | sequence |
| 84 | Unk64 nucleotide coding |
| | sequence |
| 85 | Unk66 nucleotide coding |
| | sequence |
| 86 | Unk68 nucleotide coding |
| | sequence |
| 87 | Unk71 nucleotide coding |
| | sequence |
| 88 | Unk88 nucleotide coding |
| | sequence |
| 89 | Unk89 nucleotide coding |
| | sequence |
| 90 | Unk97 nucleotide coding |
| | sequence |
| 91 | Unk106 nucleotide coding |
| | sequence |
| 92 | Unk107 nucleotide coding |
| | sequence |
| 93 | Unk108 nucleotide coding |
| | sequence |
| 94 | Unk109 nucleotide coding |
| | sequence |
| 95 | Unk110 nucleotide coding |
| | sequence |
| 96 | Unk111 nucleotide coding |
| | sequence |
| 97 | Unk112 nucleotide coding |
| | sequence |
| 98 | Unk113 nucleotide coding |
| | sequence |
| 99 | Unk114 nucleotide coding |
| | sequence |
| 100 | Unk119 nucleotide coding |
| | sequence |
| 101 | Unk120 nucleotide coding |
| | sequence |
| 102 | Oryza sativa CAO1-1 target |
| | sequence |
| 103 | C. elegans AGE1-1 target |
| | sequence |
| 104 | C. elegans AGE1-2 target |
| | sequence |
| 105 | C. elegans AGE1-7 target |
| | sequence |
| 106 | C. elegans AKT1-2 target |
| | sequence |
| 107 | C. elegans AKT1-5 target |
| | sequence |
| 108 | C. elegans AKT1-8 target |
| | sequence |
| 109 | HEK293T PIK3CA-2 target |
| | sequence |
| 110 | HEK293T PIK3CA-7 target |
| | sequence |
| 111 | HEK293T PIK3CA-8 target |
| | sequence |
| 112 | P. syringae AvrE1-2 target |
| | sequence |
| 113 | P. syringae AvrE1-4 target |
| | sequence |
| 114 | P. syringae HopAA1-1 target |
| | sequence |
| 115 | P. syringae HopAA1-3 target |
| | sequence |
| 116 | R9-TAT cell penetrating |
| | peptide (CPP) from human |
| | immunodeficiency virus |
| 117 | transportan CPP from galanin- |
| | mastoparan chimeric peptide |
| 118 | PR1a export signal from |
| | Nicotiana tabacum |
| 119 | SV40 peptide nuclear |
| | localization signal (NLS) from |
| | Simian vacuolating virus |
| 120 | nucleoplasmin peptide NLS |
| | from Xenopus |
| 121 | amino acid sequence for |
| | Unk106 and Unk107 |
| | corresponding to amino acid |
| | residues 370 to 389 of |
| | SuCas12a2 |
| 122 | amino acid sequence for |
| | Unk108 corresponding to |
| | amino acid residues 370 to 389 |
| | of SuCas12a2 |
| 123 | amino acid sequence for |
| | Unk89 corresponding to amino |
| | acid residues 370 to 389 of |
| | SuCas12a2 |
| 124 | amino acid sequence for |
| | Unk112 corresponding to |
| | amino acid residues 370 to 389 |
| | of SuCas12a2 |
| 125 | amino acid sequence for |
| | Unk113 corresponding to |
| | amino acid residues 370 to 389 |
| | of SuCas12a2 |
| 126 | amino acid sequence for |
| | Unk88 corresponding to amino |
| | acid residues 370 to 389 of |
| | SuCas12a2 |
| 127 | amino acid sequence for |
| | Unk119 and Unk120 |
| | corresponding to amino acid |
| | residues 370 to 389 of |
| | SuCas12a2 |
| 128 | amino acid sequence for |
| | Unk110 and Unk111 |
| | corresponding to amino acid |
| | residues 370 to 389 of |
| | SuCas12a2 |
| 129 | SuCas12a2 amino acid |
| | residues 370 to 389 |
| 130 | amino acid sequence for |
| | Unk114 corresponding to |
| | amino acid residues 370 to 389 |
| | of SuCas12a2 |
| 131 | amino acid sequence for |
| | Unk109 corresponding to |
| | amino acid residues 370 to 389 |
| | of SuCas12a2 |
| 132 | amino acid sequence for |
| | Unk97 corresponding to amino |
| | acid residues 370 to 389 of |
| | SuCas12a2 |
| 133 | amino acid sequence for |
| | Unk106 and Unk107 |
| | corresponding to amino acid |
| | residues 896 to 919 of |
| | SuCas12a2 |
| 134 | amino acid sequence for |
| | Unk108 corresponding to |
| | amino acid residues 896 to 919 |
| | of SuCas12a2 |
| 135 | amino acid sequence for |
| | Unk89 corresponding to amino |
| | acid residues 896 to 919 of |
| | SuCas12a2 |
| 136 | amino acid sequence for |
| | Unk112 corresponding to |
| | amino acid residues 896 to 919 |
| | of SuCas12a2 |
| 137 | amino acid sequence for |
| | Unk113 corresponding to |
| | amino acid residues 896 to 919 |
| | of SuCas12a2 |
| 138 | amino acid sequence for |
| | Unk88 corresponding to amino |
| | acid residues 896 to 919 of |
| | SuCas12a2 |
| 139 | amino acid sequence for |
| | Unk119 corresponding to |
| | amino acid residues 896 to 919 |
| | of SuCas12a2 |
| 140 | amino acid sequence for |
| | Unk120 corresponding to |
| | amino acid residues 896 to 919 |
| | of SuCas12a2 |
| 141 | amino acid sequence for |
| | Unk110 corresponding to |
| | amino acid residues 896 to 919 |
| | of SuCas12a2 |
| 142 | amino acid sequence for |
| | Unk111 corresponding to |
| | amino acid residues 896 to 919 |
| | of SuCas12a2 |
| 143 | SuCas12a2 amino acid |
| | residues 896 to 919 |
| 144 | amino acid sequence for |
| | Unk114 corresponding to |
| | amino acid residues 896 to 919 |
| | of SuCas12a2 |
| 145 | amino acid sequence for |
| | Unk109 corresponding to |
| | amino acid residues 896 to 919 |
| | of SuCas12a2 |
| 146 | amino acid sequence for |
| | Unk97 corresponding to amino |
| | acid residues 896 to 919 of |
| | SuCas12a2 |
| 147 | amino acid sequence for |
| | Unk106 and Unk107 |
| | corresponding to amino acid |
| | residues 1028 to 1049 of |
| | SuCas12a2 |
| 148 | amino acid sequence for |
| | Unk108 corresponding to |
| | amino acid residues 1028 to |
| | 1049 of SuCas12a2 |
| 149 | amino acid sequence for |
| | Unk89 corresponding to amino |
| | acid residues 1028 to 1049 of |
| | SuCas12a2 |
| 150 | amino acid sequence for |
| | Unk112 corresponding to |
| | amino acid residues 1028 to |
| | 1049 of SuCas12a2 |
| 151 | amino acid sequence for |
| | Unk113 and Unk88 |
| | corresponding to amino acid |
| | residues 1028 to 1049 of |
| | SuCas12a2 |
| 152 | amino acid sequence for |
| | Unk119 corresponding to |
| | amino acid residues 1028 to |
| | 1049 of SuCas12a2 |
| 153 | amino acid sequence for |
| | Unk120 corresponding to |
| | amino acid residues 1028 to |
| | 1049 of SuCas12a2 |
| 154 | amino acid sequence for |
| | Unk110 corresponding to |
| | amino acid residues 1028 to |
| | 1049 of SuCas12a2 |
| 155 | amino acid sequence for |
| | Unk111 corresponding to |
| | amino acid residues 1028 to |
| | 1049 of SuCas12a2 |
| 156 | SuCas12a2 amino acid |
| | residues 1028 to 1049 |
| 157 | amino acid sequence for |
| | Unk114 corresponding to |
| | amino acid residues 1028 to |
| | 1049 of SuCas12a2 |
| 158 | amino acid sequence for |
| | Unk109 corresponding to |
| | amino acid residues 1028 to |
| | 1049 of SuCas12a2 |
| 159 | amino acid sequence for |
| | Unk97 corresponding to amino |
| | acid residues 1028 to 1049 of |
| | SuCas12a2 |
| 160 | Penetratin CPP from |
| | Drosophila |
| 161 | 4x glycine-glycine-serine |
| | linker |
| 162 | P. infestans Avrblb1-2 target |
| | sequence |
| 163 | P. infestans CRE8-1 target |
| | sequence |
| 164 | L. decemlineata LdNA_17212-1 |
| | target sequence |
| 165 | L. decemlineata LdNA_31771-2 |
| | target sequence |
| 166 | Unk115 Cas12a2 |
| 167 | Unk115 nucleotide coding |
| | sequence |
| 168 | Glycine-serine |
| | linker(GGGS)n, where n is an |
| | integer of 1 or greater |
| 169 | Glycine-serine linker |
| | (GGGGS)n, where n is an |
| | integer of 1 or greater |
| 170 | Glycine-serine linker |
| | (GSGGS)n, where n is an |
| | integer of 1 or greater |
| 171 | amino acid sequence for |
| | Unk115 corresponding to |
| | amino acid residues 370 to 389 |
| | of SuCas12a2 |
| 172 | amino acid sequence for |
| | Unk115 corresponding to |
| | amino acid residues 896 to 919 |
| | of SuCas12a2 |
| 173 | amino acid sequence for |
| | Unk115 corresponding to |
| | amino acid residues 1028 to |
| | 1049 of SuCas12a2 |
| 174 | Oryza sativa CAO1-1 spacer |
| | sequence |
| 175 | C. elegans AGE1-1 spacer |
| | sequence |
| 176 | C. elegans AGE1-2 spacer |
| | sequence |
| 177 | C. elegans AGE1-7 spacer |
| | sequence |
| 178 | C. elegans AKT1-2 spacer |
| | sequence |
| 179 | C. elegans AKT1-5 spacer |
| | sequence |
| 180 | C. elegans AKT1-8 spacer |
| | sequence |
| 181 | HEK293T PIK3CA-2 spacer |
| | sequence |
| 182 | HEK293T PIK3CA-7 spacer |
| | sequence |
| 183 | HEK293T PIK3CA-8 spacer |
| | sequence |
| 184 | P. syringae AvrE1-2 spacer |
| | sequence |
| 185 | P. syringae AvrE1-4 spacer |
| | sequence |
| 186 | P. syringae HopAA1-1 spacer |
| | sequence |
| 187 | P. syringae HopAA1-3 spacer |
| | sequence |
| 188 | P. infestans Avrblb1-2 spacer |
| | sequence |
| 189 | P. infestans CRE8-1 spacer |
| | sequence |
| 190 | L. decemlineata LdNA_17212- |
| | 1 spacer sequence |
| 191 | L. decemlineata LdNA_31771- |
| | 2 spacer sequence |
| 192 | direct repeat (scaffold region) |
| | of Cas12a2 guide RNA |

We claim:

1. A composition comprising:
(i) a Cas12a2 polypeptide, or a polynucleotide encoding a Cas12a2 polypeptide, and
(ii) at least one guide polynucleotide, or a polynucleotide encoding the at least one guide polynucleotide,
wherein each guide polynucleotide comprises: (a) a portion complementary to a target sequence of a plant pest, and (b) a portion capable of binding the Cas12a2 polypeptide,
wherein the target sequence is located immediately adjacent to a PAM sequence that is recognized by the Cas12a2 polypeptide.

2. The composition of claim 1, wherein the Cas12a2 polypeptide comprises one or more conserved amino acid motifs selected from:
(a) a conserved motif 1 comprising the amino acid sequence set forth as WX₂X₃X₄X₅X₆X₇X₈X₉X₁₀X₁₁X₁₂X₁₃X₁₄KX₁₆X₁₇YX₁₉X₂₀X₂₁X₂₂X₂₃X₂₄X₂₅X₂₆FX₂₈X₂₉X₃₀W (SEQ ID NO: 46), wherein X₂, X₃, and X₄=any amino acid; X₅=Y, F, or L; X₆, X₇, and X₈=any amino acid; X₉=D, G, or N; X₁₀=Q, L, F, or M; X₁₁=I, L, V, or M; X₁₂=any amino acid; X₁₃=L, I, or V; X₁₄=any amino acid; X₁₆=D, E, or S; X₁₇=Y or F; X₁₉=K, R, L, or S; X₂₀=any amino acid; X₂₁=L, I, or M; X₂₂=any amino acid; X₂₃=K, R, or S; X₂₄=K or E; X₂₅=A, I, L, or V; X₂₆=any amino acid; X₂₈=D, E, N, or V; X₂₉=A, G, F, or V; X₃₀=F, M, or I;
(b) a conserved motif 2 comprising the amino acid sequence set forth as FKX₃X₄X₅X₆PX₈X₉X₁₀X₁₁X₁₂X₁₃X₁₄X₁₅, wherein X₃=Y, V, or P; X₄=K or I; X₅=any amino acid; X₆=I or V; X₈=F, A, V, or I; X₉=any amino acid; X₁₀=V, A, or L; X₁₁, X₁₂, and X₁₃=any amino acid; X₁₄=L, I, or V; X₁₅=A or V;
(c) a conserved motif 3 comprising the amino acid sequence set forth as FX₂X₃X₄X₅YPX₈KX₁₀AFX₁₃X₁₄X₁₅WEX₁₈X₁₉A (SEQ ID NO: 48), wherein X₂=N, S, or D; X₃=L or I; X₄=any amino acid; X₅=K, N, H, or A; X₈=I or L; X₁₀=V or S; X₁₃=D or N; X₁₄=Y or F; X₁₅=A or S; X₁₈=any amino acid; X₁₉=L, C, or V;
(d) a conserved motif 4 comprising the amino acid sequence set forth as X₁X₂EDX₅X₆X₇X₈X₉X₁₀X₁₁X₁₂, wherein X₁=I or L; X₂=I or V; X₅, X₆, and X₇=any amino acid; X₈=N or D; X₉=R or K; X₁₀=H, F, or Y; X₁₁=I, L, or V; X₁₂=I, L, or F;
(e) a conserved motif 5 comprising the amino acid sequence set forth as X₁X₂X₃X₄SX₆TSX₉X₁₀X₁X₁₂K (SEQ ID NO: 50), wherein X₁=Y, C, or S; X₂=any amino acid; X₃=I or V; X₄=any amino acid; X₆=F, L, I, or V; X₉ and X₁₀=any amino acid; X₁₁=L or I; X₁₂=any amino acid;
(f) a conserved motif 6 comprising the amino acid sequence set forth as X₁X₂X₃X₄X₅EX₇DX₉X₁₀X₁₁X₁₂X₁₃X₁₄, wherein X₁=E or A; X₂=any amino acid; X₃=I or L; X₄=E, K, or I; X₅=K, H, or R; X₇=I, V, or L; X₉=any amino acid; X₁₀=K or N; X₁₁=any amino acid; X₁₂=Y or H; X₁₃=any amino acid; X₁₄=L or F;
(g) a conserved motif 7 comprising the amino acid sequence set forth as X₁LX₃PX₅X₆NX₈D (SEQ ID NO: 52), wherein X₁=L, S, or F; X₃=L, F, or V; X₅=I, F, or L; X₆=I or V; X₈=Q or K;
(h) a conserved motif 8 comprising the amino acid sequence set forth as X₁X₂PEFX₆X₇X₈Y (SEQ ID NO: 53), wherein X₁=L or I; X₂=H or T; X₆=any amino acid; X₇=I, V, L, or M; X₈=F, S, or T;
(i) a conserved motif 9 comprising the amino acid sequence set forth as X₁RX₃X₄X₅X₆X₇X₈X₉X₁₀X₁₁X₁₂X₁₃X₁₄KX₁₆X₁₇X₁₈, wherein X₁=N or K; X₃=Y or F; X₄=S, G, or W; X₅=R, K, or S; X₆=F, L, or V; X₇=Q or E; X₈=M, L, F, or I; X₉=any amino acid; X₁₀=A, C, or G; X₁₁=any amino acid; X₁₂=F, L, or I; X₁₃=any amino acid; X₁₄=any amino acid; X₁₅=E, D, or H; X₁₆=F, Y, I, or V; X₁₇=I, L, V, or K; X₁₈=P or K;
(j) a conserved motif 10 comprising the amino acid sequence set forth as GIDX₄X₅X₆X₇X₈LAX₁₁LCX₁₄ (SEQ ID NO: 55), wherein X₄=R or S; X₅=G or W; X₆=I, L, or Q; X₇=K or N; X₈=E or Q; X₁₁=T or V; X₁₄=I, L, or V;
(k) a conserved motif 11 comprising the amino acid sequence set forth as X₁X₂ILDLX₇X₈LX₁₀X₁₁EX₁₃X₁₄X₁₅X₁₆X₁₇X₁₈X₁₉X₂₀LVDX₂₄X₂₅ (SEQ ID NO: 56), wherein X₁=R or E; X₂=any amino acid; X₇=S or T; X₈=N, D, or Y; X₁₀=R or K; X₁₁=V, I, or A; X₁₃=T, S, or K; X₁₄=T or D; X₁₅=any amino acid; X₁₆=E, D, N, or K; X₁₇=G, K, or N; X₁₈=K, N, E, or T; X₁₉=K, S, or Q; X₂₀=V, R, F, or Y; X₂₄=L or Q; X₂₅=S or A;
(l) a conserved motif 12 comprising the amino acid sequence set forth as X₁X₂X₃X₄X₅X₆X₇X₈X₉X₁₀X₁₁, wherein X₁=L or M; X₂=any amino acid; X₃=any amino acid; X₄=L, M, or Y; X₅=A, S, or P; X₆=Y or S; X₇=I, V, or D; X₈=R or S; X₉=any amino acid; X₁₀=L, N, or V; X₁₁=Q or T;
(m) a conserved motif 13 comprising the amino acid sequence set forth as X₁LX₃X₄X₅X₆X₇X₈KX₁₀GX₁₂X₁₃ANX₁₆X₁₇G X₁₉X₂₀X₂₁X₂₂ (SEQ ID NO: 58), wherein X₁=E or Q; X₃=D or E; X₄=any amino acid; X₅=any amino acid; X₆=D, E, or Q; X₇=N, D, Y, or S; X₈=L or F; X₁₀=any amino acid; X₁₂=V, I, or A; X₁₃=V or I; X₁₆=M or I; X₁₇=I or V; X₁₉=V or I; X₂₀=I or V; X₂₁=A, V, or N; X₂₂=Y, F, or H;
(n) a conserved motif 14 comprising the amino acid sequence set forth as YX₂X₃X₄X₅X₆X₇EX₉X₁₀, wherein X₂=any amino acid; X₃=V, A, or G; X₄=Y, K, R, or V; X₅=I or V; X₆=any amino acid; X₇=L, F, or I; X₉=D or N; X₁₀=L or I;
(o) a conserved motif 15 comprising the amino acid sequence set forth as AX₂X₃X₄X₅X₆X₇X₈X₉EX₁₁X₁₂LX₁₄X₁₅K (SEQ ID NO: 60), wherein X₂=G or W; X₃=L or V; X₄=G, W, or E; X₅=T or L; X₆=Y or M; X₇=any amino acid; X₈=F or Y; X₉=F, L, or M; X₁₁=any amino acid; X₁₂=Q or L; X₁₄=L or V; X₁₅=any amino acid;
(p) a conserved motif 16 comprising the amino acid sequence set forth as FX₂X₃GX₅X₆X₇X₈X₉X₁₀X₁₁X₁₂X₁₃TX₁₅X₁₆X₁₇CPX₂₀C (SEQ ID NO: 61), wherein X₂=any amino acid; X₃=any amino acid; X₅=I or V; X₆=I, F, or V; X₇=any amino acid; X₈=F or Y; X₉=V, I, or T; X₁₀=any amino acid; X₁₁=P or A; X₁₂=any amino acid; X₁₃=any amino acid; X₁₅=S or T; X₁₆=any amino acid; X₁₇=any amino acid; X₂₀=any amino acid; and/or
(q) a conserved motif 17 comprising the amino acid sequence set forth as IX₂X₃X₄DX₆X₇X₈AX₁₀X₁₁I (SEQ ID NO: 62), wherein X₂ and X₃=any amino acid; X₄=G or W; X₆=D, Q, or E; X₇=N or S; X₈=G or A; X₁₀=Y or F; X₁₁=H, L, I, or N, and wherein the Cas12a2 polypeptide does not require a tracrRNA for function.

3. The composition of claim 1, wherein the plant pest comprises a microbe, a plant parasitic nematode, an insect, a fungus, a virus, a mollusk, a spider, a scorpion, a caterpillar, an animal, a mite, a tick, or a combination thereof.

4. The composition of claim 3, wherein the parasitic nematode comprises Caenorhabditis elegans or Heterodera glycines.

5. The composition of claim 3, wherein the insect comprises Western corn rootworm (Diabrotica virgifera virgifera), Northern corn rootworm (Diabrotica barberi), Southern corn rootworm (Diabrotica undecimpunctata howardi), fall armyworm (Spodoptera frugiperda), corn earworm (Helicoverpa zea), aphids (Aphis glycines), boll weevils (Anthonomus grandis), flower thrips, onion thrips, western flower thrips, beet armyworm (Spodoptera exigua), bollworms (Helicoverpa armigera), Colorado potato beetle (Leptinotarsa decemlineata), leafhopper (Empoasca fabae), dryland wireworm (Ctenicera pruinina), Pacific coast wireworm (Limonius canus), or sugarbeet wireworm (Limonius californicus).

6. The composition of claim 3, wherein the fungus comprises Fusarium verticillioides, Fusarium graminearum, soybean rust (Phakopsora pachyrhizi), or late blight (Phytophthora infestans).

7. The composition of claim 3, wherein the virus comprises soybean mosaic virus.

8. The composition of claim 1, wherein the Cas12a2 polypeptide comprises an amino acid sequence having at least 90% sequence identity to a sequence selected from the group consisting of SEQ ID NOs: 28, 31, 32, 33, 34, 36, 37, 38, 166, 1-27, 29, 30, 35, and 39, or wherein the polynucleotide encoding the Cas12a2 polypeptide comprises a nucleotide sequence having at least 90% sequence identity to a sequence selected from the group consisting of SEQ ID NOs: 90, 93, 94, 95, 96, 98, 99, 100, 167, 63-89, 91, 92, 97, and 101.

9. The composition of claim 1, wherein the Cas12a2 polypeptide comprises an amino acid sequence having at least 95% sequence identity to a sequence selected from the group consisting of SEQ ID NOs: 28, 31, 32, 33, 34, 36, 37, 38, 166, 1-27, 29, 30, 35, and 39, or wherein the polynucleotide encoding the Cas12a2 polypeptide comprises a nucleotide sequence having at least 95% sequence identity to a sequence selected from the group consisting of SEQ ID NOs: 90, 93, 94, 95, 96, 98, 99, 100, 167, 63-89, 91, 92, 97, and 101.

10. The composition of claim 1, wherein the Cas12a2 polypeptide comprises the amino acid sequence set forth as any one of SEQ ID NOs: 28, 31, 32, 33, 34, 36, 37, 38, 166, 1-27, 29, 30, 35, and 39, or wherein the polynucleotide encoding the Cas12a2 polypeptide comprises the nucleotide sequence set forth as any one of SEQ ID NOs: 90, 93, 94, 95, 96, 98, 99, 100, 167, 63-89, 91, 92, 97, and 101.

11. The composition of claim 1, wherein the Cas12a2 polypeptide is operably fused to a cell-penetrating domain (CPP), an export signal, a nuclear localization signal (NLS), or a combination thereof.

12. The composition of claim 11, wherein:
the CPP comprises a transactivating transcriptional activator (TAT), a transportan, or a penetratin;
the export signal comprises PR1a; or
the NLS comprises an SV40 peptide or a nucleoplasmin.

13. The composition of claim 11, wherein:
the CPP is set forth as SEQ ID NO: 116, 117, or 160;
the export signal is set forth as SEQ ID NO: 118; or,
the NLS is set forth as SEQ ID NO: 119 or 120.

14. The composition of claim 11, wherein the Cas12a2 polypeptide is operably fused to a CPP, an export signal, and/or an NLS through a peptide linker.

15. The composition of claim 14, wherein the peptide linker is set forth as any one of SEQ ID NOs: 161, 168, 169, and 170.

16. The composition of claim 11, wherein the CPP, the export signal, and/or the NLS is operably fused at the N-terminus or at the C-terminus of the Cas12a2 polypeptide.

17. The composition of claim 11, wherein the operable fusion of the Cas12a2 polypeptide and the export signal is direct and not through a peptide linker.

18. The composition of claim 1, wherein the at least one guide polynucleotide is at least one guide RNA.

19. The composition of claim 1, wherein the polynucleotide encoding the Cas12a2 polypeptide is an mRNA.

20. The composition of claim 1, wherein the polynucleotide encoding a Cas12a2 polypeptide and the polynucleotide encoding the at least one guide polynucleotide are each operably linked to a promoter functional in a plant.

21. A plant, plant part, plant cell, or population of plants comprising the composition of claim 1.

22. A method for producing a modified plant with increased resistance or tolerance to one or more plant pest, the method comprising:
introducing into a plant, plant part, or plant cell the composition of claim 1,
thereby producing a modified plant with increased resistance or tolerance to the one or more plant pest, as compared to resistance or tolerance of a control plant to the one or more plant pest.

23. The method of claim 22, wherein the control plant is a corresponding plant or population of plants that does not comprise the composition.

24. A method of reducing an amount of a plant pest, the method comprising:
contacting the plant pest with a composition comprising:
(a) a Cas12a2 polypeptide, or a polynucleotide encoding a Cas12a2 polypeptide, and
(b) at least one guide polynucleotide, or a polynucleotide encoding at least one guide polynucleotide;
wherein each guide polynucleotide is capable of binding the Cas12a2 polypeptide and hybridizing to a target sequence in one or more cells of the plant pest,
wherein the target sequence is located immediately adjacent to a PAM sequence that is recognized by the Cas12a2 polypeptide,
and wherein the Cas12a2 polypeptide comprises
(i) a primary activity of cleaving the target sequence;
(ii) a secondary activity of cleaving nucleic acid molecules in a non-sequence-specific manner in the one or more cells of the plant pest,
thereby reducing the amount of the plant pest.

25. The method of claim 24, wherein a population of the plant pest is reduced as compared to a corresponding population of the plant pest that has not been contacted with the composition.

26. The method of claim 24, wherein the method further comprises selecting for individuals or cells of the plant pest that comprise (a) and/or (b).

27. The method of claim 24, wherein the plant pest that has been contacted with the composition has reduced ability to infect or destroy a plant.

28. The method of claim 24, wherein the plant pest comprises a microbe, a plant parasitic nematode, an insect, a fungus, a virus, a mollusk, a spider, a scorpion, a caterpillar, an animal, a mite, a tick, or a combination thereof.

29. The method of claim 24, wherein the target sequence is within a gene of C. elegans, P. syringae, P. infestans, or L. decemlineata.

30. The method of claim 24, wherein the PAM sequence that is recognized by the Cas12a2 polypeptide is selected from the group consisting of TTAA, TTAC, TTAG, TTCA, TTCC, TTGG, TTGA, TTGC, TTGG, TTTA, TTTC, TTTG, ATTA, ATTC, ATTG, CTTA, CTTC, CTTG, GTTA, GTTC, GTTG, TCTA, TCTC, TCTG, NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.


SEQUENCE LISTING (192 sequences)

SEQ ID NO: 1 - protein, 1232 aa, Sulfuricurvum sp.
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

SEQ ID NO: 2 - protein, 1346 aa, synthetic construct
MTHNPTILRKTLPLYKEVHFIWRGHAEITSLSLQIYLCLNMAPKKKRKVMGKNENKYQLSKTLRFGLTLKEKISNNEKTPYQSHSQFRDLIILSENRIREGISTPQNRDLPSFIHRIQNCTDFINDFIHDWWMILMHTGQIELDKDYYKSLTKKVGFVGFWYKENKKKGGKTKQPQARNIPMGELRHLCPQNTKECATYITDYWKDLLITATNKLYESSEQQKKFIKAMEQNRTDNKPNEIDLKKSFLSLVSVTMELLNPILNGQILFNKMDRLDMSKKSDNDFIDFVNDHETVRELNNDIEEIIADFKENGNNVNYCKATLNPDTALKQHNNNIPNDIATDLEELMMDSIVGNYDDVNSFMDNYVSNLSAKDKIKKIKDSNISLIYRAILFKYKMIPANVRRDIAQGMAKKLNKDEENIYSFLCEFGTLRTPQKDYADLKDKDSFNLDNYPLKVAFDFAWEGLAKAWYHDQSDFPIDPCRDFLQENFDVNLEEDQEDEYFLLYADLIELNALLSTLDKGNPADPDSIKNEALEMVEYINWNSLDKKNGNYYKKIIKNRLKSSKGNETYERIKKEISMSRGRLKNKIEKYDDLTSQYKRIAMDLGKKFASLRDKIIAANEDNKVTHYAMILEDSNCDKYLLLQKVSNNIYHCMSYDSSDPKAYYVDSITSSAIAKMIRKETNPSKIREYAELEEKERERRNVDDWCRFISKKEYDRRYQLNINNGLSFEALKKEIDSKSYILVKKNISVDSIRELVENEGCLLFPIVNKDLTKERKTTEDNQFTKDWNMIFSGSETNWRLTPEFRVTYRNPVPGYPNDKFGSKRYSRFQMNAHFVCDFIPSSNSYTSNREQIAIFKDEGEQKKRVEEFNRTLSNINQKFYVIGIDRGQKELATLCVVDQDKKIHGDFKIYTRKFNSERKQWEHYSLEGEKGTRNILDLSNLRVETTIIIDGKPERRQVLVDLSEVLVKDKEGNYTKPNKMQIKMQQMAYVRKLQFQMQANPTEVLEWYEQNPTEELIIKNLVDKENGEKGLISFYGTALVELDQTLPVSKIKEMLEEFKILKQRESKKENVQKELNNLTQLEAVDSLKAGIVANMVGVISYILKTLDYNAYISLEDLSTVQSSTEFASGISGAITKMSREEGRRIDVEKYAGLGLYNFFEMQLLRKLHRIQTDNGNILHLVPAFRAQKNYDHIMVGKEKIKNQFGIVFFVDAAATSIKCPRCGAVNEDKFNPDKQKYPDAEKGPKLRNRKEQSGKKVWVTRDKEDDDRIKCYCCGFDTKEKNEGNPFMYIKSGDDNAAYLISDLGVESYRKAYELAATVVEDRKKTLTNNLNQSNYKIRFLWHTMY

SEQ ID NO: 3 - protein, 1344 aa, synthetic construct
MTHNPTILRKTLPLYKEVHFIWRGHAEITSLSLQIYLCLNMAPKKKRKVMKNIVNNYQISKTLRFGLTQKTKIQKEGYNGEIYVSHRELADLVKISEERIKKSVSSSDKSNLELSLDKIDICLKQVGAFLSDWQQVYYRKDQVALDKDYYKILCKKIEFDGFWKDVKGQRMPNSRIINISELDTKDSLGVERLQYVLNYWKDNLVSASQKYSVVEEKIKRFKLAIKINRTDNKPDEVELRKMFLSLANIVCDTLQPLCYGQISFPKINKLDDSRADNKKLIKFATDYKSKNDLLTSIAEQKKYFEENGGNVPFCRATLNPKTALKDPNSTDNSIKGEITQLGLDSILKSFKSYLFFENSLEHMSAKEKIDLMKSSGANVVKKGLMFKYKPIPVIVHREVAHELSKDLNKTEESLSDFLRGIGQAKSPAKDYEELTDKNKFNIEAYPIKVAFDFAWESLAKAKYHSEIDLPVAACEKFLNTFFDVKPNNDKFLLYAKLQELNALISTLEYGNPSDEQSIVEKIKALSNEIKWGDFGGSGQGYKKSISDWADSKKDSDGFKIAKQKIGLFRGGLRNEIAEYYNLTQIYKKTVMQEGKLFATMRDKITGAAEQNKVTHYAAIIEDKTGDKYVLLQEVPLNKQDRIYDKMDRNGDGYVSYFVNSITSRTIAKQLRKKRMAELMKNNARGIYNNVSNIQQPALSDKEKEERNIKEWISFISEKRWDYEFNLNFKGKNFEEIKKEVDANGYELENRILSRDALEELVKNKKCLLLPIVNQDIIKESKTESNQFTKDWNSIFDGNSPWRLTPEFRVSYRKPTPDYPMSDKGDKRYSRFQMIAHFLCDYIPQGGSYVSVREQIDNYKDDKKQEIAVKDFHDRLLGKTDEQKFIEGLGGLSSLGNVTIKTKLKKQDISKEKFYVFGIDRGQNELATLCVIDQDKKIQGGFRIYTRLFNNEKKQWEHKFLEERNILDLSNLRVETTIVIDGQEKREKVLVDLSEVKVKDNSGNYVKPNKTQIKLQQLAYIRKLQFQMQTNPGRVLEWYSHNQTSDLIIDNFVDKQNGEEGLVPFFGAAVAELKDTLPIDRISDMLKQFVELKNLEKQGEDVKSKIDQLIELEPADNLKSGVVANMVGVIAFLLKKYSYKVYISLEDLSKPFKDQIVLGISGVPIGIKKGMAGRSINVEQYAGLGLYNFFEMQLLKKLFRIQQDSSHILHLVPAFRAMKNYDNVAVGKGKIKNQFGIVFFVDAAATSKTCPCCGAINDRQFAPDLRKFPNAKKIETPDGKSVWLERDKTDGKDIIRCHVCGFDTSKEYDDNPRKYIKSGDDNAAYLISAECIKAYELATILVDNK

SEQ ID NO: 4 - protein, 1290 aa, synthetic construct
MNSIKNEYQLSKTLRFGLTKKKKLLKDDCNEIIYESHTELKELVLISEKKIMESVYINQKAKLDLSVDQIDTCLSSIKNFIDSWKGIYPRADQIAIDKDYYKILCKKITFDGFWIDEKTKTKKPQSRTILLSELSKKDASGKERKQHILDYWKNNIFSAIEKYEVVSRELKQFQKALKIQRTDNKPNEVELRKLFLSLANIILDILKPLVNGQICFPKIEKLDISKTDNKNLIDFATNHKFQSDLLNEIAELQHYFEENGSNVPFCRASLNPKTIIKSKLSTDNNIDKEIKQLGLDRILNEYLSAPYFDNSIIHLSAKEKLNKIEDKKENYITRGLLFKYKPIQIMLHHEIAKTLSKEIGKSEENIIEFLGNIGQIKSPAKDYEVSKEDFNINNYPLKVAFDFAWENVARNLYHTDTHAPIDECRKFLADNFDIKIEDNNLKLYANLLELNALLSTLKYGKPKDETSIKQNIKDLLNKISWNEIGKSGQKNKTNIENWLNNKDKIDNQNGIENAKKQIGLFRGSLKNKVPKYYKLTETYKDISMKMGKIFATMRDKITDEAELNKVSHYAMIVEDDNKDKYILLQEFTDKKEECIYSKTQTHNSDFTTYSVNSITSSAIAKMIRKVKAEELRKNQYNKDTFSIEETKEEKENRIIKEWKQFLKDKQWDYEFNLDTKNKNFEELKKEIDSKCYKLNISYIDKKTITDLVENKNCLLLPIINQDLSKEEKTQNNQFTKDWDAIFSQNTPWRLTPEFRISYRKPTPNYPISDKGDKRYSRFQMIGHFLCDYIPQSNTYISNREQIANYKDNEKQEQAVQCFHDKLLGKTEKEAKNEKLIALQAKFGSISRTNITQEKKKEKFYVFGIDRGQKELATLCVIDQDKKIIDDFDIYTRSFNSKTKQWDHTFLEKRAIMDLSNLRVETTISIDGKTEKKKVLVDLSKVKVKDKQGHYSKPDKMQIKMQQLAYIRKLQFQIQTNPDVVLAWYSDNNTQDLILENFVRKDDNDNKGLVSFYGAAVEELKDTLPIEEILNMLKQFKELKEKEKAGENVKYEIDRLIQLEPVDNLKTGVVANMVGVIAFLLEKFNYQVYISLEDLSQPFDNKINGGITGVPIKTNKESGRMADVEKYAGLGLYNFFEMQLLKKLFRIQQKSTTILHLVPAFRAQKNYDHVTVGQDNIKGQFGIVFFVNANATSKTCPICGANNSEKPDKNKYPNAHKELAKDGKEVWIERDKSNGNDIIRCFVCGFDTTKTYEDNPAKFIKSGDDNAAYLISVSAIKAYELATILAIEKYK

SEQ ID NO: 5 - protein, 1255 aa, synthetic construct
MNHIYNNYQVSKTLRFGLTQKQKIRRPGYTGELYESHKVLKELVKISEEKVKNLIVPAKNEELLSSLDSVKWTLTEIREFLDQWRYIYNKSNQIALDKSYYLILSKKLGFNDEKKSRVIKMIEIKDDIKEKIINYWAFNLNESNQKLLMVNEMVNTQLKALEINRTDHKINEIELRKALQSLFNTVLDILKPLVYREISFINLEKIEKDSKNSLLEKFATDFQRKIDLLEKIRSLKTHFSENGGNVSFCRATFNPKTAIKNPKSNDNSILKEIKKLGIKDILENNENVFYFEKKLAEITAKEKLEYIIKDSESFLIRSLLFKYISIPAFLHHGIATELAPIISKEKNDLINFMISIGQIKSPAKDYADIPNKNDFNVNAYPIKVAFDYAWETVAKSQYHHDINAPVSMCKTFLDENFENCTKTKYFTLYSDLLELHTLLSTLDYGNPSMEDSIIDKANKIIAKIDNKEHKTKDKDLDKDIDKYKETIKNRLNHKNFNDKQRYSDAKKELSQFRGKLKNENDIYRKLTESYKKIAMNTGKIFAEMRDKISNASEQNKISHHALIIEDHNKDRYLFLQEFTTDKEKQIESICNDQAGQYIVYWVNSITSKSISKMLSKKRIEKLKQKKIINNSIKTSILSDAEKEARDIKEWVSFIKEKGWDIDFNLDLQNKNLEEIKKEVDAKAYKLKETLISQKTLSNLVKEGNCLLFPIINKDLVKKVKTEKNQFTKDWNSIFKKDNLWRLTPEFRVSYRQATPGYPTSDIGTKRYSRFQMTAHFLCDFLPQGTKYISNREQIENYKSSEKQKEAVEIFHQQIENDNNNVISTQSLNHLARHFGSKNIKKKHNTIEKKFYVFGIDRGQKELATLCIIDQDKKIEGPFKIYTRSFNTKTKQWEHQFYEERYILDISNLRVETSISIDGKPDQQKILVDLSYYKEGEKFIKLPKMQVKLQQLAYIRKLQYQMQRNPETVLDWSYKNTDDKSILENFVDKPNGEKGLVSFYGAAVIELKDTLPLSEIKDMLERFKELKGKEKNGEDVSQQLNELTQLKSVDHSKYGVVANMVGVIAHLLERYDYKAYISLEDLTKPYSAIDGITGQKTDAKSISGKQQDVEKYAGLGLYNFFEIQLLKKLFRIQKDSQNTLHLVPAFRATKNYENLIAGEDKVKNRFGIVYFVDPKSTSIMCPSCGKTNNSSNKEKRVVRDKKNGNDIIYCEFCGFDTRNDYKENPLKFIKSGDDNAAYIISTHTAKKAYELAKSIL

SEQ ID NO: 6 - protein, 1255 aa, synthetic construct
MSLAAFTNQYQLSKTLRFGFTQKEKVRKENFDGSIYQSHAALRELTIESERLIKGKLKSNTDTALPLEKIRACIEEIKRYTDTWSKIFTRDDQLALSKEYYRVMARKARFDAFWKNYRDVKQPQSQIVRLSSLKSKYNGKERKAYLVDYWAGNLQTVKQRLVDFEPAIRQFESALKDNRTDRKLNEVDFRKMFLSICKLVNETLVPLCNSSLCVPDLEKLLDNEASQELRDFVMMDIFQLQEQIEALKIYFGENGGYVPYGRTTLNKYTALQKPHAFDEEIEAILVKLKLSDVINNLIKQDDVSDYFENVKDKIGQLSNSSMSVIECVQLFKYKPIPVSVRYSLVEYFHRKLGIDKDELGTLLDTIGKPKSPAKDYADLQDKGDFNLYKYPLKVAFDFTWESLAKAQYHEGLNFPEVQCQKFLENIFFVNTSCEAFKTYALLLHLRGLLAKLDHEEPNDREAIIDKAISLMNEDAFPKVPLRGTKGDSANQAILSWLQLSKEEQVYKKEKKDQSYNQYEKAKNKIGLLRGEQKNKIGKYREVTEQFKDLASNFGKLFGALREKFQAKNELNKITHYGTIIEDNNQDRYVLLYPLSEGIIDLDKLFVHEESGTLTSYYVKSLTSKTLNKLIKNKGGFKDFHMDGQQPDWERVKKRWSVYKDDKAFLKYVKRCLNESEMAKAQNWGEFGWDFSSCDSFEEIEREVDKKGYSFKNDRKLSEDTVKRLVKEEKCLLLPIINQDIIVEETKLRNQFSKDWVNIFDADCTEYRLHPEFGMSYRMPTPNYPKPEQKRYSRFQMIGYFQCEIVPIKTEYLSKKEQIEIFNDADAQKEAVEKFNEIVNGSVKPNDYVVIGIDRGLKQLATLCVLNKDGAIQGGFEIYTRSFNADKKQWEHRFMDNRDILDLSNLRVETTVDGKKVLVDLSSIKVKDQRGNYTQDNQQKVKLKQLAYIRKLQYQMQVNPEKVKAFAAQHRTPQDIKDHMKELITPYKEGSHFADLPLDRIKYMLEAFCAFHTENDQTSLRELIELDAADNLKSGIVGNIVGVIAFLLKRFSYNAYISIENLTRAFYNQRDGLSEKEIPRDHDFMDQENLVLAGLGTYHYLEVQLLRKLFRIQCDAGIINLVPAFRSNDNYETTRKLSKKQGVEYVCKPFGIVHFVDPMYTSKKCPACGGTTVQRGSFKDDITCQNPLCGYGTSLDISEKIQKLISANKAGQNIHLISNGDENGAYHIALKTLKNLFGNIQNANNERRYVKSFRSK

SEQ ID NO: 7 - protein, 1234 aa, synthetic construct
MEKNEIAISEYQTQKTIRFGLTATNQNLYSEEIMKLLDISEKRVEKQAEQAKKVNNDADKNNQLRCCLDQIKEYLKTWSNIYPQIDFLAITKDFYKVISKKARFDFDKGNGSEIKLSYLQSTYYNKKRYLYIIESWKENLRKTENLYRKSDDLLKVFEEAKNQNRDDKKLNKVELRKTFLSLFNLVNESLKPLIEGNLFIVNDDKIDEQNPKHDCVSVFISKTEERRKLYDYICDLQDYFKDNGGYVPLGRVTLNKWTALQKSNNRDAEINRIIKELKINSVSIQNIEYEYNNFANNFKEKKDENGKIVKNNAGNIIWDLKADAKSVIEICQFFKYKQVPINARLNLAKRLEKSIDFLSEFGVSKSPALDYKNDKNNFNLTNYPLKIAFDYAWENCAKAKHEEIPFPKEQCEKYLKDVFDIDIECKEKCQNKECKGCEKCRGYYLNKYADLIRFKILLGRLKAEFHKTDEEKNKSNIQELRNIFRDLDYRGDKRLNKNEIQKAVNAWFDNKEQSIGRKKEDEIHLMENEKNKFSLSMQIIGQERGGLKSRISKYKALTEMFKVCASKFGKQFADLRDYFNEAYEVDKIKYRAWIIEDEKQNRFILFVNKEKEVDLTSEEGDLYFYEVKSLTSKSLVKFIKNRGAYPDFHKINNRQIDLNSGEKDSRGNFIDDVKIHWSTYKNNQKFLDKLKDCLQNSTMATVQKWSEFEFEFDFSNCDTYEKLEKEIDRKGHKLERKTISLTTITNLVENTACLLLPIVNQDLNKGNKQAKNQNQFTKDWFDIFENKKRLHPEFNIFYRFKTKDYPNTKFKNGTEKTKRYSRFQMLAHFGCEVIPQGDYLSKKEQIAIFNDDKKQTEEVKKYNKNISSDVDYVIGIDRGIKQLATLCVLDKNGVIQGGFQLFTRTFNSETKQWEHQELEKRNILDLSNLRVETTITGEKVLVDLASIQTKNGENRQKIKLKELAYIRDLQYTMQTRASDLLDFASKINSADDITENNIKNFISPYKEGEKYADLPQKEMFDLLTEWKNAEEEGKRKIAELDPADNLKSGIVANMVGVVALLCAKYKYRVRIALEDLTRAYGIQKDALSGATIFQNDEDFKEQENRRLAGVGTMQFFEVQLLKKIFKVQIDKDLHLIPAFRSIANYEKIVRRDKQNSGDEFVNYPFGIVCFVVPKYTSKRCPKCEKTNVNRKENIVICKECGFQTKEGNPYEKNNIHFITDGDQNGAYHIAKKAL

SEQ ID NO: 8 - protein, 906 aa, synthetic construct
  VARIANT 24: note = Any amino acid
  VARIANT 141: note = Any amino acid
  VARIANT 170: note = Any amino acid
  VARIANT 174: note = Any amino acid
  VARIANT 175: note = Any amino acid
  VARIANT 199: note = Any amino acid
  VARIANT 228: note = Any amino acid
  VARIANT 259: note = Any amino acid
  VARIANT 261: note = Any amino acid
  VARIANT 302: note = Any amino acid
  VARIANT 308: note = Any amino acid
  VARIANT 328: note = Any amino acid
  VARIANT 376: note = Any amino acid
  VARIANT 429: note = Any amino acid
  VARIANT 456: note = Any amino acid
  VARIANT 527: note = Any amino acid
  VARIANT 541: note = Any amino acid
  VARIANT 569: note = Any amino acid
  VARIANT 584: note = Any amino acid
  VARIANT 601: note = Any amino acid
  VARIANT 632: note = Any amino acid
  VARIANT 660: note = Any amino acid
  VARIANT 711: note = Any amino acid
  VARIANT 751: note = Any amino acid
  VARIANT 775: note = Any amino acid
  VARIANT 777: note = Any amino acid
  VARIANT 798: note = Any amino acid
  VARIANT 807: note = Any amino acid
  VARIANT 846: note = Any amino acid
  VARIANT 848: note = Any amino acid
  VARIANT 849: note = Any amino acid
  VARIANT 866: note = Any amino acid
  VARIANT 884: note = Any amino acid
  VARIANT 902: note = Any amino acid
MEKNYKSEINLSYEDILNKFNLLXRAIDIANDFKNSNNQNNFSLDEYPIKLAFDYAWENTARSLKRTIPFPKEVCKQFLKDNFDVDIDNADFKLYANLLFIADNLATIEYNNPNNEVELINEIKQAFECISFPFDKEVYKXHKEAILELLDKEKSQRDYSTILKAKQELXLLRXXLKNKIKKYRDLTQRLIDKKNSHFXIASFVGKTLATIRDGLKEENELNKISDYXVIIEDSNQDKYLLTLELNGKDIRDRIRNSLXNXEYKTYEVNSFTSKALNKFIKNPLSEDAKKFHGKYKDEYSFXYENKDXDFTYKITKVSKYDEQGKWTXYQESFLSHIKKCLIDSEISREQNWEAFGWNFAGCNTYEEIEKEVDSKXYQLTENLISMGNLKSLVKDEGCLLFPIINQDISSQKQENKNIFTLDLEKVFEXKECRIHPEFSIFYRRPIEEHKKENKSXIINRFGRLQLLANLGIEFVPRNPSFKTKKEQNRIAIDQKKQNQLVQEFNQKKVNTYFEGLDNYYIFGIDRXIKQLATLCVTDKDXVIQDFDIYTKHFNSESKKWEYKFHRKDXILDLTNLKIESDRSXNKYIVDISLFQAKDEDXNPTGTNKQNIQLKQLAYIRKLQYQMSANEEXVLNFLGKYKNKEEREQNMEELITPYKEXKNFADLPMDIFQEMFENYYRLKTDQNLSESEKKNLMKITTELDASESLKKXVVANMIGVIYYLMKKYEYKVKISLENLSNAWLFSKDGLSXDVVLNTKNDETMDLKKQDNLALAXVXTYHFFEMQLLNKLFKISTEEXVLHLVPSFXSVKNYIEIMKIKGKYVYKQFGIVYFVDPRNTSKKCPVCXKXXKKYISRVDNVVTCKNCXFDTSSDNSILINNYKKQXKNIHFIKNGDDNAAYNIXEKIR

SEQ ID NO: 9 - protein, 1029 aa, synthetic construct
MRKMFLSLANIVCEVLVPLCNGSICFPDIEKMSDKDENKYLREFAVDYKTKIDLFDSISELRKYFEENGGNVPFCRATLNPKTKIKNPSSTDNSIEAEIEKIGMEKYLQTTDEIITAINKIKQMQDSKLPLIKRALLFKYKTIPAVVQFDLAKSIGKKLNKEEQELRTIIRTIGQTASPAKDYADLQHKKEFNINQYPLKPAFDYAWESLAKAEYHNDIEFPNQICKQFLKDIFNVDTETDSYFKLYAQFLYLRELLATLEHSQPTNPEKIVKIIEELLEKINAWKEIDDKNSEKYKTAISNWLKTFNKEDNNFKNAKQKIGLFRGGLKKKAGDKSRWEYLRKPDEKEAPKLTAYNALTQIYKDIATNLGRNLADMRDKITNESELNKISHYAAIIEDKNGDKYVLLKKSDKDEEFALPCEKDAEYKTYIVNSITSSAIAKMIRKKRAADLAKNIRLKSEELNDEQKEAKNLKDWIELIKKQQYDFEFNLNLNNKNFEQIKKEIDAKCYQLQKGNISKQTLEKLINEQKEWLLLPIINQDLAKRDKSTTNQFSKDWQKIFSDKRCGYRLTPEFRISYRKPTPNYPQSQIGDKRYSRFQMIAHFLCDYIPQGNLEYKSTRQQIEIFKDYEKQEESVRNFTYKIYANNDYLIFGIDRGLKQLATLCVLNKEGKIYGGFDIYTRSFNKDKKQWEHSLSEKRNILDLSDLRVEKTITGEKVLVDLNSIKVKGNQDNQQKIKLKELAYIRKLQFKMQTEHGTVINFINKYRTPEEIQKNIHELITPYKEGEHYADLPTEKICNMLQKFKEFSDKNDGKSKRELIELDSAGELKNGIVANMVGVVAYLLEKYKYNVYISLEDLTRAYRRQTDGLDGRELFSSNDDKSVDFKDQENTALAGLGTYHFFEIQLLRKLFRIQQEDGNILHLVPAFRSVDDYEKIIRRDKKIDGNEYVDYPFGIVRFVDPKNTSKRCPLCSSININRHKNIIKCQQKACGFKTPWDKTNDKNIQYIQNGDENGAYHIAKKTLDNLNNKK

SEQ ID NO: 10 - protein, 1257 aa, synthetic construct
MDADKTTKAINEYQTQKTIRFGLTATNQNLYSEEIMKLLNISEERIIKEKVKVNNDTDKTNQLRGCLVQIKKYLKTWENIYAQIDFLAITKDYYKVISKKARFDFDKGNGSEIKLSSLQSTHNKKKRYQYIIDFWKENLRKTENLYRKSDDLLKIFEEAKNQNRDDKKLNKVELRKTFLNLFTLVNESLKPLIEGNLFIVNDDKIDEKNSKHNYVFYFISKTEERRLLYDNICTLQDYFKNNGGYVPFGRVTLNKWTALQKFNNRDIEINRIIKELKINNISTQKTDYKYNDFTENFKEKKDENGKVVKNSAGNIIWDLKANAKSVIEICQFFKYKKVPINARLNLAKRLIKDNKLKKEQENTFLSEFGVLKTPAFDYARDKENFNLTNYPLKVAFDYAWENCAKDKYEKIPFPKEQCERYLQTAFEIDATKDENKKLIDTHLNKYADLLQFKILLERFKAEFHKTNEETNKNNIQKLRNVFSGLDYHGDNRLNKNQIQKAIEAWFDNKEQNIGKKKENEKLLTENEKNNFSLSMQIIGQERGGLKNGIPKYKELTEMFKVCASKFGKQFADLRDYFNEAYEVDKIKYRAWIIEDDKKNRFVLFVNKEKAFDLTSEEGDLWFYEVKSLTSKSLVKFIKNRGAYPDFHDVKNSFHYSSIKKDWQNYKNDPEFLDKLKECLKNSKIAKDQKWAKFCWDFKQCDTYEKLEKEVDRKGYKLEGCKSEPKTISLTQLTDWVENKDCFLLPIVNQDINKGDKRTKNQNQFTKDWFDIFENKKRLHPEFNIFYRFPTKDYPNTKFKNGTEKTKRYSRFQMLAYFGCEVIPSGNHLSKKEQIAIFNNDKKQKEEVEKYNKSISSDCDYVIGIDRGIKQLATLCVLDKNGVIQGDFQIFTRTFNKQTKQWEHKELEQRNILDLSNLRVETTITGKKVLVDLSKIKDDEGNYTNLKQTIKLKQLAYIRELQYAMQTRPDDLLDFVKSINSANDITAENIKHFISPYKEGKNYDDLPKVEMFNLLKEWGNADENGKRKIAELDPADNLKSGIVANMVGVVAFLCENYNYKVRIALEDLTRAYGIQKDALNGTAIYQNDEDFKEQENRRLAGVGTMQFFEVQLLRKLFKIQVDKNLHLIPAFRSVDNYEKIVRRDKQNSGDEFVNYPFGIVCFVDPKYTSQQCPYCNNTHKHKKNDTETGKKAFYRNKGENKNSLLCEKCGVSTIEGEETLSSKNDNKKQFNIHYITDGDQNGAYHIANKVVINFQKDS

SEQ ID NO: 11 - protein, 1276 aa, synthetic construct
MEKIKNKYQVSKTLRFGLTQKEKRRKKGFVGEVYESHTELKDLTDFSVKKIRAEIKQGGNSGIPIEKIRQCLIVIRRYMNFWENIYYRCDQLSLDKDFYKKLSKKIGFEGFWHEENRKTDHRIKKPQARTIQLSELNKKDDHYKERKDYIVEFWESNINKAAERFKETESVFEKFEIAINANRDDNRPNEVEMRKMFLSLANIIYETLVPLCNGSISFPNIEKMQNNEENNNLRKFAADDEFRAGLLTQIEELKAYFEENGGNVHYCRATLNPKTVIKNPNSTDSSIADEVEKTGIERYLQNKQEIKNKIEKIKDKNLPLIERALLFKYKTVPAGVQFDLAKFLSVKLGKTEQELRTIIRCIGQIQSPAEDYAKSEDKKGFHLEQYPLKSAFDFAWESLAKAIYHKNVDFPQQQCKVFLEKNFDIKIDSNANFKLYAQLLYLRENLATLEHSKPTDPDTFEKNIRKLLDEINWSTIDKEKGSVYKNAISDWLKNKKAKDEKFGKVKQSIGLSRGRLKNKIKKFDDLTKDYKDIATQLGNAFAAMRDKITNAAELNKISHYATIIEDKNGDRYILLQKVTENEKPVGENWNKNGELKTYLVNSVTSAAISKMIRKIRTDELRKNEKMQSSITKLNEKQKEEKNINDWKNFIEEKRWDLEFKLDLKSKNFEQIKKEIDTKCYLFNTGYASQADIKQLVKEKDALLLPIINQDLASKDKIVRNQFSKDWQMIFSDNSQGYRLTPEFRISYRQPTSNYPQPEEKRYSRFQMIAHFLIDYIPQNNQYISTREQVELFKDETKQREAIEEFHKQLTPKTEAEQKAESLSALAAKFNNPNNKKQKNNASENKPDEKFYVFGIDRGQNELATLCVINQDKKIIGDFEIYIRKFNSEKKQWEHLKLENRHILDLSNLRVETTIVIDGKPEKKRVLVDLSEIKVKDKNGEYKNPDKMQTKMRHLAYIRKVQFQIQNNPEGVLDFLKKFKTKNETIYNLVDKENGEKGLISFYGAGNTNEDLPKDDIWKILQKFQELKNKTGDDNVKKEIKELIELEPVDNLKNGVVANMVGVIAYLLEKFDYQVYIALEDLTKPFNEDIKDGTTGVKGNYKGEGKRADVEKYAGLGLYNFFEMQLLKKLFRIQQENDNVLHLVPAFRAVKNYENLIAGKGKIKNQFGIVYFVDANSTSKTCPVCSSIPDKNNSNNKNAKGKKIINKKGNESVIWVERDKSNGNDIIRCYACGFDTTKNYSENPLKYIKSGDDNAAFIISTLGIKAYELAKTLVANK

SEQ ID NO: 12 - protein, 1276 aa, synthetic construct
MKSFENSCQISKTLRFGATLKEDKKKCKSHEELQEFVDISYKTMKSSATIAESLNEIELVKKCERCYSEIIKFHEAWGQIYCRTDQIAVYKDFYRQLARKARFDVGDQNSQLITLSSLSSKHNVYQGLKRSQHITNYWKDNIARQKSFLKDFSQQLHQYKRALENSDKAHTKPNLINFNKTFSILANLVNEIVIPLSNGAISFPNISKLEDGEESRHLIEFALNDYSDLVGSIGELKDAIATNGGYTPFAKVTLNHYTAEQKPHVFKNDIDAKIRELKLIELVEKLKNKTSKQIEEYFSKFDKFKIYDDRNQSVIIRTQCFKYKPIPFLVKHELAKYIAEDEDAVLKVLDAIGATRSPAHDYAHNEDVFDLKHYPIKVAFDYAWEQLANGVYTTVSFPEEKCREYLNAIYGCEVTKEPVFKFYADLLYIRKNLAVLEHKNNLPSNPEEFICKIENTFEKIVLPYKIKEFETYKKAILTWINDGRGHEKYTDSKRELGLIRGGLKGRIGAQKMFRKNKKGELIPYYENPYTKLTNEFKNISSSYGKTFAELRDKFKEKSEITKITHFGIIIEDNNKDRYLLANGLQHDNTDQSNTQVEAILGKLNTSLEFTTYQVKSLTSKILIKLIKNHTTSPNAKSPYADFHTSKIHVDWTKIKKEWDTYKSNHSLLQYVKDCLTNSTMAKNQNWAEFGWDLESCNSYEAIEHEIDQKSYILQKHTISKASIKSLVENGCLLLPIVNQDITSQGRKDKNQFSKDWKQIFEDSKEYRLHPEFAVSYRTPIKDYPKDKRYGRLQFICAFNCEIIPQNGEFINLKKQIENFNDEDIQKNNVAEFNNKVNEALLGKEYVVIGIDRGLKQLATLCVLNKRGKILGDFEIYKKEFVRTENRSKNYWKHTLAETRHILDLSNLRVETTVEDNKVLVDQSLTLVKKNRDTPDEKATEENRQKIKLKQLSYIRKLQYAMQTNEQAVLDLLKDNSDDEEFKKRVEGVISPFGEGQEYADLPIDTMRAMIQDLQGVIAKGNKQTEKNKIIELDAADSLKQGVVANMIGVVNFILAKFNYEAYVSLEDLSRAYNVAKSGYDGRYLPSTSQDPDMDFKEQQNQMLAGLGTYQFFEIQLLKKLQKIQSNNTVLRFVPAFRSADNYRNIIKLNPKYDNTEYVHKPFGIVHFIDPKDTSSKCPVCGKTGKKNVDRNEKKNNILLCKACGFRTVWELKQPENIKSEGYCFSEDEIQKTIKNNQKQMEIKKLSDKNIHYIHNGDDNGAYHIALKSVENLKHKE

SEQ ID NO: 13 - protein, 1297 aa, synthetic construct
MKSIVNNYQISKTLRFGLTQKTKIQKEGYNGEIYVSHRELCDLVKISEERIKKSVSASDKSNLELSIDIIDTCLKQIGVFLSDWQQVYYRKDQVALDKDYYKILCKKIEFDGFWKDDRGQRMPNSRIINISELDKRDSLGVERLHYILNYWKDNLVSASQKYSAVEEKIKRFKSAIKINRTDNKPDEVELRKMFLSLANIVCDTLQPLCYGQISFPKINKLDDSRTDNKKLIKFATEYKSKNDLLTSIAEQKKYFEENGGNVPFCRATLNPKTAIKDPNSTDNSIKGEIAQLGLDSILKSFKSYLFFENSLENMSAKEKIDLMKSDGASIIKKGLMFKYKPIPVIVHREVAQELSEDLNKTEESLSDFLRGIGQAKSPAKDYEELTDKNEFNIEAYPIKVAFDFAWESLAKAKYHSEIDLPVDSCKKFLKTFDVTPDDANFLLYAQLQELNALISTLEYGYPSDEQSIVKKIKALSNEIQWEKVSDRNGQEYKRSISDWADGKRDSDGFKIAKQNIGLFRGGLRNKIDKYYSLTQIYKKTVMDEGKIFAIMRDKIIGAAEQNKVTYYAAIIEDNTGDKYVLLQELPLNGQDRIYDKMTRDGDGYVCCFVNSITSRTIAKQLRKKRMAELKKNKAWGAYNDNVSNTQQPVLSDEEKEERNIREWKSFISEKRWDCEFNLNFKGKNFEEIKKEIDAKGYELENRRLSREALDELVKNNKCLLLPIVNQDIIKENKTESNQFTKDWNSIFDDNSPWRLTPEFRVSYRKPTPDYPVSSKGDKRYSRFQMIAHFLCDYIPNSDSYVSVREQIENYKDDKKQETAVKDFHNRLLGKTEEQKIIDRLGAFQGWGNVIIKKTKPEKQEGSKEKFFVFGIDRGQNELATLCVIDQDKKIQGGFKIYTRSFNSEKKQWEHKFLEERNILDLSNLRVETTIVIDGQEKKEKVLVDLSEVKVKDHFGNYVKPNKMQVKLQRLAYIRKLQFQMQTNPERVLEWYSHNQTNDLIINNFVDKQNGEKGLVPFFGAAVAELKDTLPIDRISEMLKQFVELKNLEKQGEEVKSKIDQLIELEPADNLKSGVVANMVGVIAFLLEKYSYQVYISLEDLSKPFENQIVLGISGVPIGINKGMAGRSVNVEKFAGLGLYNFFEIQLLKKLFRIQRDSCHILHLVPAFRAMKNYDNVAVGKGKIKNQFGIVFFVDAAATSKTCPCCGANNSKKFEPDFRKFPNAKKFETPDKKSVWLERDKSDGKDIIRCHVCGFDTSKEYDDNPRKYIKSGDDNAAYLISAEGVKAYELATTLVDNK

SEQ ID NO: 14 - protein, 1315 aa, synthetic construct
  VARIANT 20: note = Any amino acid
  VARIANT 28: note = Any amino acid
  VARIANT 81: note = Any amino acid
  VARIANT 140: note = Any amino acid
  VARIANT 151: note = Any amino acid
  VARIANT 234: note = Any amino acid
  VARIANT 248: note = Any amino acid
  VARIANT 259: note = Any amino acid
  VARIANT 265: note = Any amino acid
  VARIANT 293: note = Any amino acid
  VARIANT 375: note = Any amino acid
  VARIANT 492: note = Any amino acid
  VARIANT 521: note = Any amino acid
  VARIANT 525: note = Any amino acid
  VARIANT 526: note = Any amino acid
  VARIANT 550: note = Any amino acid
  VARIANT 610: note = Any amino acid
  VARIANT 612: note = Any amino acid
  VARIANT 653: note = Any amino acid
  VARIANT 659: note = Any amino acid
  VARIANT 679: note = Any amino acid
  VARIANT 727: note = Any amino acid
  VARIANT 807: note = Any amino acid
  VARIANT 878: note = Any amino acid
  VARIANT 892: note = Any amino acid
  VARIANT 920: note = Any amino acid
  VARIANT 935: note = Any amino acid
  VARIANT 952: note = Any amino acid
  VARIANT 983: note = Any amino acid
  VARIANT 1011: note = Any amino acid
  VARIANT 1062: note = Any amino acid
  VARIANT 1094: note = Any amino acid
  VARIANT 1095: note = Any amino acid
  VARIANT 1099: note = Any amino acid
  VARIANT 1121: note = Any amino acid
  VARIANT 1123: note = Any amino acid
  VARIANT 1144: note = Any amino acid
  VARIANT 1172: note = Any amino acid
  VARIANT 1181: note = Any amino acid
  VARIANT 1205: note = Any amino acid
  VARIANT 1209: note = Any amino acid
  VARIANT 1229: note = Any amino acid
  VARIANT 1273: note = Any amino acid
  VARIANT 1291: note = Any amino acid
  VARIANT 1310: note = Any amino acid
MINLENFKNLYEVRKTVRFXLNQPNKKXDFKTHLEFKDFTEKSFQNVENELASNGKYNIGDEEQLIKKISAFVEQLKIQLXYWKVFYQRYDLIAINKDYYKILARKAKFDAIWEISKSNKYIKVKQPQASQISLSSLKIXNRSDSIIQYWXEIIEKTDYLLNIFKPKLEQYERAIKDANNAHIKPDSINFRKTFLQLLKLTKEFLQPLSDRSIIFEFSKKKVSKEIEKISEFAXEKNNTKIYNVLKNXEELRQYFEANXSQVSYXRVSLNYYTAVQKPNNFDQEIKKSIDDLXIINFLKKSDSQIIDYLHQGSKQKIKLLLTSKHPYPIELLQLFKVKPIPFSVKYNLAKFVEKNYKSETNLSYEDILNKFNLLXRAIDIANDFKNSNHKGNFSLDEYPVKLAFDYAWENTARSLKRDISFPKKVCERFLKDNFDIDVDNADFKLYANLLFIADNLATIEYNNPNNEAELINEIKQAFECMSFSFEKKVYEXHKKAILELLDKEKSQRDYSTISKAKQELXLLRXXLKNKIKKYRDLTQRLIDKKNSHFXIASFIGKTLATIRDGLKEENELNKISHYGVIIEDNNQDKYILISKLEGKDRRDKIVQKLXKXDIKVYQVNSFTSKALNKFIKNPLSEDAKKFHGKYKDEYGFXYENKNXDFTYKVKNVSEYDEQGKWIXYQEEFLNHIKKCLIDSEISREQNWTAFGWSFDGCNNYEEIEKEIDSKXYQLTENSISKGNLESLVKDEDCSLFPIINQDISSQKQENKNIFTLDFEKVFERKECRIHPEFSIFYRKPIEEHKKENKSXIINRFGRLQLLANLGIEFVPRNLSFKTKKEQNRIAIDQKKQNQLVQEFNQEKVNTYFEGLDNYYIFGIDRXIKQLATLCVTDKGXVIQDFTIFTKHFNQASKIWEYRENRKEXILDLTNLKIESDKNXNTYIVDISLFDAKDDNXRSTGTNKQNIQLKQLAYIRKLQYQMSANEKXVLDFIAKYISKDEREQNIKELITPYKEXKKFADLPMDIFQEMFENYYRLKTDQNLSEFEKKNLMKITTELDASENLKKXVVANMIGVIYYLMKYYDYKVKITLENLNQSFXXQVDXINNHYVDIKNNFMYQENQALAXVXTYHFFEMQLLKKIFKISTEEXILHLVPSFGSVKNYFKDIEKYSLIVNIXRNEKYNQFXVVHFVKPDNTSKKCPVCLNTQTTXKKEXMPIINRNYKKSNIFYCERCXFQSIHSHCQEENIKDSNDFHYSVEEVERIEMKNKEAIEKYKKQXKNLHFIKNGDDNAAYNIXEKIRELPKKSDVKNTSISXNISFS

SEQ ID NO: 15 - protein, 1276 aa, synthetic construct
MKSFENSCQISKTLRFGATLKEDKKKCKSHEELQEFVDISYKTMKSSATIAESLNEIELVKKCERCYSEIIKFHEAWGQIYCRTDQIAVYKDFYRQLARKARFDVGDQNSQLITLSSLSSKHNVYQGLKRSQHITNYWKDNIARQKSFLKDFSQQLHQYKRALENSDKAHTKPNLINFNKTFSILANLVNEIVIPLSNGAISFPNISKLEDGEESRHLIEFALNDYSDLVGSIGELKDAIATNGGYTPFAKVTLNHYTAEQKPHVFKNDIDAKIRELKLIELVEKLKNKTSKQIEEYFSKFDKFKIYDDRNQSVIIRTQCFKYKPIPFLVKHELAKYIAEDEDAVLKVLDAIGATRSPAHDYAHNEDVFDLKHYPIKVAFDYAWEQLANGVYTTVSFPEEKCREYLNAIYGCEVTKEPVFKFYADLLYIRKNLAVLEHKNNLPSNPEEFICKIENTFEKIVLPYKIKEFETYKKAILTWINDGRGHEKYTDSKRELGLIRGGLKGRIGAQKMFRKNKKGELIPYYENPYTKLTNEFKNISSSYGKTFAELRDKFKEKSEITKITHFGIIIEDNNKDRYLLANGLQHDNTDQSNTQVEAILGKLNTSLEFTTYQVKSLTSKILIKLIKNHTTSPNAKSPYADFHTSKIHVDWTKIKKEWDTYKSNHSLLQYVKDCLTNSTMAKNQNWAEFGWDLESCNSYEAIEHEIDQKSYILQKHTISKASIKSLVENGCLLLPIVNQDITSQGRKDKNQFSKDWKQIFEDSKEYRLHPEFAVSYRTPIKDYPKDKRYGRLQFICAFNCEIIPQNGEFINLKKQIENFNDEDIQKNNVAEFNNKVNEALLGKEYVVIGIDRGLKQLATLCVLNKRGKILGDFEIYKKEFVRTENRSKNYWKHTLAETRHILDLSNLRVETTVEDNKVLVDQSLTLVKKNRDTPDEKATEENRQKIKLKQLSYIRKLQYAMQTNEQAVLDLLKDNSDDEEFKKRVEGVISPFGEGQEYADLPIDTMRAMIQDLQGVIAKGNKQTEKNKIIELDAADSLKQGVVANMIGVVNFILAKFNYEAYVSLEDLSRAYNVAKSGYDGRYLPSTSQDPDMDFKEQQNQMLAGLGTYQFFEIQLLKKLQKIQSNNTVLRFVPAFRSADNYRNIIKLNPKYDNTEYVHKPFGIVHFIDPKDTSSKCPVCGKTGKKNVDRNEKKNNILLCKACAFRTVWEFKQPENIKNEKHCFSEDGIKKTAEDNNKKIKEKNLSDKNLHYIHNGDDNGAYHIALKSVENLKHKK

SEQ ID NO: 16 - protein, 1342 aa, synthetic construct
MLTKFANLYELTKTVRFGLTPKNSYKKVSDFFELTEKSLESVQAEIVEREKEKINITSTEESIKKIRAYVDELKKQSAQWKTIYQREDMIAVTKEYYKKLEKEAGFDGFWEDRGKKQPQTSEIMLSALSKEYNNKPRREYIITYWALLLQKTEQLNSYFEPLLEKYEESLSRKEQAHLKPNLVDFRKQFLSLLNVSNKWLMPVINQSIVFPKIHNASKGEQNRKVKDFISEEGRQKRIQLQSIGRSLRDFFEANGSRVPFGKATLNYFTARQKPNRFDSEIDALMLDLEIEEIVRKVKDLNGEELKNYFKYQTYATGDKLDAFLGHSYMTLIEKIQLFKPKPIPASVRFSLAERLSKKMNLPMEKVTAIFDEIGNPIDIAREYEQALDQKNFDLNKYPINIAFDYAWESCASLIKGRISEGDFPKAQCLAILKKFNADGDDFKLYANLRYIRDELAPIEHNNPSADIEKELVYNIQKTIGIIYSSMPSEYQKYLDIILKWVEMPKIERNSSDSNFIFAKQKIGLLRGGLKNKINKHAEITNKFKNLSMAFGRSCSDLRDKLREEHELSKIKYYSVLIEDSKKNRHLLMLPLETGDQKMSAVEIENRLDILENKFVEDAPSANAYIASKVSSFTSKALVKMLKNPKGSEDFHIGSEKYKLHDYNNKIKKEWKIYQDDSKFLSYAKKCLSESKMAMNQHWEKFGWNFEGCKTYADLEKEVDIKGYSLEKKYISFENTEMLVYEQGCLLFPIVNQDYASEVCQNIFTGNKNAFTIEIEKGLQEQDGYLIHPEFTIFYQKPTEDYKKSNRYGRFQLSANFSLEVKKISDNFKTKKEKLKNIRDKDSFQKDVQIFNESINAQLRINEDVYFYGIDRGINELATLCLLNNKNKIQDFIVFKKEKKKRNSENHLREAGDFYTYVDFEKQNILDLTNIKVETWNMRDEIHPNSNFLKQAFKKIEADPEMKMIATQLGYGINEKKQCTILVEYPSDRSNTLKLRMNHFQRALQFATSHSEKRDIVGGIAERNFEGTDLENPSDEVKKQIIQDLFGLGSDFLIDEKKAGVINELQTKKFRFEKIYSRNQQLFLEMFEPLIQQLIEWYQHKNDPDYEKTGKAKYEEMEDLDTLKRGVTANMVGVFSFLMQKFPGVIVLENIAKEKLEKPVQSKLDEHTREVESHQEAEHHRWAGVELYRYMEKKLVQKFSNWRQPTGEIFNLTPPFANLESLNKQTMTINSERIENMFQFGIFCYTPAEYTTKTCPCCNKYHIRNRKKGNTFDSIICKNEHCGFDTRYNEPLHESFNPARNVEIALRLNPEFSEALPKIRSGDQSAAFNIAKKGLIIMENKLC

SEQ ID NO: 17 - protein, 874 aa, synthetic construct
MKECKSFLKESFQVDTDKNPHFKLYAQLIHLKEFLSTLESARPKDTQSIVKETQNLLSQINWDALGKDKNDKRTGNDYKISISSWLELRPKNDEKFQKAKQQIGLFRGGLKNKIKNFNDLTQAYKAVAMKLGKAFGDMRDKITGAAELNKISHYAAIIEDKNADRYILLKKSQLSGGSQKPFQSNDSGDLLIYTVNSVTSAAIGKMLRKMRAEELKKNEKNRQPLPELTDEKKEKESIERWKSFIKEKGWVSEFELNLDNKTFEQIKKEVDSKSYSLDVGRLSKNALEKLIHNEECLLLPIINQDLASEGKIAKNQFTKDWQRLFADEPQNLRLTPEFRISYRQPTPNYPDPGEKRYSRFQMMAHFLVDYIPKIDSYISIREQTETFKNPEKQKKAVAQFHQRLNPKSKSESDPIALLSAKFGSQINKNQGRKASTEEAKQKEDFFVFGIDRGQKELATLCVIDQDKKIIGDFEIYTRQFNAEKKRWEHSLLPERRHILDLSNLRVETTLSIDGIDQKKRVLVDLSEVKVKDKEGNYSLPNKMQTKMRHLAYIRKLQFQLQSNPDGVLDFLSKHPEKENALDNLLDKEEDEKGLISYYGAGNASEDLPKEELWNILVNFRELKNQLQEDPENQALDQELRALIELEPVDNLKYSVVANMVGVIAYLMEKFNYQVYIALEDLTNPFAPTTVSGITGVSNKNRQGEGKKSDVEKYAGLGLYNFFEMQLLKKLKRIQQENGNVLHLVPAFRAVKNYENVIAGKDKIKNQFGIVFFVDADSTSKMCPVCESTNDKQPNKSKYPEAKKEKSREGKEVWVERDKFNGKDRIRCYVCGFDTDKDYDENPLKYIKNGDDNAAYIIATLGIKGYELSLADLNP

SEQ ID NO: 18 - protein, 1228 aa, synthetic construct
MEPIKNEFQLLKALGFGLTAKHSEKKNKTHEELMDLIQLSEERIKGKTKPTNEITEKKMLEIISECVFLMKHHINTWEKVYNRADQITLTKEYYKELAKKACFDAFWTDNKDNKQPKSQLIKLSSLKKPRYENYERMGYILRYWRTNIQNLKRKIHDFELLLSQYKLALSKQDKAHTKPQLVDFRKMFLSIVALIHETLIPLNNNSIQFLQLDKLPDGEDSETVKTFAIEEREERKYLAAKIKETKEYFEENGGYVPYGRVTLNPYTAKQRPHIFNEEIADIIKDLGVEKLIKKLQSINDISKYFEFAESDNDKRKLMKDSSYSIIERVQFFKYKPIPASVCFMLADFLQRTKKLEKENVLDLLNKIGMQRSISKEYKDKKSINLEQYPLKIAFDYAWENLAGRIHNSTIPFSDQQCVDFLQQFGVDINNDNFRLYADLLFVKNNLATLENPKNQPNDGSEFIDNIKRTFENIKLSKENNKHKDVILNWLNKNTEEQKELKKGNAQLFKDYEKAKEKLGQLRGMQKNSCPKYKSLTEEFKNLAIDFGKKFADLRDKLREESELNKITHFGVIVEDSQKDRYILLGKPEDKLIHKFLNNDPNGNLKTYNVKSLTSKALSKIIKNKGAYKAFHSSQVKIDRKEIKQKWQEYQDHGDFINYVKDCFANSPMSKEQKWSEFQCKFDHCQKYEEIEKELDAKAYLLKEQNISLQTIKKLVEQEDCLLLPFVNQDITSKTRERKNQFSKDWDMMFKKDNGYRLHPEFRIFYRQPTPDYPKSGEKRYSRFQMTARLLCEFIPQSDNYISHKQQIKTFNNKDKQEEAVKEFNDKLHHPDNFYVFGIDRGINQLAVLCVLNKEGQIQEGFKIYTRSFDIEKKQWIHELSEKRNILDLSNLRVETTINNEKVLVDLSGIKIKNKEGYQDNRQKIKLKQLAYIRKLQYQMHAAPQRTLAFINKYHTPEDMKKHIQEDIKELITPYKEGVNYSDLPIEKICDMLQQFKQYYDKEDKKSTRELTELDSADELKTGIVANMVGVIAYLLKEYDYKVYISFEDLTRAFGRQKDGLDGRELPHSNEDKTVDFKGQENAALAGLGTYHYFEMQLLRKLFRIQQENKDILHLVPAFRSVDNYEKIVRRDKKSDNDEYVNYPFGIVRFVDPRNTSRKCPNCGEIKKQIITRSKNKITCRKCGFITGNNNHDKIKDNKKDKNLHYITNGDDNGAYHIALKTLKNLKGQ

SEQ ID NO: 19 - protein, 1210 aa, synthetic construct
MEKDKITAISEYQTQKTIRFGLTATNQNLYSEEIMELLNVSEKTIKEQVEKVNNDIDKNNQLRYCLEQIKKYIETWDNVYYQIDFWAITKDYYKVIAKKAKFDFDKGNGSEIKLSSLQSAHNNRKRYLYIMDFWKENLYKTENLYRKSDNLLKVFEEAEKQNRNDKKLNEVELRRTFLSLFNLVNESLKPLIEGNLFIVNDDKIDEQNSNHKFVEDFISKTDERKQLYDCITELQKFFNGNGGYVPFGRITLNKWTAQQKPHIPKDKNGNNEIDKIISFLKPESVSMQNIDYEYDAFEDNFEQIDNKWKLKQNAKSVIEICQFFKYKKIPVNARLNLAKRLNKDIHFLSDFGVSKSPALDYKNDKDNFNLANYPLKVAFDYAWENCAKAKYETITFPEQKCKDYLKDVFDLNVDSNTDFTKYALLLRFKILVGRIKAEENTRKENIETVRSLLKDVKDNLSKEKDKTITSVENWLTFKEKQTDKKEKYENQDEFSKVMQTIGQERGGLKSRISKYKTLTEMFKVCASKFGKQFADLRDYFNEAYEVDKIKYRAWIIEDGKLNRFVLLADKTKDLNLENDKNGNLKLYEVKSLTSKSLVKFIKNKGAYPDFHIVNNRQIYLNSGGKDNRGNFIDDVKIHWSIYQNNQKFLNKLKDCFQNSKMAIDQNWNEFNFDFSNCDTYEKIEKEIDRKGYILQQNSISLDKVKDLIKNQECFLLPIINQDINREDKQAKNQFSKDWNDIFVNTKRLHPEFNLFYRFPTKDYPNTKFKNGTEKTKRYSRFQILAHLGCEVIPQGNYLSKKEQIAIFNDDEKQKGEVKKYINICSDFDCVIGIDRGIKQLATLCVLDKNGSIQGDFKIFTRTFNHDTKQWEHHFLEKRNILDLSNLRVDSPITICCNIPDDNHKKVLVDLASIQTKNGENQQKIKLKKLAYIRELQYAMQTQDLSEFVNSLKSADDITQDKIKNFISPYKEGPPYADLPKQEMFDLLKEWENADDEEKQVIAELDSADNLKSGIVANMVGIVAFLCEKYNYKVRIALEDLTRSYGIQKDALSGTTIFQNDEDFKEQENRRLAGVGTMQFFEMQLLRKLFKIQVDKNLHLIPAFRSVANYEKIVRRDKQNSGDEFVNHPFGIVCFVDPKYTSKRCPKCGKTNVDRKENIVICKEKSCGFQTKEGNPFEKNNIHFITDGDQNGAYHIAKKVLKKDSNNETKK

SEQ ID NO: 20 - protein, 1268 aa, synthetic construct
MINLESFKNLYEVKKTVSFWLNQPSKKSDINKTHWLLKDLVEISFESEKKLINNENNHVLIDTEKVLIEKLQQYVNWLEVQLKNWKWVYQSYDLIAINKDYYKILASKAKFDAMWEITKFDKKSNKYIKVKQPQASQISLSSLKIWNSSDSIIQYWWEISQKTDYLLNIFKPKLEQYEKAINDANSAHIKPDSIDFSKTFLQLLKLTKEFLQPLLDSSIIFELSKKKVSQEIEKISEFAWEKNNTKIYNVLKNWEELSQYFEANWSQVPYWSVSLNYYTAVQKPNNFDQEIKKAIDDLWIINFLKKSDSQMIDYLHQGSKQKIKLLLTSKHPYPIELLQLFKVKPIPFSVKYNLAKFIEKNYKNEISLSYEEILDKLNLLWSAIDIANDFKNSNNQNNFSLDEYPVKLAFDYAWENTASSLKSTIPFPKEVCKQFLKDNFDVDVDNADFKLYANLLFIADNLAIIEYNNPNNEVELINKIKQAFDSIDFSFDKESYWWYKNDVLVLLNKAKSQSDYSTILKAKQELWLLSWWLKNKIKKYSDLTQSLIDKKNSHFWIASFVGKTLAKISDRLKEENELNKISHYGVILEDKNQDKYLLILQLDGKDTSEKISQKLGNGDIKVYQVNSFTSKALNKFIKNSWSEDAKKFHWDFSYKHKEVSIYDEKGKWTWYQESFLSHIKKCLIDSEISSEQNWEAFGWNFAGCNTYEEIEKEIDSKWYQLTENSILKETLGKLLKDEGCSLFPIINQDISSQKQENKNIFTLDFEKVFEWKEYKIHPEFSIFYSKPMEEHEKENKSWIINRFWKLQLLANLGIEFIPSNSSFKTKKEQNSIAIDQKKQNQLVQEFNQEKVNTYFEGLDNYYIFGIDSWIKQLATLCVTDKDWVIQSFDIYTKFFDSKEKIWKYTKTSTEGVLDLTNLKIESDKNWNTYIVDISLFDAKDEDWNPTGTNKQNIQLKQLAYISKLQYQMSANEEWVLTFLGKYKNKEESEQNMKGLITPYKEWKNFADLPMDIFQEMFENYYSLKTDQNLSESEKKNLMKITTELDASESLKKWVVANMIGVIYYLMKKYEYKVKISLEDLSNAWFFSKDGLSWDVVLNTKNDETMDLKKQDNLALAWVWTYHFFEMQLLKKLFKISTEEWVLHLVPSFWSVKNYIEIMKIKGKYVYKQFGIVYFVDPSNTSSKCPVCWKWWKKYISSVDNIVTCKNCWFDTSSDNSILINNYEKQWKKLHFIKNWDDNGAYNIWEKISELPKKSDVKNTSISWNISFS

SEQ ID NO: 21 - protein, 906 aa, synthetic construct
MEKNYKSEINLSYEDILNKFNLLWSAIDIANDFKNSNNQNNFSLDEYPIKLAFDYAWENTASSLKSTIPFPKEVCKQFLKDNFDVDIDNADFKLYANLLFIADNLATIEYNNPNNEVELINEIKQAFECISFPFDKEVYKWHKEAILELLDKEKSQSDYSTILKAKQELWLLSWWLKNKIKKYSDLTQSLIDKKNSHFWIASFVGKTLATISDGLKEENELNKISDYWVIIEDSNQDKYLLTLELNGKDISDSISNSLWNWEYKTYEVNSFTSKALNKFIKNPLSEDAKKFHGKYKDEYSFWYENKDWDFTYKITKVSKYDEQGKWTWYQESFLSHIKKCLIDSEISSEQNWEAFGWNFAGCNTYEEIEKEVDSKWYQLTENLISMGNLKSLVKDEGCLLFPIINQDISSQKQENKNIFTLDLEKVFEWKECKIHPEFSIFYSKPIEEHKKENKSWIINRFGKLQLLANLGIEFVPSNPSFKTKKEQNSIAIDQKKQNQLVQEFNQKKVNTYFEGLDNYYIFGIDSWIKQLATLCVTDKDWVIQDFDIYTKHFNSESKKWEYKFHSKDWILDLTNLKIESDSSWNKYIVDISLFQAKDEDWNPTGTNKQNIQLKQLAYISKLQYQMSANEEWVLNFLGKYKNKEESEQNMEELITPYKEWKNFADLPMDIFQEMFENYYSLKTDQNLSESEKKNLMKITTELDASESLKKWVVANMIGVIYYLMKKYEYKVKISLENLSNAWLFSKDGLSWDVVLNTKNDETMDLKKQDNLALAWVWTYHFFEMQLLNKLFKISTEEWVLHLVPSFWSVKNYIEIMKIKGKYVYKQFGIVYFVDPSNTSKKCPVCWKWWKKYISSVDNVVTCKNCWFDTSSDNSILINNYKKQWKNIHFIKNGDDNAAYNIWEKIR

SEQ ID NO: 22 - protein, 910 aa, synthetic construct
MEKNYKSEINLSYEDILNKFNLLWSAIDIANDFKNSNNQNNFSLDEYPIKLAFDYAWENTASSLKSTIPFPKEVCKQFLKDNFDVDIDNADFKLYANLLFIADNLATIEYNNPNNEVELINEIKQAFECISFPFDKEVYKWHKEAILELLDKEKSQSDYSTILKAKQELWLLSWWLKNKIKKYSDLTQSLIDKKNSHFWIASFVGKTLATISDGLKEENELNKISDYWVIIEDSNQDKYLLTLELNGKDISDSISNSLWNWEYKTYEVNSFTSKALNKFIKNPLSEDAKKFHGKYKDEYSFWYENKDWDFTYKITKVSKYDEQGKWTWYQESFLSHIKKCLIDSEISSEQNWEAFGWNFAGCNTYEEIEKEVDSKWYQLTENLISMGNLKSLVKDEGCLLFPIINQDISSQKQENKNIFTLDLEKVFEWKECKIHPEFSIFYSKPIEEHKKENKSWIINRFGKLQLLANLGIEFVPSNPSFKTKKEQNSIAIDQKKQNQLVQEFNQKKVNTYFEGLDNYYIFGIDSWIKQLATLCVTDKDWVIQDFDIYTKHFNSESKKWEYKFHSKDWILDLTNLKIESDSSWNKYIVDISLFQAKDEDWNPTGTNKQNIQLKQLAYISKLQYQMSANEEWVLNFLGKYKNKEESEQNMEELITPYKEWKNFADLPMDIFQEMFENYYSLKTDQNLSESEKKNLMKITTELDASESLKKWVVANMIGVIYYLMKKYEYKVKISLENLSNAWLFSKDGLSWDVVLNTKNDETMDLKKQDNLALAWVWTYHFFEMQLLNKLFKISTEEWVLHLVPSFWSVKNYIEIMKIKGKYVYKQFGIVYFVDPSNTSKKCPVCWKWWKKYISSVDNVVTCKNCWFDTSSDNSILINNYKKQCKFFLHKKTSLFSLVFQIYHDNVPKCN

SEQ ID NO: 23 - protein, 1271 aa, synthetic construct
MLESFKDLYEVKKTESIWLTQPNKKWEKISHLELNELVKTSLESIKSDIDKDDNSIIKNEKNLIENSSQFVNELNVQFWTWKNIYKSYDLISVNKDYYSILAKKAKFDAFWKEGRTKYPKANQITLSSLQKWGSKEKIIEYWKEIIESSYYLISIFTQKLDQYENAVSNPEKLSHTKPDFTDFSKIFLQLLKVSEDYLKPLFDKSINFKTWKKLESEEDIKEINLFISEENNKNIYSLLKLWSDISEYFEENWAQVPYKKVSLNYYTAVQKPNNFEWEIKKAIEDLWIVEFLNKSDSQIINYLHQGSKQKKKLLLTSKHPYPIELLQLFKVKPIPFSVKYNLAKFMEKNYKNEINLSYEDILNKFNLLWSAIDIANDFKNSNNQNNFSLDEYPVKLAFDYAWENTASSLKSTIPFPKEVCKQFLKDNFDVDIDNADFKLYANLLFIADNLATIEYNNPNNEVELINEIKQAFECISFPFDKEVYKWHKEAILELLDKEKSQSDYSTILKAKQELWLLSWWLKNKIKKYSDLTQSLIDKKNSHFWIASFVGKTLATISDGLKEENELNKISDYWVIIEDSNQDKYLLTLELNGKDISDSISNSLWNWEYKTYEVNSFTSKALNKFIKNPLSEDAKKFHGKYKDEYSFWYENKDWDFTYKITKVSKYDEQGKWTWYQESFLSHIKKCLIDSEISSEQNWEAFGWNFAGCNTYEEIEKEIDSKWYQLTENSILKETLGKLLKDEGCSLFPIINQDISSQKQENKNIFTLDLEKVFEWKECKIHPEFSIFYSKPIEEHKKENKSWIINRFGKLQLLANLGIEFIPSNSSFKTKKEQNSIAIDQKKQNQLVQEFNQEKVNTYFEGLDNYYIFGIDSWIKQLATLCVTDKDWVIQSFDIYTKFFDSKEKIWKYTKTSTEGVLDLTNLKIESDKNWNTYIVDISLFDAKDEDWNPTGTNKQNIQLKQLAYISKLQYQMSANEEWVLTFLGKYKNKEESEQNMKGLITPYKEWKNFADLPMDIFQEMFENYYSLKTDQNLSESEKKNLMKITTELDASESLKKWVVANMIGVIYYLMKKYEYKVKISLEDLSNAWFFSKDGLSWDVVLNTKNDETMDLKKQDNLALAWVWTYHFFEMQLLKKLFKISTEEWVLHLVPSFWSVKNYIEIMKIKGKYVYKQFGIVYFVDPSNTSSKCPVCWKWWKKYISSVDNVVTCKNCWFDTSSDNSILINNYEKQWKKLHFIKNWDDNGAYNIWEKISELPKKSDVKNTSISWNISFS

SEQ ID NO: 24 - protein, 1268 aa, synthetic construct
MINLESFKNLYEVKKTVSFWLNQPSKKSDINKTHWLLKDLVEISFESEKKLINNENNHVLIDTEKVLIEKLQQYVNWLEIQLKNWKWTYQSYDLIAINKDYYKILASKAKFDAMWEITKFDKKSNKYIKVKQPQASQISLSSLKIWNSSDSIIQYWWEIIEKTDYLLNIFKPKLEQYEKAINDANSAHIKPDSIDFSKTFLQLLKLTKEFLQPLLDSSIIFELSKKKVSQEIEKISEFAWEKNNTKIYNVLKNWEELSQYFEANWSQVPYWSVSLNYHTAVQKPNNFEQEIKKAIEDLWIVEFLNKSEQEITSYLHQDSKQKIQLLLTSKHPYPIELLQLFKVKPIPFSVKYNLAKFIEKNYKNEISLSYEEILDKLNLLWSAIDIANDFKNSNNQNNFSLDEYPVKLAFDYAWENTASSLKSTIPFPKEVCKQFLKDNFDVDVDNADFKLYANLLFIADNLATIEYNNPNNEAELINEIKQAFDSIDFSFDKESYWWYKNDVLVLLNKAKSQSDYSTILKAKQELWLLSWWLKNKIKKYSDLTQSLIDKKDSHFWIASFVGKTLAKISDRLKEENELNKISHYGVILEDKNQDKYLLISQLDGKDTSEKISQKLGNGDIKVYQVNSFTSKALNKFIKNSWSEDAKKFHWDFSYKHKEVSIYDEQGKWIWYQEEFLNHIKKCLIDSDISSEQNWKAFGWNFSCCNTYEEIEKEIDSKWYQLTENSILKETLGKLVKDEGCLLVPIINQDISSQKQENKNIFTLDFEKVFEWSEYSIHPEFSIFYSKPMEEHKKENSSWIINRFGSVQLLANLGIEFLPSNSSFKTKKEQNSVAIDQKKENKLVREFNQEKVNTYFEGLDNYYIFGIDSWIKQLATLCVTDKGWVIQDFDIYTKHFNSGSKSWEYKFHSKDWILDLTNLKIESDSSWNKYIVDISLFQAKDEDWNPTGTNKQNIQLKQLAYISKLQYQMSANEEWVLTFLGKYKNKEEKEQNMKELITPYKEWKNFADLPMDIFQEMFENYYSLKTDQNLSESEKKNLMKITTELDASESLKKWVVANMIGVIYCLMKKYEYKVKISLEDLSNAWFFSKDWLSWGVVLNTKNDETMDLKKQDNLALAWVWTYHFFEMQLLKKLFKISTEEWILHLVPSFGSVKNYTEIFKDKGKYVYKQFGVIYFVDPSNTSSKCPVCWKWWKKYISSIDNVVTCKNCWFDTSSDNSILIKNNKKQWKKLHFIKNGDDNGAYNIWEKISELPKKSDVKNTSISWNISFS

SEQ ID NO: 25 - protein, 1135 aa, synthetic construct
MLSKKNKKWIKQPQANQISLSSLHDKKDLIINYWENNISESDFLINIFEPNLKKYLDALANPDKYSHTKPDLIDFSKTFLQLLNVNKEYLQPLFDKSIIFETGKKGESKYINEINEFAWNENNLKIKELLDLWEKISQYFESNWLQVPYWSVSLNYYTAVQKPNNFDDEIKKWIKDLWIIEFLSKEESEIQVYLNQESKEKIKLLKDSKEPYSIELIQLFKPKAIPFSVKYNLAKYIEKNYKKELGLKYEDILYKFDLLWSSYDIWKDYSECTEKNTFSLMKYPIKLAFDYSWENLASSLKKDIDFPKEICKNFLKDNFDVDINNSSFKLYADLLFIAENLATIEYNNPSNEKEIIDNIINTFQIINFEINGYKKKDYENHKTAILSLLEQGKNESDFKSNVFIEAKQKLWLLSWWQKNLIAKYKELTEKLVDKKDIHIWIASFIWSNLANISDGLKEEHELNKITDYWIIVEDNNNDKYLLLLSLAWKDNSEQLKSKLWDGEIKVYEINSFTSKALNKFIKNPWWEDAKKFHWNFNYKYSDISTYSHEWKWIGYKPDFIKHIKDSLCNSEIAKEQKRVEFGWDFDKCNTYEEIIKEIDSKWYNLINNTISKKNLEYLIENDNCLLLPLINQDISSKKTINKNAFTIDFESAIDWKSWYSIHPEFSVFYSTPMQEYSKENKFWIVNRFGSLELLANIGIEYVPSNKNYTTKKEQNKISLDKDKQNNLIKEFNEEKVDNYFDSLDNFYVFGIDSWIKQLATLCVMDKAWVIQDFDIYTKHFNNTQKKWEYKKNSTEWILNLTNLKVESDQNWTPFLVDISLFESKNENWKSFGTNKQNEKLKQLAYVSKLQYQMSFNNESVLNFIKKHTTQEAKAENIWELITPYKEWKQFADLPIDTFEEMFENFHQLKTNENLSELEKQNLMKLTTELDASEELKSGVVANMIGVICYLMKKYHYKVKIVLENLSNAWYFSEDWLTWDALLNSKNDWITDFKKQENLALAWVWTYHFFETQLLKKIFKISIEEKILHLVPSFWSVKNYTYLIKNNKSQYVFRQFGIVYFVSPKNTSFKCPICLKSGSEYITKNDNIITCKSCGFVSWKDNDEILKKYTKTHLNIHFIKNWDDNGAYHIWKKLLK

SEQ ID NO: 26 - protein, 1288 aa, synthetic construct
MIMENIKNNYQLSKTLRFGLTQKQNGNSSNTDNVYHSHSALKELVDISENRIKKNVSTEGATEMQLSIESIRKCMIMIEQFIKDWKRVYYRSDQISLDKDFYKKLSKKIGFEAFWFERNKKTQQRIKKPQSCIIALSELSKRDNFGKERQEYIVEYWENNLLKSTERYEEVSEKLEQFELALKINRTDNRPNEVELRKMFLSLVNMVREVVEPLCLGQISFPKLEKLADNSKNKQLRKFATDYQSKSDLLTQISELKKYFEENGGNVPYCRATLNPLTAVKNPKSTDSSILDEIKKLKLDVILRDYQSVALFDNSIRDLTASQKMQLLNQNNEGLIKRGLLFKYKPIPAIVQYEIAKVLSAELNKDEQELRNFLRDIGQVKSPAKDYAELQDKKDFDINHYPLKVAFDFAWESLAKSVYHPDIDFPEEQCKTLLREVFQVDENNENFKFYAQLLELRSLLATLEHGKPTEVITIENEVKKILENIDWSKFGDRGKNYKSAIENWIHNRNKKDFKGDYFKKAKQQIGLTRGRQKNLIKKYDEITKSYKDIAMKMGKTFAEMRDKITGAAELNKVSHYAMIVEDTNQDKYVLLQEFVENNKDRIYAKSTPQNEDFKAYSVNSVTSSAIAKMIRKIRIDKLQANERNNNRQQAPELSETQKEARNIKEWKDFIAEKRWNYEFDLKLDNKNFEQIKKEIDSKCFKLETKYMSEEVLVDLVKNQNCLLLPIINQDLAKKIKSESNQFTKDWNAIFAQNTPWRLTPEFRVSYRKPTPNYPKSERGDKRYSRFQMIGHFLCDFIPKTADYISNREMIANFKDDEKQKQTIINFHERLNPKSENEKMNMLLAKFGNKNSNQSKETKKEEKFYVFGIDRGQKELATLCVIDQDKKIIGDFNIYTRSFNTQNKQWEHQLLDKRHILDLSNLRVETTIVIDGKPDVRKVLVDLSEVKVKDKNGNYTKTDKMQVKMQQLAYIRKLQFQMQTNPDTVLEWYNKNQTKEAILNNFVDKPNGEKGLVSFYGSAVEELKDTLPIERIEKMLQQFKSLKNEEKEGKDVKAEIDKLIQLEPVDNLKAGVVANMVGVIAHLLKEFNYQVYISLEDLSNPFGSHVIDGTTGTHSKTNKGEGKRADVEKYAGLGLYNFFEMQLLKKLFRIQQDSQNILHLVPAFRAVKNYENIIAGKDKIKNQFGIVFFVDANSTSKMCPVCNSTNETNREYPNAKKGTSKDDKEVWVERDKSNGDDIIRCFVCGFDTTKKYEENPLKFIKSGDDNAAYIISAFGIKAYELAKSVIDNK

SEQ ID NO: 27 - protein, 1294 aa, synthetic construct
MEAIKNNYQLSKTLRFGLTQKSKTRKDGFTGEIYQSHNELKDLVKSSEDRIKKSVSTDEKSEMSLSVDKIRCCLVMISDFLSSWQQVYSRADQIALDKDYYKILCKKIGFDGFWVDERYDRKNDKTVRTKKPQSRTINLSELDKKDDKGIERRQYLLTYWRDNLINAADKFEVVTEKLKQFEDALNINRTYNKPNEVELRKLFLSLTNIVQETLQPLCLGQICFPKLEKIDDSRTENKHLIDFATDYQSKSDLLSEISELKKYFEENGGNVPYCRATLNQKTAVKNPNSTDNSIDSEIKKLGLDKILKENKDALYFANKIYSLSAKEKLSKLDDKTTGLIERSLLFKYKPVPAIVQYEIAKTLSETINKSEEDLLEFLRSIGQTKSPTKDYADLQDKNDFDLDAYPLKVAFDFAWENLARSIYHSDADIPVAVCEGFLKKNFGIDKSNADFKLYAQLQELKAVLATLEYGNPTNRQTFINEATKLLSPISWDKIGRNGNQNKYSIEKWLKTLTKDDKDYKDAKQQIALFRGRLKNNIKTFDDITKYFKSVAMEMGRTFAQMRDKITGAAELNKVTHYAIIIEDQNFDKYVLLQEFVDKKENRIYAKTDRHHSDFTTYSVNSVTSGSIAKMLRKKRMDELNRNNRNSFEQKPELSEEQKEQRNIREWKEFIEDKRWDLEFQLNLSNKTFEQIKKEVDAKCYELDINYISQETLSDLVNKKGCLLLPIVSQDIAKENKTEGNQFTKDWNAIFTQETPWRLTPEFRVSYRKPTPNYPVSDKGDKRYSRFQMIAHFLCDYLPKSDSYISNWEQIANYKDDKLQEKAVKEFNADLRGRTEEEKQSESVNALLAHFGNQNKKQKPVERPKEKFYVFGIDRGQKELATLCVIDQDKKIVGDFDIYTRSFNSEAKQWEHKLLEKRVILDLSNLRVETTIVIDGKPEKKKVLVDLSEVKVKDKDGKYSKPNKMQVKMQQLAYIRKLQFQMQTNPDAVLEWYANNKTKEQIMSNFVDNENGDKGLVSFYGTAVEELNETLPIDKIEEILKKFQELKDKEKQGETVKLEIDKLVQLEPVDNLKNGVVANMVGVIAYLLQNLDYQVYISLEDLSKPFSGQIIGGIAGVPTKTNKEEGRRADVEKYAGLGLYNFFEMQLLKKLFRIQQDSQNILHLVPPFRAMKNYDHVAVGKGKVKNQFGIVFFVDADATSKTCPCCGSSNNKPNLKMYPNAKKGLSKEGKEVWVERDKSEGNDIIRCFVCGFDTTKDYSENPLRYIKSGDDNAAYLISAEGIKAYELATTLVNNK

SEQ ID NO: 28 - protein, 1237 aa, synthetic construct
METIEYQFSKTLRFGLTLKDHERKNKTHQTFNDLIGVSAQRIKEDASRDHDKTEQQLVTSVAACVKLMHQYLDAWEKIYRRTDQLALTKDFYKQMAKKACFDAYWLDNKERKQPQSQIIAISSLRKKHDEKERKDYILDYWADNISITKQRIHEFEKVIDQFKKALENKSMAHNKPHLVDFRKMFLSLTRLCNETLIPICNDSICFPALDKLQDNARHEAIKTFASEDEREERKGLSLSIKDIKEYFEENGGYVPLGKVTLNRYTAEQKPNNFKEDIKKKINDLRLTDLIQKLINLSDEEIKEYFEFNGKQKKQLIDDTRLSVVERVQLFKYKPIPAAVRFMLAEYLHKNNLLDKERVMTLFEIIGKPRSIGEEYTKLKDTSDFDLFQYPLKPAFDYAWENVARNLRNDKANAYPKEQCIRFLENIFEVSTSTTAFILYADLLFIKDNLSTLEHEKNSPKDKDQFIENIKRTFKNINYGIEQKEYIKHQQTILDWINKKEDAQKELKNANDNSYKNYENAKQQFGLLRGRQKNSIRQYKDLTETFKTLAVSFGKNFAELREKLREENEINKITHFGIIVEDSKLERYVLLSKLDEDKTLSIDHLLIDEPKGELKSYQVKSLMPKSLEKIIKNKGGYKDFHTSSKYINFIEMKRDWANYKNKPELVDYVKDCLQHSTMSKDQHWAAFGCDFTTCNSYEAIERELEIKAYRLKASHISITTVQKLVNEENCILLPFVNQDITSAKRELKNQFSKDWDMLFENNNDYRLHPEFRIVYRQPTPDYPLNKRYSRFQLIAHLMCEYIPQSVEYISRKQQIQIFNDKNEQKKQVDAFNERVKPSGEYYVLGIDRGLKQLATLCVLNNEGQIQGGFEVFTRTFDSVKQEWKHNLLEKRAILDLSNLRVETTVNGDKVLVDLASILVKDEQHNYTKDNQQKIKLKQLAYIRKLQFQMQHEPQKVLNFIKDYTTPQAVGEKIGELITPYKEGTHYDDLPIEKIYDMLQQFHQFTDEGNETAKKELTELDSADDLKTGVVANMVGVIAFMLEKYHYNAFVSLENLCRAFGFAKDGLNGELLVSTAVDNTVDFKDQENLVLAGLGTYHYFEMQLLKKLFRIQTGNDIIHLVPAFRSVDNYETIRKLSTKTKDALYTCKPFGVVHFIDPMYTSKKCPACDSINVSRFSKQGDIISCNKCGFQTRWDTQTTLKNNALLQSYKKQNLNLHYILNGDDNGAYRIALKTFANLT

SEQ ID NO: 29 - protein, 1393 aa, synthetic construct
MTKYQLTKTLRFGLTKVRKKTKLVAGKEVDAKYLSHEELDDLVMRSEYNLIKRNVLEWSKKKESDKSNYIFDELDKRFEEIIEIEDRDQRNAEYVEFLNEFFKEVNHQTINQLDESTFINKIGDCSKAIKEYLLSWGKVCRRIDKITVRKDYFKILARKTFFKYESKVGKKRTPLPSEVKLSGQKGNNYFDEPINEGISQFWQNRVAKALNLHSQLESMLFDYKKAIETEKHNQENPKGDNGSFDKLHLVDFRKMFLSVCSLVMDSLRPIVNELIIVSDNVSKDEDKYILDFVNDKKTQWDLFQQIENLQTICKDNGENIFFGKATFNKYTSEQAPNHRNNDIAKVLRELKIEKFVSDYIDLDQEAINRKIYQSTQSRLENLNNPQISPIIRAQYFKYKPIPTLVRFGLAKELAKQQGKKYSDRLKGIQELFRIFGSSKSPALDYKNNRTDFSLDNYPIKVAFDYAWEMCARSEYAQKPVDFPKSICEKFLEKFFECKSNEKYQQSFVTYARLLKINEDLATLEHFENEPPKDIESIYQDAQRYLDEVGNLCSNEDRAAIAKWYEEYNKLWTKGDHKKLKEWIVSKSSIITNFTQAKMHLGQKRGSQKTFVLKSYFHSSYGKIRDNNRFVNSNVTEVFKTIASTFGKSFATIREYFNEESEVNKIEYGAVIIKDKNGDKYLLLQKKNEGGIDMPIFNKSDENGDCDLYQVKSLTSKTVRKIIASPNKYNDFFVNNDGKKIIYPDKTDFKYKINPYDKEEVKKRKRELYNNDLVRPIIYSLTQSKFANKQNFEKYFDWTKALKQCSNIEQLYKTIDQKGYSLNPSKISKEQIADLVNNMNCYLLPIVNQNITAKTKNDTNQFTKDWNKIFNEVDKDYRIHPEFTMFYRYPTPDYPKFGEKRYSRFQMNVNFLMEVIPADGEYCSRKEQIEIYNAPKDNENCQKNVVERFNNKIKALKPSYFIGIDRGINELATLCVIDKEGKIVGDFEIYKREFDSNLKRQKYTSIETRDILDLSYLRVEKDENGESRLVDLSESEVWIDSLDHDKGKRANKQIVHLKHLYYLRCIAHLLQSSDYKSIVLEKLKDCNNLKDEEIKKVFEKDKFVDSYKGGEAYTDLPYDEIRKLISDYQEIEQSNQTESEKSKALNTLCQLDASEYLKKGVVANMIGVVVYVLKKYNYDAYISLENLCYAYGYSKDTLSGYSITSTKEDPYLDFKDQENAKLAGLGTYSFFEVQLLKKLFKLQIEENTELIPAFRSVDNYEKIFLLKNIDNKIYQFGIVYFVDPKYTSLCCPICGEHGKKNVDRKKHTKKYDEDELVCKQCGFHTNLSHIETRVMEDKTIKNSYDECNLKAIVSGDANAAYNIAIRLGKNIYSTIADKVKDLHHEGKKYIIVKG

SEQ ID NO: 30 - protein, 1370 aa, synthetic construct
MAGKEVDAKYLSHEELDDLVMRSEYNLIKRNVLEWSKKKESDKSNYIFDELDKRFEEIIEIEDRDQRNAEYVEFLNEFFKEVNHQTINQLDESTFINKIGDCSKAIKEYLLSWGKVCRRIDKITVRKDYFKILARKTFFKYESKVGKKRTPLPSEVKLSGQKGNNYFDEPINEGISQFWQNRVAKALNLHSQLESMLFDYKKAIETEKHNQENPKGDNGSFDKLHLVDFRKMFLSVCSLVMDSLRPIVNELIIVSDNVSKDEDKYILDFVNDKKTQWDLFQQIENLQTICKDNGENIFFGKATFNKYTSEQAPNHRNNDIAKVLRELKIEKFVSDYIDLDQEAINRKIYQSTQSRLENLNNPQISPIIRAQYFKYKPIPTLVRFGLAKELAKQQGKKYSDRLKGIQELFRIFGSSKSPALDYKNNRTDFSLDNYPIKVAFDYAWEMCARSEYAQKPVDFPKSICEKFLEKFFECKSNEKYQQSFVTYARLLKINEDLATLEHFENEPPKDIESIYQDAQRYLDEVGNLCSNEDRAAIAKWYEEYNKLWTKGDHKKLKEWIVSKSSIITNFTQAKMHLGQKRGSQKTFVLKSYFHSSYGKIRDNNRFVNSNVTEVFKTIASTFGKSFATIREYFNEESEVNKIEYGAVIIKDKNGDKYLLLQKKNEGGIDMPIFNKSDENGDCDLYQVKSLTSKTVRKIIASPNKYNDFFVNNDGKKIIYPDKTDFKYKINPYDKEEVKKRKRELYNNDLVRPIIYSLTQSKFANKQNFEKYFDWTKALKQCSNIEQLYKTIDQKGYSLNPSKISKEQIADLVNNMNCYLLPIVNQNITAKTKNDTNQFTKDWNKIFNEVDKDYRIHPEFTMFYRYPTPDYPKFGEKRYSRFQMNVNFLMEVIPADGEYCSRKEQIEIYNAPKDNENCQKNVVERFNNKIKALKPSYFIGIDRGINELATLCVIDKEGKIVGDFEIYKREFDSNLKRQKYTSIETRDILDLSYLRVEKDENGESRLVDLSESEVWIDSLDHDKGKRANKQIVHLKHLYYLRCIAHLLQSSDYKSIVLEKLKDCNNLKDEEIKKVFEKDKFVDSYKGGEAYTDLPYDEIRKLISDYQEIEQSNQTESEKSKALNTLCQLDASEYLKKGVVANMIGVVVYVLKKYNYDAYISLENLCYAYGYSKDTLSGYSITSTKEDPYLDFKDQENAKLAGLGTYSFFEVQLLKKLFKLQIEENTELIPAFRSVDNYEKIFLLKNIDNKIYQFGIVYFVDPKYTSLCCPICGEHGKKNVDRKKHTKKYDEDELVCKQCGFHTNLSHIETRVMEDKTIKNSYDECNLKAIVSGDANAAYNIAIRLGKNIYSTIADKVKDLHHEGKKYIIVKG

SEQ ID NO: 31 - protein, 1335 aa, synthetic construct
MEQYQLTKTIRFGLTKVRKEKKHLSHEELDELVMVSEERIKKEHPQAENQLDEQSFVKKIGDCSKAIKEYLLSWGKVCRRIDKITVRKEFFKILARKTFFKYESKVGKKRTPLPSEVKLSGQKGNNYYDEPINEGISQFWQNRVSKALKLHSQLESMLFDYKKAIETEKHNQENPKENNDQFDKLHLVDFRKMFLSVCSLVMDSLRPIVNELIIVSDNVSKDEDKYILDFVNDKSKQWDLFKQIEDLQNLCKDNGGNIPFGKATFNKYTSEQAPNHRDNDIHKVIRELKIEEFVSDFIGLEQEDIYRKIYQSTQHSLVNLNKPSISPIIRAQFFKYKPIPVLVRFGLANYLNKQQGKKYSNRLKDIQELFRIFGTSKSPELDYSDKNNRTEFSLDKYPIKVAFDYAWERCARSKYAQKPVDFPKEICVTFLETYFEYNSKEKNREAFEIYAHLLKVNECLATLEHFENEPPKDIKSLWQDVQNHLDKVGKLCSNEDRKAITQWYEEYKNLWAKGNYKKLKKWIESKSYTVTNFTQAKMHLGQKRGSQKTMVLKSYFHPTYGKIKDGNRFINSNVTEVFKNIASTFGKSFATIRDYFNEESEVNKIEYGAVIIKDKKGDKYLLLQKKNEGGIDMPVFNESDGNGDYDVYQVQSLTYKTVNKIYNSTKYPEFFAINGEKAIYAPNRPQRFKDDQEKNTFNEKKLQSLKKCLTESDFMTNTTENYLQKFNWTEEINNCTDFEPLAKIVDQKGYYLKSYKISKEQIAELVNNQNCYLLPIVNQNITAKTKNDTNQFTKDWNKIFNDEYKDYRLHPEFTMFYRYPTPDYPCPGEKRYSRFQMNVNFLMEVIPSEGEYVSRKEQIESFNTPKQDKEDNDNENSQAKKVAQFNDNINTKKPSYIIGIDRGINELATLCVINSEGKIVAVDENGLIKDEFDIYVKHFDKDNKCWIHNIKPKTATDNKPRTILDLSNLRVETTIDGKQVLVDLSSDENGIQVNSKQIVHLKRLYYLRCLSYLLQSSDYKSIILEKLKDVNNMTDDAIYEVFKNDKFIDSYKGGVQYTDLPYDEIRHLISTYQEIDQSNKTDSEKQSELNTLCQLDATESLKKGVVANMIGVVVYILKKLNYDAYISLENLCRALYFSKDSLSGYTIENTSVNPDLDFKDQENAKLAGLGTYSYFEIQLLKKLFKLQIDEKQFLVPGFRSVENYEKIVKLGKVKHSIYQFGVVHFVEPANTSLKCPICGANGKRIKYNPNYDEDELVCKKCGFRSNISKIQNSKIMEDSVIKTYYDNHNYKAIISGDTNAGFNIALRLLMNLNTQIENAINHLHKTGKNYHSVNK

SEQ ID NO: 32 - protein, 1248 aa, synthetic construct
MKNLTQFVNLYQLSKTLKFGLTLRNKIRKNGFEGEIYESHTELQELIKISEQKIIKETTDKNKEIMTFTELPLDEIRKCLDDMHKYLDDWEQFYNRYDQIAVLKDYYRKLERKARFDGFWREKNIKNKIQNNESETIKKPQSQVIKLSSLNNEYENKKRRDYITDYWNENIQKAKRKFYEVNSVLKQFEVANEQNRDDKKLNEVVLRKLFLSFTNLINDTLEPLCNGSICFPDIEKLTNSKTDEQLQRFVFDDGFKKILSEQIENLKIYFAINGGYVQYGRVTLNKYTALQQPNKVDEDIKNIIKELGLLEFVKKYENTEQIINYIKNIKDKKQELNANNLSLIEKVQLFKYKTIPAGVQPSLIAYLARTEKKDKKTLRELFYAIGQPQSPSKDYKELQNKTDFNLYKYPLKVAFDYAWESLAKSKYNPHIDFPDVKCKEFLKDIFGTDISVNDNFKLYSALLFVRENLATLDHGNPNDKNIHVNKVENTFKEIKDRLAKKEYKKEYKALEIICKWHKNSATIEQSEYEAAKQTIYEAAKQTIGQLRGRQKNQISKFKELTDSFKKLAPKFGKAFANLRDKFNEEYEINKISHCGVIVEDRNNDQYLLLSQLNDNRENASDIFELEADPNGELKIYQVKSLTSKTLLKFLKNKKGSNTGFHINENWTFPKGKWDVINKDKIFLNYVIQRITNSSMAKEQKWSNFKWDFRRCDSYEAIAKEVDAKGYILESVNISKLTLNKLITEKKCLLLPIVNQDLTRQDKKTKNQFTKDWIKIFESNNCYRLHPEFKISYRYPTPNYPKPEEKRYSRFQMISHLLCEYIPQNDNYKSRKEQVKIFNDKVAQKESVEQFNQQFEITDDYYIFGIDRGIKQLATLCILNKNGQIQGDFEIYTREFDKVNKQWKHTILEKRNILDLSDLRVETTVEGKKVLVDLSKVKLHSGNENKQTIKLKRLAYIRMLQYQMQHEQDKVLRFINQYKTIDEIEKNIRDLISPFKEGKQYADLPTEKIKDMLIQFGELSKNDSDKSKKELCTLCELDAVDDFKTGAVANMIGVIAYLLEKYKYNVYISLEDLTRAFRLQRDRLTNNILQSTNKDNTVDFKDQENLVLAGLGTYHYFEIQLLRKLFRIQRNSEGDILHLVPTFRSVDNYEKIVRRDKKTDNDKYVNYPFGIVRFVDPKYTSKKCPICDKTNTTRKDNVLICNSCNAVSGEYETDNENRHYITNGDDNGAYHIALKALSLRKSKNLEKKK

SEQ ID NO: 33 - protein, 1242 aa, synthetic construct
MEKYQITKTVRFGLTATNSNLYSDELKDLIETSEIKIKESLKNKSHNSLQIEQLRSCLNGVKEYLKTWNNVYSQIDFLGISKDYYKVISRKARFDFDNKGLGSEVKLASLQSKYNSKKRIQYILDFWEDNFQKTEILYRKSDELLKVFEEAEKQKRDDKKLNEVELRKTFLSLFNLVNESLKPLVEGNLFTINDDKIDSRNQNHEVIADFISNTKVRTELYESITELQNFFRDNGGYVPFGRATFNQWTALQKADKNGEREIDKIIKQLNLETVSMANIDYKYNTFTKNFEQGGQVWKIKQNAKSVIELCQFFKYKKVSITTRLNLAKRLNKTNNFLSEFGISKSPALDYKKDKENFNLANYPLKVAFDYAWENCAKAKHESITFPELQCKDYLHNVFGVDANKDKNGKIKNEELNKYADLLQFKILLGRLKAEFHKAAEETNKNNIRKLKNIFENLDYSGVQDFNKNKIKEIVEVWFANKEKNIGKKKEEMIPLTEKKKDDFSKAMQIIGQERGGLKSRIKKYKTLTEMFKVCASRFGKLFADLRDYFNEAHEVDKIKYRSWILEDGKQNRFVLLVDKAKDLELENEENGELKLYEVKSLTSKSLIKFIKNKGAYPDFHSLNSFNSDEIKKNWTNHKANINFLKNLKSALENSLMAINQNWKEFNFDFSRCDTYEQIEKEIDRKGYILKQQNISLNTIKKSINEEKSEKINNSKKLPSLLFPIVNQDINREAKQEKNQFTKDWFEIFAEENNLHKKRLHPEFHLFYRFPTKNYPNTKFKNGKEKSKRYSRFQMLAHFGLEVFPQGDYISKKEQIEIFNDDKKQKEAVEKYNNSIVSEVEYIIGIDRGIKQLATLCVLNKNGVIQSGFQIYTPSFNHDTKQWEHSFLGKRNILDLSNLRVETTIKNEKVLVDLASIQTKKGENQQKIKLKQLAYIRELQYSMQTRQVELLEYAKTLNSAEDITEEKIKIFISPFKEGSHYEHLPKQEIYNLLNEWQNADETRKRKIQELDPTDSLKSGIVANIVGVIAFFCEKYNYKVRISLEDLTRAFSIQKDALTGTPIHRNDEDFKEQENRRLAGVGTMQFLEMQLLKKLFKLQSEKNKHLIPAFRSVANYEKIVRRDKENGGDEFVNYPFGIVTFVDPRNTSQKCPYCNNIARKEDDAFYRNAGENKNSLLCKKCGLSTIKGKENKSNQDDSKNQFNIHFITDGDQNGAYHIALKTLENLHRLNTPKVTKHTKTKWKK

SEQ ID NO: 34 - protein, 1201 aa, synthetic construct
METNKTTKAINEYQTQKTIRFGLTVTNNNLYSENIVKLLKCSEEKIKEQLKKTQTDDLQNQRLRCCLIEIKEYLKTWNNVFSQIDFLAITKDYYKVLSRKAKFDYDKGNGSEIKLSSLQSKQSKYNDKKRYQYILDFWHENFIKVENLYRKSDDLLKVFEEAANQNQDDKKLNKVDLRKTFLSLFNLVNETLKPLIEGNLFIVNDDKIDEHNSKHNFVSDFIVKTEERKQLHDCITDLQDLFKANGGYVPFGRATINKWTALQKSNHKDDEIKRIIRELKIENISMQNIDYKYKYDSFAENFKQIYNKEGEKVWVLQFDANSVIKVCQYFKYKKVPINARLNIAERLIKEKSWQREKKNDFLSEFGISKSPALDYKNDKENFNLANYPLKVAFDYAWENCAKAIYETTTFPKEHCEKYLKEVFDLDIANNACFTKYALLLRFKILICRIKSEETTQIQNIEAVRGILDEINKNISGRQDFSKAKIITEINNWLSFKEKQTDKKEKYSNQDNFSLAMQIIGQERGGLKSRIEKYKTLTDMFKVCASKFGKQFADLREYFQEAYEVDKIKYRAWIIEDEKQNRFVLFANKEREIDLTSEEGNLYFYEVKSLTSKSLVKFIKNRGAYADFHKLKNNFNYEKIKRDWQYYKNDKYFIQNLKDALRNSKMAIDQNWAEFKFDFTKFNTYEDIEKEIDRKGYKLVCKTVSLNTLKDFVENKGCLLLPIINQDINKDDKQAKNQFTKDWNSIYDNKKRLHPEFNLFYRFPTQDYPNTKFSNGTEKTKRYSRFQMLAHFGCESVPKGDYLSKKEQIAIFNDDAKQKDAVEKFNNSIASDFEYIIGIDRGIKQLATLCVLNKNGQIQGDFEIYTRTFENKQWKHTLSEKRNILDLSNLRVETTIDGNKVLVDLASITTKNGENQQKIKLKQLAYIRELQYSMQTRRDDLLDFAKGLQSADDILKDIRNFIVPFKEGGQYADLPNERIYNLLKEWRDADDEAKRKIAELDPAQDLKSGIVANMIGVVAFLCEKYGYKVRISLEDLTRAFGIQKDALSGIAIAPNDEDFKEQENRRLAGVGTYQFFEMQLLKKLFKTQVDKNLHLVPAFRSVDNYEKIVRRDKKTNGDEYVNYPFGIVRFIDPKYTSKRCPKCGKTDVNRNQKTNIVKCNNCEYETKAGNSSEANNIHFITDGDQNGAYHIAQKALKIQKEQ

SEQ ID NO: 35 - protein, 1304 aa, synthetic construct
MKQIKNQYQLSKTLRFGLTQKNKTKKENYAGEIYKSHSELSDLVEISEQRIKDSVSTNKNSESSLPVDAIGKCLNQISEFLKGWQQVYQRTDQIALDKDYYKILCKKIGFDGFWFDKKNGRKTKKPQARIISLLELEKKDDKETERKQYILDYWQENFINAVEKYNVVSEKLKQFEVALKINRTDNKPNEVEFRKLFLSLVNIICDTLKPLCFQQICFPRLEKIDNSKIDNKNLIDFAIDYQSKNELLSLISKLKSYFEENGGNVPYCRATLNPKTAVKNPESTDNSIESEIKKLGLDKIIKNNKDAFSFSYNLYNNTAEDKKSKLKDDENGGLIERSLLFKYKSIPATVRFEIAKTLSKPDGKTEEEILEFLRDIGQLESPAKDYADLKEKDNFNIEKYPLKVAFNFAWEGLARAKYHPEAVFPTEICKQYLKNHFKITEDNKDFVMYAKLLELNAVLSTLEKAKPTDEKKFSVAAKKLLEEIEWEKVGKNGSKNKEAITKWLQTKSKTDKNFKSAKQEIGLFRGRIKNNIRIKNNIKSEYSEITNVFKNIAEEMGKTFAEMRDKISGAAESNKISHYAMIIEDNNKDKYVLLQEFVENKNERIYAKSDSQKSDFKAYSVNSITSGAIVKMLKKIRTDKLKESNNFANTQPELTSKEKEKRNIKEWKKFINEKGWNLEFGLKLENKTLEEIKKEVDAKCYKFDIKYFDKETLSDLVKNKNCLLLPIVNQDLAKKEKNESNQFTKDWNAVFPQDTPWRLTPEFRISYRKPTPNYPKSDKGDKRYSRFQMIGHFLCDYIPKTDSFISNRQQIENYKDDERQELAVKKFNAALRGRTKNEEYKEQLNELAAKYSKNGQQKINVKTNEKFYVFGIDRGQKELATLCIIDQDKKIIGPHKIYTRSFNSEKKQWEHKFLEERHILDLSNLRVETTVFIDGKPEKTKVLVDLSEVKVKDKVTGEYTKPDKMQIKMQQLAYIRKLQFKMQNEPEAVLAWYEKNSTEDLILKNFVDNEDGTNNGLVSFYGAAIEELKETLPIERIVDMLKEFKTIKKEEGKLTKEDEEGREKNKRKMDKLVQLEPVDNLKNGVVANMVGVIAFLLQKFDYQVYISLEDLSKPFSSKIISGIDGVPIRVEKEEGRRADVEKYAGLGLYNFFEMQLLKKLFRIQQDSENILHLVPAFRAMKNYDHIAVGKGKVKNQFGIVFFVDAEATSKTCPRCGSTNQKPNKKDYPNAQQARLSNDKEGWIDRDKSNGNDIIRCFVCGFDTTKEYTENPLKYINSGDDNAAYLISAEGVKAYELATTLADNI

SEQ ID NO: 36 - protein, 1292 aa, synthetic construct
MKNITNKYQITKTLRFGLSQKGKTKKEGFDGEIYQSHQEFNKLVSVSEARIKKSVTTEQKTELALSIDNVARCLNNISDFLINWQRVYYRTDQIALDKDYYKIMCKKIGFEGFWFETNRRTQQKIKKPQSRIISLSALDKKDGLGKERKQYILDYWKENLLSAAEKYEVVSEKLKQFQDALNINRTDNKPNEIELRKLFLSLTHIVYDILQPLCYGQICFPKIEKLDNTKEDNKKLIEFASDYQSKSDLLSEIAELKQYFEENGGNVPFCRATLNPKTLVKNPKSTDNSINEEIKDLGLKEILKTYKDVLNYNNYLESLSAKQKLQLLNDRNTSIITRSLLFKYKPISANVQFDIAKTLSPEVGKGEEDLRAFLRGIGQPKSPAKDYADLQNKSDFNIEAYPLKVAFDFAWESLARAIYHADSDLPMDACKNFLQDNFKVKNDDTNLKLYAQLQELKAVLSTLENGNPNNAAAFRLKATNLLNEIPWKTVGNYGQQNKDEISKWLNNGKNKDDYKKAKQQIGLFRGRLKNNIQGFDNITQTNKNIAMKMGRTFATMRDKITGAAELNKVSHYAMIIEDRNTDRYVLLQPFTENEQDRIYSQTDYNNGDYTTYEVNSITSGAIAKMLRKARIDELSKNDNNRNLTSQPELTEEKKEKRNIKEWKNFIENKRWDLEFQLKLNEKNFEQIKKEVDTKCYNLRTKKINKTTLEDLVNKSDCLLLPIVNQDLAKEEKTNGNQFTKDWNSIFAQNTPWRLTPEFRVSYRKPTPDYPISDKGDKRYSRFQMIGHFLCDYIPKSDKYISNREQILNYKNDELQKKAVKDFHEDLKGKTEEENQNESMNALMAKFGNVNKKQKATTVEKPKEKFYVFGIDRGQKELATLCVIDQDKKIVGDFDIYTRSFNSERKEWEHTFFEKRHILDLSNLRVETTASIDGKAEKKKVLVDLSEIKVKDKNGNYSKPDKMQIKMQQLAYIRKLQFQMQTNPEGVLAWFKENSTKDLIINNLVDKKNGEKGLISFYGSAIEKMEDTLPVDRIEEMLQKFAALKKQEKEGEDVKLSIDQLVQLEPVDNLKNGVVANMVGVIAYLLQKFNYQVYISLEDLSNPFGSQITGGIAGVPLKQGKDEGRRMDVEKYAGLGLYNFFEMQLLKKLFRIQQDSCNILHLVPAFRAQKNYDHVAVGKEKVKGQFGIVFFVDANATSKTCPVCGTTNNKPNNQKYPNAKKGLSADGKEVWLERDKSNGNDIIRCFVCNFDTTKEYTENPLKYIKSGDDNAAYLISAAGIKAYELATTLINNQ

SEQ ID NO: 37 - protein, 1261 aa, synthetic construct
METLNQFTGLYSLSKTMRFGLTLKEKKPKNDSIAVESLYQSHQDLKELVELSDKRIIEEKKPEPPVENLGNPPIEKLRDCLNSMQKYLNDWRKVYTRYDQLAVLKDFYRKLERKARFDGFWKDKKGQNQPQSQEIKLSSLKHKSGEKEIKDCIVTYWGENIRKANEKWHQVDSVLKQFEEAKRKNRDDKKLNQVELRKLFLSLANLVNDTLVPLCQRSITFPNADKLSDNARDKSVLDFIGDNEIREHLLDKITKLKEYFQDNGGYVPFGRVTLNQYTAMQKPNKTDKEIEDAIKNLGLSIIKSQNFDAFEHIEEATDKVERLNTVSLPLVERAQYFKDKTIPVGVRDSLAKYLAKDDTAKEKELIDLFEKIGMPKRPAKDYSDPTLKEKFDLRKYPLKVAFDYAWETVASKELHDDILKNKCKKYLKDIFDVDTDKSIFFNIYSDLNYMKIILSRIEYPTQNQLSKDNFLEWNRKVITILDGDDFSHFNKNADGSTDKKMNTAKTYVKTWLDKLEANIEQFDGQDFKKFYEDFKKKNKNSCKDFDDAKRDIGLKRGGLKQIIEETETFTDKKTGKQKPKYKDSKYKELTEAFKSIAVDFGKHFATLRDKFNEENEINKIEYYGVIVEDENADRYLLLSKLSESREEIKNIFPDKAEGLKTYKVKSLTSKTLTKLVKNKGAYKDFHISDMRVDFKKIKEEWSAYKNDQAFLKYLKKCLTDSSMAQAQNWSEFGLDFDKCNTYEEVEKELDGKAYLLQETRLSKATITNLVKNKGCYLLPIINQDLAREDRTAKNQFTKDWKQIFENKKHYRLHPEFNMAYRQPTPNYPNSEIGDKRYSRFQMIANFMCEIVPQSTSYATRKEQIQTFNDNNKQQKAVKDFDSKFKLSDSYFIFGIDRGIKQLATLCVLDQGGVIRGGFEIYTRHFDGNKKQWVHTSLERRNILDLTNLRAETTIDGKKVLVDLSKVEIKNQTDNKQNIKLKQLAYIRKLQYQMQTNPEKVKNMSDEDIENDLKDIITPYKEGTHYADLPIENIKAMLDRFKVLYGKTDQQSKQELKELCELDAADNLKGGIVANMVGVIAHLMEQYNYRVKISLENLTTSFVNQSDGLNEYFISRGMDFKEQENAALAGLGTYQFFEMQLLKKIFRIQQDDGNVLHLVPAFRSKEDYEKIIRRDKNDGDEYVNYPFGLVTFVDPRYTSRKCPICGKTDVKRNDNIITCKKCGAVSGKYSFDDKNRQFITNGDENGAYHIALKTRKEVHNEN

SEQ ID NO: 38 - protein, 1223 aa, synthetic construct
MDKENSFKGFTNLYEVRKTVRFGLTQPNKKGELKTHLEFDDLINKSFENIKKDVKSRDKPNFKEKELIEKINQFINGLEKQLGNWKQIYERYDVISVNKDYYKILARKAKFDAFKKDKKPQASQIKLSSLQKDNRKDNIIRYWGNIITRSDYLINIFKPKLEQYLNAVNNPNNSSHTKPDLIDFRKVFLQFLKVNEEYLQPLFDKSIQFETGKKENSEEIKKINTFSGDENNKEINYLIDLGKEIREYFEANGSQVPYGKVSLNYYTALQKPNNFGEDIRKGVENLGIIKFLNKSEEDIKNYLKQNSKEKINLLNNAKNHYFIELIHLFKPKTIPFSVKYNLAKYLEKNFNLKYEDILNKFDLLGKSVDIGKDYLECKEKEKFSLEKYPIKSAFDYSWENLARNLKRDVDFPKSVCEKFLKDNFDIIINNSSFNLYANLLFIAENLATIEYGNPNNENEIIESIKNTFDDIKFESNKQEYDGYKKEILNILNQEKSKRNYKNILTAKQRLGLLRGQQKNKISKYYNLTQSFKKIASFIGKTLATIREGLKEENELNKITDYGIIIEDKNQDKYILTLKLDGKDIREKIKSKLWDGEYKVFEINSFTSRALNKFIKNPLGEDSKKFHGDYKYKHKEVSIYKDVKWIGYKEEFLIHLKDSLVNSQIAKEQNWKAFGWNFDNFNTYEKIEKEIDKKGYKLIKNSISKENLEYLINEEKCLLFPLINQDISSKKEQNKNEFTKDFNKAFLGIGYRIHPEFSIFYRQPDEENKKINKSGIINRFGRLQLLANIGIEYIPQNNDYKTRKEQNKISLDQTNQNELVQNFNKEKVNKYFDSLDDYYIFGIDRGIKQLATLCITNKNGIIQSYEIYTKYFNNNSKKWEYKKNRIEGILDLTNLKIESDKDGNKFLVDLSLFEAKDENGNSTGTNKQNIKLKQLAYIRKLQYQMSSNEKGVLNFLKKYQTKEERQNNIKELITPYKEGHHFEDLPVNIFEEMFENYEKLKNDKTLSEIEKQNLMKLTIELDSSEDLKKGVIANMIGVIVYLMKKYDYKVKIAVENLNQSFMGQNDGLNNSYISIKTNFKDQENGALAGMGTYHFFENQLLRKLYKVSVEEGILHLVPFFNSLDNVNKLNFEKEKILWVQTENYRKFGIVSFVRPHNTSKRCPICKSINVKRKDNITTCSDCGFITGKDNNIVIKKYKKEGLNLDLIKNGDDNGAYNICCKIGL

SEQ ID NO: 39 - protein, 1239 aa, synthetic construct
MRFGLTQPNKKGELKTHIEFSDLVNKSFENIKKEVNSKDKSKFDTRKELIDKINQFISGLENQLGDWKNMYERYDLISVNKDYYKILARKAKFDAFKKDKKGVKQPQANQIKLSSLRYNKELIINYWGNIISRSDYLINVFKPKLEQYLNAVNNPNNSSHTKPDLIDFRKVFLQLLKISEEYLQPLFNKSIQFETGKKENSGDIKRVNDFSGNENNKEINDLLDLGKEIREYFEANGSQVPYGKVSLNYYTAVQKPNNFDKEIKEGIKDLGIIEFLKKSEEDIKNYLKQDSKEKIYLLNNSKNPYSIELIQLFKPKTIPFSVKYNLSKYLEKNYNLKYEDILNKFDLLGKSVDIGKDYLECKDKEKFSLEKYPIKSAFDYSWENLARSLKRDVDFPKNVCEKYLNDNFNINVGNSSFNLYANLLFIAENLATIEYGKPNNEKEIIDSIKETFLELSDEIEKNNKKNEVENIIKYLNLNTDERKNIKDLQKKYFKNLDTKEQNILNIFDSFTKSKQSLGLLRGQQKNKIDKYRNLTQKLVDKKDSHIGIASFIGRTLASIREGLKEENELNKITDYGIIIEDKNQDKYILTLKLNGKDTREKIKNNLGNGEYKVFEINSFTSKALNKFIKNPLGEDSKKFHGYFQYKHREVSIYDENEKWVGYKEEFLKHLKHSLINSQIAVEQNWKDFGWNFDNCDTYEKIEKEVDKKGYKLIETSISKENLENLIHKEDCLLFPLINQDISSKKEENKNDFTKNFEKVFLGDGYRIHPEFSIFYRQPNEENLKPNKSGIINRFGRLQLLANIGVEYIPQNNDYTTRKEQNKISIDQTKQNESVQKFNKEKVNPYFDSLEDYYIFGIDRGIKQLATLCITNKKGVIQNFDIYTKHFNDNSKNWEYKNNRTEGILDLTNLKVESDKEGNKYLVDLSLFEAKDENGNLTGTNKQNVKLKQLAYIRKLQYQMSSNEEGVLSFLNKYKTKEERQNNIKELITPYKEGHHFEDLPMNIFEEMFENYEKLKNNKTLSEGEKQNLMKLTTELDASEDLKKGVVANIIGVIVHLMKEYDYKVKIAIEDLSNAWYFSKDGLSGDSILNSKIDEEMDLKKQDNLALAGVGTYHFFEMQLFKKLFKISVEKGILHLVPSFGNVRNYTDLLKEKYKYQYQQFGVIYFISPKFTSSKCPICGKGGKKHIKRENNVITCKECGFVSGKDNSINIKNNKKEGLNLDLIKNGDDNGSYNIGGKIK

SEQ ID NO: 40 - RNA, 14 nt, synthetic construct
  source 1..14: mol_type = other RNA
  misc_difference 6: note = a, c, u, g, unknown or other
  misc_difference 7: note = a, c, u, g, unknown or other
  misc_difference 8: note = a, c, u, g, unknown or other
tctacnnngtagat

SEQ ID NO: 41 - RNA, 15 nt, synthetic construct
  source 1..15: mol_type = other RNA
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
tctacnnnngtagat

SEQ ID NO: 42 - RNA, 16 nt, synthetic construct
  source 1..16: mol_type = other RNA
  misc_difference 6: note = a, c, u, g, unknown or other
  misc_difference 7: note = a, c, u, g, unknown or other
  misc_difference 8: note = a, c, u, g, unknown or other
  misc_difference 9: note = a, c, u, g, unknown or other
  misc_difference 10: note = a, c, u, g, unknown or other
tctacnnnnngtagat

SEQ ID NO: 43 - DNA, 14 bp, synthetic construct
  source 1..14: mol_type = other DNA
  misc_difference 6: note = a, c, t, g, unknown or other
  misc_difference 7: note = a, c, t, g, unknown or other
  misc_difference 8: note = a, c, t, g, unknown or other
tctacnnngtagat

SEQ ID NO: 44 - DNA, 15 bp, synthetic construct
  source 1..15: mol_type = other DNA
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
tctacnnnngtagat

SEQ ID NO: 45 - DNA, 16 bp, synthetic construct
  source 1..16: mol_type = other DNA
  misc_difference 6: note = a, c, t, g, unknown or other
  misc_difference 7: note = a, c, t, g, unknown or other
  misc_difference 8: note = a, c, t, g, unknown or other
  misc_difference 9: note = a, c, t, g, unknown or other
  misc_difference 10: note = a, c, t, g, unknown or other
tctacnnnnngtagat

SEQ ID NO: 46 - protein, 31 aa, synthetic construct
  VARIANT 2: note = Any amino acid
  VARIANT 3: note = Any amino acid
  VARIANT 4: note = Any amino acid
  VARIANT 5: note = Y, F, or L
  VARIANT 6: note = Any amino acid
  VARIANT 7: note = Any amino acid
  VARIANT 8: note = Any amino acid
  VARIANT 9: note = D, G, or N
  VARIANT 10: note = Q, L, F, or M
  VARIANT 11: note = I, L, V, or M
  VARIANT 12: note = Any amino acid
  VARIANT 13: note = L, I, or V
  VARIANT 14: note = Any amino acid
  VARIANT 16: note = D, E, or S
  VARIANT 17: note = Y or F
  VARIANT 19: note = K, R, L, or S
  VARIANT 20: note = Any amino acid
  VARIANT 21: note = L, I, or M
  VARIANT 22: note = Any amino acid
  VARIANT 23: note = K, R, or S
  VARIANT 24: note = K or E
  VARIANT 25: note = A, I, L, or V
  VARIANT 26: note = Any amino acid
  VARIANT 28: note = D, E, N, or V
  VARIANT 29: note = A, G, F, or V
  VARIANT 30: note = F, M, or I
WXXXXXXXXXXXXXKXXYXXXXXXXXFXXXW

SEQ ID NO: 47 - skipped

SEQ ID NO: 48 - protein, 20 aa, synthetic construct
  VARIANT 2: note = N, S, or D
  VARIANT 3: note = L or I
  VARIANT 4: note = Any amino acid
  VARIANT 5: note = K, N, H, or A
  VARIANT 8: note = I or L
  VARIANT 10: note = V or S
  VARIANT 13: note = D or N
  VARIANT 14: note = Y or F
  VARIANT 15: note = A or S
  VARIANT 18: note = Any amino acid
  VARIANT 19: note = L, C, or V
FXXXXYPXKXAFXXXWEXXA

SEQ ID NO: 49 - skipped

SEQ ID NO: 50 - protein, 13 aa, synthetic construct
  VARIANT 1: note = Y, C, or S
  VARIANT 2: note = Any amino acid
  VARIANT 3: note = I or V
  VARIANT 4: note = Any amino acid
  VARIANT 6: note = F, L, I, or V
  VARIANT 9: note = Any amino acid
  VARIANT 10: note = Any amino acid
  VARIANT 11: note = L or I
  VARIANT 12: note = Any amino acid
XXXXSXTSXXXXK

SEQ ID NO: 51 - skipped

SEQ ID NO: 52 - protein, 9 aa, synthetic construct
  VARIANT 1: note = L, S, or F
  VARIANT 3: note = L, F, or V
  VARIANT 5: note = I, F, or L
  VARIANT 6: note = I or V
  VARIANT 8: note = Q or K
XLXPXXNXD

SEQ ID NO: 53 - protein, 9 aa, synthetic construct
  VARIANT 1: note = L or I
  VARIANT 2: note = H or T
  VARIANT 6: note = Any amino acid
  VARIANT 7: note = I, V, L, or M
  VARIANT 8: note = F, S, or T
XXPEFXXXY

SEQ ID NO: 54 - skipped

SEQ ID NO: 55 - protein, 14 aa, synthetic construct
  VARIANT 4: note = R or S
  VARIANT 5: note = G or W
  VARIANT 6: note = I, L, or Q
  VARIANT 7: note = K or N
  VARIANT 8: note = E or Q
  VARIANT 11: note = T or V
  VARIANT 14: note = I, L, or V
GIDXXXXXLAXLCX

SEQ ID NO: 56 - protein, 25 aa, synthetic construct
  VARIANT 1: note = R or E
  VARIANT 2: note = Any amino acid
  VARIANT 7: note = S or T
  VARIANT 8: note = N, D, or Y
  VARIANT 10: note = R or K
  VARIANT 11: note = V, I, or A
  VARIANT 13: note = T, S, or K
  VARIANT 14: note = T or D
  VARIANT 15: note = Any amino acid
  VARIANT 16: note = E, D, N, or K
  VARIANT 17: note = G, K, or N
  VARIANT 18: note = K, N, E, or T
  VARIANT 19: note = K, S, or Q
  VARIANT 20: note = V, R, F, or Y
  VARIANT 24: note = L or Q
  VARIANT 25: note = S or A
XXILDLXXLXXEXXXXXXXXLVDXX

SEQ ID NO: 57 - skipped

SEQ ID NO: 58 - protein, 22 aa, synthetic construct
  VARIANT 1: note = E or Q
  VARIANT 3: note = D or E
  VARIANT 4: note = Any amino acid
  VARIANT 5: note = Any amino acid
  VARIANT 6: note = D, E, or Q
  VARIANT 7: note = N, D, Y, or S
  VARIANT 8: note = L or F
  VARIANT 10: note = Any amino acid
  VARIANT 12: note = V, I, or A
  VARIANT 13: note = V or I
  VARIANT 16: note = M or I
  VARIANT 17: note = I or V
  VARIANT 19: note = V or I
  VARIANT 20: note = I or V
  VARIANT 21: note = A, V, or N
  VARIANT 22: note = Y, F, or H
XLXXXXXXKXGXXANXXGXXXX

SEQ ID NO: 59 - skipped

SEQ ID NO: 60 - protein, 16 aa, synthetic construct
  VARIANT 2: note = G or W
  VARIANT 3: note = L or V
  VARIANT 4: note = G, W, or E
  VARIANT 5: note = T or L
  VARIANT 6: note = Y or M
  VARIANT 7: note = Any amino acid
  VARIANT 8: note = F or Y
  VARIANT 9: note = F, L, or M
  VARIANT 11: note = Any amino acid
  VARIANT 12: note = Q or L
  VARIANT 14: note = L or V
  VARIANT 15: note = Any amino acid
AXXXXXXXXEXXLXXK

SEQ ID NO: 61 - protein, 21 aa, synthetic construct
  VARIANT 2: note = Any amino acid
  VARIANT 3: note = Any amino acid
  VARIANT 5: note = I or V
  VARIANT 6: note = I, F, or V
  VARIANT 7: note = Any amino acid
  VARIANT 8: note = F or Y
  VARIANT 9: note = V, I, or T
  VARIANT 10: note = Any amino acid
  VARIANT 11: note = P or A
  VARIANT 12: note = Any amino acid
  VARIANT 13: note = Any amino acid
  VARIANT 15: note = S or T
  VARIANT 16: note = Any amino acid
  VARIANT 17: note = Any amino acid
  VARIANT 20: note = Any amino acid
FXXGXXXXXXXXXTXXXCPXC

SEQ ID NO: 62 - protein, 12 aa, synthetic construct
  VARIANT 2: note = Any amino acid
  VARIANT 3: note = Any amino acid
  VARIANT 4: note = G or W
  VARIANT 6: note = D, Q, or E
  VARIANT 7: note = N or S
  VARIANT 8: note = G or A
  VARIANT 10: note = Y or F
  VARIANT 11: note = H, L, I, or N
IXXXDXXXAXXI

SEQ ID NO: 63 - DNA, 3699 bp, Sulfuricurvum sp.
  source 1..3699: mol_type = genomic DNA
atgcttcacgctttcactaatcagtatcaactttctaaaacattgagattcggagcaactctgaaagaagacgagaaaaaatgcaagagtcatgaggaacttaaaggatttgtagatatttcatatgaaaacatgaaatcttccgctacaatcgctgaaagtttgaacgaaaatgaacttgtgaaaaaatgcgaaaggtgttattctgagatcgtgaaatttcataacgcttgggagaaaatctactacaggacagatcaaattgctgtctataaagatttctataggcaactgtcaagaaaagctagatttgatgccggtaagcaaaattcacaactgataaccttagcttccctttgcggtatgtaccaaggagctaagttaagtagatacataaccaattattggaaagataacattactaggcagaaatcatttcttaaagatttttcccaacagttacatcaatacactcgtgcactggaaaagtctgataaggctcatacaaaacctaatctgatcaacttcaataagacctttatggtgttggccaatctcgtgaacgaaatagttattcctctttctaatggagccatctcttttccaaacatctctaagctggaggacggggaagagtcccatcttatagaatttgcactcaatgactattctcagttgtctgaattaattggtgaattgaaggatgcaatagccactaacggtggttacacaccatttgcaaaggtgacccttaatcattatacagcagaacagaaaccacacgtatttaaaaatgatattgatgctaaaatacgtgagcttaagttgattgggttggttgagaccttgaaaggaaaatccagtgaacagattgaggaatacttctcaaatttagacaagtttagcacatacaacgataggaaccaatcagtaatcgtaagaactcaatgctttaagtataaacccattccttttttggttaagcatcaacttgcaaagtacatttcagaaccaaacggttgggatgaagacgccgtagctaaggttctggatgctgttggagctattcgttctccagcacatgattacgctaataaccaagaggggtttgatttaaaccattatcctattaaagtcgctttcgattatgcttgggagcagttggctaattctttgtataccaccgtgacttttccccaagaaatgtgcgaaaaatatttaaatagtatctacggttgtgaagtctccaaggagcctgtatttaaattctatgctgatctgctttatatcaggaagaatctggctgtactcgaacataagaacaatctgcccagtaatcaggaagagttcatatgtaagatcaacaacacatttgagaacatcgtgttaccatataagatttctcaatttgaaacttataagaaggatatacttgcctggataaacgatgggcatgaccataaaaaatatactgatgcaaaacagcaattaggttttattaggggtggactcaagggtaggattaaggcagaagaagtgtcccagaaagacaaatatggaaaaatcaagtcttattatgagaacccttacactaaactcaccaacgaatttaagcaaatatcctctacttatgggaagaccttcgctgagttaagagacaaatttaaagagaagaatgagatcaccaaaattacccacttcggtattataatagaagataaaaacagagacagatatttacttgcaagcgagttgaagcacgaacaaatcaaccacgtcagtactatccttaacaagttagataaatcatctgaatttattacctatcaagttaagagccttacaagcaaaacattgattaaattgattaaaaatcacaccacaaagaagggagccatttcaccatatgctgattttcacaccagtaaaaccggattcaacaagaatgaaatcgaaaagaattgggataattataagagagaacaggtattggttgagtatgtcaaagattgtctgaccgatagtactatggcaaaaaaccagaactgggcagagttcggttggaattttgagaaatgcaactcctatgaggatatcgaacacgaaatcgaccaaaaatcatatttgctgcagagcgatacaattagcaagcagagtattgcttccctcgtggaggggggctgtcttctccttcctataattaaccaagatataacaagcaaggagaggaaggataaaaatcaattttcaaaagattggaaccatattttcgaaggttccaaagaattccgtctccacccagagttcgcagttagctacaggacacctattgaagggtatccggtacagaagaggtacgggcgtctgcagttcgtttgcgcttttaatgcacacatcgttccacaaaatggtgagttcatcaatttgaaaaagcagatcgagaactttaacgatgaagacgttcagaaacgtaatgtgactgaattcaataaaaaggtgaatcatgcactttccgacaaagaatacgtcgttattggtattgatagaggcctcaaacagcttgccacactctgtgttttagacaaaagaggtaaaattcttggagattttgagatctacaaaaaggaatttgtgcgtgctgaaaaaagaagcgagagtcattgggaacacacacaagcagaaaccagacatatcttggatctttccaatttgcgtgtggagacaacaatagagggtaaaaaggttctcgtggaccagagcctcacacttgtgaaaaagaatcgtgatacaccagatgaggaagctactgaagaaaataaacagaaaatcaagttgaagcagctcagctatattagaaaattgcagcataagatgcagactaacgaacaggacgttttagatttaattaataatgaaccatcagatgaagaatttaagaaaagaatcgaggggcttatttccagttttggagaaggacagaagtacgctgaccttccaattaatactatgagagaaatgatctctgatctccagggagttatcgctagaggaaacaaccaaacagagaaaaataaaattattgaattagatgctgcagacaaccttaaacaaggtattgtagctaacatgatcggaattgttaattacatcttcgctaagtattcatacaaggcttacatctctcttgaggatttgtcaagagcctatggaggtgcaaagtccggttatgacggaaggtatctgccatcaacttcacaagacgaggatgtagatttcaaggaacagcagaatcagatgcttgcaggtttgggtacctaccaattcttcgagatgcagcttctgaaaaaacttcaaaagattcagagtgataacaccgttctgagattcgtgcccgctttcagatctgcagataactatagaaatattttgagacttgaggaaactaaatataagtctaagccgttcggcgttgttcatttcatagatccaaagtttacatcaaagaaatgccccgtctgtagcaaaacaaatgtatacagggacaaggatgacatcttggtttgcaaagagtgcggttttaggagcgactcccaattaaaagaaagagagaataacattcattatattcacaacggggacgataacggtgcataccacatcgcccttaagagcgttgagaatcttattcagatgaagtaa

SEQ ID NO: 64 - DNA, 3894 bp, synthetic construct
  source 1..3894: mol_type = other DNA
atgggaaaaaatgaaaacaaataccagttatcaaaaacattacgttttggactaacactaaaagagaagatttcgaataatgagaaaacaccttaccaaagccattctcaattcagagatttaattatcctttcagaaaacagaatcagagaaggtatcagcactccccaaaaccgtgacttaccctcttttatccacagaattcaaaattgcaccgattttatcaatgatttcatccatgattggtggatgattctgatgcacacaggacaaattgaattggacaaagactactataaaagcctaactaaaaaagtgggatttgtaggtttctggtacaaagagaacaaaaaaaagggtggaaaaacgaagcaaccacaagccagaaacataccaatgggtgagcttagacatctttgtccccaaaatactaaggagtgtgccacatacatcaccgattattggaaagatcttttgataacagcaaccaacaaactgtatgaaagcagtgaacaacaaaagaaattcatcaaagcgatggagcagaatcgcacggataacaaacctaacgaaatagatctgaaaaaatcttttttgagtctggtatctgtgacaatggaactgctaaaccctattctaaacgggcagatactattcaacaaaatggacaggctcgatatgtctaaaaaaagcgacaatgatttcattgatttcgtcaatgatcacgaaacggttcgtgaattaaacaatgacattgaagaaataatcgctgatttcaaagaaaacggcaataatgtaaactattgcaaagcgacacttaatccagacacagcattaaagcaacacaacaacaatattccaaatgatatagcaactgatttggaagagctaatgatggacagcatcgtcggaaattatgatgatgtcaactcttttatggataattatgtttcaaatttatctgcaaaagataaaataaaaaaaatcaaagactccaatataagccttatttatcgcgctatcctcttcaaatacaagatgattccggcaaacgtaagaagagatatagcccaaggaatggccaaaaagctcaataaagacgaagagaatatctacagttttctatgcgagtttggcactttgcgtactccacaaaaagactacgctgatctgaaggacaaggacagtttcaatcttgacaactatccattgaaagtggcgtttgatttcgcatgggagggattagcaaaagcttggtatcacgaccaatctgattttcctattgatccatgtcgtgattttctacaagaaaattttgatgtgaacttagaggaagaccaagaagacgaatattttcttctgtatgcagatcttatcgagctaaacgccttattatcaactctcgacaaagggaatcctgcagaccccgactcaataaaaaatgaagcattggagatggtcgaatacataaattggaattcacttgacaaaaaaaatggaaactactacaaaaaaataatcaaaaaccgtctgaaatcttcgaagggcaacgaaacgtacgaaagaataaaaaaagagatttccatgagccgtggacgtcttaagaacaaaatagaaaaatatgacgatctcacttctcaatacaagcgtattgccatggatttaggaaagaaattcgcctctttacgagacaaaattatagctgcgaatgaggataacaaggtgactcactatgcaatgattcttgaggattccaattgcgacaaatatctcttgttgcaaaaagtaagcaataacatttatcattgtatgagttacgattcttcagatcccaaagcttactatgtcgactctatcacatcttctgcaatagcaaagatgatcagaaaggaaacgaatccatccaagataagagagtatgctgaattagaagagaaagaaagagagagacggaacgttgatgattggtgcaggtttataagcaaaaaagaatacgacaggaggtatcagttgaatattaacaatggtttatcttttgaggcactaaaaaaagagatcgattccaagagttatattttggtgaaaaagaacattagtgttgattctatccgtgaacttgttgaaaatgaaggatgccttcttttccctatcgtcaataaagacctcacaaaggaaaggaaaacaacagaagacaatcaattcacaaaagattggaatatgatattctctgggtctgaaaccaattggagattaacccctgaattcagagtgacgtatagaaatccagtaccaggatatcctaatgacaaatttgggtcgaaaagatattcaagattccagatgaacgctcattttgtatgcgattttattccatcctctaattcatacacatccaacagagagcaaatcgccatattcaaagacgagggagaacagaaaaagagagtagaggaatttaatcgcaccctatcaaacatcaatcaaaaattctatgtcattggtattgatagagggcaaaaagagcttgccacactttgtgtcgtcgaccaggataagaaaatacacggagattttaagatctacactcgcaaattcaattccgaaagaaagcaatgggagcattattctctcgaaggagaaaaagggacaagaaacatccttgatctatctaaccttcgtgttgagacgaccattataattgatggcaaaccagaaaggagacaagtgcttgtcgatctgagtgaggtgttggtgaaggataaagaaggaaattacacaaagcccaacaagatgcagatcaaaatgcaacaaatggcttatgttcgaaagcttcagttccaaatgcaggcaaatccaaccgaggttttggaatggtatgagcagaatccaacagaagagttgatcataaagaatctcgtcgacaaagagaacggagaaaagggacttatctctttctacggaacagcacttgtagaactagaccagacacttcccgtctcaaaaataaaagagatgctcgaggaatttaagatactgaagcaaagagaaagcaaaaaggagaatgtgcaaaaagagctgaataaccttacacaacttgaagctgtcgacagtctgaaagcaggaattgtcgcaaacatggttggtgtgatctcatatatcttaaagacactggattataatgcctatatctcactcgaagatttgtctacagtgcaatccagcaccgaatttgcaagtggaatatcaggagcaatcaccaaaatgtcgagagaagaaggaagaagaatagatgtggagaaatatgccggtcttggattgtataatttcttcgagatgcaattgctacgaaaactacaccgcatacaaacagacaacggaaacattcttcatcttgtccctgctttccgtgcccaaaagaattacgaccacatcatggtaggaaaagaaaaaataaagaaccaatttggaatcgtgttctttgtagacgcggcagctacatctatcaaatgcccacgctgtggagcggtcaatgaggataaattcaatccagataaacaaaagtacccagatgcagaaaaagggccaaaattgagaaatcgcaaggagcagtcaggcaaaaaagtatgggtaaccagagacaaggaagacgacgaccgtatcaaatgctattgctgtggattcgacacaaaggaaaagaacgaaggaaatccttttatgtatatcaagagtggcgacgacaatgcagcctatcttatatcagatttaggagttgagtcttacagaaaagcttatgaattagctgcgactgtagtggaagacagaaaaaaaacattaactaacaatttaaatcaaagcaactacaaaattagatttttatggcacactatgtattag

SEQ ID NO: 65 - DNA, 3888 bp, synthetic construct
  source 1..3888: mol_type = other DNA
atgaaaaacattgtaaataactatcagatttcaaaaactttgcgttttgggttaacgcaaaaaacgaaaattcaaaaggaagggtataatggagaaatatatgtaagtcacagagaattggctgatttggttaaaatttcagaagaaagaattaaaaaaagtgtttcttctagtgataagtcaaatcttgagttatcactagataaaattgatatttgtcttaaacaagttggagctttcctatcggattggcagcaagtatattatagaaaagatcaagttgcattggacaaggattactataaaattctttgtaaaaagattgagtttgatggcttttggaaagatgtcaaaggtcaaagaatgcctaattcaaggataataaatatatctgaattagatacaaaggatagcttgggtgttgagagattgcaatatgtattgaattattggaaagacaatttggtcagcgcatctcaaaaatattcggtagtagaggagaaaataaaacgatttaagttggctattaaaataaacaggacagataataagcctgatgaagttgaacttagaaaaatgtttttatctctggcaaatattgtttgtgatacgctccaaccattgtgttatggccaaattagttttccaaaaataaataagttggacgattctagagctgataacaaaaaattaataaaatttgcgacagattataagtctaaaaatgacttgctcactagcattgctgagcaaaagaaatattttgaggaaaatggaggcaatgttccattttgccgggcgaccctaaatcccaagacagctttaaaggaccccaattcaacagataacagcattaaaggtgaaattactcagttaggactagattcaatattaaagagttttaaatcttatttattctttgaaaacagtttggagcatatgtcagcaaaagaaaaaattgatttaatgaagagtagtggagctaatgttgttaaaaaagggcttatgtttaagtacaaaccaattcctgtcatagtacacagagaagttgctcatgagttgagcaaagatttaaataaaaccgaggaatcattaagtgattttttgcgaggtattggacaagctaaaagtcctgcaaaggattatgaagagttgacagataaaaataaatttaatattgaggcatacccaataaaggttgcatttgattttgcttgggagagtttagcaaaggctaaatatcacagtgaaattgatttgccagtagctgcatgtgagaaattcttgaatactttttttgatgtgaaacctaataacgataaattcttgttatacgctaaacttcaagaacttaatgctcttatatctactttggagtatggaaacccttctgatgaacaatccattgtagaaaaaatcaaggctctgtctaatgaaataaaatggggcgattttggcgggagcgggcaggggtacaaaaaatcaatatctgattgggctgacagcaaaaaagattctgacggtttcaaaattgcaaaacaaaaaataggcctttttagagggggattaaggaatgaaatagctgagtattacaacttaacgcaaatatataaaaagactgtaatgcaagagggaaagctatttgcaactatgcgagataagataacaggtgcagccgaacagaataaagttactcattatgcggccattatagaggataaaacaggagataaatatgtgttgttacaagaggtgccattgaataagcaagacagaatttatgacaagatggaccgtaatggagatggatatgttagctattttgttaattctatcacgtcgcgcactattgctaaacaattaaggaaaaagagaatggcggaactaatgaagaataatgcgcgtgggatatataataatgttagtaatattcaacaaccggctctttccgataaagaaaaggaggaaaggaatataaaggaatggataagttttataagtgagaagagatgggattatgagtttaatttgaactttaaaggaaaaaactttgaggaaatcaagaaagaagtagatgctaatggctatgaattagaaaacagaatcttgagcagagacgctcttgaagagttagtaaaaaataagaaatgcttattgttgccaattgttaatcaggatattataaaggagagtaaaacggaaagcaatcaatttactaaagactggaattctatatttgacggtaattctccatggcgtttaactcctgaatttagagtgtcttaccgcaaacctactcctgattatcctatgtctgataaaggagacaagcgttactcgcgtttccaaatgattgcccattttttatgcgattacattcctcaaggtggtagttatgtatcagttagggaacaaatagataattataaggatgataaaaagcaagaaattgctgttaaggatttccatgataggttattgggaaaaactgatgaacaaaaatttatagaggggttaggtgggttgtcaagtttgggtaatgttaccataaaaactaaactcaaaaagcaagatatttctaaagagaaattttatgtttttggaattgacagaggacaaaatgaacttgcaacactttgtgtcattgatcaggataaaaaaatacagggcggtttcagaatatatacaagattatttaataatgagaaaaaacagtgggagcataaattcctggaagagcgcaatattttggatttatcaaatttacgtgtagaaaccactattgttattgatggacaggaaaaaagagagaaagtgttagtggatttaagtgaggtaaaggttaaagacaattcgggaaattatgtaaaacctaataagacacaaataaaactccaacagcttgcatatatccgcaaacttcaatttcaaatgcagacaaacccaggcagagttctagaatggtattctcataatcagaccagtgatttgattattgacaattttgtagataagcaaaacggagaggaaggacttgttccattttttggagctgcagtagcagaattaaaagatactttacctatcgataggatttccgatatgctaaaacagtttgtggaactaaagaacttggagaagcaaggggaagatgtgaagtcaaaaatcgaccagcttattgaacttgaaccagctgataatctaaagtctggtgtggttgcaaatatggttggtgtaattgcatttcttcttaaaaagtatagttataaggtctatatttcattagaagatttgtcaaagccatttaaagaccaaattgttttggggattagcggggtgccaattggtattaagaagggtatggcaggtagaagtattaatgttgaacaatatgcaggtcttggactttataatttttttgaaatgcaattgttaaagaaattattccggatacagcaggatagttctcatatcttgcaccttgtacctgcttttagggctatgaaaaattatgataatgtagcagtaggaaaagggaaaattaagaatcagtttggaatcgtattctttgtggatgctgctgcaacatcaaagacatgtccttgctgtggtgctattaacgatagacaatttgcacctgatttaagaaaattccctaatgctaaaaaaattgagaccccagatggaaagtctgtttggttggaaagggataaaactgacggtaaggatattataaggtgccacgtatgcggatttgatacttcaaaagagtacgatgacaatccacgtaaatacataaaaagtggtgatgataatgcagcttatttgatttctgctgagtgtattaaggcatacgaactagcaacaatactagttgataataaataa

SEQ ID NO: 66 - DNA, 3873 bp, synthetic construct
  source 1..3873: mol_type = other DNA
atgaacagcatcaagaatgaatatcaactctcaaaaactctgcgttttgggttgacaaaaaagaagaaactattaaaagacgattgtaatgaaataatttatgaaagccatacagaattaaaagagttagtactcatttcagaaaaaaaaattatggaatctgtttatatcaatcaaaaagcaaaactagatttgtcagttgatcaaatagatacgtgtctttcttcaattaaaaattttattgattcatggaaaggcatatatccaagagcagaccaaatagctattgataaagactattacaaaattctatgtaagaaaataacgtttgatggcttttggatagatgaaaagactaaaacaaaaaaacctcaatcacgcactatcttactttctgaacttagcaaaaaagatgcttcaggtaaagaaagaaagcaacatatcttagactattggaagaataatattttttctgcaatagagaaatatgaagttgtgagtagggaactaaaacaatttcaaaaagccttaaagatacaaagaacagacaataaacctaatgaggttgaactaagaaaattatttttatcacttgcaaatattattcttgatattctaaaaccacttgtaaatggtcaaatttgtttcccaaaaattgagaaattagacatttcaaaaaccgataataagaatctaattgactttgcaacaaaccacaaatttcaaagtgatttacttaacgaaattgcagaattacaacattactttgaagaaaatggaagcaatgtgccattttgcagagcaagtctaaatccaaaaactataataaaatcaaagctatcaacagataataacattgataaggaaataaaacaattaggacttgatagaattttaaatgaatatctatctgctccgtactttgataattctattatacatttgtcagctaaagaaaaactcaataaaatagaagataagaaagaaaattatattactcgtggtttgttgttcaaatataagccaattcaaataatgctacatcatgagattgcaaaaacattaagtaaagagattggtaagagcgaggaaaatataatagaattcttgggcaatattggtcaaataaaaagtccagcaaaggattatgaagtgagtaaagaagattttaatataaataattatccgcttaaagttgcttttgattttgcatgggagaatgtagcaagaaatttataccatacggatactcatgctcctattgatgagtgtagaaaattcttagcagataattttgatattaaaatagaggataacaatttaaagttgtatgcaaatttgctggaattaaatgctttattgtcaaccttaaaatatggaaagccaaaagatgagacaagcatcaaacaaaatataaaagatctactaaacaaaatttcatggaatgaaattggtaaaagtggtcaaaaaaataaaacaaatattgaaaattggcttaataataaagacaaaatagacaatcaaaatggaatagaaaatgctaaaaagcagataggtttgtttagaggatccttaaaaaataaagtacctaaatattataaactcacagaaacctacaaagatatttccatgaaaatgggaaaaatatttgctacaatgcgtgataaaataactgatgaagcagaactaaacaaagtttctcattatgcaatgattgttgaagatgacaataaagacaaatatatcttattacaagaatttacagacaagaaggaggagtgtatttattctaaaacgcaaacacacaatagtgattttacgacttattctgtcaattcaataacttcaagtgccattgcaaaaatgattcgtaaagtaaaagctgaagagctaagaaagaatcaatataataaagatactttctcaattgaagaaacaaaagaagaaaaagaaaatcgaatcattaaagaatggaaacaattcttaaaagataagcaatgggactatgaatttaacctggacacaaagaataaaaactttgaagaactaaaaaaagaaatagattccaaatgctataaattaaatatatcctatattgacaaaaaaacaattactgatttggtagaaaacaaaaattgtcttttgttacctattattaaccaagacttgtcaaaagaagaaaaaacacaaaacaatcaattcacaaaggattgggatgctattttctcacaaaatacaccttggagattgactccggaatttagaatttcttatcgtaagcctacacctaattatcctatttctgataaaggagataaacgttattctcgttttcaaatgattggacattttctttgtgattatattccacaaagcaacacgtatatctctaatagagaacaaatagctaattataaagataatgaaaagcaggaacaagctgtccaatgtttccatgataaattattaggcaagacagaaaaagaagcaaaaaatgagaaacttattgctttgcaagcaaaatttggttctataagtagaacaaacataacacaagaaaagaaaaaggagaaattttatgtttttggcattgaccgaggacaaaaagagttggcaacactttgtgtgattgatcaagataagaaaatcatagacgactttgatatttacacccgttcatttaattctaaaacaaaacaatgggatcataccttcttagaaaaacgtgccataatggatttatcaaatttgagagtcgaaacaacaataagcattgatggtaaaacagaaaagaaaaaggttttggttgatttgagtaaagtaaaagtaaaagataaacaagggcattattcaaagccagataagatgcaaataaaaatgcagcaacttgcttatattcgcaaactgcaatttcaaatacaaaccaatccagatgtcgtacttgcttggtattctgataataatactcaagatttaatattagagaactttgtcagaaaagacgacaatgacaacaaaggtcttgtgtctttttatggagcagcagtagaagaattgaaagatacattacccattgaagaaattttgaatatgttaaagcaattcaaggaactaaaagagaaagaaaaagctggtgagaatgtaaaatatgaaatagatagattgatacaattggaacctgtggataacttaaaaactggtgtcgttgctaatatggttggtgtaatagcctttttgttagagaaattcaattatcaagtatatatatcacttgaagatttatcccagccatttgataataaaataaatggtggtattactggtgtacctataaaaacaaataaagagtcaggaagaatggctgatgttgaaaaatatgccggattaggattatataacttctttgaaatgcagttgcttaaaaaactatttcgtattcaacaaaaaagcacaactattttacatttagtccctgcatttagagctcagaaaaattacgatcatgtaacagttggacaagataatataaaggggcaatttggtattgtattctttgtaaatgcaaatgccacatctaagacatgtcctatttgtggtgcaaataacagcgaaaaacctgacaaaaataaatatcctaatgctcataaagaattagcaaaagatggaaaagaagtatggatagaaagggataaatcaaatggcaacgacataataagatgttttgtttgtggttttgatacaacaaaaacatacgaagataatccggctaaattcataaaaagcggtgatgacaacgcagcttatttgatttcagtatcagcaattaaagcctatgagttagcaacaatattagctatagaaaaatataaataa

SEQ ID NO: 67 - DNA, 3768 bp, synthetic construct
  source 1..3768: mol_type = other DNA
atgaatcatatctataataactatcaagtatcaaaaactcttcgttttggattgacacaaaaacaaaaaatcagaagacctggttatacaggtgaattgtatgaaagtcataaagtcctaaaagaattggttaaaatttcagaagaaaaagttaaaaacttgatagtccctgcaaaaaatgaagaattactatcttctttagattctgttaagtggactcttacagaaatacgagagtttttagatcaatggcggtatatatataacaaatcaaaccaaatagccttagacaaaagctattacttaattctatccaaaaagttaggatttaatgatgaaaaaaaatctcgagtgattaaaatgatagaaataaaggatgatataaaagaaaaaatcattaattattgggcttttaacttaaatgaatcgaatcaaaaacttttgatggtaaatgaaatggtcaatacacagttaaaagcattagaaataaatagaactgaccataaaatcaatgaaattgaactaaggaaagctttacaatctcttttcaatacagtcttagacattttgaaacctctggtttacagagaaatttcatttataaatttagaaaaaatagaaaaagattctaagaattctttgctagaaaaatttgccactgattttcaaagaaaaatcgatttattagaaaagattagatcgttaaagacacattttagtgaaaatggagggaatgtatctttttgtagggcaacttttaacccaaaaacagcgattaaaaaccctaagtcaaatgataatagtatcttaaaagaaataaagaaacttggaataaaagatattttagaaaacaatgaaaatgttttttattttgaaaaaaagcttgctgaaattacagcgaaagaaaaattagaatatataataaaagatagtgaatcctttttgattcgctcacttttatttaaatatatttctattcctgcttttttacaccatggaattgcaactgaattagctcccattatttctaaagaaaaaaacgatttaataaactttatgatttcaatagggcaaataaaaagtccagcaaaagattatgctgatataccaaataaaaatgatttcaatgtaaatgcttaccccattaaagttgcttttgattatgcttgggaaaccgtagctaaaagccagtatcatcatgacataaatgctcctgtttcaatgtgtaaaacttttttagatgaaaacttcgaaaactgtacaaaaacaaagtattttactttatattctgaccttttagaacttcatactctattatcgaccttagattatggaaatccttcaatggaagactctataatagataaagcaaataaaataattgctaaaatcgacaataaagaacacaaaacaaaagataaagatttagataaagatatagataaatataaagagactattaaaaacagattaaatcataaaaattttaacgataaacaacgctatagtgatgcaaaaaaagagctctctcaatttcgtggtaaactaaaaaatgaaaacgatatttatagaaaattaacagagtcctataaaaaaatagctatgaatactggaaaaatatttgccgaaatgagagataaaataagcaatgcaagtgaacaaaacaagataagccaccacgctttaattatagaagatcataataaggatagatatttgtttttacaagaattcactacagacaaagaaaagcaaatagaatcaatatgtaatgatcaggctgggcaatatattgtttattgggttaattcaatcacctctaaaagtatttcaaaaatgttaagtaaaaaacgaatagagaaattaaagcaaaagaaaattataaataattctataaaaacctccatcctcagtgatgcagaaaaagaagctcgggatataaaagaatgggtgtccttcataaaagagaagggatgggacattgactttaatttagatttacagaataaaaatctagaagaaataaaaaaagaagtcgatgcaaaagcttataaattaaaagaaacacttattagtcagaaaactctatccaatttagtaaaagagggtaattgtttactcttccctattattaataaagatctagtaaaaaaagtaaaaacagaaaaaaatcaattcactaaagattggaattctatttttaagaaggataatctgtggcgcttgactccagagtttagagtttcataccgacaagctacacctggatatcctacatcagatattggaactaaacggtactctaggtttcaaatgacagctcattttttatgtgatttcttgccccaaggaactaaatatatttccaatcgagaacagattgaaaactacaagtcatcagaaaaacaaaaagaagcggtcgaaatttttcaccaacaaattgaaaatgataacaataatgttattagcacacaaagtctaaaccatttggctagacattttggcagtaaaaacattaaaaagaaacataatactatagaaaaaaaattctatgtctttggtattgatagagggcaaaaggagcttgcaactctttgcatcatagatcaagataaaaaaatagaaggaccttttaaaatttatactcgaagttttaatacaaaaacaaagcaatgggagcaccagttttatgaagaaaggtatatcttggatatatctaatctaagagtcgaaaccagtatttctatagatggaaaaccagaccaacaaaaaatacttgtcgatttaagttactataaagaaggagaaaagtttatcaagcttccaaaaatgcaagtcaaactgcaacaattagcttatattcgaaaacttcaatatcaaatgcaaagaaatccggagacggttcttgactggagctataaaaatacagacgataaatctatattagaaaactttgtagataaaccaaacggtgaaaaaggccttgtttctttttatggggcagctgttatagaacttaaagataccctacctcttagtgaaattaaggatatgctcgaaagatttaaagagttaaaaggaaaagaaaaaaatggagaagatgtttcacaacaattaaatgaactcacacaattaaaatctgtagaccactccaagtatggtgtcgtagccaatatggtaggtgtaatagcccatcttttagaaagatatgactataaagcttatatttcattagaagatttaactaaaccttacagtgctattgacggtatcactggacaaaaaaccgacgctaaatcgataagtggcaaacaacaagatgttgaaaaatatgccggtcttggattatacaacttctttgagatacagttactcaaaaaactttttagaattcaaaaagacagccagaacacattgcacttagtccctgcttttagagccacaaaaaactatgaaaatctcatcgctggtgaagataaagttaaaaatcgatttggaattgtctattttgtagacccaaagtcaacttcaataatgtgccctagttgcggaaaaactaataatagtagtaacaaagaaaaaagggttgtacgagacaaaaaaaacggaaatgacataatatactgtgaattctgtggatttgacaccagaaacgattacaaagagaacccactcaaatttataaaaagtggcgatgataacgctgcatatataatatcaacacacacagccaaaaaagcttacgaattagctaaatctattttataa

SEQ ID NO: 68 - DNA, 3768 bp, synthetic construct
  source 1..3768: mol_type = other DNA
atgagcttagcagcatttaccaatcagtatcaactcagtaagacacttaggtttggttttacacaaaaagagaaagtaagaaaagaaaattttgatggttcgatttatcaaagccatgcggcgcttagagagcttaccattgaatctgagagattaattaagggaaaacttaaatccaataccgatacggcgcttccattagaaaaaataagagcttgtattgaagagataaaacgttatacagacacatggagtaaaatcttcacccgcgacgatcagctcgctctttcaaaagaatactatcgcgtgatggctcgaaaagcccggttcgatgctttttggaaaaattatcgtgatgtaaagcaacctcaaagtcaaattgtcaggctttcttccttaaagagtaagtataatggaaaagaacgtaaagcctatttggtcgattactgggctggtaacctgcaaaccgtcaagcaaaggcttgttgactttgaacccgcaatccgacaatttgaaagtgctctaaaggacaaccgaacggatcgtaagctcaatgaagtggacttcagaaagatgttcctctctatatgcaaattggtcaatgaaacattagtgccactttgtaattcatctctttgtgttcccgacttggaaaaactgttggataatgaagctagccaagaacttcgtgatttcgtaatgatggatatctttcagttgcaggaacagattgaggcgctgaagatatattttggagagaacgggggctacgttccatacggcagaactacgctgaataaatacactgcactgcaaaaacctcatgcttttgacgaagagatagaggcaattttagtaaagcttaaattgtcagatgtcattaataacttaatcaaacaggatgatgtttctgattattttgaaaatgtaaaagacaagattgggcaactatctaacagtagtatgtccgtaatagagtgcgtacagctgttcaagtataagccgatacctgtgtctgttcggtattcgttagtggagtactttcatcgtaaacttggtatagataaggatgaactcggaaccctcttagacaccatagggaagccgaaaagcccagcaaaggattatgctgacttgcaagacaaaggcgatttcaatctctacaaatatccactaaaggtggcgtttgattttacgtgggaaagcctggcaaaagcacaatatcatgaggggctgaattttcccgaggtacaatgtcaaaaatttttagaaaatattttttttgtcaatacttcatgtgaagcttttaagacttacgccctattgctacatttgcgaggattgctcgccaaacttgatcatgaagagcctaatgacagggaggctatcatagacaaagcaatcagtcttatgaatgaagatgcttttccgaaagtacctttacgtgggacaaaaggcgattctgcaaaccaagcgatactttcctggcttcaattatcgaaagaggagcaagtctacaaaaaagagaaaaaagaccagagttataaccaatacgaaaaagctaagaacaaaatcgggctactacgtggtgaacagaagaataagattggtaagtatagagaagttacggaacaatttaaagacttagcgtccaattttggaaaactttttggagcacttcgtgagaaattccaggcgaaaaatgaattaaataaaatcacccattacggtacgattatagaagacaacaaccaagaccggtatgttttattgtatcccttaagtgaagggataatcgatttggataaacttttcgtacatgaggagtcaggaacgttaacgtcgtattatgtcaaatcccttacatcgaaaaccttaaacaagctcataaaaaataaaggtggttttaaagattttcatatggatggccaacagcctgattgggaaagagtgaagaaaaggtggtctgtgtataaagatgataaagcattcttgaaatatgttaagcgatgcctcaacgaatccgaaatggccaaagctcaaaattggggtgaatttggttgggatttttcatcctgcgatagttttgaagaaattgaaagagaggttgataaaaaagggtatagctttaagaatgatcgcaaactatccgaagatacggtcaaacgtctagttaaagaggagaaatgtcttttgcttcccatcattaatcaagatatcattgttgaagaaaccaaattgcgcaatcaattttcgaaagattgggtaaatatttttgatgccgactgcactgaatatcgcctacatcctgaatttgggatgtcatatcgtatgcctacaccaaattatccgaaaccggagcaaaagcgatactccaggtttcagatgatcggttattttcagtgcgaaattgtccctataaaaacagaatacttgtcgaagaaagaacagatcgagatctttaatgatgcagatgcccaaaaagaggccgtcgaaaaatttaatgaaatagtaaatggtagcgtcaaacccaatgattacgtggtaatcggaattgacagaggtctcaagcaattagccacgctatgtgtcttgaataaagacggagcaatacaaggtggttttgaaatctatacccggagctttaatgccgataaaaagcaatgggaacatcgttttatggataaccgggatatactcgacttaagtaatcttcgggtggaaacaacggtggatgggaagaaagtattagtggacttgtcgtcgataaaggttaaagatcaacggggaaattatacccaagacaaccaacaaaaagtaaagttaaagcagttggcttatatccgcaaattgcaatatcagatgcaggttaatccagaaaaagtaaaagcgttcgcagcgcaacatagaactcctcaggatattaaagatcatatgaaggagctcatcactccttacaaagagggaagccactttgctgatttacctttggatcgtattaaatatatgttggaagcattttgtgcttttcacacggaaaatgatcaaaccagtttacgggagttgattgaattggatgcagctgataatttaaagtcaggtatagttggaaacatagtaggggtaatcgcatttctccttaagcgattcagttacaacgcatatatatctatagaaaatctgacgagagcattttataatcagcgagacggattgtctgaaaaggaaattccgagagatcatgattttatggatcaggaaaatctggtccttgctggtttgggaacttatcactatctcgaagtgcaattattgcgaaagttgttcaggattcaatgtgatgcagggatcatcaatttggtgccagcttttcgtagcaatgataactacgaaactaccaggaagctttcgaaaaaacagggggtagaatacgtttgcaaaccgtttggtatcgtccattttgttgatccgatgtacacttccaaaaaatgtccggcctgcggaggcacaactgtacagcgaggcagttttaaggatgatattacttgccaaaatccattatgcggatacggtacttcccttgatataagcgaaaaaatccaaaagttgatttcagctaataaggctggtcaaaatattcatcttatcagtaatggagatgagaacggcgcctaccatatagctctgaagaccttaaagaatctttttggaaatatacaaaatgcgaataatgaaaggaggtatgtgaagagctttagaagcaagtaa

SEQ ID NO: 69 - DNA, 3705 bp, synthetic construct
  source 1..3705: mol_type = other DNA
atggaaaagaacgaaatagcaattagtgaatatcaaacacaaaaaaccattagatttggtttgaccgccacaaatcaaaatctgtattccgaagaaatcatgaaattattggatatttcagaaaaaagagtagaaaaacaagctgaacaagcaaaaaaagtaaataatgatgccgataaaaacaatcaattaagatgttgtttggatcaaataaaagagtatctgaaaacttggagtaatatttatccacaaattgattttttggcaattacaaaagatttttacaaggtaatttcaaaaaaagcaaggtttgattttgataaaggaaacggttctgaaataaaattatcctacttacaatcaacttattacaacaagaaacgctatctatatattatagagtcttggaaggaaaatttgcggaaaacagaaaatttatatcggaagtcagatgatttgctgaaagtatttgaagaagccaaaaatcaaaaccgtgacgataaaaaactgaataaagtggaattgcgaaaaacttttttaagtttgtttaatcttgtaaatgagagtctgaaaccattaatcgaaggtaatctgtttattgtaaatgacgacaaaattgacgaacaaaatccaaagcatgattgtgtatccgttttcatttcaaaaacagaagaaagaaggaaattatatgattatatctgtgatttgcaggattatttcaaagacaatggaggctatgtgccattgggtagagtcacattaaataaatggactgctttacaaaaatctaataatagagatgcggaaatcaatagaattattaaggaattaaagattaatagtgtttccatacaaaatattgagtatgaatacaataattttgctaataattttaaagaaaagaaagacgaaaatggtaaaattgtcaaaaacaatgcagggaatattatatgggatttaaaagcagatgcaaaatctgttatcgaaatctgtcagtttttcaagtataagcaagtgccaataaacgcccgcttaaatcttgcaaaaaggttggagaaaagtatcgactttttaagtgaatttggagtttctaaatcacccgcattggattacaaaaatgacaaaaacaactttaaccttacgaattatccgttaaaaattgcctttgattacgcttgggaaaattgcgcaaaagccaaacacgaagagattccttttccgaaagagcaatgtgaaaagtatttgaaagatgtttttgatatagatattgaatgtaaagaaaagtgccaaaacaaagaatgtaaaggttgcgaaaaatgcagaggatattatttaaacaaatatgcagatttaatacgattcaaaattttactcggaagattgaaagcggaatttcataaaacagacgaagaaaaaaataaaagcaacatacaagaattgagaaatatttttagagacttagattatcgtggtgacaaaagattaaataaaaatgaaattcaaaaagcagtcaatgcttggtttgacaacaaagaacaaagtattgggagaaagaaagaagacgaaatacatttaatggagaacgaaaaaaacaaattttccttatccatgcaaattatcggtcaggagagaggcggattaaaaagtagaatttcaaaatacaaagcattaactgaaatgtttaaagtttgtgcttcaaaatttggaaaacaatttgccgatttgcgagattatttcaatgaagcgtatgaggttgataaaattaaatatcgtgcctggattattgaagatgaaaaacaaaaccgtttcattttatttgtaaataaagaaaaagaggttgatctaacttcagaagaaggtgatttatatttttatgaagtaaaaagtttgacatccaaatctcttgtaaagtttataaaaaacagaggcgcttatccggattttcataaaatcaacaatcgtcaaatagacttaaattcaggcgaaaaagatagtcgtggtaattttattgacgatgtaaaaatacattggagtacctataaaaacaatcaaaagtttttagataagttaaaagattgtttgcaaaattcgacaatggctactgtccaaaagtggagtgagtttgagtttgagtttgatttttcaaattgtgatacttacgaaaagttagaaaaagaaattgatagaaaaggacataaacttgaacgaaaaacaatttcgctaactactattacaaacttggttgaaaacacagcatgtttattacttcctattgtaaatcaagatcttaataaaggaaacaaacaagcaaaaaatcaaaaccaatttacaaaagattggtttgatatttttgaaaataaaaagcgtttacatcctgaatttaacatattctatcgcttcaagacaaaagattatccgaatacaaaatttaaaaatggaacagaaaaaacaaaacgttattctcgtttccaaatgctggctcattttggctgtgaagttattcctcaaggcgattatttaagcaaaaaagaacagattgccatttttaacgatgataaaaaacaaacagaagaggtgaaaaaatataacaaaaatatttcttcggatgttgattatgtgataggaattgataggggaataaagcaactggcaacactttgcgttttagataaaaatggagtaattcaaggcggtttccaactttttacacggacttttaactcagaaacaaaacaatgggaacaccaagaattagaaaaacgcaatatcttagatttatccaatttgcgggttgaaacaacaattacaggtgaaaaagtgttggttgatttagcaagtattcaaaccaaaaacggcgaaaaccggcagaaaatcaaactaaaagagttggcttatattcgtgatttacaatatactatgcaaacaagagcaagcgatttgttggattttgcaagcaaaataaattctgctgacgatataacagaaaataatattaaaaactttatttcgccatacaaagaaggggaaaagtatgccgatttaccccaaaaagagatgtttgatttgcttacagaatggaaaaatgcagaggaggaaggaaagcgaaaaattgccgaacttgaccctgctgacaatttgaagtcagggattgttgccaatatggttggagttgttgcattgctttgtgcaaaatataaatacagagttcgcattgctttggaagacctaacaagagcgtatggcatccaaaaagatgcattaagcggagcaacaatttttcaaaatgacgaagattttaaggagcaagaaaatcgcaggcttgccggcgtaggaacaatgcaattttttgaagtacaacttttaaaaaaaatatttaaagtacaaattgacaaagacttacatcttattcccgcatttcgcagcatagccaattacgaaaaaattgttcgtagagacaaacagaatagcggcgatgaatttgtcaattatccttttggaattgtgtgttttgttgtcccaaaatatacttcaaaaaggtgtccgaaatgtgagaaaacaaatgtcaatagaaaggaaaatattgttatttgtaaagaatgcggctttcaaaccaaagaaggaaatccttacgaaaaaaataatattcattttattacagacggagaccaaaatggagcgtatcacattgcaaaaaaagcattgtaa

SEQ ID NO: 70 - DNA, 2721 bp, synthetic construct
  source 1..2721: mol_type = other DNA
atggagaaaaattataagagtgaaatcaatttatcttatgaagacattctaaataaatttaatttattatgaagagctattgatatcgcaaatgattttaaaaattccaataatcaaaataacttttctcttgatgaatatcctataaagttagcctttgattatgcttgggaaaatacagcaagaagcttgaaaagaactatacctttccccaaagaggtatgtaaacaatttttaaaagataattttgatgttgatatcgataatgcagattttaaattatatgccaatcttttattcatagcggataacctagcaactatagaatataataacccaaataatgaagtagaacttataaatgaaataaaacaagcatttgaatgcattagctttccttttgataaagaagtatataaatgacataaagaagctattttggaattattagataaagaaaaatctcaaagagactattccactatattaaaagcaaaacaagaattgtgattattgagatgatgattgaaaaataagattaaaaaatacagagatttaacacagagattaatagacaaaaagaattcacatttttgaatcgcgagttttgtgggaaaaacacttgctacaataagagatggtttaaaggaagaaaatgaactaaataaaattagtgattactgagtaattattgaggatagtaatcaagataaatatctacttactctagagctaaatgggaaagatataagagatagaattagaaatagcctatgaaactgagaatataagacatatgaagtaaattcctttacttcaaaagcattaaataaatttatcaaaaatcctttatcagaagacgcaaaaaaatttcatgggaaatataaggatgaatatagtttttgatatgaaaataaagattgagatttcacttataaaataacaaaagtttcaaaatatgatgaacaaggaaaatggacatgataccaggaaagttttttaagtcatataaaaaagtgtttaattgactctgagatttcaagagaacaaaattgggaagcctttggttggaattttgctggttgtaatacttatgaagagatagaaaaagaagttgattctaaatgatatcagttgactgaaaaccttatttcaatggggaatttaaaaagcttggtaaaagatgaaggttgcttgttatttcctattattaatcaagatatttcaagtcaaaaacaagaaaataaaaacatttttactttagaccttgaaaaggtatttgagtgaaaagaatgtaggattcatccagaattttctatcttttatagaaggccaatagaagaacacaagaaggagaataaatcatgaatcataaatcgttttggaaggttacaattattggcaaatcttggtatagaatttgttccaagaaatccttcttttaaaacaaagaaagaacaaaatagaattgcgattgatcaaaagaagcaaaatcagttagtgcaagagtttaaccaaaaaaaagtaaatacatattttgaaggattagataattattatatttttgggatagatagatgaatcaaacaactagcaactttgtgtgttactgataaagattgagttatacaagattttgatatttatacaaagcactttaatagtgaaagtaaaaaatgggaatacaaatttcatagaaaagattgaatattagatttaactaaccttaagattgagtctgatagaagttgaaataaatacatagtagatatatctttatttcaagctaaagatgaagattgaaatcctacaggtacaaataaacaaaatatccaacttaagcaacttgcatatatcagaaaattacaatatcaaatgtctgcaaatgaagaatgagttttaaattttttgggaaaatataagaacaaagaagaaagagaacaaaatatggaagaactaattactccatataaagaatgaaaaaattttgcagatttaccaatggatatttttcaagaaatgtttgaaaattattatagattaaaaactgatcaaaatttgtctgaatctgagaagaaaaatcttatgaagatcactacagagcttgatgcaagtgaaagcttgaaaaaatgagtagtcgcaaatatgataggagtgatttattatttgatgaaaaaatatgaatataaagtgaagatttctcttgaaaatctttcaaatgcttggcttttttcaaaagacggactttcttgagatgttgtgctaaatacgaaaaatgatgaaacaatggatttaaagaaacaagataatctagcgcttgcttgagtttgaacatatcatttctttgagatgcaattattgaataagttatttaaaatttctaccgaagaatgagttttgcatttagtcccaagtttttgaagtgtaaaaaattatatagagattatgaagataaaaggaaaatatgtatacaaacaatttggcattgtttatttcgttgatccaagaaatacaagtaaaaaatgtcctgtttgttgaaagtgatgaaaaaaatatatctcaagagttgacaatgttgtaacatgtaagaattgttgatttgatacatcatcagacaattccatccttataaataattataaaaaacaatgaaaaaatattcattttataaaaaatggagatgataatgcagcttacaatatctgagagaaaatacgttaa

SEQ ID NO: 71 - DNA, 3090 bp, synthetic construct
  source 1..3090: mol_type = other DNA
atgcgtaagatgtttttgtctcttgcgaatattgtctgcgaagtcttggtacctttatgcaacggctcaatttgctttcctgatattgaaaagatgtcagataaagatgagaacaagtatcttcgcgagtttgcggttgattataaaacaaaaatcgatttatttgattctatctcagaattgcgaaaatattttgaagaaaacggcgggaatgttcctttttgccgggcaacgcttaatcctaaaacaaaaataaaaaatccaagttcaacggataatagcattgaagctgaaattgagaagattgggatggaaaaatatttgcaaacaacagatgaaataattactgctattaataaaataaaacaaatgcaggattccaaactgccgcttattaagcgggcacttctatttaaatataaaactattccggcggttgttcagtttgatttggcaaaaagtatcggcaaaaaattaaacaaagaagaacaagaattaagaactattattcgtactatcggacaaactgcaagccctgcaaaagattatgcagatttgcaacataaaaaagagttcaatatcaatcaatatccgttaaagcctgcttttgattatgcctgggaatcactcgcaaaagcggaatatcataatgatattgaatttccaaatcaaatttgtaaacaatttctaaaagatatttttaatgttgatactgaaacagatagttatttcaaactttatgcgcaatttttatatttgcgggagcttctagcaacattagaacatagccaacctacaaatcctgagaaaattgtgaagattatagaggagttattggaaaaaataaatgcgtggaaagaaattgatgataaaaatagcgaaaagtacaaaactgccattagcaattggttaaaaacattcaataaagaagataataattttaaaaatgcaaaacaaaaaatcggtttatttcgcggcggattaaagaagaaagccggagacaaaagcagatgggaatatttgagaaaacctgatgaaaaagaagctccaaaattaactgcatataacgcattaacgcaaatatataaagatattgcaacgaacttaggcagaaatcttgcagatatgcgagataaaattactaacgagtcagaacttaataaaatttctcattatgcagcaattatagaggataagaacggcgataaatatgttcttttgaaaaaatcagacaaagatgaagaattcgctttgccatgtgaaaaagacgcagaatataagacatacatcgttaattcaataacttcttctgcgattgctaaaatgattcgtaaaaagagagctgctgatctagctaaaaatattaggctaaaaagcgaagaactgaatgatgaacaaaaagaggctaaaaacttaaaagactggattgaattgataaaaaaacagcagtatgatttcgaatttaatttaaatttgaataataaaaattttgagcaaataaaaaaagaaattgatgcaaaatgttatcaattacaaaagggtaatatttcaaaacaaactcttgaaaaattgattaatgagcaaaaagaatggctgcttcttcctatcatcaatcaagatttagcaaaaagagataaatcgactacaaatcaattttctaaagattggcaaaaaattttttccgataaaaggtgtggatacagattaacaccagagtttagaatctcttatcgcaagccaacacctaattaccctcaatctcagataggggataaacgatattcccgttttcagatgatagcacattttctttgcgattatattccgcaaggtaatctagaatataaatcaacgcgccaacaaatagaaatttttaaagattatgagaagcaggaagaatcagtaagaaattttacttataagatatatgctaataatgattatttaatatttggcattgaccgcggactaaagcaactggctacactttgtgtcttaaacaaagaagggaaaatatatggtggttttgatatttacacacgtagctttaataaagataagaaacaatgggagcattctttaagtgaaaagcgtaatattttagatttatctgatcttcgcgtagaaaaaacgattacaggagaaaaagtattggttgatttaaacagtataaaagtgaaaggtaatcaagataaccagcagaaaataaagcttaaagaattagcatatataagaaaattgcagtttaaaatgcaaacggaacatggaacagtaataaactttattaataaatatagaacacctgaagaaatccagaaaaatattcatgagttgataactccttacaaagagggtgaacattacgccgatttgcctactgaaaaaatttgtaatatgctgcaaaaattcaaagagttttcagataaaaatgatggaaaatcaaagagagagcttatagagcttgattctgccggcgaactcaaaaatggtatagttgctaatatggtaggtgtagtagcatatttgttagaaaagtataaatataatgtttatatttctttggaagatttaacaagagcatataggcgtcaaactgatggtttggacggcagggaattgtttagttcaaatgatgataaatctgtagattttaaagatcaggaaaatactgctttggcaggccttggcacttatcatttttttgaaatacaattactgagaaaactatttcgcatacagcaagaagatggaaatattctgcatttagttccggcattccgtagtgtggacgattatgagaagattattcgaagagacaaaaaaatagatggcaacgaatatgtggattatccgtttggaattgtgcgttttgttgaccctaaaaatacttctaaaagatgtccgctttgtagcagtataaatataaatagacacaaaaatattataaagtgccaacaaaaagcttgtggctttaaaactccatgggataaaacgaacgataaaaatattcaatatattcaaaatggcgatgaaaatggagcgtatcacatagcaaaaaagacattggataatttaaataataaaaaataa

SEQ ID NO: 72 - DNA, 3774 bp, synthetic construct
  source 1..3774: mol_type = other DNA
atggacgcagacaagaccaccaaagccattaacgaatatcaaacacaaaaaaccattagatttgggttaactgctacaaaccaaaatctgtattctgaagaaatcatgaaattattgaatatttcagaagaaagaataataaaagaaaaagtaaaagtaaataatgataccgataaaaccaatcaattaagaggttgtttggttcaaataaaaaaatatctgaaaacttgggaaaatatttatgcacaaattgattttttggcaattacaaaagattattataaggtaatttcaaaaaaagcgaggtttgattttgacaaaggcaacggttctgaaataaaattatcttccttacaatcaactcataacaaaaagaaacgctatcagtacattatagatttttggaaagaaaatttgcgcaaaacagaaaatttatatcggaagtcagacgatttgctgaaaatatttgaggaagccaaaaatcaaaatcgtgatgacaaaaaattgaataaagtagaattgcgaaaaacttttttaaatttgtttactcttgtaaatgaaagtttgaagccattaattgaagggaatctgtttattgtaaatgacgataaaattgatgaaaaaaactcaaaacataattatgtgttctatttcatttcaaaaactgaagaaagaagactgttgtatgacaatatctgtacattgcaggattatttcaaaaataatggaggttacgtgccatttggtagggtaacattaaataaatggacggctttgcaaaaatttaataatagagatatagaaatcaatagaattattaaggaattaaagattaataatatttctacgcaaaagactgattataaatataatgactttactgaaaattttaaagaaaagaaagatgaaaatggtaaagttgttaaaaacagtgctggtaatattatctgggatttaaaagctaatgccaaatctgttatcgaaatctgccaatttttcaagtataaaaaagtaccaataaacgcccgcttaaatcttgcaaaaagattgataaaagacaacaaattaaaaaaagaacaagaaaatacgtttttaagtgagtttggtgtgttaaaaacgcctgcttttgattatgcaagagacaaagaaaactttaatctcacaaattatccgctaaaagttgcctttgattacgcctgggaaaattgcgcaaaagacaaatacgaaaagattccttttcccaaggagcaatgtgagagatacctgcaaaccgcttttgaaatagatgcgactaaagacgaaaataaaaaattgatagatacacatttgaataaatatgcagatttactacaattcaaaattttactcgaaagatttaaagcagaatttcataaaacaaacgaagaaacaaataaaaacaacattcaaaaattgagaaatgtttttagcggtttagattaccatggagacaataggttaaataaaaatcagattcaaaaagcaatcgaagcttggtttgacaacaaagagcagaacattggaaaaaagaaagaaaatgaaaaacttttaacagagaatgaaaaaaacaacttttccttatccatgcaaattatcgggcaagagagaggcggattaaaaaacggaattccaaaatacaaagaattaacggaaatgtttaaggtctgtgcttcaaaatttggaaaacaatttgccgacttgcgagattatttcaatgaagcgtatgaggttgataaaattaaataccgagcctggattattgaagatgacaaaaagaatcgttttgttttatttgtcaataaagaaaaagcgtttgacttaacttcagaagaaggtgatttatggttttacgaagtaaaaagcctgacttcaaaatcgctggtaaaattcataaaaaacagaggcgcttatccggattttcatgatgttaagaacagttttcattacagtagcataaagaaagattggcaaaactataaaaacgatccagagtttttagataaattaaaagaatgtctgaaaaattctaaaatagcaaaagaccaaaagtgggcgaagttttgttgggattttaagcaatgcgatacttacgaaaaattagaaaaagaagttgatagaaaaggatataaacttgaaggttgtaaatctgaaccaaaaacaatttcattaactcaacttacggattgggtcgaaaacaaagactgttttttgcttcctattgtaaaccaagacattaataaaggggacaaacgaactaaaaatcaaaaccagtttacaaaagattggtttgatatttttgaaaacaaaaagcgtttgcatcctgaatttaacatattctatcgcttcccaacaaaagattatccgaatacaaaatttaaaaatggaacagaaaaaacaaaacgatattctcgtttccagatgctggcttattttggttgtgaggttattccctctggcaatcatttaagcaaaaaggagcagattgcaatttttaataatgataaaaaacaaaaagaagaggtggaaaaatataacaaaagtatttcttcggattgtgactatgttataggaattgaccgaggaataaagcaacttgcaacgctttgtgttttagataaaaatggagtaattcagggcgatttccaaattttcacacggacttttaacaagcaaacaaagcaatgggaacacaaagaattagaacaacgcaatattttggatttatcaaatttacgggttgagacaacaattacaggtaaaaaagttttggtggatttaagcaaaataaaagacgatgaaggtaactataccaatctgaaacagacaattaaactcaaacaattggcttatatccgtgaattacaatatgctatgcaaacaagaccagacgatttgttagactttgtaaaaagtataaattctgctaatgatataactgcagagaatattaagcattttatttcgccatacaaagaagggaaaaattatgatgacttgccaaaagtagaaatgtttaatttgcttaaagagtgggggaatgctgatgaaaacggaaaacgaaaaatcgccgaacttgaccctgctgacaatttgaagtccgggattgttgctaatatggttggcgttgttgcatttctttgtgaaaactataactataaggtccgtattgctttggaggatttaacaagagcttatggtatccaaaaagacgcattaaatggaacagcaatttatcaaaacgatgaagattttaaggaacaggaaaatcgcagacttgccggcgtaggaacaatgcaattttttgaagttcaacttctaagaaaattatttaaaattcaagttgataaaaacttacatcttattcccgcatttcgaagcgtagataattatgaaaaaattgttcgtagagataaacagaatagtggcgatgagtttgtcaattatccgtttggaattgtttgttttgttgacccgaaatacacaagtcaacaatgtccatattgcaataatacgcataaacataagaaaaatgatacggaaacagggaaaaaagcattttatagaaataaaggagaaaacaaaaactctctattatgcgaaaaatgcggagtttcaacaattgaaggagaggaaacgctgagcagtaaaaatgataataaaaaacaatttaatattcactacattaccgacggagatcaaaatggagcgtatcatattgctaacaaggtagttataaactttcaaaaggattcataa

SEQ ID NO: 73 - DNA, 3831 bp, synthetic construct
  source 1..3831: mol_type = other DNA
atggaaaaaattaaaaacaaatatcaagtgtcaaaaacattgcgttttggattaacgcagaaagagaaacgccgtaaaaaaggtttcgttggagaagtgtatgaaagtcatacagaattgaaagatttgacagatttttcagtcaaaaaaatcagggcagaaatcaaacaaggcgggaactccggtattccaatagaaaaaatacgtcaatgtttgattgttattaggagatatatgaacttttgggaaaacatttattatcgatgcgaccagttgtctcttgataaagatttctacaaaaagctatcaaaaaaaatcggctttgaaggattttggcatgaggaaaataggaagacagatcacagaatcaaaaaacctcaggcaagaacaattcagttatctgaattgaacaaaaaagacgaccactataaagaacgtaaagattatatcgttgagttttgggaaagcaatataaacaaagctgccgagagatttaaggaaacggaaagtgtttttgaaaagtttgaaatagcaattaatgcgaaccgtgatgacaaccgtcccaatgaagttgagatgcgaaaaatgtttttatcacttgcaaatattatctatgaaacgcttgtgccgctatgtaacgggtcaatatctttcccgaatatagaaaagatgcagaataatgaagaaaataataacttgcgtaaatttgcggctgatgacgaatttagggcagggttgcttactcaaatagaagagttgaaagcatattttgaggaaaacggaggcaatgttcattattgcagggcaacgctaaatcccaaaaccgtaataaaaaatccaaattctaccgatagcagtatcgcagatgaagttgaaaaaacaggcattgaacgatacctgcaaaataaacaagaaattaagaacaaaatagaaaaaattaaagacaaaaatttaccactgattgagcgagcattattattcaaatataaaacggtgcctgccggcgttcagtttgacttggcgaagtttttatctgtaaaattgggtaaaaccgaacaagaattaagaacgattattcgttgtatcggacaaatacaaagccccgccgaggattatgcaaaatctgaagacaaaaaagggtttcatctggaacaatacccgttaaaatccgcgtttgatttcgcttgggaatctttagcaaaagcgatttatcataaaaatgttgatttcccgcaacagcaatgtaaagtgtttttggaaaaaaattttgacataaaaattgacagcaatgctaatttcaaactctacgcgcaacttttatatttgcgggaaaatttggcaactttggaacacagtaagccgacagatcccgacacttttgagaaaaatattagaaaattacttgacgagattaattggagcactatcgacaaagaaaaaggttcagtttataaaaacgcaatctctgattggttaaaaaacaaaaaagcaaaagatgaaaaattcggaaaagtgaaacaaagcatcggtttgtcccgcggtagattgaaaaataaaattaaaaaatttgatgatttaaccaaagactataaagatattgcaacgcagttaggcaatgcgtttgcggcaatgcgcgataaaatcaccaacgctgctgaactcaataagatttcacattacgcaactattattgaggataaaaacggcgacagatatattttgttacaaaaagtaacagagaatgaaaaacctgttggagaaaactggaataaaaacggcgaattaaaaacctatttggtaaattccgtcacatccgccgcaatcagcaaaatgattcgcaagataagaacggatgaattacgcaaaaacgaaaaaatgcaatcttcaatcactaaattaaacgaaaagcaaaaggaagaaaaaaacataaatgattggaaaaattttattgaagaaaagagatgggatttagaattcaaattagacttgaaaagcaaaaactttgagcaaatcaaaaaggaaatagacacaaaatgctatctctttaacacaggatacgccagtcaagcagatatcaaacagttagtaaaagaaaaagacgctttgttgctgcctattattaatcaggatttggcaagcaaagataaaattgtacgaaatcaattctccaaagattggcagatgatattttctgacaactcgcaaggttatcgtcttacacctgaatttagaatctcataccgacaacctacttcgaattatccgcaacctgaagagaaaagatattctcgttttcaaatgattgcacattttttgattgattatattccgcaaaataatcaatacatttcaacgcgcgaacaagtagaattgttcaaagatgaaacaaagcaaagggaagcaattgaagaatttcataaacagttaacacccaaaaccgaagctgaacaaaaagctgaaagcttgtctgcgttagctgcaaaattcaataacccaaataacaaaaaacaaaaaaataatgcatcggaaaataaacctgacgaaaaattttacgtttttggaattgacagaggccaaaatgaacttgcaacactttgcgtaattaatcaagacaaaaaaattattggtgattttgaaatttatatcagaaaatttaattcggaaaaaaagcaatgggaacatttaaaacttgaaaatcgccatattttagatttgtctaatttgagagtcgaaacgacaattgtaatagacggcaagcccgaaaagaaaagggttttggtggatttaagtgaaattaaagtcaaagacaaaaatggcgaatataaaaatccggataagatgcagacaaaaatgcggcacttggcatatatccgcaaagtacaatttcaaatacaaaataaccccgaaggagttttggattttctaaaaaaattcaaaacaaagaacgaaaccatttacaacttggttgataaggaaaatggagaaaaaggtttgatttcattttatggggccggcaacacaaatgaagatttacctaaagatgacatctggaaaatcttgcaaaaatttcaagagttaaaaaataaaacaggtgacgacaatgttaaaaaagagattaaagaattgattgaacttgagccggttgataatctaaaaaacggtgttgtagctaatatggtcggcgtgattgcttatttgttagaaaaattcgactatcaggtttatattgctctggaagatttgacaaaaccgttcaacgaagatattaaagacggcacaactggggtgaaaggcaattacaaaggcgaagggaaacgggctgatgttgaaaaatatgccgggctcggactgtataacttctttgaaatgcagctacttaaaaagttatttcgtattcaacaggaaaatgataatgttttgcatttagtacctgcgtttcgtgcggtaaaaaattatgaaaatttgatcgcaggtaaaggtaaaattaaaaaccaatttggaattgtttattttgttgatgcaaactcaacgtcaaaaacttgtcctgtctgtagttctatacctgataagaataattcgaataacaaaaatgcaaaagggaaaaaaataataaataagaaaggaaacgaaagtgttatttgggttgagcgagataaatctaatggcaacgacattatccgttgttatgcctgtggttttgatacaacaaaaaattattccgaaaatccgctaaaatacattaaaagcggtgatgacaatgctgcatttattatttcaactttaggaattaaggcgtatgaattagcaaaaactttggtggcgaataaataa

SEQ ID NO: 74 - DNA, 3831 bp, synthetic construct
  source 1..3831: mol_type = other DNA
atgaaaagttttgaaaacagctgtcaaataagtaagactttgcgttttggagccacattaaaagaggataaaaaaaaatgtaaatcccatgaagagcttcaagagtttgttgacatatcatataaaactatgaaaagtagcgcaacaatagctgaaagcctaaatgaaatagagttagtcaaaaaatgtgagcgatgttatagcgagattataaaatttcatgaagcttgggggcaaatctattgtcgaacagaccaaattgcagtatacaaagatttttatcgtcaactggcaagaaaagctagatttgatgtaggcgatcaaaatagtcagctgattaccctctcatcattgagcagtaagcataatgtgtatcaaggactaaaaagatcgcaacatattacaaattattggaaagataatattgctcgtcagaagagttttttaaaagattttagccaacagctccatcagtacaaaagagctcttgaaaatagcgataaagcgcatacaaaaccaaatcttatcaattttaataaaacattttcgatacttgcaaatcttgtaaatgaaatcgtgattccactgtctaatggtgcgatttcatttcctaatatcagtaaacttgaggatggagaggagagccgccatctaatagagtttgcactgaatgattacagcgatttggtcggctcgataggtgagttaaaggatgctattgctacaaatggagggtacacaccgtttgcgaaggtcacactcaaccactataccgcagagcaaaaaccgcatgttttcaaaaatgatattgatgccaaaattagagaattaaaacttatagagttggtagaaaaattaaaaaataaaacttctaaacaaattgaggagtatttttctaagtttgacaagttcaagatctatgatgatagaaaccaatcagtcataattagaacgcaatgttttaaatacaaaccgataccttttttggtcaaacacgaattagcaaagtatattgcggaggatgaagacgctgtattaaaggtgttggatgccattggtgcgacaagaagtcctgcgcatgattatgcacacaatgaagatgtatttgatttaaaacattacccaataaaagtcgcttttgattatgcttgggaacagttggcaaacggtgtttatactacggtctcttttccagaagagaagtgtagagagtatcttaacgctatctacgggtgtgaagtcactaaagaacctgtttttaaattttatgcggatttgctttatattagaaaaaatttagccgtccttgaacataaaaacaatttgccgagtaatccagaagagtttatttgcaagattgaaaatacatttgaaaagatagtattgccatataagattaaggaatttgagacatataaaaaggctattttaacatggattaatgacggtcgtggtcatgaaaaatatactgattccaaaagagagcttggtctaatacggggtggattgaaaggtcgaattggggctcaaaaaatgtttcgtaaaaacaaaaagggagaattaataccttactacgaaaatccatatacaaaattaaccaatgaatttaaaaatatatcttcaagttatggcaagacgtttgcagagttgcgagataagtttaaagaaaaaagtgagataacaaaaattacccattttgggattatcatagaggataacaataaagataggtatcttctagccaatggattgcaacacgataatactgatcaaagcaacactcaagtagaagcgatactgggcaaactcaatacctctttagaatttaccacctatcaagtgaaatctttaacctccaaaatattgattaaacttatcaaaaatcataccacttcacctaatgccaaatccccgtatgcagattttcatacttcaaagatacatgttgactggactaagattaaaaaagagtgggatacttataagagcaatcattcattattgcaatatgttaaagattgtcttaccaactccacaatggcaaaaaaccaaaactgggcagagtttggatgggatttagagagctgcaacagttatgaagcaattgaacatgagattgatcaaaaatcatacatattgcaaaaacacacaatatcaaaggcatcaataaagtctcttgtggagaatgggtgtttattgttgcctattgtcaatcaagatattacctcgcaagggcgaaaagataaaaaccagtttagtaaagattggaaacaaatttttgaagactcaaaagagtacaggcttcatccggagtttgcggtctcttataggacacctataaaggattatccaaaagataaacgatacggcagattacagtttatctgtgcctttaactgcgagattatccctcaaaatggggaatttatcaatctcaaaaaacagatagagaattttaatgatgaagatattcaaaagaacaatgttgctgagtttaacaataaagtaaacgaagctttattgggcaaagagtatgtagttatagggatagataggggcttaaagcagcttgccacactgtgcgtccttaataaaagagggaaaattttaggcgactttgagatttacaaaaaagagtttgtccgtacagaaaacagaagtaagaattattggaagcataccctagcggagacaaggcatatccttgatctatctaacttgcgtgttgaaactacagttgaggacaataaggtactggtcgatcaaagccttacgcttgtgaaaaaaaatagagatacccctgatgaaaaagcgacagaagagaacagacaaaaaatcaaactcaaacagttgtcatatataagaaagctccaatatgcgatgcaaactaacgagcaagctgttttggatttactaaaagataactccgatgatgaagagttcaaaaagagagttgagggtgttatctctccttttggagaggggcaagagtatgcggatttgccgattgataccatgagagcgatgatacaagatttacaaggtgttatagcaaaaggcaataaacaaacagaaaaaaacaaaatcatcgagcttgacgctgccgatagtctaaaacagggcgttgtagccaatatgattggcgtagtgaattttattttagcaaaattcaattatgaagcctatgtgtcgttagaagatttaagcagggcatataacgtggcaaaaagtgggtatgatggacggtatctgccatcgactagccaagatcctgatatggactttaaagagcaacaaaaccagatgttagcagggttgggaacctatcagttttttgagatacaactgctaaaaaaattacagaaaatacagagtaataatactgttttgcgttttgttcctgcctttagaagtgccgacaattatagaaatattattaaattaaatccaaaatatgataacacagaatatgtgcataaaccgtttggtattgttcattttattgatccaaaagatacttcaagcaaatgccccgtatgtggcaaaacaggtaaaaaaaatgtagatcgaaatgaaaaaaagaataatatcttgctatgtaaagcgtgtggatttagaacagtttgggagcttaaacagccagaaaatatcaagagtgagggatattgttttagcgaagatgagattcaaaagacgataaaaaacaatcaaaagcaaatggagataaaaaagctttcagataaaaatattcactatatccacaatggagatgacaacggagcatatcatatcgctttaaagagtgtagaaaacttaaagcataaggaataa

SEQ ID NO: 75 - DNA, 3894 bp, synthetic construct
  source 1..3894: mol_type = other DNA
atgaaaagcattgtaaataactatcagatttcaaaaactttgcgttttggattaacgcaaaaaacgaaaattcagaaggaagggtataatggagagatatatgtaagtcacagagagctctgtgatttggttaaaatttcagaagaaagaattaaaaaaagtgtttcagctagtgataagtcaaatcttgagttatcaatagatataattgatacatgtcttaaacaaattggagttttcctatcggattggcagcaagtatattatagaaaagatcaagttgcattggacaaggattattataaaattctttgtaaaaagatagagtttgatggtttttggaaagatgacagaggtcaaagaatgcctaattcaaggataataaatatatctgaattagataaaagggatagcttgggtgttgagaggttgcattatatattgaattattggaaagacaatttggtcagcgcatctcaaaaatattcggcagtagaggagaaaataaaacgatttaagtcggctataaaaataaataggacagataacaaaccagacgaagttgaacttagaaaaatgtttttatctctggcaaatattgtttgtgatacgcttcaacccttgtgttatggccaaattagtttcccaaaaataaataagttggacgattctagaactgataacaaaaagctaataaaatttgcaacggaatataagtctaaaaatgacttgctcactagcattgcagagcaaaagaaatattttgaggaaaatggcggcaatgtcccattttgccgagccaccctaaatcctaagacagccataaaagaccctaattcaacagataacagcattaaaggtgaaatagctcagttaggcctagattctatattgaagagttttaaatcttatttattctttgaaaacagtttggagaatatgtcagcaaaagaaaaaattgatttaatgaagagcgatggagctagtatcattaaaaaagggcttatgtttaagtacaaaccaattcctgtcatagtacacagagaagttgctcaggaattaagtgaagatttaaataaaactgaggaatcattaagtgattttttgcgaggtattggacaagctaaaagtcctgcaaaggattatgaagagttgactgataaaaatgaatttaatatagaggcatacccaataaaggttgcatttgattttgcttgggaaagtttagcaaaggctaaatatcacagtgaaatagatttgcctgtagattcttgtaagaagttcctgaagacttttgatgtgacaccggatgacgctaattttctattatatgctcaacttcaagaactcaatgctcttatatctacgttagagtatggatatccttctgatgaacaatccattgtaaaaaaaatcaaggctctgtctaatgaaatacaatgggagaaagtttctgacaggaacgggcaagaatacaaaagatcaatctctgattgggctgatggcaaaagagattctgacggttttaaaattgcaaaacaaaacataggtctttttagagggggattaaggaataaaatagataagtattacagtttaacgcaaatatataaaaagactgtgatggatgagggaaagatatttgctatcatgcgagataagataataggtgcagccgaacagaataaagttacttattatgcggcgattatagaggataacacaggagataaatatgtgttattacaggagttgccattgaacggacaagacagaatttatgacaaaatgacccgagacggagatggatacgtttgctgttttgtcaattccattacatcacgcactattgctaaacaattaaggaagaagagaatggcggagctaaagaagaataaagcgtggggggcatataatgataatgttagtaatactcaacagccggtactttctgatgaagaaaaagaagaaagaaatataagggaatggaaaagttttataagtgagaagagatgggattgtgagtttaatttgaactttaaaggaaaaaactttgaggaaatcaagaaagaaatagatgcaaaaggatatgaattagaaaacaggcgcttgagcagagaagcgcttgatgagttagtaaaaaacaataaatgcttattgttgccaatcgttaatcaggatattataaaagagaacaaaacggagagtaatcaatttaccaaagattggaattctatttttgacgataattctccgtggcgtctaacccctgaatttagagtatcttaccgcaaacctactcccgattatcctgtatctagtaagggagacaagcgttactcgcgtttccaaatgattgcccattttttatgcgattacattcctaatagtgatagttatgtatcagttagggaacaaattgagaattataaagatgataaaaagcaagagactgctgttaaggatttccataataggttattgggaaaaactgaggagcaaaaaataatagatagattgggtgcatttcagggttggggtaatgttatcataaaaaagactaaaccagaaaagcaagaaggttctaaagaaaaattttttgtttttggaattgacagggggcaaaatgaacttgctaccctttgtgttatagaccaagataaaaaaattcaaggaggctttaaaatatatactcgttcattcaatagcgagaaaaaacaatgggaacataaattcctggaagagcgcaatatcttggatttgtcaaatttacgtgtagaaactactattgttattgatgggcaggaaaaaaaggagaaagtgttagtggatttaagtgaggtaaaagtaaaagaccattttggaaattatgtaaaacctaataagatgcaagttaaacttcaacggttggcatatatccgcaaacttcaattccaaatgcagacgaatcctgaaagagttctagaatggtattctcataatcagactaatgatttgattattaacaattttgtagataagcaaaacggggagaaaggacttgttccattttttggagctgctgttgcagaattaaaagacacattacctatcgataggatttccgagatgttaaaacagtttgtggaactaaagaatttggagaagcaaggggaggaggtgaagtcaaaaattgaccaacttattgaacttgaaccggctgataatctaaagtctggtgtggttgcaaatatggttggtgtaattgcatttcttcttgaaaagtacagctatcaggtctatatttcattagaagatttgtcaaagccatttgaaaaccaaattgttttgggaatcagtggggtaccaattggtattaacaaaggcatggctggtagaagtgttaatgtcgaaaaatttgcaggtcttgggctttataatttttttgaaatacaattgttaaagaaattattccggatacagcgggatagttgtcatatcttgcaccttgtacctgcttttagggccatgaaaaattatgataatgtggcagtaggaaaagggaaaattaagaatcaatttggaattgtattctttgtggacgctgctgcaacatcaaagacatgtccttgttgtggcgctaacaatagtaaaaagtttgaacctgattttagaaaattccctaatgctaaaaaatttgagactccagataaaaagtctgtttggttggaaagagataaatctgatggtaaggatattataaggtgccatgtatgtggatttgatacttcaaaagagtacgatgataatccacgtaaatacataaaaagtggggatgataatgcagcttatttgatctctgctgagggtgttaaggcatatgaactagctacaacgttagttgataataaataa

SEQ ID NO: 76 - DNA, 3948 bp, synthetic construct
  source 1..3948: mol_type = other DNA
atgattaatttagaaaattttaaaaatttgtatgaagtgaggaagacagtgaggttctgattaaatcagcctaacaaaaaatgagattttaaaactcatttagaatttaaagattttactgaaaagtcttttcagaatgtcgaaaatgagctagcatcaaatggtaagtataatatcggtgatgaagaacagcttattaaaaaaatatctgcttttgttgaacaactaaaaattcaactatgatactggaaagttttttatcaaagatatgatttaatagctataaataaagactactataaaatattggcaagaaaggcaaaatttgatgctatctgggagattagtaaatcaaataaatatataaaagttaaacaacctcaagcaagtcaaatttctttatcatcattaaagatttgaaataggagtgatagtattattcaatattggtgagagataattgagaaaacagattatttactcaatatttttaaacctaaattagaacaatacgagagggctattaaagatgctaataatgctcatattaaacctgatagtattaactttagaaaaacctttttacaacttttgaaactaactaaggaatttttacaacctttatctgacagatcaattatttttgagttttcaaagaagaaagtttctaaagaaattgaaaagatttctgagtttgcttgagaaaaaaataatacaaaaatttataatgtattgaaaaattgagaagaacttagacaatattttgaagcgaattgatcccaagtttcatattgaagagtaagtctaaactattatacggcggtacaaaaaccaaataattttgatcaagaaattaaaaaatctatagacgatttatgaataataaattttttgaagaaaagtgatagtcaaattatcgattatcttcatcaaggatcaaaacagaaaataaaattgttacttacttcaaaacatccttatcctattgagttattacagttatttaaagtaaagcctattcctttttcagtaaaatataatttggcaaaatttgtagagaaaaattataaaagtgaaaccaatttatcttatgaagacattctaaataaatttaatttattatgaagagctattgatatcgcgaatgattttaaaaattctaatcataaaggcaatttttcccttgatgaatatcctgtaaagttagcctttgattatgcttgggaaaatacagcaagaagtttaaaaagggatatttctttcccaaagaaagtgtgtgagagatttttaaaagataattttgatattgatgttgataatgcagattttaaattatacgccaatcttttattcatagcggataatctagcaactattgaatataataatccaaataatgaagcagaacttataaatgaaataaaacaggcatttgaatgcatgagcttttcttttgaaaaaaaagtatatgaatgacataaaaaagctatcttggaattattagataaggaaaaatcccaaagagactattctactatatcaaaagcaaaacaagaattgtgattattaagatgatgactgaaaaataagattaaaaaatacagagatttaacacagagattaatagacaaaaagaattcacatttttgaattgctagttttataggaaaaacacttgctacaataagagatggactgaaagaagaaaatgaattaaataaaattagtcattatggagtgattattgaagataataatcaagataaatatattcttatatcaaaacttgaaggaaaagacagaagagataagattgttcaaaagctttgaaagtgagacataaaggtgtatcaagtaaattcttttacttcaaaagcattaaataaatttatcaaaaatcctttgtcagaagacgcaaaaaaatttcatgggaaatataaagatgaatatggtttttgatatgaaaataaaaattgagatttcacttataaagtaaaaaacgtttcagaatatgacgaacaaggaaaatggatatgatatcaggaagaatttttaaatcatataaaaaagtgtttaattgattctgagatctcaagagagcaaaattggacggcctttggttggagttttgatggttgtaataattatgaagagatagaaaaagaaattgattctaaatgatatcagttgaccgaaaattctatttcaaaggggaatttagaaagcctcgtgaaagatgaagattgttcgttgttccctatcattaatcaagatatttcaagtcaaaaacaagaaaataaaaatatttttactttagattttgaaaaagtatttgagagaaaagaatgtaggattcatccagaattttctatcttttatagaaagccaatagaagaacacaagaaggagaataaatcatgaatcataaatcgttttggaaggttacaattattagcaaatcttggtatagaatttgttccaagaaatctttcttttaaaacaaagaaggaacaaaatagaattgcgattgatcaaaagaaacagaatcagttagtgcaagagtttaatcaagaaaaagtaaatacgtatttcgaaggattagataattattatatttttggaatagatagatgaataaaacaattagcaactctatgtgttactgataaaggttgagttattcaggattttaccatctttacaaagcattttaaccaagcctctaaaatatgggaatatagagaaaatagaaaggaatgaattcttgatttgacaaatcttaaaattgaatctgataaaaattgaaatacttatatagttgatatttcattatttgatgcaaaggatgataattgaagatctacaggaacaaataaacaaaatattcaattaaaacaacttgcatatattagaaaattacaatatcaaatgtctgcaaatgaaaaatgagttttggatttcatagcaaaatatataagtaaggatgaaagagaacaaaatataaaagaactcattactccatataaagaatgaaaaaaatttgcagatttgccaatggatatttttcaagaaatgtttgaaaattattatagattaaaaactgatcaaaatttgtctgaatttgagaagaaaaatctcatgaagatcactacagaacttgatgcgagtgaaaacttgaaaaaatgagtagttgcaaatatgatcggagtgatttattatttgatgaaatattatgattataaggtaaaaattacccttgaaaatttaaatcaatctttttgatgacaggttgattgaataaataatcattatgtagatataaagaataatttcatgtaccaagaaaatcaagctttggcttgagtctgaacatatcatttctttgaaatgcaattattaaaaaaaatatttaaaatttctactgaagaatgaattttacatttggttccaagttttggaagtgtgaaaaattactttaaagatattgaaaaatactcattgattgtcaatatttgaagaaatgaaaaatataaccaattttgagttgttcactttgtaaaacctgataatacaagtaaaaaatgtcctgtttgtttaaatacgcaaacgacttgaaaaaaagaatgaatgccgatcataaatagaaattacaaaaaatctaatatattttattgtgaaagatgctgattccaatcaatacacagtcattgtcaagaagaaaatattaaagatagtaatgattttcattattctgtagaagaagtagaaagaatagagatgaaaaataaagaagcaatagaaaaatataaaaaacaatgaaaaaatcttcattttataaaaaatggagatgataatgctgcatataatatttgagagaaaataagagaacttccaaaaaaatcagatgtaaagaatacctcgatttcatgaaatatttctttttcatag

SEQ ID NO: 77 - DNA, 3831 bp, synthetic construct
  source 1..3831: mol_type = other DNA
atgaaaagttttgaaaacagctgtcaaataagtaagactttgcgttttggagccacattaaaagaggataaaaaaaaatgtaaatcccatgaagagcttcaagagtttgttgacatatcatataaaactatgaaaagtagcgcaacaatagctgaaagcctaaatgaaatagagttagtcaaaaaatgtgagcgatgttatagcgagattataaaatttcatgaagcttgggggcaaatctattgtcgaacagaccaaattgcagtatacaaagatttttatcgtcaactggcaagaaaagctagatttgatgtaggcgatcaaaatagtcagctgattaccctctcatcattgagcagtaagcataatgtgtatcaaggactaaaaagatcgcaacatattacaaattattggaaagataatattgctcgtcagaagagttttttaaaagattttagccaacagctccatcagtacaaaagagctcttgaaaatagcgataaagcgcatacaaaaccaaatcttatcaattttaataaaacattttcgatacttgcaaatcttgtaaatgaaatcgtgattccactgtctaatggtgcgatttcatttcctaatatcagtaaacttgaggatggagaggagagccgccatctaatagagtttgcactgaatgattacagcgatttggtcggctcgataggtgagttaaaggatgctattgctacaaatggagggtacacaccgtttgcgaaggtcacactcaaccactataccgcagagcaaaaaccgcatgttttcaaaaatgatattgatgccaaaattagagaattaaaacttatagagttggtagaaaaattaaaaaataaaacttctaaacaaattgaggagtatttttctaagtttgacaagttcaagatctatgatgatagaaaccaatcagtcataattagaacgcaatgttttaaatacaaaccgataccttttttggtcaaacacgaattagcaaagtatattgcggaggatgaagacgctgtattaaaggtgttggatgccattggtgcgacaagaagtcctgcgcatgattatgcacacaatgaagatgtatttgatttaaaacattacccaataaaagtcgcttttgattatgcttgggaacagttggcaaacggtgtttatactacggtctcttttccagaagagaagtgtagagagtatcttaacgctatctacgggtgtgaagtcactaaagaacctgtttttaaattttatgcggatttgctttatattagaaaaaatttagccgtccttgaacataaaaacaatttgccgagtaatccagaagagtttatttgcaagattgaaaatacatttgaaaagatagtattgccatataagattaaggaatttgagacatataaaaaggctattttaacatggattaatgacggtcgtggtcatgaaaaatatactgattccaaaagagagcttggtctaatacggggtggattgaaaggtcgaattggggctcaaaaaatgtttcgtaaaaacaaaaagggagaattaataccttactacgaaaatccatatacaaaattaaccaatgaatttaaaaatatatcttcaagttatggcaagacgtttgcagagttgcgagataagtttaaagaaaaaagtgagataacaaaaattacccattttgggattatcatagaggataacaataaagataggtatcttctagccaatggattgcaacacgataatactgatcaaagcaacactcaagtagaagcgatactgggcaaactcaatacctctttagaatttaccacctatcaagtgaaatctttaacctccaaaatattgattaaacttatcaaaaatcataccacttcacctaatgccaaatccccgtatgcagattttcatacttcaaagatacatgttgactggactaagattaaaaaagagtgggatacttataagagcaatcattcattattgcaatatgttaaagattgtcttaccaactccacaatggcaaaaaaccaaaactgggcagagtttggatgggatttagagagctgcaacagttatgaagcaattgaacatgagattgatcaaaaatcatacatattgcaaaaacacacaatatcaaaggcatcaataaagtctcttgtggagaatgggtgtttattgttgcctattgtcaatcaagatattacctcgcaagggcgaaaagataaaaaccagtttagtaaagattggaaacaaatttttgaagactcaaaagagtacaggcttcatccggagtttgcggtctcttataggacacctataaaggattatccaaaagataaacgatacggcagattacagtttatctgtgcctttaactgcgagattatccctcaaaatggggaatttatcaatctcaaaaaacagatagagaattttaatgatgaagatattcaaaagaacaatgttgctgagtttaacaataaagtaaacgaagctttattgggcaaagagtatgtagttatagggatagataggggcttaaagcagcttgccacactgtgcgtccttaataaaagagggaaaattttaggcgactttgagatttacaaaaaagagtttgtccgtacagaaaacagaagtaagaattattggaagcataccctagcggagacaaggcatatccttgatctatctaacttgcgtgttgaaactacagttgaggacaataaggtactggtcgatcaaagccttacgcttgtgaaaaaaaatagagatacccctgatgaaaaagcgacagaagagaacagacaaaaaatcaaactcaaacagttgtcatatataagaaagctccaatatgcgatgcaaactaacgagcaagctgttttggatttactaaaagataactccgatgatgaagagttcaaaaagagagttgagggtgttatctctccttttggagaggggcaagagtatgcggatttgccgattgataccatgagagcgatgatacaagatttacaaggtgttatagcaaaaggcaataaacaaacagaaaaaaacaaaatcatcgagcttgacgctgccgatagtctaaaacagggcgttgtagccaatatgattggcgtagtgaattttattttagcaaaattcaattatgaagcctatgtgtcgttagaagatttaagcagggcatataacgtggcaaaaagtgggtatgatggacggtatctgccatcgactagccaagatcctgatatggactttaaagagcaacaaaaccagatgttagcagggttgggaacctatcagttttttgagatacaactgctaaaaaaattacagaaaatacagagtaataatactgttttgcgttttgttcctgcctttagaagtgccgacaattatagaaatattattaaattaaatccaaaatatgataacacagaatatgtgcataaaccgtttggtattgttcattttattgatccaaaagatacttcaagcaaatgccccgtatgtggcaaaacaggtaaaaaaaatgtagatcgaaatgaaaaaaagaataatatcttgctatgtaaagcgtgtgcctttagaacagtttgggagtttaaacagccagaaaatatcaagaatgagaaacactgttttagtgaagatgggattaaaaagacggcagaagacaataacaaaaaaataaaagaaaaaaatctttcagataaaaatcttcactatatccataatggagatgacaacggagcgtatcatattgctttaaaaagtgtagaaaacttaaagcataagaaataa

SEQ ID NO: 78 - DNA, 4029 bp, synthetic construct
  source 1..4029: mol_type = other DNA
atgttgacaaaatttgccaatctttatgagttaacaaaaacagtgcgatttggcctcacaccgaaaaatagctataaaaaggtgtcggatttttttgagttgacagaaaagtccttggaaagcgttcaagctgagatagttgaacgcgaaaaagaaaaaataaacataacaagcacagaggaatccataaaaaaaattcgagcatatgttgacgagctcaaaaaacagagtgctcaatggaagacaatttatcaaagagaagacatgattgctgttacgaaagaatattataaaaagctcgaaaaagaagcgggttttgacggtttttgggaggacagggggaaaaagcagccacaaacgagcgaaataatgctatcagcgcttagtaaagaatataataacaaaccaaggagagagtatattataacctattgggcgttgcttttgcagaagacagagcaacttaatagttattttgagccactcttggaaaaatatgaggaatctttatctcgaaaagaacaggcacatctgaagccaaatctggtggattttcgaaaacaatttttgtcattgttgaatgtgagtaacaaatggcttatgccagtcatcaatcaatctatagttttccctaaaatccataatgctagcaagggcgaacaaaatagaaaagtaaaagattttatcagtgaagaagggcgacaaaaaagaatacaattgcaatctatcgggcgaagtttgcgtgatttttttgaggcgaatggaagtcgggttcctttcggaaaagcgacattaaattatttcactgctaggcaaaaaccaaatagatttgatagcgaaatagacgctctaatgttggatttagaaatagaagaaatagtcaggaaagtaaaagatctaaatggagaagaattaaaaaactattttaaatatcagacatacgcaacaggagataaattagatgcattcttaggtcatagttatatgacgcttattgaaaaaattcaacttttcaaaccaaaaccaattccggctagcgttcgtttttccttagcggaaagactttcaaaaaaaatgaatcttccgatggaaaaagtcaccgctatttttgatgagattggaaatcctatcgatattgctagggaatatgaacaagcacttgatcagaaaaattttgatttgaataaatatcctattaatattgcctttgattatgcttgggagagttgtgcgagtttaattaaagggagaatatctgagggtgacttcccaaaagcacaatgcttggcgattttgaaaaaatttaatgcagatggcgatgattttaaattatatgctaatcttcgctatatcagagatgagttggctcctatcgaacataataacccgagtgcggatatagaaaaagaactagtctataatattcagaagacaatagggataatttactcgtccatgccatcggaatatcaaaaatatcttgatattattttgaagtgggttgaaatgccaaaaatcgaaagaaactcaagtgattctaactttatttttgccaagcaaaaaatcggattgttgcgtggtggattgaaaaataaaataaataagcacgctgaaatcacaaataagtttaagaatttatcaatggcattcggtagaagttgctctgatttgcgagacaaattacgcgaagaacatgagctttcaaaaataaaatactacagtgtcctaatagaagattcaaagaaaaacaggcatcttcttatgcttccattagaaacgggtgatcaaaaaatgtccgctgttgaaattgaaaacagattggatattttggagaataaatttgtcgaagatgctccaagtgcaaatgcatatatcgcaagtaaagtatcatcgtttacctctaaagctctggtaaaaatgttaaaaaatcctaagggaagtgaggactttcatattggtagtgaaaaatacaaactgcatgattataataacaagattaaaaaagaatggaaaatatatcaagatgattcaaaatttttgagctatgcaaaaaagtgcttatctgaatcaaaaatggccatgaatcaacattgggaaaagttcggttggaattttgaggggtgcaaaacctatgcagatttggaaaaagaagttgatataaaaggctattctttggaaaagaaatatatttcttttgagaataccgaaatgcttgtgtatgagcagggttgtcttctattccccattgtcaatcaggactatgcaagtgaggtgtgtcaaaatatttttacaggcaataagaatgcttttaccatcgagattgaaaaaggactgcaggaacaagatggctatcttattcatcctgagtttaccattttctatcaaaaaccaaccgaagattataaaaaatccaatcgatatggtcgatttcagctttctgctaattttagcctagaggtgaaaaaaataagcgataattttaaaactaaaaaagaaaagctaaaaaatatccgagataaagatagttttcaaaaagatgtgcaaatattcaacgaaagcataaatgcgcagcttagaattaatgaggatgtatacttctatggaattgatagggggataaacgagctggcgacactttgccttctgaataataaaaataaaatccaagattttattgttttcaagaaagaaaaaaagaaaagaaattctgaaaatcatctaagagaggcaggtgacttctatacgtatgttgattttgaaaaacaaaacattctagatctcacaaatattaaggttgagacttggaatatgcgagatgaaattcatcctaattcaaatttcttgaaacaggcttttaaaaaaatagaggcagatcctgaaatgaaaatgatagcgactcaattaggatatggtattaatgaaaaaaaacaatgcacaattttagtggagtacccgagcgatagaagcaacactttaaagctccgaatgaatcattttcaaagagccttgcaatttgctacttctcactcagaaaaacgagacattgttgggggaatagcagaaagaaattttgagggaacggatcttgaaaacccgagcgatgaagtgaaaaaacaaatcattcaagatctcttcgggcttgggtcagactttttaattgatgaaaagaaggcgggtgtcattaatgagctacagacaaaaaaatttagatttgaaaagatatattcccgcaatcagcagttattcttggaaatgtttgaaccattgatccagcaattgattgaatggtatcagcataaaaatgatcctgattatgagaagacaggaaaggcgaaatatgaagagatggaagatttagacactttgaagcggggtgtgacggcaaatatggtgggtgtattttcatttttgatgcaaaaatttcccggcgtgattgtgttggaaaatattgccaaagaaaaattagaaaaacctgttcaaagcaagctcgatgaacacacgcgagaagttgaaagccatcaggaagcggagcaccatcgttgggcgggggtggagctttatcgctatatggaaaaaaaattggttcaaaaattcagcaattggaggcagccaacaggagaaatttttaacttgactcctccgtttgctaatctggaatcattaaataaacaaacaatgacaataaattcagaaagaattgaaaatatgtttcagtttggaatattttgctatactcccgctgaatatacaacaaaaacttgcccgtgttgtaataaatatcatattaggaatagaaaaaaaggaaatactttcgacagtattatttgcaaaaatgaacattgcggttttgatacgcggtataatgaacctctgcacgaatcatttaatcctgcgcgtaatgtggaaatcgcattgagattaaatcctgaattcagtgaggctttaccaaagattagaagtggtgatcagagtgctgcattcaatatcgccaaaaaaggtctaattattatggaaaataaattatgttag

SEQ ID NO: 79 - DNA, 2625 bp, synthetic construct
  source 1..2625: mol_type = other DNA
atgaaggagtgtaaatcttttctaaaagaatcatttcaggtagacacggacaagaatcctcattttaaactgtatgcccagcttatccatttaaaagaattcctctctaccttggaatctgcccggcccaaagacacacagagcattgttaaagagactcagaatttgctgagccaaatcaactgggatgctttaggaaaagacaaaaatgacaaaaggactggcaatgattacaaaatttcgatttcctcttggttggaattaaggccgaaaaacgatgaaaaattccagaaagcgaagcagcagattggcctttttcgaggtgggctgaagaataaaataaagaatttcaatgaccttactcaagcatacaaagcggttgctatgaagcttggtaaggcctttggagatatgcgcgacaaaatcactggtgcggccgaactcaataaaatatcgcattatgcagcaattattgaggataagaatgcagaccggtatattctactgaagaaaagtcagctaagcggaggttcccaaaaaccattccagtcaaacgattccggagatttattaatctacacagtaaattcagttacatccgccgccatcggaaagatgctccgaaaaatgagggcagaggaactgaaaaaaaatgaaaaaaatagacaacctcttcctgagttaacagatgaaaaaaaggaaaaggagagtattgaaagatggaaaagctttataaaggagaagggctgggtttccgaattcgagttaaatttggacaataaaacctttgagcagattaaaaaggaagtcgattcaaaatcttatagtcttgatgtcggaagactcagcaaaaatgcacttgaaaaattaattcataatgaagaatgccttcttttaccaattattaatcaggatctggcaagtgaaggaaagattgccaaaaatcagttcacaaaggattggcaaagattgtttgccgatgagccgcagaatttgcgactgactccagaattcagaatctcttatcgacagccaaccccaaactatccagaccccggagagaagcgatactcacggttccagatgatggcgcatttcttagtagattacatcccaaagattgatagttacatttccattcgagaacagacagaaacattcaagaatccagaaaaacaaaaaaaagcagtggcgcagtttcatcaacgtctgaatcctaagagtaaatccgaatcagaccccatcgctcttctatcagcaaaatttgggagccaaattaacaagaaccaagggcgaaaggcgagcacggaggaagctaagcagaaagaggatttttttgtttttggcattgaccgaggacaaaaggagcttgctaccttatgcgtaattgatcaggacaagaaaatcattggtgacttcgaaatctatacccgccagtttaatgcagaaaagaaacggtgggagcatagtttgttacctgaacgccgtcacattcttgatctttccaatttgcgggttgaaacaactctttcgattgatggaattgatcaaaagaaaagggttttggttgacttgagtgaagttaaagtaaaagacaaagagggaaattattctctgccgaataagatgcaaacaaagatgcgccatctggcatatatccggaagctgcaatttcaactgcaaagtaatcctgacggggttttagattttctgtcaaaacacccggaaaaggaaaatgctttagacaatttattagataaagaagaagacgaaaaagggctgatttcgtattacggcgcaggcaatgcgagtgaggatctgccaaaagaggaactctggaatatactcgtcaattttagagaactgaaaaatcaactacaagaagatccggaaaaccaagccttagatcaagaactgagagcactgatagaattggaaccagtcgataacctgaagtacagtgtggtagccaatatggtgggtgtaatagcctatttaatggaaaagttcaactatcaggtttacatcgcattggaagatctaacaaacccgtttgcacctaccaccgtaagtggcatcaccggtgttagtaacaagaatcgccaaggcgaaggaaagaagagtgatgtcgaaaaatacgccgggcttggtctttacaacttctttgagatgcaattgttaaaaaagctgaagcgtatccagcaggaaaatggcaatgttctgcacctggttccggcgttcagggcagtaaagaattatgagaatgtaatcgcaggcaaagacaagattaaaaatcaatttggaattgtcttttttgtagatgccgacagcacttccaaaatgtgccctgtttgtgaatcgacaaatgataagcaaccgaacaaatcaaaatatccagaggcaaagaaggaaaaatccagagaaggaaaagaagtgtgggttgaaagagataaattcaatggcaaagacagaatccgctgttatgtttgcggttttgacacggacaaagattacgatgagaatcccttgaaatatattaagaacggcgatgataatgccgcttatattattgcgacccttgggattaagggatatgaactctcattagctgatttaaacccctaa

SEQ ID NO: 80 - DNA, 3687 bp, synthetic construct
  source 1..3687: mol_type = other DNA
atggaacctattaaaaatgaattccaactactcaaagcattaggatttggtttaactgccaaacattctgaaaagaaaaataaaacccatgaagaattaatggacttaatccaactatctgaagaaagaattaaaggaaaaacaaagcccaccaacgagattactgaaaagaaaatgcttgaaataatctctgagtgtgtttttttgatgaaacatcatataaacacatgggaaaaggtttacaaccgagcagatcaaataacgctaactaaagaatattacaaagaattggctaaaaaagcctgttttgacgccttttggacagataataaagacaataaacaacccaaatctcaattaattaagctttcttcactgaaaaaacccagatatgaaaattacgaaagaatgggatatatattacggtattggagaactaacatacaaaatctaaaaagaaaaatacatgattttgagttgctattatcccaatacaaattagcattatctaaacaagataaagctcatactaaaccgcaattagtcgatttcaggaaaatgtttctttctatagtcgcgttaatccatgaaactcttatacctttgaataataattccatacaattccttcaattagataaattgccggatggagaggacagcgaaaccgtaaaaacttttgctatagaagaaagagaagagcgtaaataccttgccgcaaaaatcaaagagacgaaggaatattttgaagaaaacgggggatatgttccctacggcagagttacattaaatccctataccgccaaacaaagaccacatattttcaatgaagagattgctgatattatcaaagacttaggtgttgaaaaattaataaaaaaattacaaagtataaatgacatatcaaaatattttgaatttgccgaatctgacaacgacaaaaggaaactcatgaaagattcttcatattccattattgaacgagttcagttttttaaatataaacctatcccagcttctgtctgctttatgttggctgattttttacaaagaacgaaaaaattagaaaaagaaaatgtattggatcttcttaataagataggaatgcaacgaagtatatcaaaagaatataaagataaaaaaagtattaatttagagcagtaccctttgaaaattgcgtttgattatgcttgggaaaatcttgccggacgcatacataattcaacaataccattctctgatcagcaatgtgtcgattttttgcagcaatttggagttgatatcaataatgataatttcagattgtatgcagacttactttttgtaaaaaataatttggctacattggaaaacccaaagaaccaacccaatgacgggtctgaatttattgataatattaaaagaacctttgaaaatataaaactgtcaaaagaaaataacaagcacaaagatgttattttaaactggttgaataaaaacactgaagaacaaaaagaattgaaaaaaggaaacgctcaattattcaaagattatgaaaaggcaaaagaaaaattagggcaattaagaggaatgcaaaagaacagttgtccaaaatataaatccttaactgaagaatttaaaaatctggcaattgactttggcaaaaaattcgccgatttgcgcgacaaactcagagaagaaagcgaattaaataaaataactcattttggagtaattgttgaagattcgcaaaaagatcgttacattttattgggaaaaccggaagacaagttaatacataaatttttaaataatgatcctaacggaaatttaaaaacctataatgttaagtcattaacttcaaaagcattgagcaaaattattaaaaacaaaggcgcttataaagcatttcattcatctcaggtaaaaattgatcgaaaagaaataaaacaaaaatggcaagagtatcaagaccatggcgattttattaattatgtgaaagattgttttgcaaattccccaatgtctaaagaacaaaaatggtcagagtttcaatgcaaattcgatcattgccaaaaatatgaagaaatagaaaaagaattagatgcaaaggcttatctgttaaaagaacaaaatatttctttgcaaacaatcaaaaaacttgtagaacaggaagattgtttgctgcttccttttgtaaatcaagatataacttcaaaaacccgagaacggaaaaatcaatttagtaaagattgggacatgatgtttaaaaaagataacggttatcgtttacatccagaattcaggatattttatcgccagcctacgcccgactatcctaaatcgggcgaaaaacgttactctcgttttcaaatgactgctcgcttactttgtgaatttattcctcagtccgataattatatttcacacaaacaacaaattaaaacttttaataataaagataaacaagaagaagccgttaaagagttcaatgacaaattacatcatcctgacaacttttatgtttttggtattgaccgaggaattaatcaattagccgtactttgcgtattaaacaaagaggggcaaatacaagaaggttttaagatatatacgcgaagttttgatattgaaaaaaaacaatggatacatgaattgtccgaaaaaagaaatattttggatttgtctaacttgcgggtagaaactacaatcaataatgaaaaagtattggttgatttaagcggcattaaaataaaaaacaaggaaggctatcaagataaccgacaaaaaattaaattaaagcaattggcatatattcgtaaattgcaatatcaaatgcacgcagcgccgcaaagaacattggcttttataaacaaatatcatacaccagaagatatgaaaaaacatatacaggaagatataaaagaattgattactccgtacaaagaaggagttaattacagtgatttgccaatcgaaaaaatctgcgatatgttgcaacaatttaaacagtattatgacaaagaggataaaaaatccacaagagaactaactgaacttgattctgctgatgaattaaaaaccggaatagtggctaatatggtaggcgttatcgcctatttattaaaagaatatgattacaaggtttatatttcatttgaagatttgactcgtgcattcggtcgtcaaaaagacggattagacggcagagaattaccccattcaaatgaagacaaaactgtagattttaaaggccaggaaaatgcggcattagccggactcggaacctatcactatttcgaaatgcaactacttagaaaattattccgcatccaacaggaaaacaaagatattttgcatttagtgccggcattcagaagcgttgacaattatgaaaaaattgtccgacgagacaagaaatcagacaatgatgaatatgttaattatccgtttggtatagtgcggtttgtcgatccgaggaatacctctcgaaaatgccctaactgtggtgagattaaaaaacaaataataacacgaagtaaaaacaaaataacttgccgtaaatgcgggtttataacaggcaataacaaccatgacaaaataaaggataataaaaaagataaaaacttacattatattaccaatggcgatgataatggcgcataccatatcgcattaaagacattaaaaaatctaaaaggtcaataa

SEQ ID NO: 81 - DNA, 3633 bp, synthetic construct
  source 1..3633: mol_type = other DNA
atggaaaaggacaaaattacagcaattagtgaatatcaaacacaaaaaaccattagatttggcttaactgcgacaaatcaaaatctgtattccgaagaaatcatggaattattgaatgtttcggaaaaaacaataaaagaacaagtagaaaaagtaaataatgatattgataaaaacaatcaattaagatattgtttggagcaaataaaaaaatacatagaaacttgggacaatgtttattatcaaattgatttttgggcaattacaaaagattattataaggtaattgcaaaaaaagcaaagtttgattttgacaaaggaaatggctctgaaataaaattatcttccttgcaatcggctcataacaacaggaaacgctatctctatattatggacttttggaaagaaaatttgtataaaacagaaaatttatatcggaagtcagacaatttgttgaaagtatttgaagaggcggaaaaacaaaatcgcaatgataaaaaattgaatgaggtggaattgcgaagaacttttttgagtttatttaatcttgtaaatgaaagtctgaagccattaattgaaggaaatctgtttattgtaaatgacgacaaaattgacgagcaaaattcaaatcacaaatttgtggaggattttatctcaaaaacagacgaaagaaaacagttatatgactgtattacggaattgcaaaaattttttaacggcaatggcggttatgttcctttcggcagaataacactaaataaatggacggctcaacaaaaaccgcatattccaaaagataaaaatggcaataatgaaatagacaaaattatttcctttttaaaaccagaaagtgtttcgatgcaaaatattgattatgagtacgatgcatttgaagataattttgaacagatagataataagtggaaattaaaacagaatgcaaaatctgttatcgaaatctgtcaatttttcaaatataaaaaaatcccagtaaacgctcgcttaaatcttgcaaaacgcttgaataaggacattcactttttaagtgattttggggtttcaaaatcaccggctttggattacaaaaacgataaagataatttcaatctcgccaactatccgctaaaagttgcctttgattacgcttgggaaaattgcgccaaagcaaaatatgaaacaataacttttcctgaacagaaatgtaaggattatttgaaagatgtttttgatttaaatgttgatagcaacactgattttaccaaatatgctttattgttgcgtttcaagattttagtcggaagaattaaagcagaagaaaatacacgaaaagaaaatattgaaactgtaagaagcttattgaaagatgtaaaagataatctttcaaaagaaaaagacaaaacgataacatccgttgaaaattggctcactttcaaagaaaagcaaacagataaaaaggaaaaatacgaaaatcaagacgaattttccaaagtgatgcaaactatcggacaagagcgtggcggattaaaaagtagaatttcaaaatataaaacattaactgaaatgtttaaagtttgtgcttcaaaatttgggaaacaatttgccgatttgcgagattatttcaatgaagcgtatgaggttgataaaattaaatatcgagcttggattattgaagatggaaaactaaatcgttttgtattgcttgccgataaaacaaaagatttgaatttagaaaatgataaaaacggcaatttgaaactttacgaggtaaaaagtttaacctcaaagtcacttgtgaaatttatcaaaaacaaaggtgcttatcctgattttcatatagtcaacaatcggcaaatatatctaaattcaggtggaaaagataatcgtggtaactttattgacgatgtaaaaatccattggagcatatatcaaaacaaccaaaagtttttaaacaaactaaaagactgttttcaaaattcaaaaatggcaattgaccaaaattggaacgaattcaattttgatttttcaaattgcgatacatacgaaaaaatagaaaaagaaattgatagaaaaggatatattcttcaacaaaattctatttcattagataaggtaaaagatttgataaaaaatcaagaatgttttttgctgcctatcatcaatcaagatattaacagagaagacaagcaggcaaaaaatcaattttcaaaagattggaacgatatttttgtaaatacaaaacgcttgcatccggaatttaatcttttctatcgctttccaacaaaagattatccgaatacaaaattcaaaaatggaacagaaaagacaaaacgatattctcgtttccaaatactagctcatttgggttgtgaagttattcctcaaggtaattatttaagcaaaaaagagcagatagccatttttaacgatgatgaaaaacaaaaaggggaagttaaaaaatatatcaatatttgttctgattttgattgtgttataggaattgacagaggaataaagcaacttgcaacgctttgtgttttagataaaaatggcagtatacaaggtgattttaagatatttacacgaacttttaatcacgatacaaaacaatgggaacatcattttcttgaaaaacgcaacattttggatttatcgaatttaagagtagattccccaatcactatttgttgtaatattcctgacgataatcacaaaaaagttttggttgatttggcaagtattcaaacgaaaaatggcgagaatcaacaaaaaatcaaattgaaaaaactcgcttacatccgtgaattacaatatgccatgcaaacgcaagatttgtccgaatttgtaaatagtttgaaatctgccgacgatattacacaagataaaatcaaaaactttatatcaccatacaaagaagggcctccttatgcagacttaccaaaacaagaaatgtttgatttgcttaaagaatgggaaaatgcagatgatgaagaaaaacaagtaattgccgaacttgattccgctgataatttgaaatcagggatcgttgctaatatggttggtattgtggcatttctttgcgaaaaatataactacaaagttcgtattgctttggaagatttaacaagaagttatggtatccaaaaagatgcgttaagcggaacaacaatttttcaaaatgatgaagatttyaargagcaagaaaatcgaagacttgcyggygtaggaacaatgcaattttttgaaatgcaacttctaagaaaattatttaaaattcaggttgataaaaatttacatcttattcccgcatttcgaagtgtagccaattacgaaaaaattgttcgtagagataaacagaatagcggcgatgaatttgtcaatcatccttttggaattgtgtgttttgttgacccaaaatatacatcaaaaaggtgtccgaaatgtggaaaaacaaatgtcgatagaaaagaaaatattgttatttgtaaagaaaagagttgcggctttcaaaccaaagaaggaaatcctttcgaaaagaacaatattcatttcattactgacggcgaccaaaatggtgcttatcatattgcaaagaaagtattgaaaaaagacagcaacaacgaaacaaaaaaataa

SEQ ID NO: 82 - DNA, 3807 bp, synthetic construct
  source 1..3807: mol_type = other DNA
atgattaatctagaaagttttaaaaacttgtatgaagtgaggaaaacagtcagattttgattaaatcaacctagaaagaaaagtgatattaataaaacgcattgactattaaaagatttggttgagatttcttttgagagagaaaaaaaactaattaataatgaaaataatcacgtactaattgatacagagaaagttcttatagagaaattacagcaatatgtaaattgactagaagtacaactaaaaaactggaaatgagtatatcaaagatatgatttaatagctataaataaagactactataaaatattggcaagaaaagcaaagtttgatgctatgtgggagattactaaatttgataaaaaatcaaataaatatataaaagttaaacaaccccaagcaagtcaaatttctttatcatcattaaagatttgaaatagaagtgatagtattattcaatattggtgagagataagtcagaaaacagattatttacttaatatttttaaacctaaattagaacaatacgagagggctattaatgatgctaatagtgctcatattaaacctgatagtattgactttagaaaaacctttttacaacttttgaaactaactaaggaatttttacaacctttattggatagatcaattattttcgagctttcaaagaagaaagtttctcaagaaattgaaaagatttctgagtttgcttgagaaaaaaataatacaaaaatttataatgtattgaaaaattgagaagaacttagacaatactttgaagcaaattgatcccaagttccatattgaagagtaagtctaaactattatacagcagtacaaaaaccaaataattttgatcaagaaattaaaaaagctatagacgatttatgaataataaattttttgaagaaaagtgatagtcaaatgatcgattatcttcatcaaggatcaaaacagaaaataaaattgttacttacttcaaaacatccttatcctattgagttattacagttatttaaagtaaaacctattcctttttcagtaaagtataatttggcaaagttcattgagaaaaattataagaatgaaatcagtttatcttatgaagagattttagataaattgaatttgttatgaagagctattgatatcgcaaatgattttaaaaattccaataatcaaaataacttttctcttgatgaataccctgtcaagttagcctttgattatgcttgggaaaatacagcaagaagtttaaaaagaactatacctttcccaaaagaggtatgtaaacaatttttaaaagataattttgatgttgatgtcgataatgcagattttaaattatatgccaatcttttattcatagcggataatctagcaattattgaatacaataatccaaataatgaagtagaacttataaataagataaaacaagcatttgactctatagacttttcttttgataaagaaagatattgatgatataaaaatgatgttttagtattgctaaataaggcaaaatctcaaagagactattccactatattaaaagcaaaacaagaattgtgattattgagatgatgactgaaaaataagattaaaaaatacagagatttaacacagagattaatagacaaaaagaattcacatttttgaatcgcgagttttgtaggaaaaacacttgctaaaataagagatcgactcaaggaagaaaatgaactcaataaaatttctcattacggagtaattcttgaagataaaaatcaagataaataccttcttatattacaacttgatggaaaagatacaagagaaaaaatatctcaaaaacttggcaatggagacataaaggtgtatcaagtaaattcttttacttcaaaagcattaaataaatttatcaaaaattcttgatcggaagatgcaaaaaaatttcactgagattttagatataaacataaagaagtttcaatatatgatgaaaaaggaaaatggacatgatatcaggaaagttttttaagtcatataaaaaagtgtttaattgactctgagatttcaagagaacaaaattgggaagcctttggttggaattttgctggttgtaatacttatgaagagatagaaaaagaaattgattctaaatgatatcagttgactgaaaactctattttaaaagagactttaggaaaattgctgaaagatgaaggttgttcgttatttcctattattaatcaagatatttcaagtcaaaaacaagaaaataaaaatatttttactttagattttgaaaaggtatttgagtgaaaagaatataggattcatccagaattttctatcttttatagaaagcccatggaagaacacgagaaggagaataaatcgtgaatcataaatcgtttttgaaggttacaattattggcaaatcttggtatagaatttattccaagaaattcttcttttaaaacaaaaaaagaacaaaatagaattgcgattgatcaaaagaaacaaaatcagttagtccaagagtttaaccaggaaaaagtaaatacatattttgaaggattagataattattatatttttgggatagatagatgaatcaaacaattggcaacattatgtgttactgataaagattgagttatacagtcctttgatatttacacaaaattttttgattctaaagaaaaaatttggaaatatacaaaaactagaacggaaggtgttcttgatttgacaaatcttaagatcgaatctgataaaaattgaaatacttatatagttgatatttcgttatttgatgcaaaggatgaagattgaaatcctacaggtacaaataaacaaaatattcaacttaagcaactggcatatatcagaaaattacaatatcaaatgtctgcaaatgaagaatgagttttaacttttttgggaaaatataagaacaaagaggaaagagaacaaaatatgaaaggactaattactccatataaagaatgaaaaaattttgcagatttaccaatggatatttttcaagaaatgtttgaaaattattatagattaaaaactgatcaaaatttgtctgaatctgagaagaaaaatcttatgaagatcactacagagcttgatgcaagtgaaagcttgaaaaaatgagtagtcgcaaatatgataggagtgatttattatttgatgaaaaaatatgaatataaagtgaagatttctcttgaagatctttcaaatgcttggtttttttcaaaagacggactttcttgagatgttgtgctaaatacgaaaaatgatgaaacaatggatttaaagaaacaagataatctagcgcttgcttgagtttgaacatatcatttctttgagatgcaattattgaagaagttatttaaaatttctaccgaagaatgagttttgcatttagtcccaagtttttgaagtgtaaaaaattatatagagattatgaagataaaaggaaaatatgtatacaaacaatttggcattgtttatttcgttgatccaagaaatacaagtagaaaatgtcctgtttgttgaaagtgatgaaaaaaatatatctcaagagttgacaatattgtaacatgtaagaattgttgatttgatacatcatcagacaattccatccttataaataattatgaaaaacaatgaaaaaaacttcattttataaaaaattgagatgataatggtgcatataatatttgagagaagataagagaacttccaaaaaaatcagatgtaaagaatacctcgatttcatgaaatatttctttttcatag

SEQ ID NO: 83 - DNA, 2721 bp, synthetic construct; identical to SEQ ID NO: 70
  source 1..2721: mol_type = other DNA

SEQ ID NO: 84 - DNA, 2733 bp, synthetic construct
  source 1..2733: mol_type = other DNA
atggagaaaaattataagagtgaaatcaatttatcttatgaagacattctaaataaatttaatttattatgaagagctattgatatcgcaaatgattttaaaaattccaataatcaaaataacttttctcttgatgaatatcctataaagttagcctttgattatgcttgggaaaatacagcaagaagcttgaaaagaactatacctttccccaaagaggtatgtaaacaatttttaaaagataattttgatgttgatatcgataatgcagattttaaattatatgccaatcttttattcatagcggataacctagcaactatagaatataataacccaaataatgaagtagaacttataaatgaaataaaacaagcatttgaatgcattagctttccttttgataaagaagtatataaatgacataaagaagctattttggaattattagataaagaaaaatctcaaagagactattccactatattaaaagcaaaacaagaattgtgattattgagatgatgattgaaaaataagattaaaaaatacagagatttaacacagagattaatagacaaaaagaattcacatttttgaatcgcgagttttgtgggaaaaacacttgctacaataagagatggtttaaaggaagaaaatgaactaaataaaattagtgattactgagtaattattgaggatagtaatcaagataaatatctacttactctagagctaaatgggaaagatataagagatagaattagaaatagcctatgaaactgagaatataagacatatgaagtaaattcctttacttcaaaagcattaaataaatttatcaaaaatcctttatcagaagacgcaaaaaaatttcatgggaaatataaggatgaatatagtttttgatatgaaaataaagattgagatttcacttataaaataacaaaagtttcaaaatatgatgaacaaggaaaatggacatgataccaggaaagttttttaagtcatataaaaaagtgtttaattgactctgagatttcaagagaacaaaattgggaagcctttggttggaattttgctggttgtaatacttatgaagagatagaaaaagaagttgattctaaatgatatcagttgactgaaaaccttatttcaatggggaatttaaaaagcttggtaaaagatgaaggttgcttgttatttcctattattaatcaagatatttcaagtcaaaaacaagaaaataaaaacatttttactttagaccttgaaaaggtatttgagtgaaaagaatgtaggattcatccagaattttctatcttttatagaaggccaatagaagaacacaagaaggagaataaatcatgaatcataaatcgttttggaaggttacaattattggcaaatcttggtatagaatttgttccaagaaatccttcttttaaaacaaagaaagaacaaaatagaattgcgattgatcaaaagaagcaaaatcagttagtgcaagagtttaaccaaaaaaaagtaaatacatattttgaaggattagataattattatatttttgggatagatagatgaatcaaacaactagcaactttgtgtgttactgataaagattgagttatacaagattttgatatttatacaaagcactttaatagtgaaagtaaaaaatgggaatacaaatttcatagaaaagattgaatattagatttaactaaccttaagattgagtctgatagaagttgaaataaatacatagtagatatatctttatttcaagctaaagatgaagattgaaatcctacaggtacaaataaacaaaatatccaacttaagcaacttgcatatatcagaaaattacaatatcaaatgtctgcaaatgaagaatgagttttaaattttttgggaaaatataagaacaaagaagaaagagaacaaaatatggaagaactaattactccatataaagaatgaaaaaattttgcagatttaccaatggatatttttcaagaaatgtttgaaaattattatagattaaaaactgatcaaaatttgtctgaatctgagaagaaaaatcttatgaagatcactacagagcttgatgcaagtgaaagcttgaaaaaatgagtagtcgcaaatatgataggagtgatttattatttgatgaaaaaatatgaatataaagtgaagatttctcttgaaaatctttcaaatgcttggcttttttcaaaagacggactttcttgagatgttgtgctaaatacgaaaaatgatgaaacaatggatttaaagaaacaagataatctagcgcttgcttgagtttgaacatatcatttctttgagatgcaattattgaataagttatttaaaatttctaccgaagaatgagttttgcatttagtcccaagtttttgaagtgtaaaaaattatatagagattatgaagataaaaggaaaatatgtatacaaacaatttggcattgtttatttcgttgatccaagaaatacaagtaaaaaatgtcctgtttgttgaaagtgatgaaaaaaatatatctcaagagttgacaatgttgtaacatgtaagaattgttgatttgatacatcatcagacaattccatccttataaataattataaaaaacaatgtaggttttttcttcacaaaaaaactagactttttagtctagtttttcaaatatatcatgataatgttccaaagtgtaactag

SEQ ID NO: 85 - DNA, 3816 bp, synthetic construct
  source 1..3816: mol_type = other DNA
atgttagaaagttttaaagatttatatgaggtgaggaagacagaaagaatctgacttactcagccaaataaaaaatgagaaaaaatatctcacttagagcttaatgaactagtaaaaacatcacttgaaagaataaaaagtgatatagacaaagatgataattctataataaaaaatgaaaaaaatttaattgagaattcaagacaatttgtaaatgaattaaatgttcagttttgaacttggaaaaatatctataaaagatatgacttaatttcagtaaacaaagattattatagaatactagctaggaaagcaaaatttgatgctttttggaaagaaggacgcacaaaatacccaaaagctaatcaaattactttatcatcattacaaaaatgaggaagaaaggaaaagataatagaatattggaaagaaataattgaaagaagttattatttaataagtatttttacacaaaagctagatcaatatgaaaatgcagtctcaaatccagaaaaattatcccacacaaaacctgattttacagattttagaaaaatatttctacaacttctaaaagtaagtgaagactatttaaagcccttatttgataaatcaattaattttaaaacttgaaaaaaattagaatcagaagaagatataaaagaaataaatttatttatatctgaggaaaataacaaaaacatctattcattactaaaattatgaagtgatataagagaatattttgaagaaaattgagcacaagtaccatataaaaaggtaagtttaaattactatacagcagtacaaaagccaaataactttgaatgagaaattaaaaaagctatagaggatttatggatagttgagttcttgaataaaagtgatagtcaaattatcaattatcttcatcaaggatcaaaacagaaaaaaaaattattacttacttcaaaacatccttatcctattgagttattacagttatttaaagtaaaacccattcctttttcagtaaagtataatttggcaaaatttatggagaaaaattataagaatgaaatcaatttatcttatgaagacattctaaataaatttaatttattatgaagagctattgatatcgcaaatgattttaaaaattccaataatcaaaataacttttctcttgatgaataccctgtcaagttagcctttgattatgcttgggaaaatacagcaagaagtttaaaaagaactatacctttcccaaaagaggtatgtaaacaatttttaaaagataattttgatgttgatatcgataatgcagattttaaattatatgccaatcttttattcatagcggataacctagcaactatagaatataataacccaaataatgaagtagaacttataaatgaaataaaacaagcatttgaatgcattagctttccttttgataaagaagtatataaatgacataaagaagctattttggaattattagataaagaaaaatctcaaagagactattccactatattaaaagcaaaacaagaattgtgattattgagatgatgactgaaaaataagattaaaaaatacagagatttaacacagagattaatagacaaaaagaattcacatttttgaatcgcgagttttgtgggaaaaacacttgctacaataagagatggtttaaaggaagaaaatgaactaaataaaattagtgattactgagtaattattgaggatagtaatcaagataaatatctacttactctagaactaaatgggaaagatataagagatagaattagaaatagcctatgaaactgagaatataagacatatgaagtaaattcctttacttcaaaagcattaaataaatttatcaaaaatcctttatcagaagacgcaaaaaaatttcatgggaaatataaggatgaatatagtttttgatatgaaaataaagattgagatttcacttataaaataacaaaagtttcaaaatatgatgaacaaggaaaatggacatgataccaggaaagttttttaagtcatataaaaaagtgtttaattgactctgagatttcaagagaacaaaattgggaagcctttggttggaattttgctggttgtaatacttatgaagagatagaaaaagaaattgattctaaatgatatcagttgactgaaaactctattttaaaagagactttaggaaaattgctgaaagatgaaggttgttcgttatttcctattattaatcaagatatttcaagtcaaaaacaagaaaataaaaacatttttactttagaccttgaaaaggtatttgagtgaaaagaatgtaggattcatccagaattttctatcttttatagaaggccaatagaagaacacaagaaggagaataaatcatgaatcataaatcgttttggaaggttacaattattggcaaatcttggtatagaatttattccaagaaattcttcttttaaaacaaaaaaagaacaaaatagaattgcgattgatcaaaagaaacaaaatcagttagtccaagagtttaaccaggaaaaagtaaatacatattttgaaggattagataattattatatttttgggatagatagatgaatcaaacaattggcaacattatgtgttactgataaagattgagttatacagtcctttgatatttacacaaaattttttgattctaaagaaaaaatttggaaatatacaaaaactagaacggaaggtgttcttgatttgacaaatcttaagatcgaatctgataaaaattgaaatacttatatagttgatatttcgttatttgatgcaaaggatgaagattgaaatcctacaggtacaaataaacaaaatattcaacttaagcaactggcatatatcagaaaattacaatatcaaatgtctgcaaatgaagaatgagttttaacttttttgggaaaatataagaacaaagaggaaagagaacaaaatatgaaaggactaattactccatataaagaatgaaaaaattttgcagatttaccaatggatatttttcaagaaatgtttgaaaattattatagattaaaaactgatcaaaatttgtctgaatctgagaagaaaaatcttatgaagatcactacagagcttgatgcaagtgaaagcttgaaaaaatgagtagtcgcaaatatgataggagtgatttattatttgatgaaaaaatatgaatataaagtgaagatttctcttgaagatctttcaaatgcttggtttttttcaaaagacggactttcttgagatgttgtgctaaatacgaaaaatgatgaaacaatggatttaaagaaacaagataatctagcgcttgcttgagtttgaacatatcatttctttgagatgcaattattgaagaagttatttaaaatttctaccgaagaatgagttttgcatttagtcccaagtttttgaagtgtaaaaaattatatagagattatgaagataaaaggaaaatatgtatacaaacaatttggcattgtttatttcgttgatccaagaaatacaagtagaaaatgtcctgtttgttgaaagtgatgaaaaaaatatatctcaagagttgacaatgttgtaacatgtaagaattgttgatttgatacatcatcagacaattccatccttataaataattatgaaaaacaatgaaaaaaacttcattttataaaaaattgagatgataatggtgcatataatatttgagagaagataagagaacttccaaaaaaatcagatgtaaagaatacctcgatttcatgaaatatttctttttcatag

SEQ ID NO: 86 - DNA, 3807 bp, synthetic construct
  source 1..3807: mol_type = other DNA
atgattaatctagaaagttttaaaaacttgtatgaagtgaggaaaacagtcagattttgattaaatcaacctagaaagaaaagtgatattaataaaacgcattgactattaaaagatttggttgagatttcttttgagagcgaaaaaaaactaattaataatgaaaataatcacgtactaattgatacagagaaagttcttatagagaaattacagcaatatgtaaattgactagaaatacaactaaaaaactggaaatgaacatatcaaagatatgatttaatagctataaataaagactactataaaatattggcaagaaaagcaaaatttgatgctatgtgggagattactaaatttgataaaaaatcaaataaatatataaaagttaaacaaccccaagcaagtcaaatttctttatcatcattaaagatttgaaatagaagtgatagtattattcaatattggtgagagataattgagaaaacagattatttacttaatatttttaaacctaaattagaacaatacgagagggctattaatgatgctaatagtgctcatattaaacctgatagtattgactttagaaaaacctttttacaacttttgaaactaactaaggaatttttacaacctttattggatagatcaattattttcgagctttcaaagaagaaagtttctcaagaaattgaaaagatttctgagtttgcttgagaaaaaaataatacaaaaatttataatgtattgaaaaattgagaagaacttagacaatattttgaagcaaattgatctcaagttccatactgaagagtaagcttaaattaccatacagcagtacaaaaaccaaataattttgaacaagaaattaaaaaagctatagaggatttatgaatagttgagttcttgaataaaagtgaacaggaaattacaagctatttacatcaagattcaaaacaaaaaatacaattattacttacttcaaaacatccttatcctattgagttattacagttatttaaagtaaaacctattcctttttcagtaaagtataatttggcaaagttcattgagaaaaattataagaatgaaatcagtttatcttacgaagagattttagataaattgaatttgttatgaagagctattgatatcgcaaatgattttaaaaattccaataatcaaaataacttttctcttgatgaataccctgtcaagttagcctttgattatgcttgggaaaatacagcaagaagtttaaaaagaactatacctttccccaaagaggtatgtaaacaatttttaaaagataattttgatgttgatgtcgataatgcagattttaaattatatgccaatcttttattcatagcggataatctagcaactattgaatacaataatccaaataatgaagcagaacttataaatgagataaaacaagcatttgactctatagacttttcttttgataaagaaagatattgatgatataaaaatgatgttttagtattgctaaataaggcaaaatctcaaagagactattccactatattaaaagcaaaacaagaattgtgattattgagatgatgactgaaaaataagattaaaaaatacagagatttaacacagagattaatagacaaaaaggattcacatttttgaatcgcgagttttgtaggaaaaacacttgctaaaataagagatcgactcaaggaagaaaatgaactcaataaaatttctcattacggagtaattcttgaagataaaaatcaagataaataccttcttatatcacaacttgatggaaaagatacaagagaaaaaatatctcaaaaacttggcaatggagatataaaggtgtatcaagtaaattcttttacttcaaaagcattaaataaatttatcaaaaattcttgatcggaagatgcaaaaaaatttcactgagattttagatataaacataaagaagtttcaatatatgacgaacaaggaaaatggatatgatatcaggaagaatttttaaatcatataaaaaagtgtttaattgattctgatatttcaagagaacaaaattggaaagcctttggttggaatttttcttgttgtaatacttatgaagagatagaaaaagaaattgattctaaatgatatcagttgactgaaaactctattttaaaagagactttaggaaaattggtgaaagatgaaggttgcttgttagttcctattattaaccaagatatttcaagtcaaaaacaagaaaataaaaatatttttactttagattttgaaaaggtatttgagtgaagagaatatagaattcatccagaattttctatcttttatagaaagcccatggaagaacacaagaaggagaatagatcatgaatcataaatcgttttggaagagtacaattattggcaaatcttggtatagaatttcttccaagaaactcttcttttaaaacaaaaaaagaacaaaatagagttgcgattgatcaaaagaaagaaaacaagttagtgcgagagtttaatcaagaaaaagtaaacacatattttgaaggattagataattattatatttttgggatagatagatgaatcaaacaattggcaactttatgtgttactgataaaggttgagttattcaggattttgatatttatacaaagcactttaatagtggaagtaaaagatgggaatacaaatttcatagaaaagattgaatattagatttaactaaccttaagattgagtctgatagaagttgaaataaatatatagtagatatatctttatttcaggctaaagatgaagattgaaatcctacaggtacaaataaacaaaatattcaacttaagcaacttgcatatatcagaaaattacaatatcaaatgtctgcaaatgaagaatgagttttaacttttttgggaaaatataagaacaaagaggaaagggaacaaaatatgaaagaactaattactccatataaagaatgaaaaaattttgcagatttgccaatggatatttttcaagaaatgtttgaaaattattatagattaaaaactgatcaaaatttgtctgaatctgagaagaaaaatcttatgaagattactacagagcttgatgcaagtgaaagtttgaaaaaatgagtagtcgcaaatatgataggagtgatttattgtttgatgaaaaaatatgaatataaagtgaagatttctcttgaagatctttcaaatgcttggtttttttcaaaagactgactttcttgaggtgttgtgctaaatacgaaaaatgatgaaacaatggatttaaaaaaacaagataatttagcacttgcttgagtttgaacctaccatttctttgagatgcaattgttgaagaagttatttaaaatttctaccgaagaatgaattttgcatttagtcccaagttttggaagtgtaaaaaattacacagagatttttaaggataaggggaaatatgtatataaacagtttggagttatttattttgttgatccaagaaatacaagtagaaaatgtcctgtttgttgaaagtgatgaaaaaaatatatctcaagaattgataatgttgtaacatgtaagaattgttgatttgatacatcatcagacaattctattcttataaaaaataataaaaaacagtgaaaaaaacttcattttataaaaaatggagatgataatggtgcatataatatttgagagaagataagagaacttccaaaaaaatcagatgtaaagaatacctcgatttcatgaaatatttctttttcatag

SEQ ID NO: 87 - DNA, 3408 bp, synthetic construct
  source 1..3408: mol_type = other DNA
atgctttcaaaaaaaaacaaaaaatgaattaaacaacctcaagcaaatcaaatctctttatcatctttacatgataaaaaggatttaataataaattattgggagaataatatttcggaaagcgattttttaataaacatctttgaaccaaatttaaaaaaatatttagatgcacttgctaatcctgataaatattcacatacaaagcctgatttgatagactttagaaaaacatttctacaattattaaacgtaaacaaagaatatttgcaaccattatttgataaatctatcatatttgaaactggaaaaaagggagagtctaaatatataaatgagattaatgagtttgcttgaaatgaaaacaatttaaaaattaaagaattactagatttatgagaaaaaattagacaatattttgaatctaactgactacaagttccttattgaagagttagtttgaactattatacagcggtgcagaagccaaacaactttgatgatgaaataaaaaaatgaataaaagacctatgaattatagaatttttaagaaaagaggaaagtgaaatacaagtttatttaaatcaagaatcaaaagaaaaaataaaattattaaaagattccaaagaaccttattctattgagttaatacaactttttaaaccgaaagccattcctttttcagtaaaatacaatttagcaaaatatatagaaaaaaattacaaaaaggagcttggtttaaagtatgaagatattttgtataagtttgacttactttgaagatcttatgatatttgaaaagattattctgaatgcacagaaaagaacacattttcacttatgaaatatccaattaagttagcttttgattattcttgggaaaatttagcaagaagcttaaaaagggacatagattttccaaaagaaatttgcaaaaactttttaaaagataattttgatgtagatataaacaattctagttttaaattgtatgcggatttattatttattgctgaaaatttagcaaccatagaatacaataatccaagtaatgaaaaagagattatcgataacatcataaacacctttcaaatcataaattttgaaataaatggatataaaaagaaagactatgaaaatcataaaaccgcaattttgtctttattggaacaaggaaagaatgaaagagactttaagagcaatgtttttatcgaagcaaaacagaaactatgactcttgagatgatgacaaaaaaatctaattgcaaaatataaagaactaactgaaaaactagttgataaaaaggatattcacatctgaatagcaagttttatatgaagaaatttggcgaatataagagatggattaaaagaggaacatgaactaaataaaataactgactattgaataattgttgaggacaacaataatgacaaataccttttacttttaagacttgcttgaaaagataatagagagcaacttaaaagtaaattatgagatggcgagataaaagtgtatgagattaattcttttacttccaaggcattaaataaattcatcaaaaacccttgatgagaagatgcaaaaaaatttcattgaaattttaattataaatatagagatatttcaacttattctcatgaatgaaaatggattggttataaaccggattttattaaacatattaaagattcgctttgtaattctgaaatagcaaaagaacagaaacgggttgagtttggatgggattttgataagtgtaatacctatgaggaaattataaaagaaatagattcaaaatgatataatttaataaacaacactatctcaaaaaagaatctcgaatatttaattgaaaatgataattgtctattactccctcttataaatcaagatatttctagtaaaaaaacaataaataaaaacgcttttaccattgattttgaaagagccatagattgaaagtcatgatatagaattcaccctgaatttagtgtcttttatagaacacctatgcaagaatattcaaaagaaaataagttttgaattgttaatcgttttggaagattggaattgttggcaaatattggaatagaatatgtgccttcaaacaaaaattatactactaaaaaagaacagaataaaatctcattagataaagataaacaaaataatttaataaaagaattcaacgaagaaaaggtagataactattttgatagtttggataatttctatgtttttggaatagatagatgaatcaaacagttagctacactctgtgtaatggataaagcttgagttattcaggatttcgatatttatacaaaacatttcaataatactcagaaaaaatgggaatataaaaaaaatagaactgaatgaattttgaacttaacaaatttaaaagttgaatcagatcaaaactgaaccccatttctagtagatatttcattatttgaatcaaaaaatgaaaattgaaaaagttttggaactaataaacaaaatgagaaattaaaacagttggcttatgtaagaaaactacagtatcaaatgtcatttaataatgaatcagtattaaattttataaaaaaacatacgactcaagaggcaagggctgagaatatatgagaactgataactccatataaagaatgaaaacaatttgccgatttacctatagatacttttgaagaaatgtttgaaaattttcatcagctaaagacaaatgaaaacttatctgagttagaaaaacaaaatttaatgaagcttactactgaacttgatgcaagtgaagaattaaaaagaggagttgttgccaatatgattggggttatttgttatttaatgaaaaaatatcattataaagtaaagattgttcttgaaaacttatctaatgcttggtatttttctgaagattgattaacatgagatgctttgctaaattcaaaaaatgattgaataactgattttaaaaaacaagaaaatcttgcgcttgcttgagtatgaacttatcatttttttgaaacacaacttttgaaaaaaatatttaaaatatctattgaagaaaaaatccttcatttagttcccagtttttgaagtgtaaaaaattatacctatctaataaaaaataataaatcacaatatgtatttcgacaatttggtattgtttattttgtaagtccaaagaacactagcttcaaatgtcctatttgtctaaaatcaggaagagaatacataacaaggaacgataatataataacttgtaagagttgtggttttgtttcatgaaaagacaatgatgaaatattaaaaaaatataccaaaactcatctcaacatacattttatcaaaaattgagatgataatggggcgtatcatatatgaaaaaaactgctgaaatag

SEQ ID NO: 88 - DNA, 3885 bp, synthetic construct
  source 1..3885: mol_type = other DNA
atggaagcaataaaaaacaactatcaattaagcaaaaccctgcgttttggattaacacaaaaaagtaaaacaagaaaagatggttttacaggagaaatctaccaaagtcataacgaattaaaagacttggtaaaaagttctgaagaccgaattaaaaaatccgtatcaacagacgaaaaatccgagatgagtttgtctgttgataaaattagatgttgtcttgtgatgatttcggattttcttagtagctggcaacaagtttattctcgtgccgaccagattgccttagataaagactattacaaaatcctttgcaaaaaaattggctttgacggtttctgggttgatgagcgatatgatagaaaaaacgacaaaacagttagaacaaaaaagccacaatcccgaaccataaatctttccgaattagacaagaaagatgacaaaggcatcgaacgcagacaatatctcctcacttattggcgggataatctgataaatgcagcagataaatttgaagtagttactgaaaagttgaaacagtttgaagatgctttgaatattaaccgaacctataataaaccgaatgaagtagaattgcgaaaactattcttgtcattgacgaatattgttcaagaaacactgcaaccgctttgtctgggacaaatttgttttcctaaattggaaaaaatagatgattcaagaactgaaaataaacacttgattgattttgcaaccgattatcaatccaaaagtgatttgctttctgaaatttcagaattaaaaaaatactttgaagaaaatggtggtaacgtgccttattgccgagcaactcttaatcaaaaaacagctgtaaagaatcccaattctaccgacaatagcatagactctgaaattaaaaagctcggtcttgacaaaatattgaaagagaataaagatgctttgtattttgccaataaaatttacagtttgtctgccaaagaaaaactctcaaaattagatgataaaactaccggattgatagaacgtagcttactgtttaaatataagcctgttcctgctattgtacaatacgaaatagctaaaactttaagcgaaaccatcaataaaagcgaagaagatttattggaatttcttcgcagtattggacaaacgaaaagtcctacaaaggattatgccgatttacaggataaaaacgatttcgatttagatgcttatccgctaaaagtagcatttgatttcgcttgggagaacttggcaagaagtatttatcatagtgatgcggatattccggttgctgtttgtgaagggtttcttaaaaaaaactttggcattgataaaagcaatgcagatttcaagttatatgcacaattacaggaattaaaggctgtgttggcaacattggaatacggaaatccaacaaataggcaaacgtttataaatgaagcaacaaaattgttatccccaatatcttgggataaaatcggtcgaaacggaaatcaaaataaatattctattgaaaaatggctgaaaactctaacaaaagatgataaagattataaagatgccaaacagcaaatagccttgtttagaggacgattgaagaataatattaagacttttgatgacattacgaaatattttaaatccgttgctatggaaatgggcagaacctttgcccaaatgcgtgataaaataaccggtgcggcagaacttaacaaagttacccattatgccataattatcgaagaccaaaattttgataaatatgttttattacaggagtttgtcgataaaaaggagaatagaatatatgcaaaaacagacagacatcacagcgattttacgacttattcggttaattcggttacttcaggctctattgccaaaatgctcaggaaaaagagaatggatgaattgaatagaaataacagaaatagttttgaacagaaacctgaattatctgaagaacaaaaagaacagcgtaacattagagaatggaaagagtttatagaagataaacgctgggacttggaattccaattaaatctcagcaataaaacgtttgagcaaattaaaaaagaagttgatgccaaatgctatgaattggatataaactatatcagccaagaaactttatccgatttggtaaataaaaagggttgtttgctattgccgattgttagtcaggacatagcaaaagaaaataaaacagaaggcaatcaatttaccaaagattggaacgctattttcactcaagaaactccttggagactaactccggaatttagagtttcttatagaaaaccaacgccaaattatccggtttccgacaaaggagacaaacgctactcccgcttccaaatgatagctcatttcttatgcgattatcttccaaaatccgatagctatatttctaattgggaacaaattgcaaattataaagatgataaattgcaggaaaaagcggtaaaagaatttaatgcagatttgagaggacgaacggaagaagaaaaacaaagtgaatcggtgaatgcgttgcttgcccattttggaaaccagaacaaaaaacaaaaacctgttgagcgacctaaagaaaaattttatgtctttggcattgaccgcggacaaaaagaattggctacgctgtgtgtaatagaccaagacaagaaaattgtcggtgattttgatatttatacccgttcatttaactccgaagcaaaacaatgggaacataaattacttgagaaacgtgttattctcgatttatctaatttgcgtgtggaaacaactattgtaatagacggaaagccggaaaagaaaaaggttttggtggatttgagcgaagtaaaggtaaaagacaaagatggtaagtattcaaagccgaataaaatgcaggtaaaaatgcagcagttggcgtatattcgtaaactgcaattccaaatgcagaccaatccggatgctgtattagaatggtatgcaaacaacaagaccaaagaacaaatcatgtctaactttgtagataatgaaaacggtgataaagggttggtttctttctatggaactgccgttgaggaactgaatgaaaccttgccgattgataaaatcgaagaaatactcaaaaaatttcaagagctgaaagataaagaaaaacaaggtgaaaccgttaaattggaaattgataaacttgttcagctagagccggtagataatttaaaaaacggagtggttgccaatatggttggcgttattgcttatttacttcaaaatcttgattatcaggtctatatttcactcgaagatttatcaaaaccattcagtggtcaaattataggaggaatcgccggtgtgccaacaaaaacaaataaagaggaaggtcggcgtgccgatgtggaaaaatatgccggattaggtctttacaatttttttgaaatgcaactgctcaaaaaactattccgcattcagcaagacagccaaaatattttgcatttagtgccgcctttcagagcaatgaaaaattatgaccatgttgccgttggcaaaggcaaagtaaaaaatcagtttggtatagtcttttttgtggatgctgatgccacttctaaaacctgtccatgctgcggttcatcaaataataagccaaatctgaagatgtatccaaacgctaaaaagggactatcaaaagaagggaaggaagtttgggtagagcgtgataaatcggaaggtaatgacattatcagatgcttcgtttgcgggtttgataccacaaaagattattccgaaaatccgcttagatacattaaaagcggagatgataatgccgcctatctgatttctgcagaaggaattaaggcttatgaattagcaacaacattagtgaacaacaaataa

SEQ ID NO: 89 - DNA, 3861 bp, synthetic construct
  source 1..3861: mol_type = other DNA
atggaaaatatcaaaaacaattatcaattatccaaaacacttcgttttggcttaacacaaaaacaaaatggaaatagctcaaatacagataatgtttatcatagccatagtgctttaaaagaattagtggacatttccgaaaacagaattaaaaaaaatgtttctacagagggagcaaccgaaatgcaattatccattgaaagcattagaaaatgtatgattatgattgagcagtttattaaagactggaaaagagtatattatagatcagaccaaatttctttagataaagatttttacaaaaagctaagtaagaaaattgggtttgaagcattttggtttgaaagaaataaaaaaactcaacaaaggataaaaaaacctcaatcttgcataattgctttgtctgaactctcaaaaagagataattttggaaaagaacgtcaagaatatattgttgaatattgggaaaataacttgctaaaatctacagaaagatacgaagaagtaagtgaaaaattagaacagtttgaattagctcttaaaatcaatcgaacagacaatcgcccgaatgaggtagaattgcgaaaaatgtttctatcgcttgtaaatatggttcgagaagtagttgagccactctgtttgggacaaatttcttttcctaaattagagaaattagcagacaattctaaaaataaacaactacgaaaatttgcaacagattatcaatcaaaaagtgatttattgacacaaatttctgaattgaaaaaatattttgaagagaacggtggcaatgtgccgtattgcagagctacgctcaatccgcttactgctgtaaaaaatcctaaatctactgatagtagtattcttgatgaaatcaagaaattaaaattagatgttatcttaagagactaccaaagtgttgctctttttgataattccattcgagatttgacagcatcacagaaaatgcaattgcttaaccaaaataacgaaggccttataaagcgtggtttactattcaagtacaaacccattcctgctattgtgcaatatgaaattgcaaaagtattgagtgctgaactcaacaaagacgaacaagaattacgaaattttttaagagatattggtcaggttaaaagccctgccaaagactacgcagaattgcaagataaaaaagattttgacatcaatcattatcctttaaaagttgcttttgactttgcgtgggaatcgcttgctaaatctgtttatcatcccgatatagatttcccagaggaacaatgcaaaacattactaagggaagttttccaagtagatgaaaacaacgaaaattttaaattttatgcccaacttttagagctacggtcgttacttgccactttagaacacggaaaaccaacagaggtaataacaattgaaaacgaagtaaagaaaattctcgaaaatattgactggagtaaatttggagatagaggcaaaaactacaaatcggctattgaaaattggatacacaatagaaacaaaaaagattttaaaggcgactatttcaaaaaagccaagcaacagataggtttaacacgtggtagacaaaaaaatttaataaaaaagtatgacgaaataacaaagtcttacaaagacattgcaatgaaaatgggcaaaacttttgccgaaatgcgggataaaattaccggtgcagccgaactcaataaagtatcgcattacgcaatgattgtggaagataccaatcaagacaagtatgttttattgcaagaatttgtagaaaataataaggatagaatttatgcaaaaagtactcctcaaaatgaagattttaaagcatattcggtcaattctgttacttcttcggctatcgcaaaaatgattagaaaaataagaattgacaagctacaagcaaatgaacgaaacaataatagacaacaagcaccagaactatcagaaactcaaaaagaggcaagaaatatcaaagaatggaaagattttattgcggaaaaacgatggaattatgagttcgatttaaaattggacaataaaaattttgagcaaataaaaaaagaaatagactcgaaatgctttaaattagaaaccaaatatatgagcgaggaagtacttgttgatttggtcaaaaatcaaaattgcctgcttttaccaattatcaatcaagatttagctaaaaaaataaaatccgagagtaaccagtttaccaaagattggaatgccatttttgcacaaaacacaccttggcgacttacgccagaatttagagtgtcttacagaaaacctactccaaattatcccaaatccgaaagaggtgataaaagatattctcgttttcaaatgataggtcatttcttatgcgattttatccctaaaactgctgattatatttcaaatagagagatgattgctaattttaaagatgatgaaaaacaaaaacaaacgattataaattttcatgaaagattaaaccctaaatctgaaaatgaaaaaatgaacatgttgttagctaaatttggaaataaaaattcaaatcaatctaaagaaacaaaaaaagaagaaaaattttatgtgtttggaattgaccgcgggcaaaaagaacttgcaacactttgcgtaatagaccaagataaaaaaattattggagattttaatatttacacccgctccttcaacacccaaaacaagcaatgggagcatcaacttttagacaaaagacatattttagacttatctaatctgcgagtcgaaacaactattgtaattgatggtaagcctgatgtgcgaaaagtattagttgatttgagcgaagtgaaagtgaaagacaaaaatgggaattacacgaaaacggataaaatgcaagtgaaaatgcaacagttagcatacatacgcaaacttcaatttcagatgcaaacgaatcctgatactgtgttggaatggtacaataaaaatcaaacaaaggaggcgattttaaataactttgttgataaaccaaatggcgaaaaaggcttagtctctttttatgggtctgctgttgaggaattaaaagatactttgccgattgaaagaattgaaaagatgctgcaacaatttaagtctctaaaaaatgaagaaaaagaagggaaagatgtaaaggctgaaattgacaaattgatacaacttgaacctgtagataacttgaaagcaggtgtagtggctaatatggttggtgtgattgctcatttattaaaagaatttaattatcaagtatatatatcattagaagatttatctaatccttttggtagccatgttatagatggaaccaccggaactcattcaaaaacgaataaaggtgaaggtaaaagagccgatgtagaaaaatatgcagggctgggtttgtataacttttttgaaatgcaattactcaaaaaactgttccgaatacagcaagatagccaaaacattttacatttagtacccgcatttagagccgtaaaaaattatgaaaacatcattgcgggaaaagataaaattaaaaaccaatttggaatagtattttttgtagatgccaattctacttcaaaaatgtgtcctgtttgtaattctaccaatgaaactaatagagagtacccaaatgcaaaaaaaggaacttctaaagatgataaagaagtttgggtagaacgagataaatcaaacggagacgacataattcgctgttttgtgtgtgggtttgacacaactaaaaaatatgaagaaaatccactaaaattcattaaaagtggtgatgataatgcagcgtatataatttctgcttttggcataaaggcttatgaattagctaaatcagtaattgataacaagtaa

SEQ ID NO: 90 - DNA, 3747 bp, synthetic construct
  source 1..3747: mol_type = other DNA
atgaaaaatttaacacaatttgtaaatttgtaccaactctcaaaaacattgaaattcggattaacattacgaaataaaataagaaaaaatggttttgaaggagaaatttatgaaagtcatactgaattacaggaacttataaaaatttcagagcaaaaaatcattaaggaaacaactgataaaaataaagaaataatgacatttacagaattgcccttagatgaaattcgtaaatgtcttgacgacatgcataaatatctcgatgattgggaacaattttataacagatatgaccaaatagcagtacttaaagattattatcgaaaattggaacgtaaagcaagatttgacggtttttggagagaaaaaaatattaagaacaaaatacaaaataatgaatctgaaactatcaaaaaaccgcagtctcaagttatcaaattgtcaagtttgaacaatgaatatgaaaataagaaacgtcgagattacataaccgactactggaatgaaaatattcaaaaagcaaaacgaaaattttatgaagttaattctgtattaaaacaatttgaagtagctaatgaacaaaatagagacgataaaaaattgaacgaagtagtgttacgtaaattatttttatcttttacaaatcttattaacgatacacttgaacctctttgtaatggctctatttgttttcctgatattgaaaaattaacaaacagcaaaactgacgaacaattacagagattcgtttttgatgacggattcaaaaaaatattatcagaacaaatagaaaatttgaaaatttactttgcaataaacggcggatatgtccaatatggtagagtaacattaaataaatataccgcgttacaacaacccaataaggtagatgaagatataaagaatataattaaagaactgggtttgttggaatttgtaaaaaagtatgaaaatactgaacaaatcattaactatataaaaaatattaaagataaaaaacaagaactaaacgctaataatttgtcattgatagaaaaagtacaattatttaaatacaaaactattccagctggagtacaaccttcgcttatagcatatctggcacgaacagaaaaaaaagataaaaaaacactcagagaactattttacgcaatcggtcagccacaaagtccgtcaaaagattataaagaattacaaaataaaacggattttaatttgtacaaatatcctttgaaggttgcattcgattatgcgtgggagtcattggcaaaaagtaaatataacccacatatagattttccagatgtcaaatgtaaagaatttttaaaagatatttttggtacggatatatctgttaatgataatttcaagttatattccgcacttttgtttgttcgtgaaaatcttgcaacattagatcatggtaatccaaacgataaaaatattcatgttaacaaagtagaaaatacatttaaagagatcaaagatagattggcaaaaaaagaatataaaaaagaatataaagccttagaaattatttgtaaatggcataaaaattcagcaaccattgaacaatcagaatatgaggcagcgaaacaaaccatatatgaggcagcgaaacaaaccattggacaattaagagggcggcaaaaaaaccaaatatctaaatttaaagaattgacggattcattcaaaaagttggctccaaaatttggtaaagcatttgctaatcttagggataaatttaacgaagaatatgaaataaataaaatttctcattgcggagttattgtagaagatcgcaataacgatcaatatttgttattgtctcaattaaatgataatagagaaaatgcatctgatatttttgagttagaagctgatcccaacggtgagttgaaaatttatcaggtaaagtcattgacctctaaaacgttgttgaaatttctcaaaaacaaaaaaggttccaatactggatttcatatcaatgaaaattggacgtttccaaaagggaaatgggatgttattaataaggataaaatttttcttaattatgtaatacaacgtattacaaattcgagtatggcgaaagagcaaaaatggagtaactttaaatgggattttaggcgatgtgattcatacgaagcgatagccaaagaagtagatgccaaaggatatattttagaatctgtcaatatttctaaattgacactgaacaaattgataacagaaaagaaatgtctgttactacctattgttaatcaagacttaacaagacaagataaaaaaacaaaaaatcaatttacgaaagattggataaagatttttgagagtaataattgttaccgattacatcctgaatttaaaatatcttatcgatatccaactcctaattatcctaaaccggaagaaaagcgttattcccgttttcagatgatttcgcatttactttgcgaatatattccacaaaacgataattataaatcacgtaaagaacaagttaaaatctttaatgataaagttgctcaaaaagaatctgtagaacaatttaaccaacaatttgaaataacagatgattattatatttttggaattgatcgcggcataaaacaattagcaacactttgtatattaaataaaaatggacaaatacaaggagactttgaaatatatactcgtgaatttgataaagtcaataaacaatggaaacacactattcttgaaaaacgaaatattttagatttgtctgatttacgggttgagacaacagttgagggtaaaaaagtattggtcgatctgagtaaagtgaagttacacagtggaaatgaaaataagcaaactataaaacttaaacggttggcatatattcgtatgttacaatatcaaatgcagcatgaacaagataaagtattaagatttataaatcaatacaaaacaattgatgagatagaaaaaaatattagagatttaatttcaccttttaaggaaggaaaacaatatgccgacttacctacagaaaaaataaaagatatgcttatacaatttggtgaattatcaaagaatgatagcgataaatctaaaaaagaattgtgtacactttgtgaattagatgccgtagatgattttaaaaccggtgctgttgctaatatgataggtgtaattgcttatctactagaaaaatataaatataacgtttatatttcgttagaagatttgactcgtgcatttagactacaaagggatagattaacaaataatattttacaaagtaccaataaagacaatactgtagatttcaaagatcaagaaaatttagtattagcaggattgggaacttatcactattttgaaatacaattactaagaaaattgtttcgtatccagcgaaatagtgaaggagacattttacatttagttccgacatttcgtagcgtagataattacgaaaaaattgttcgcagagataaaaaaacagataatgataaatatgtgaactatccctttggaattgtgcggtttgttgatccgaaatatacttctaaaaaatgtcctatttgtgataaaacgaacaccacaaggaaagataatgttctgatttgtaattcttgcaatgcagtatctggagaatatgaaacagataatgaaaatagacattatattaccaatggcgatgataatggtgcataccatatagctttaaaagcattaagtttaaggaagtcaaaaaatcttgaaaagaaaaagtaa

SEQ ID NO: 91 - DNA, 4182 bp, synthetic construct
  source 1..4182: mol_type = other DNA
atgactaagtatcaattaacaaagaccctacgatttggactcaccaaggtgagaaaaaaaactaaacttgtggcagggaaagaggtggatgcaaaatacctcagccatgaggagttggatgacttggtgatgaggtcggagtataatctcatcaaaaggaatgtgcttgaatggtctaaaaaaaaggaaagcgacaaatctaattatatctttgatgaacttgacaagagatttgaagaaataatcgaaatcgaagacagagatcaacgcaatgccgaatacgttgaatttttgaatgagttcttcaaagaggtaaaccatcagactataaaccaattggatgaatcgacttttataaataaaattggtgattgtagtaaagctataaaggagtatttgttaagttggggaaaagtatgcaggcgcattgataaaatcacagttagaaaggattacttcaagatacttgctcgtaagacttttttcaaatacgaatcaaaagttgggaaaaagaggacgcctctgccttccgaagtcaaactatcgggacaaaaaggcaataattattttgatgaaccgataaacgaaggcatttctcaattttggcaaaatagagttgcaaaagcattaaacctacattcgcaacttgagtcaatgctttttgattacaaaaaagcaatagaaacagaaaaacacaatcaggaaaacccgaaaggagataatggttcatttgacaagctgcaccttgtagattttagaaaaatgtttctatcagtatgcagtcttgttatggatagtttgcgcccaattgttaatgaacttatcatagtgtcggataatgtttcgaaagacgaggataagtatattttggactttgtcaatgacaaaaaaacgcaatgggatttgttccaacaaatagagaatttacagactatatgcaaagataacggtgaaaatatcttctttgggaaagcaacgttcaataaatacacttctgaacaagccccaaatcatcgaaataatgatattgccaaggttctccgagaactgaaaattgaaaaatttgtgtctgattatattgacttggatcaagaagcaataaatcgaaaaatttatcaatcaacccaatcccgtttggagaacttgaataatccacagatttctcctattatccgcgcacagtatttcaaatacaaaccaattccaacattagtaagattcggacttgcgaaagaattggccaaacagcaaggaaaaaaatattccgatcgattgaaaggcatccaagaattgtttagaatttttggatcttcaaaaagtccggcattggattacaaaaacaatagaactgatttctcattggacaattatccaatcaaagttgcatttgattatgcatgggaaatgtgtgcccgttctgaatacgctcaaaaaccagtcgattttccaaaatcaatttgtgaaaaattcttggaaaaattttttgaatgcaaaagtaacgaaaaatatcaacaatcatttgtgacttatgcccgtttattgaaaatcaatgaagacttggcaactttggaacattttgaaaatgagccacccaaagatatcgaatcaatttaccaagatgcgcaaagatatctcgatgaagtggggaatttatgctcaaatgaggatagagcagcaatcgcaaaatggtacgaagaatacaacaagttatggacaaaaggcgaccataaaaagttaaaagaatggattgtgtcaaaatcatctatcattactaattttacgcaggcgaagatgcatttgggacaaaaaagaggaagccaaaaaacttttgttcttaaatcatatttccattcttcttatggaaaaattagagataacaatcgatttgtaaactctaatgttaccgaagtgtttaaaacaatagccagcactttcggcaaatcgtttgccacaatcagagagtattttaacgaagaaagtgaagtaaacaaaatagaatacggcgctgtgattatcaaagataaaaacggtgacaaatatcttttgcttcaaaagaaaaatgaaggtggtattgatatgcctatattcaataaatctgatgaaaatggtgattgtgacctttatcaggttaaatctctgacatccaaaacagttaggaaaattattgcatcacccaacaaatataacgatttttttgttaacaatgatggcaaaaaaatcatttatccagacaaaacagatttcaaatacaagattaatccatatgacaaagaagaagtcaaaaaacgcaaaagagaattgtacaataatgatttagtgcgaccaattatatatagtttgactcaatctaaatttgccaacaaacaaaattttgagaaatattttgattggactaaggctttaaaacaatgctcaaacattgagcaattgtacaaaacaatcgaccagaaaggctattccttgaacccttccaaaatcagcaaagagcaaatcgcggatttggttaacaatatgaattgttatctcttgccgattgtaaatcaaaatattacagcaaagacaaagaacgacacaaatcaattcaccaaagattggaataagattttcaatgaggtagataaggattatcgtattcatcctgaatttacgatgttctatcgttatcctacgccggattatcctaaatttggagaaaagaggtattcccgtttccaaatgaatgtaaactttctaatggaagttattccggctgatggcgaatactgttcacgaaaggagcaaatagaaatatacaatgctcccaaagacaacgaaaattgtcaaaaaaatgtagttgagagattcaacaataaaattaaagccctaaagccatcgtatttcataggcattgaccgtggcattaacgaattggcgacattgtgcgtaatagataaagaaggtaaaatcgttggcgattttgagatatacaaaagagaatttgactcaaatctgaaacgtcagaaatatacatcaatagagactcgcgacattttagacctgtcgtacttgcgtgttgaaaaggacgaaaatggtgaatctcgtttagtagatttgtcagaatcggaggtatggatagacagccttgatcacgacaaaggaaaaagagccaacaaacaaatagttcacctcaaacatctgtactatttgcgttgcatcgcacatttgttgcagtcgtcagattacaaatctattgtattagaaaaactcaaagattgcaacaatctgaaagatgaggaaataaaaaaggtattcgagaaagataaatttgttgattcctacaaaggtggcgaagcatatacagatttgccatacgatgaaatcagaaagttaatttctgattatcaggaaattgagcaatcaaaccaaacagaatcggagaagtccaaagcattaaatacactttgccagttggacgcatctgaatatctcaaaaagggtgttgttgccaatatgataggtgttgtcgtatatgttttgaaaaagtataattacgatgcgtatatctctctcgaaaatctttgctatgcatacggatatagcaaagacacattgtcaggatattcaattacaagtactaaggaagatccatatttagatttcaaagatcaagaaaacgcgaaattagcaggattaggaacttatagtttctttgaggttcaacttctgaaaaaacttttcaaacttcaaattgaagaaaatacagagttgattccagctttccgcagcgtggacaactacgagaaaatatttttgttaaaaaatatagataacaaaatttatcagttcggtattgtgtattttgttgatccaaaatatacaagcctatgttgccctatttgtggtgaacatggcaaaaaaaatgttgataggaagaaacatacaaaaaaatatgacgaagatgaattagtatgcaagcaatgcggttttcatacaaatctttcccatatcgaaacaagggttatggaagacaaaacaataaaaaatagttacgatgagtgtaatttgaaagccattgtttctggagatgcaaatgccgcttataacattgcaattaggttgggcaaaaacatatactcaacaattgcagacaaagtaaaggatttgcatcacgaagggaaaaaatacattatcgtaaaaggataa

SEQ ID NO: 92 - DNA, 4113 bp, synthetic construct
  source 1..4113: mol_type = other DNA
gtggcagggaaagaggtggatgcaaaatacctcagccatgaggagttggatgacttggtgatgaggtcggagtataatctcatcaaaaggaatgtgcttgaatggtctaaaaaaaaggaaagcgacaaatctaattatatctttgatgaacttgacaagagatttgaagaaataatcgaaatcgaagacagagatcaacgcaatgccgaatacgttgaatttttgaatgagttcttcaaagaggtaaaccatcagactataaaccaattggatgaatcgacttttataaataaaattggtgattgtagtaaagctataaaggagtatttgttaagttggggaaaagtatgcaggcgcattgataaaatcacagttagaaaggattacttcaagatacttgctcgtaagacttttttcaaatacgaatcaaaagttgggaaaaagaggacgcctctgccttccgaagtcaaactatcgggacaaaaaggcaataattattttgatgaaccgataaacgaaggcatttctcaattttggcaaaatagagttgcaaaagcattaaacctacattcgcaacttgagtcaatgctttttgattacaaaaaagcaatagaaacagaaaaacacaatcaggaaaacccgaaaggagataatggttcatttgacaagctgcaccttgtagattttagaaaaatgtttctatcagtatgcagtcttgttatggatagtttgcgcccaattgttaatgaacttatcatagtgtcggataatgtttcgaaagacgaggataagtatattttggactttgtcaatgacaaaaaaacgcaatgggatttgttccaacaaatagagaatttacagactatatgcaaagataacggtgaaaatatcttctttgggaaagcaacgttcaataaatacacttctgaacaagccccaaatcatcgaaataatgatattgccaaggttctccgagaactgaaaattgaaaaatttgtgtctgattatattgacttggatcaagaagcaataaatcgaaaaatttatcaatcaacccaatcccgtttggagaacttgaataatccacagatttctcctattatccgcgcacagtatttcaaatacaaaccaattccaacattagtaagattcggacttgcgaaagaattggccaaacagcaaggaaaaaaatattccgatcgattgaaaggcatccaagaattgtttagaatttttggatcttcaaaaagtccggcattggattacaaaaacaatagaactgatttctcattggacaattatccaatcaaagttgcatttgattatgcatgggaaatgtgtgcccgttctgaatacgctcaaaaaccagtcgattttccaaaatcaatttgtgaaaaattcttggaaaaattttttgaatgcaaaagtaacgaaaaatatcaacaatcatttgtgacttatgcccgtttattgaaaatcaatgaagacttggcaactttggaacattttgaaaatgagccacccaaagatatcgaatcaatttaccaagatgcgcaaagatatctcgatgaagtggggaatttatgctcaaatgaggatagagcagcaatcgcaaaatggtacgaagaatacaacaagttatggacaaaaggcgaccataaaaagttaaaagaatggattgtgtcaaaatcatctatcattactaattttacgcaggcgaagatgcatttgggacaaaaaagaggaagccaaaaaacttttgttcttaaatcatatttccattcttcttatggaaaaattagagataacaatcgatttgtaaactctaatgttaccgaagtgtttaaaacaatagccagcactttcggcaaatcgtttgccacaatcagagagtattttaacgaagaaagtgaagtaaacaaaatagaatacggcgctgtgattatcaaagataaaaacggtgacaaatatcttttgcttcaaaagaaaaatgaaggtggtattgatatgcctatattcaataaatctgatgaaaatggtgattgtgacctttatcaggttaaatctctgacatccaaaacagttaggaaaattattgcatcacccaacaaatataacgatttttttgttaacaatgatggcaaaaaaatcatttatccagacaaaacagatttcaaatacaagattaatccatatgacaaagaagaagtcaaaaaacgcaaaagagaattgtacaataatgatttagtgcgaccaattatatatagtttgactcaatctaaatttgccaacaaacaaaattttgagaaatattttgattggactaaggctttaaaacaatgctcaaacattgagcaattgtacaaaacaatcgaccagaaaggctattccttgaacccttccaaaatcagcaaagagcaaatcgcggatttggttaacaatatgaattgttatctcttgccgattgtaaatcaaaatattacagcaaagacaaagaacgacacaaatcaattcaccaaagattggaataagattttcaatgaggtagataaggattatcgtattcatcctgaatttacgatgttctatcgttatcctacgccggattatcctaaatttggagaaaagaggtattcccgtttccaaatgaatgtaaactttctaatggaagttattccggctgatggcgaatactgttcacgaaaggagcaaatagaaatatacaatgctcccaaagacaacgaaaattgtcaaaaaaatgtagttgagagattcaacaataaaattaaagccctaaagccatcgtatttcataggcattgaccgtggcattaacgaattggcgacattgtgcgtaatagataaagaaggtaaaatcgttggcgattttgagatatacaaaagagaatttgactcaaatctgaaacgtcagaaatatacatcaatagagactcgcgacattttagacctgtcgtacttgcgtgttgaaaaggacgaaaatggtgaatctcgtttagtagatttgtcagaatcggaggtatggatagacagccttgatcacgacaaaggaaaaagagccaacaaacaaatagttcacctcaaacatctgtactatttgcgttgcatcgcacatttgttgcagtcgtcagattacaaatctattgtattagaaaaactcaaagattgcaacaatctgaaagatgaggaaataaaaaaggtattcgagaaagataaatttgttgattcctacaaaggtggcgaagcatatacagatttgccatacgatgaaatcagaaagttaatttctgattatcaggaaattgagcaatcaaaccaaacagaatcggagaagtccaaagcattaaatacactttgccagttggacgcatctgaatatctcaaaaagggtgttgttgccaatatgataggtgttgtcgtatatgttttgaaaaagtataattacgatgcgtatatctctctcgaaaatctttgctatgcatacggatatagcaaagacacattgtcaggatattcaattacaagtactaaggaagatccatatttagatttcaaagatcaagaaaacgcgaaattagcaggattaggaacttatagtttctttgaggttcaacttctgaaaaaacttttcaaacttcaaattgaagaaaatacagagttgattccagctttccgcagcgtggacaactacgagaaaatatttttgttaaaaaatatagataacaaaatttatcagttcggtattgtgtattttgttgatccaaaatatacaagcctatgttgccctatttgtggtgaacatggcaaaaaaaatgttgataggaagaaacatacaaaaaaatatgacgaagatgaattagtatgcaagcaatgcggttttcatacaaatctttcccatatcgaaacaagggttatggaagacaaaacaataaaaaatagttacgatgagtgtaatttgaaagccattgtttctggagatgcaaatgccgcttataacattgcaattaggttgggcaaaaacatatactcaacaattgcagacaaagtaaaggatttgcatcacgaagggaaaaaatacattatcgtaaaaggataa

SEQ ID NO: 93 - DNA, 4008 bp, synthetic construct
  source 1..4008: mol_type = other DNA
atggaacaatatcaattaacaaagacaatacgattcggactcaccaaagtaagaaaagagaagaaacacctcagccacgaggagttggatgaattggtgatggtgtcagaggaaagaatcaaaaaagaacatcctcaggctgaaaaccaattagacgaacagtccttcgtaaaaaaaattggtgattgtagcaaagccataaaagaatatttgttaagttggggaaaagtatgccgacgcattgataaaattacagtaagaaaagagtttttcaagattcttgctcgcaagacctttttcaaatatgaatcaaaagttggtaaaaaaaggacacctctaccttccgaagttaaattatcgggacaaaaagggaataattattatgatgaaccgattaacgaaggcatttctcaattttggcaaaacagagtttcaaaggcattgaaactacattcacaacttgaatcaatgctttttgattacaaaaaagcaatagaaacagaaaaacacaaccaagaaaaccccaaagagaacaatgatcaattcgacaaactgcatcttgtagattttagaaaaatgtttctatctgtatgcagccttgttatggatagtttgcgcccaattgtaaatgaacttatcattgtgtcggataatgtatcgaaagacgaggataagtatattttggactttgttaatgacaaatcgaaacaatgggatttgtttaaacaaatagaggatttacagaatctttgcaaagataacggcgggaatatcccctttggaaaagcaacgttcaataaatacacctcggaacaagccccaaatcatcgagataatgatattcacaaagttatccgagaactaaaaattgaagaatttgtttctgattttataggcttagaacaagaggatatctatcgaaaaatttatcaatcgactcaacacagtttggtgaacttgaacaaaccaagtatttctcctatcattcgcgcacagttttttaagtacaaaccaattccagtattagtcagattcggactcgcgaattatttaaacaaacagcaagggaaaaaatactcaaatcgattgaaagacatccaagaattatttaggatttttggaacatcaaaaagtcccgagttagattattctgacaaaaacaatagaactgaattttctttggacaagtatccaatcaaagttgcatttgactacgcttgggagaggtgtgctcgttcaaaatatgctcaaaagcctgtcgattttcccaaagaaatatgtgtaacctttttggaaacatatttcgaatacaatagtaaagaaaaaaatcgtgaagcatttgaaatatatgcacatttattgaaagtcaacgaatgtttagcgactttggagcactttgagaatgagccgcccaaagatattaaatcactttggcaagatgtgcaaaaccatcttgacaaagtggggaaattatgctcaaatgaggatagaaaagcaataacccaatggtatgaagaatacaaaaatctttgggcgaaaggcaactataaaaagttgaaaaaatggattgagtcaaaatcatataccgttactaatttcactcaggcgaaaatgcatttgggacaaaaaagaggaagccaaaaaacaatggttctgaaatcatattttcaccctacttatggaaaaataaaagatggcaaccgatttataaactctaatgtaaccgaagtgttcaaaaacatagcgagtacttttggtaaatcttttgccaccatcagagattattttaacgaggaaagtgaagtaaataaaatagaatatggtgctgtaattatcaaagataaaaaaggcgacaagtatcttttgcttcaaaagaaaaatgaaggcggtatagatatgcctgtattcaacgaatctgatggaaatggtgattatgatgtttatcaagttcaatctttgacttataaaacagtaaacaaaatttacaattcaacaaaatatcctgaattctttgcaataaatggcgaaaaagcaatttacgcgcctaataggccacaacgattcaaagatgatcaagagaagaatacttttaatgaaaagaaattgcagtcgttgaaaaagtgtttgacagaatctgattttatgacaaacaccaccgaaaattatttgcaaaagttcaattggacagaagaaatcaacaactgcacagatttcgaaccacttgccaaaatagttgatcaaaagggatattacctaaaatcctacaaaatcagcaaagagcaaatagcagaattggttaacaatcagaattgttatctcttgccgattgtaaaccaaaatattacagcaaagacaaagaacgacacaaatcaattcacaaaagattggaacaagattttcaatgatgaatataaagactatcgtcttcatcctgaatttactatgttctatcgttatcctacgcccgattatccttgtcctggcgaaaaaagatattctcggtttcaaatgaatgttaacttcctaatggaagtaattccttccgaaggagaatacgtttcacgaaaggaacagatagagtctttcaatacacccaaacaagacaaagaagataacgataatgaaaatagtcaagcaaaaaaagtagcacaatttaacgataacattaatacaaaaaagccttcatacattattggcatcgaccgaggtattaatgaacttgccactttgtgtgttattaactctgagggcaaaatcgtcgcagttgacgaaaatggattgataaaagacgagtttgatatttatgtaaagcatttcgataaagacaacaaatgttggattcataacatcaaaccaaaaacagcaaccgataataaaccaagaacaatacttgacttgtcgaatttgagggttgagacaactattgacggcaaacaagtattggttgacttgtcatccgacgaaaatggtatacaagtcaacagcaaacaaatagttcacctcaaacgtctttattatttacgttgcctatcctatttattgcaatcatctgattacaagtcaataattttggaaaaactgaaagacgttaataatatgaccgatgacgcaatatatgaagttttcaaaaacgacaaatttattgattcatacaaaggaggtgtacaatatacagatttgccatacgatgaaattagacatttgatttctacttatcaagaaattgaccaatctaataaaacagattcggagaagcaaagcgaattaaacacactttgtcagttagatgcaacagaatctctcaaaaaaggtgttgtcgccaatatgataggtgttgttgtatatattctaaaaaaactaaattacgatgcatatatttctctcgaaaatctatgtcgggctctctattttagcaaagattcactgtcaggttacactattgaaaataccagcgtaaatccagatttagatttcaaggatcaagagaatgcaaaattggcaggcttaggaacttatagttactttgaaattcaactcttaaagaaattgtttaaacttcaaatagatgaaaagcaatttttggttccgggatttcgtagcgttgaaaactacgaaaaaatagtaaagttaggaaaggttaaacattctatttatcaatttggagtagtgcattttgtagaacccgccaatacaagtcttaaatgcccaatttgtggtgctaatggtaaaaggataaagtataatcctaattatgacgaagatgaattagtttgtaaaaaatgtggtttccgttccaatatttctaaaattcaaaacagcaaaattatggaagactctgtaattaaaacctattatgataaccataattataaggcaataatttcaggagataccaatgccggatttaacattgctcttagattgctcatgaacctcaacacacaaattgagaatgcaattaaccatttgcataaaacaggaaaaaactatcacagtgtaaataagtaa

SEQ ID NO: 94 - DNA, 3747 bp, synthetic construct; identical to SEQ ID NO: 90
  source 1..3747: mol_type = other DNA

SEQ ID NO: 95 - DNA, 3729 bp, synthetic construct
  source 1..3729: mol_type = other DNA
atggaaaaataccagattactaagacagtgagatttgggttgacagctacaaattcaaatttgtattccgatgaattaaaagatttgattgaaacttcggaaattaaaatcaaagaatcattaaaaaataaaagtcacaattctctacaaatcgaacagttaaggagttgtttgaacggagtaaaggaatatctgaaaacttggaataatgtttatagccaaattgattttttgggaatatctaaagactattataaagtaatttcaagaaaagcaagatttgattttgataacaaaggacttggttccgaagtcaaacttgcttctctgcaatcaaagtataatagcaaaaaaaggattcaatatattttggatttctgggaagacaatttccagaaaacagaaattctatatcgcaagtcagatgaattattgaaagtttttgaagaagcagaaaagcaaaaacgtgatgataaaaaactgaatgaggtagaattacgtaaaacttttttaagtttatttaatttggtgaatgaaagtttaaaacctttagtagagggaaatttatttactataaacgatgataagatagatagcagaaaccagaatcatgaagtaatcgccgattttatttcaaacactaaagtcagaactgaattatatgagagtataaccgaattacaaaattttttcagagataacggtggttatgttccctttggtagagcgaccttcaatcaatggacagctttgcaaaaagcagataaaaatggagaaagggaaatagataaaattataaaacaactgaatctagaaaccgtttcaatggcgaacattgattataaatataatactttcacaaaaaattttgaacaaggaggacaagtttggaaaatcaaacaaaatgcaaaatctgttattgaactctgccagtttttcaaatataaaaaggtatcgattacaactcgtttaaatcttgccaagcgactgaataagactaataactttttaagtgaatttggaatttcaaaatcacctgctcttgactacaaaaaagataaagaaaacttcaatctagcaaattatccactgaaagtagcatttgattacgcttgggaaaattgtgcaaaagcaaaacacgaaagcattacatttccagagctgcaatgtaaggattatttgcataatgtgtttggtgtggatgcgaataaggataaaaatggaaaaataaaaaatgaagagttaaacaaatatgcagatttattgcaattcaaaatattgcttggaagactaaaagcagaatttcacaaagcagctgaagaaaccaacaaaaataatattcgaaagctaaaaaatatttttgaaaatttagattacagtggtgtgcaagattttaataaaaataaaatcaaagagattgttgaagtctggtttgccaataaagaaaagaatattggaaaaaagaaagaggaaatgattcctttaacagaaaaaaaaaaagatgatttttccaaagccatgcaaattatcggacaagagcgtggggggctgaaaagcagaatcaaaaaatacaaaacattaacggaaatgtttaaggtttgtgcttcaagatttgggaaattatttgccgatttacgagactattttaatgaggcacatgaagttgataaaataaaatatcgttcttggattctagaagatggaaaacaaaatcgatttgttttacttgtcgataaagcaaaagacttggagttagaaaatgaagaaaatggtgaattgaaactttatgaagtaaaaagtttaacttccaaatcactaataaaatttattaaaaacaaaggagcctatcctgattttcacagtttaaatagcttcaattctgatgaaataaagaaaaattggacaaaccataaagccaatataaactttctaaaaaatttgaaatctgcattagaaaattccctaatggctattaaccaaaattggaaagaatttaattttgatttttcaaggtgtgatacttatgaacaaattgaaaaagaaattgatagaaaaggatatattcttaaacaacaaaatatttcattgaatactatcaaaaaatcaatcaatgaagaaaaatcggagaaaataaacaacagcaaaaaattaccaagtttattatttccaattgtaaaccaagatataaacagagaagccaagcaagaaaaaaatcaattcacaaaagattggtttgaaatatttgctgaagaaaataatttacacaaaaagcgtttgcatccagagtttcatttattctatcgctttccaacaaaaaactatccaaatacaaaatttaaaaacggcaaagaaaaatcaaaacgatattctcgctttcaaatgcttgcacattttggtttagaggtatttcctcagggtgattatataagtaaaaaagaacaaatcgaaatttttaatgatgataagaagcaaaaagaagcagttgaaaaatacaataatagtattgtttctgaagttgaatatatcattggtattgatagaggaataaagcaattagccacactttgcgtattaaataaaaatggggtgattcaaagtggttttcaaatttacacacccagttttaatcatgacacaaaacaatgggaacattcttttttaggaaaaagaaatatattagatttatctaatttaagagtggagactactattaaaaacgaaaaagttctagtagatttagcaagtattcaaacaaagaaaggagaaaatcagcaaaaaatcaaacttaaacaactcgcttacattcgtgagttgcaatattctatgcaaaccagacaagtggaattgttagaatatgcaaagactttaaattccgcagaagatattaccgaagaaaaaattaaaatctttatttcaccatttaaagagggaagtcattatgaacatttacccaaacaagaaatatataatttattgaatgaatggcaaaatgcagatgaaacgagaaaacgcaagatacaagaactagaccccactgatagtttgaaatctggaattgtggcaaatatagttggagtgattgctttcttttgtgaaaaatacaactacaaagttcgaatttcattagaagatttaactcgtgcttttagcattcaaaaagatgctttaactggtactcccattcacagaaatgatgaagatttcaaagaacaagaaaatcgaagacttgcaggtgtaggaacaatgcagtttttagaaatgcaactcttgaagaagttgtttaaacttcaatctgaaaaaaataaacatttaattcctgcgtttagaagtgttgctaactatgaaaaaattgttcgtagagataaagagaacggtggtgatgaatttgttaattatccttttggaatagtaacttttgttgatccaagaaatactagtcaaaaatgtccttattgcaataatatagcacgaaaagaagatgatgcattttatagaaatgcaggggaaaataaaaattctctgttatgcaagaaatgtggtttatcaactattaaaggaaaagaaaataagagcaaccaagatgatagtaaaaatcagtttaatattcatttcatcactgacggagaccaaaatggagcttatcatattgctttgaagactttagagaatcttcatcgtttaaatacacctaaagtaacaaagcatactaaaacaaaatggaaaaaatga

SEQ ID NO: 96 - DNA, 3606 bp, synthetic construct
  source 1..3606: mol_type = other DNA
atggagactaacaaaacaacaaaagcaattaatgagtaccaaactcaaaaaacgattagattcggactaacagttacaaataacaatttgtattccgaaaacattgtaaaattattaaaatgctctgaagaaaaaataaaagagcaattaaagaaaacacaaaccgatgatttacaaaaccagaggttaagatgttgtttgattgaaatcaaagaatatctaaaaacgtggaataatgttttttcacaaattgattttttggcaataacaaaagattattacaaagtattatcaagaaaagcaaagtttgattatgataaaggcaatggttcggaaatcaaactttcttccttacaatcaaaacaatcaaagtataatgacaaaaaacgttatcagtatattctggatttttggcatgaaaattttattaaagttgaaaatttgtatcgcaaatcagacgatttattaaaagtatttgaagaagccgcaaatcaaaatcaagatgacaaaaaacttaataaagtggatttgcgaaaaacttttttgagtttattcaatctcgtaaatgaaactctaaaaccgctgattgagggtaatttatttattgtcaatgacgataaaattgacgaacataattcaaagcacaattttgtatcagattttattgtaaaaacagaagaaagaaaacaattgcatgattgtattactgatttacaggatttgttcaaggctaatggcggatatgtgccttttggcagggcgaccattaacaaatggactgctctgcaaaaatctaatcataaagatgatgaaattaaaagaattatcagagagttaaagattgaaaatatctcaatgcaaaatattgattataaatataaatacgatagttttgctgaaaattttaagcaaatatataataaagaaggggagaaagtttgggttttacaatttgatgctaattctgttatcaaagtatgccaatacttcaaatataaaaaagtgccaattaatgctcgtctaaacattgcagaaaggctgataaaagaaaaaagttggcaaagggaaaaaaagaatgattttttaagcgaatttggcatttcaaaatcacctgctttagactacaaaaacgataaagaaaacttcaatctcgcaaactatccgttaaaagttgcttttgattatgcttgggaaaattgtgcaaaagcaatttacgaaacaacaacattcccaaaagaacattgtgaaaaatatttgaaagaggtttttgatttagatatagcaaacaatgcttgttttacaaaatatgctttattgttaagattcaaaatcttaatttgcagaataaaatctgaagaaactactcaaatacaaaatattgaggcagtaagaggtattttagacgaaatcaataaaaatattagtggtaggcaagatttttcaaaagccaaaattattacagaaatcaataattggctttcctttaaagaaaaacaaaccgacaagaaagaaaaatactctaatcaagataacttttctttggcaatgcaaattattgggcaagaacgcggtggtttaaaaagtagaattgaaaaatacaaaactttaacggatatgtttaaagtatgcgcttcaaaatttggaaaacaatttgccgatttgcgcgaatatttccaagaagcgtatgaagttgataaaatcaaatatcgcgcttggattattgaagacgaaaagcaaaaccgttttgttttatttgcgaacaaagaaagggaaattgatttaacgtctgaagaaggtaatttgtatttttatgaagtaaaaagtttaacttctaaatctctcgtgaagtttatcaaaaacagaggtgcttatgcggattttcataaactaaaaaataatttcaattatgaaaaaataaagagagattggcaatactataaaaatgacaagtattttatccaaaatctgaaagatgctttacgcaactccaaaatggctattgaccaaaattgggcagagtttaaatttgatttcacaaagtttaatacttacgaagatatagaaaaagaaattgatagaaaggggtataaacttgtctgcaagacagtttcacttaatacgcttaaagattttgttgaaaacaaaggatgtctcttgttgccaattataaatcaagatattaataaagacgataagcaagccaaaaatcaatttacaaaagattggaatagtatttatgataataaaaagcgtttgcaccctgaatttaacttattctatcgcttcccaacccaagattatccgaatacaaaattcagcaacggaacggaaaagacaaaacgatattcgcgtttccaaatgcttgctcattttggttgtgaatctgttccgaaaggagattatctaagcaaaaaagagcagattgccattttcaacgatgacgcgaaacaaaaagatgcagttgaaaaattcaacaatagcattgcttcagattttgagtatattatcgggattgaccgaggcataaaacaacttgcaacgctttgtgttttgaataaaaacggacagatacaaggagattttgaaatatatacccgaacatttgaaaataaacagtggaaacatactttatcggaaaagcgtaacattttagatttatcgaatttaagagttgaaacaacgattgacggcaacaaagttttagttgatttggcgagtattacgacaaaaaacggtgaaaatcagcaaaaaataaaactcaaacaactcgcttatatcagggaattgcaatattcaatgcaaacaaggagagatgatttgcttgattttgcaaaaggattgcaatctgccgatgatattttgaaagatataagaaatttcattgtgccattcaaggaaggagggcaatatgcagatttacccaacgaaagaatctataatttactaaaagaatggcgagatgccgatgatgaagcaaaacgcaaaatagcagaacttgaccctgcgcaagatttgaaatccggaattgttgccaatatgattggcgtggttgcatttctctgcgaaaaatatggatataaagttcgtatttctttggaggatttaacgagagcatttggcattcaaaaagacgctttaagcggaatagcaattgctccaaatgatgaagattttaaagaacaagaaaatcgtaggcttgccggtgtaggaacttatcagttttttgaaatgcagttgttgaaaaagttattcaaaacgcaggttgataaaaatttacatttagttcctgcttttcgaagtgtggataattatgagaaaattgttcgtagagataaaaagacgaatggcgatgaatatgtaaattaccctttcggtattgtgcgatttattgacccaaaatatacttcaaaaagatgcccgaaatgtggaaaaacagatgttaatcgaaatcaaaagaccaatattgtaaaatgcaataattgtgagtatgaaacaaaagcaggaaattcttccgaagctaataacattcattttattacagatggcgaccaaaatggagcatatcatattgcccaaaaagcattaaaaattcaaaaagaacaataa

SEQ ID NO: 97 - DNA, 3915 bp, synthetic construct
  source 1..3915: mol_type = other DNA
atgaaacagattaaaaatcaatatcaattatcaaaaacgttgcgttttggattgacacaaaaaaacaaaacaaaaaaagaaaattatgctggggaaatctataagagccacagtgaattatctgatttagtagaaatctcagagcaaaggattaaagattctgtatcaacaaataaaaactcagagtcgagtttacctgttgatgctattggtaaatgtcttaatcaaatttctgagttccttaaaggctggcaacaagtatatcagcgtaccgaccaaatagcattagataaggattattataaaatcctttgcaaaaaaattggttttgatggtttttggtttgataagaaaaacggaagaaagacaaaaaagccacaagctcgtattattagccttttagaattggaaaaaaaggacgacaaagaaactgaacgtaaacaatatattcttgattattggcaagaaaattttataaatgctgtggagaaatacaatgttgtcagcgaaaaattaaaacaatttgaagttgcgctaaagataaatagaacggacaataaacccaatgaagtggaatttaggaaactatttctatcgttagtaaatattatttgcgacacacttaaaccgctttgttttcaacagatttgttttccaaggttggaaaaaatagacaattcgaaaatagacaataaaaatttaattgattttgcgatagattaccagtccaaaaacgaattgctttcgctaatatccaaactcaagagttactttgaagaaaacggaggtaatgtgccgtattgccgagctacactcaatcccaaaacagcagttaaaaatccggaatctacagataatagcatagaatcagagataaaaaaacttggacttgataaaattattaaaaataataaagatgcattttccttttcctataatttatacaacaatactgccgaagataaaaaatcaaaattaaaagatgatgaaaatggtggattgatagaacgcagtttactgtttaaatacaagtcgattcctgcaactgttcgattcgaaatagccaaaacattgagcaaaccagacggtaaaaccgaagaagagatattggaatttttacgcgatataggacaactagaaagtcctgcaaaagattatgctgatttaaaggaaaaagacaattttaacatagagaagtatccactgaaagttgcctttaattttgcatgggaaggacttgcccgagcaaaatatcatccagaagctgtttttccgaccgaaatatgtaaacaatatctcaaaaatcattttaagattacagaggataataaagattttgtgatgtatgcaaaactgctggagttgaatgctgttttatctacattagagaaagcaaagcctaccgatgaaaagaaatttagtgttgccgcaaaaaaattattggaagaaatagaatgggaaaaagttgggaaaaatggatccaaaaataaagaagctataacaaaatggttgcagacaaaatctaagacagacaaaaattttaaatcagccaaacaagaaatcggtttgtttagaggtagaataaaaaacaatattagaataaaaaacaatattaagagtgaatactctgaaattacgaatgtatttaagaacatagcagaggaaatgggtaaaacatttgccgaaatgcgcgataaaataagcggtgcggcagaatcaaacaagatttcgcattatgctatgattatagaggataacaataaggacaaatatgttctactacaagaatttgttgaaaataaaaatgaacgaatatatgcaaaatcagatagccaaaagagtgattttaaggcatattcggttaattctatcacttcaggtgcaattgtcaaaatgctcaaaaaaattagaactgacaaattgaaggaaagtaataattttgccaatacacaaccggaattgactagcaaggaaaaagaaaaacgcaatattaaagaatggaaaaagttcattaatgaaaaaggatggaacttggaatttggactaaaattagaaaataaaactttagaagaaattaagaaagaagttgatgccaaatgctataaatttgatattaagtattttgacaaagagactctttccgatttagtgaaaaacaaaaattgtttattactaccaattgtcaatcaagatttggcgaaaaaagaaaaaaacgaaagtaaccagtttacgaaagattggaatgctgtttttcctcaagatacgccttggcgtttaactccggagttcagaatttcttatcgcaagcccacacctaattaccctaaatcggataagggcgataagcgttattcgcgttttcagatgattggacatttcctttgcgattatattccgaaaactgatagctttatttccaaccgccaacaaattgaaaattataaagatgatgaacggcaagaattagcagtaaaaaaatttaatgcagctttgcgagggagaacaaaaaatgaggaatataaagagcaattaaatgaattggcggcaaagtattctaagaatggacagcagaaaataaatgtaaaaactaacgaaaaattttacgttttcggtatcgaccgagggcaaaaagaattggcaacgctttgtattattgaccaagacaaaaaaattatcggtccccataaaatttatacccgttcgttcaactctgaaaaaaaacaatgggaacataaatttttagaagaacggcatattcttgacttgtctaatttgcgtgttgaaactaccgtttttattgatggaaaaccagaaaaaacaaaagtgctggttgatttgagcgaggtgaaagtgaaagacaaagttaccggagaatataccaaacccgacaaaatgcagattaaaatgcagcaacttgcctacattcgtaaacttcaattcaaaatgcagaacgaaccggaagctgtattggcatggtatgaaaaaaattctacagaagatttgattttgaaaaattttgtagataacgaagatggtacgaataacggattggtttctttttatggtgcagccatagaagaactgaaagagactttgcccatagagcgaattgttgatatgcttaaggaattcaaaactataaaaaaagaagagggtaaactcactaaagaggatgaagagggaagggaaaaaaataagcgcaaaatggataaattggtacaattagagcctgttgataatttgaaaaacggtgtcgttgcgaatatggttggtgtgattgcctttttgcttcaaaaatttgattatcaagtttatatctccctcgaagatttgtcaaaaccatttagcagtaaaattatcagtggtattgacggtgttccaattagagttgaaaaagaagaaggacgccgtgctgatgttgaaaaatatgccggactcggactttataatttctttgagatgcaattgctgaaaaaacttttccgcattcaacaggacagtgagaatattctacatttggtaccggctttcagagctatgaaaaattacgaccatatagccgttggaaagggtaaagtaaaaaatcaatttggtattgtgttttttgtagatgcggaggctacttcaaaaacttgtccgcgctgcggttcgactaatcaaaaaccaaacaaaaaagattatcctaatgctcaacaagcaaggttaagcaatgacaaagaagggtggattgaccgtgacaagtcaaatggcaacgatattattcgttgttttgtatgcggtttcgatacaacaaaggaatataccgaaaatccattgaaatacataaatagtggtgatgacaatgcggcgtatttgatttctgccgaaggcgtcaaagcttatgaattggcaacaacgttagctgataatatataa

SEQ ID NO: 98 - DNA, 3879 bp, synthetic construct
  source 1..3879: mol_type = other DNA
atgaaaaacattacaaacaagtatcaaattactaagacattacgttttggcctatcacaaaaagggaaaacaaaaaaagaaggatttgatggagaaatttatcaaagccatcaagaatttaataaattggttagcgtttctgaagcaaggattaaaaaaagtgtaacgacagaacaaaaaacagaattggctttatcaattgataatgttgcacgttgtttaaataacataagtgattttcttataaattggcaacgggtgtattaccgaactgaccaaattgcattagacaaagattattacaaaattatgtgtaagaagattggatttgaaggattctggtttgaaacaaatagacgcactcaacaaaaaataaagaagccacaatcacgtataatcagtctttctgcgcttgataaaaaggatggtttaggcaaggaacgcaaacaatacattttagattattggaaagagaatcttttgtctgccgctgaaaaatatgaagttgttagcgaaaaattgaagcaatttcaagatgcattaaatattaatagaacggacaataaaccaaacgaaattgagcttcgtaaactattcttatcgctcacccatattgtttatgatatacttcagccactttgttatggtcaaatttgttttcccaaaatcgagaaacttgacaacacaaaagaagacaacaaaaagttgattgaatttgcttccgattatcaatcaaaaagcgatttactatccgaaatcgcagaattgaaacaatattttgaggaaaatggtggtaatgtacccttttgccgagccacattaaatccaaaaacacttgtaaaaaatccaaaatcaactgacaatagtattaatgaagaaataaaggatttaggattaaaagagatcttgaaaacatacaaagatgtcttaaactacaacaactatctcgaaagtctatccgcaaaacaaaagctccaattgcttaacgacagaaacacaagtataataacacgcagtttgctgttcaaatataaaccaatttcagccaatgtacaatttgatatagctaaaactctaagcccggaagttggcaagggtgaagaagatttgcgtgctttcttacgtggaattggtcaacctaaaagccctgcaaaagattatgctgatttacaaaacaaatccgatttcaatattgaagcctacccgcttaaagtagctttcgattttgcatgggaaagtttagcaagagcaatatatcatgccgattcagacttgcctatggatgcatgcaaaaattttcttcaagacaattttaaagtaaaaaacgatgatacaaacctcaaattatatgctcaactgcaagaattgaaagctgtgctatcaacattggaaaatggaaatccgaataatgcggctgcttttcgactaaaagctacaaatttgttgaacgagataccttggaagacggttggaaattatggacaacaaaataaagacgaaatttccaaatggttaaataatggtaaaaacaaggatgactataaaaaagcaaaacaacaaataggattattcagaggccgattaaaaaacaacattcaaggttttgataacatcacccaaacgaacaaaaacattgccatgaaaatgggcagaacctttgccacaatgcgcgataaaataaccggcgcagccgaactcaacaaagtgagccactatgctatgattattgaagacagaaacaccgacagatatgttttattgcagccgtttacagaaaacgagcaagacagaatctattcacaaacagattacaacaacggcgattacaccacatacgaagtaaattctataacgtcaggtgcaattgccaaaatgctacgtaaagcaagaatagacgagttgagcaaaaatgacaataacagaaacctcacttcgcaacccgaactgaccgaagaaaaaaaagaaaaacgcaatattaaagagtggaaaaattttattgaaaataaacgttgggatttagaatttcagttaaaattaaatgagaaaaattttgagcaaatcaagaaagaagttgatactaaatgttataatttgagaactaaaaaaattaataaaacaacgcttgaagatttagtaaacaaaagtgattgtttgctgttgcctattgtaaatcaggatttagcgaaagaagaaaaaactaacggcaatcaatttactaaagattggaattcaatttttgcacaaaacactccgtggcgtttgacaccggaattcagggtttcgtatcgtaaaccaactcccgattatccaatatcggataagggtgacaaacgttattctcgtttccaaatgataggtcattttctttgtgattatattccgaaaagtgataaatacatttcaaatagagaacaaattttaaactacaaaaacgacgagttacaaaagaaggcagtcaaagattttcatgaagatttaaaagggaaaaccgaagaagaaaaccaaaatgaatcgatgaatgcgctaatggctaaatttggcaatgtcaataaaaaacagaaagcaacaaccgtagaaaagcccaaagaaaaattttatgtatttggtattgaccgtggacaaaaggaattggctacgctttgcgtgattgaccaagacaaaaagattgtgggcgattttgatatttacacccgcagttttaattcagaacgtaaagaatgggaacatacattctttgaaaaacgccatattttggatttgtccaatttgcgagtggaaaccactgcttcaattgatggaaaagcggaaaagaaaaaagttttggtcgatttgagcgaaattaaagtcaaggataaaaacggcaactattccaaacccgacaaaatgcaaataaaaatgcagcaattagcatacattcgcaagttgcagtttcagatgcagacaaaccctgaaggtgttttagcgtggttcaaagagaattcaacgaaggacttaattattaataatctcgttgataaaaaaaatggtgaaaaaggtttgatttcgttctacggttcggctattgaaaaaatggaagacactttgcccgttgacagaattgaagaaatgcttcaaaaatttgcagctttgaaaaaacaggaaaaagaaggtgaagatgtaaaactatccattgatcaacttgtgcaattagagcctgttgataatttgaaaaacggcgtcgtagcaaatatggttggcgtaatcgcctatctgttgcaaaaatttaattatcaagtatatatatcattagaagacttatcgaatccatttggaagtcaaataacaggtggaattgcaggcgtaccgttgaagcaaggtaaagacgaaggaagacggatggatgtagaaaaatatgccggtcttggtctgtataatttctttgaaatgcaactactcaaaaagctattccgtattcaacaagatagttgtaatattttacatttggttcctgcttttagagcccaaaaaaactatgaccacgttgccgtaggaaaagaaaaggtaaaagggcaatttggaatcgttttctttgttgatgccaatgccacttcaaagacttgccctgtttgcggaacgacaaataataagccaaacaaccaaaagtatcctaatgcgaaaaaaggactttcagcagatggaaaagaagtttggttggaacgcgacaaatcgaatggaaatgatattatccgttgttttgtttgtaactttgatactacaaaagaatatacagaaaaccctcttaaatacattaaaagcggcgacgacaatgcagcttatctaatttcggcggctggaataaaagcatacgaattagcaacaacactcataaacaaccaataa

SEQ ID NO: 99 - DNA, 3786 bp, synthetic construct
  source 1..3786: mol_type = other DNA
atggaaacattaaatcaattcacgggactttattccctgtcaaaaactatgcggtttggtttgacgctcaaagaaaagaaacccaaaaacgattccatagcagtggaatccctctatcaaagccaccaagatttgaaagagttggttgagttatcggacaagagaattatcgaagaaaaaaagccagaaccacctgttgaaaatcttggcaatccgccgattgagaaactacgcgattgtctgaattcgatgcaaaagtatctcaatgattggcgaaaagtctatacaagatatgaccaactcgcagtcttgaaggatttttatagaaaattagaaagaaaagcaagatttgacggattttggaaagacaaaaaaggacaaaatcagccccaatcgcaagagataaaactctcctcactcaagcacaaaagcggagaaaaggaaatcaaagattgcattgtcacatattggggagaaaacatacgaaaggcaaacgagaaatggcatcaggttgattcggttttaaagcaatttgaagaggcaaagcgcaaaaacagagatgacaaaaaactcaatcaagttgaacttcgtaagttgtttctgtcattggcgaaccttgtcaatgatacacttgtaccgttatgtcagagatctatcactttcccaaatgcggataaactctccgacaatgcaagagacaaaagcgtactcgactttattggtgacaacgagatcagagaacatctgcttgataagattaccaagctcaaagagtattttcaagacaatggtggctatgtgccatttggtcgcgtaactctcaaccaatatacggccatgcagaaaccaaacaagaccgacaaagagatagaggacgcaatcaaaaacttaggactttcaatcataaaatcacaaaactttgatgcctttgagcacatagaagaggcgacagacaaagtggaaaggcttaatacggtatctttgccccttgtggagcgggcgcaatacttcaaggacaaaacgattcctgtcggagttcgtgattcattggcaaaatatttggcgaaagacgacactgctaaagaaaaagaacttatcgatttgtttgaaaaaataggcatgcccaaaagacccgcaaaagactacagtgatccaactctcaaagagaagtttgacctgcgcaaatatccgctcaaggttgcatttgattacgcttgggagacagtagcaagcaaagagttacacgatgatattttgaaaaacaaatgcaaaaaatatttgaaagatatttttgacgtcgatactgataaatccatatttttcaacatctattccgatcttaattatatgaaaatcattttatcaagaatcgaatacccaactcaaaatcaactatcaaaagataattttcttgaatggaatagaaaagtaataactatcttagatggcgacgactttagccacttcaataaaaatgcagatggctcaaccgacaagaaaatgaatacagcaaaaacctatgtcaaaacgtggcttgacaaacttgaagccaacatagaacaattcgacggacaagacttcaaaaaattttatgaggattttaaaaagaaaaataagaattcatgtaaagattttgatgacgcaaaaagggatataggattaaagcgcggcggattaaaacaaatcattgaagagacagaaacttttacggacaaaaaaacaggaaaacaaaaaccaaaatacaaagacagcaaatacaaggaattaaccgaggcattcaagagtattgccgtggattttggcaaacattttgccacccttcgcgacaaattcaatgaagaaaacgaaatcaacaagattgaatactacggtgttatcgtcgaagatgaaaatgccgatcgctatcttttgctttcaaaactaagcgaaagtcgcgaggagataaaaaatatctttcctgataaagcagaggggttgaaaacctacaaggtaaaatctctaacctcaaagacactaacaaagctcgtcaaaaacaagggggcatacaaagattttcatatatccgacatgcgcgtagattttaaaaaaatcaaagaagagtggagtgcctacaaaaacgatcaggcttttttgaaatacctcaaaaaatgcctcaccgattccagcatggcacaagctcagaattggtctgaatttggcttggattttgacaaatgcaacacttacgaagaggtagaaaaagagctcgacggcaaagcatatctgctgcaagaaacgcgcctctccaaagcaacaatcactaacttggtcaaaaacaaaggctgctacctcttgcctatcatcaatcaagatttggcgcgagaagaccgcacggcaaaaaatcaatttaccaaagattggaagcagatatttgaaaacaaaaaacattatcgcctgcatccggagttcaatatggcatacagacagccgaccccgaactaccccaactcagagatcggcgacaaaagatattcgcgctttcagatgattgcaaattttatgtgcgagatagttccgcaaagcacaagctacgctacgcgcaaagagcaaatccaaaccttcaacgacaacaataaacaacaaaaagccgttaaagactttgacagcaaatttaaactctccgacagctattttatctttggtatcgacagaggcatcaaacagttggctacgctttgcgtattggatcaaggcggagttatacggggtggatttgaaatctacacgcgacatttcgacggtaataaaaagcagtgggtccatacctctctggagaggcgaaatattttggacttaacaaatctgcgtgcggaaaccacaatagatggtaaaaaagtgctagtagatttgagcaaagtagagatcaaaaatcaaacagacaacaagcaaaatatcaaactcaagcagcttgcttatatcagaaaattgcaatatcaaatgcaaaccaaccccgaaaaagtaaagaatatgtctgatgaagatatcgaaaatgacctaaaagatattattactccatataaagaaggaactcattatgctgatttgccgatagagaatatcaaagcaatgctggatcgcttcaaagttctctacggcaaaaccgaccagcagtccaaacaagaactgaaagagctttgcgagctggatgccgcagataatctcaaaggcggaatagtggcaaacatggtgggtgtcattgcgcatctgatggagcaatacaactacagggtcaagatttcactcgaaaacctaacaacatcatttgtcaaccaatcagatgggcttaacgagtatttcatttcgcgaggtatggatttcaaagaacaagagaatgcggcattggcaggtttgggaacataccaattttttgagatgcaactgctcaaaaaaatattccgcatacaacaagatgatggtaatgttttacatttagttccggcatttagaagcaaagaggattatgaaaaaatcattcggagagacaaaaatgatggcgatgagtatgtaaattatccgtttgggctggtaacttttgttgatccaagatatactagtcgcaaatgccctatatgcggaaaaacagatgtaaaaagaaatgataatataatcacttgcaaaaaatgcggtgccgtatcaggaaaatactcattcgatgataaaaataggcaattcatcaccaacggcgacgaaaacggcgcatatcatatagcattaaaaacaagaaaggaggtgcacaatgaaaactaa

SEQ ID NO: 100 - DNA, 3672 bp, synthetic construct
  source 1..3672: mol_type = other DNA
atggataaagaaaatagttttaaagggtttacgaatttgtatgaagtaaggaaaacagtgaggtttggactaacacaaccaaataaaaaatgagaattaaaaactcatttagaatttgatgatttaataaataaatcttttgaaaacataaaaaaagatgtaaaatcaagagataaaccaaattttaaagaaaaagaactaattgaaaaaataaatcagtttattaattgattagaaaaacaattatgaaattggaaacaaatttatgaaagatatgatgtaatatctgtaaataaagattactataaaatacttgcaagaaaagcaaaatttgatgcttttaaaaaagataaaaaaccacaagcaagtcaaattaaattatcatcattacagaaagataatagaaaagataatataataagatattggtgaaacattattacaagaagtgattatttaataaatatttttaaaccaaaattagaacaatatttaaatgctgttaataatccaaataatagttctcatactaaacctgatttaatagattttagaaaagtatttttacaatttttaaaagtaaatgaagaatatttacaacctctatttgataaatctatacaatttgaaacttgaaaaaaagaaaattctgaagagatcaaaaaaattaatactttttcttgagatgaaaataataaagaaattaattatttgattgatttatgaaaagaaattagagaatattttgaagcaaattgaagtcaagtgccttactgaaaagtgagtttaaattattatacagcattacaaaaaccaaataattttggtgaagatattcgaaaatgagttgaaaatttatgaataataaaatttttgaataaaagtgaagaagatataaaaaattatttaaaacaaaattcaaaagaaaaaataaatttattaaataatgcaaaaaatcattactttattgaattaatacatctttttaagccaaaaacaattcctttttcagtaaagtataatttagcaaaatatttagaaaaaaattttaatttaaaatatgaagatattttaaataaatttgatttactttgaaagtcagttgatatttgaaaagattatcttgaatgtaaagaaaaagaaaagttttcacttgaaaaatatcctattaaatcagcttttgattattcttgggaaaacttagcaagaaatttaaaaagagatgttgattttccaaaaagtgtttgtgaaaagtttttaaaagataattttgatataattattaataatagtagctttaatttatatgcaaatttgctttttattgctgaaaatttggcaacaatagaatattgaaatccaaataacgaaaatgaaattatagagagtataaaaaatacatttgatgatataaaatttgaatctaataaacaagagtatgattgatataaaaaagaaattttaaatattctaaatcaagagaaaagtaaaagaaattataaaaatatattaacagcaaaacaaagattatgattattaagatgacaacaaaaaaataaaatttcaaaatattataatttaacacaatctttcaaaaaaatagcaagttttatttgaaaaactttggctacaataagagaatgattaaaagaagaaaacgaattaaataaaattactgattattgaataataattgaggataaaaatcaagataaatatattttaactttaaaacttgattgaaaagatataagagaaaaaataaaaagtaagttatgggattgagaatataaagtttttgaaattaattcttttacatcaagggcactcaataaatttataaaaaatcccttatgagaagactcaaaaaaatttcattgagattataaatataaacataaggaagtttcaatttacaaagatgtaaaatggatttgatataaagaagaatttttaattcatttaaaagattctttagtaaattctcaaattgcaaaagaacaaaattggaaagctttttgatggaattttgataattttaatacttatgaaaaaattgaaaaagaaattgataaaaaatgatataaattaataaaaaactctatttcaaaagaaaacttagaatacttaataaatgaagaaaaatgtttattatttccattaataaatcaagatatttcaagtaaaaaagaacaaaataaaaatgaatttacaaaagattttaacaaagcatttttatgaatttgatatagaatacatccagaatttagtattttttacagacaacctgatgaagaaaacaaaaaaataaataaatcttgaattataaaccgtttctgaagattgcaattacttgcaaatatttgaattgaatatattccacaaaataatgattataaaacaagaaaagaacaaaataaaatttcattagaccaaacaaatcaaaacgaattagttcaaaattttaataaagaaaaagtaaataaatattttgatagtttagatgattattatattttttgaattgataggtgaataaaacaattagctacattatgtattacaaacaaaaattgaattattcaaagttatgagatttatacaaaatattttaataataattctaaaaaatgggaatataaaaagaatagaattgaatgaattttagatttaacaaatttaaaaattgaatcagataaagattgaaataaatttttagttgatttgtctctatttgaagcaaaagatgaaaattgaaattcaacttgaacaaataaacaaaatataaaattaaagcaattagcttatataagaaagttacaatatcaaatgtcttcaaatgaaaaatgagttttgaattttttaaaaaagtatcaaacaaaagaagaaagacaaaataatataaaagaattaataactccttacaaagaatgacatcattttgaagatttgccagtaaatatttttgaagaaatgtttgaaaactatgaaaagttgaaaaatgataaaactttatcagaaatagaaaaacaaaatttaatgaaacttacaattgagcttgattctagtgaagatttaaaaaaatgagttattgcaaatatgatttgagtgatagtttatttaatgaaaaaatatgattataaagtaaaaattgcagttgaaaatttaaatcaatcttttatgtgacaaaatgattgattaaataacagttatatttcaataaaaacaaattttaaagatcaagaaaattgagctttagcttgaatgtgaacttatcatttttttgaaaatcagttattaagaaagttatataaagtttcggttgaagaatgaatattacatttagttccattttttaattctttagataatgtaaataaattaaattttgaaaaagaaaaaattttatgggttcaaactgaaaactatagaaagttttgaatagttagttttgtaagaccacataacacaagtaaaagatgtcctatttgtaaatcaataaatgtaaagagaaaagataatattacaacttgtagtgactgttgatttataacttgaaaagataataatatagttataaaaaaatataaaaaagaatgattaaatttagatttaattaaaaattgagatgataattgagcttataatatatgttgtaaaatttgactctaa

SEQ ID NO: 101 - DNA, 3720 bp, synthetic construct
  source 1..3720: mol_type = other DNA
gtgaggtttgggcttacccaaccaaataaaaaatgagaattaaaaactcatatagaatttagtgatttagtaaataaatcttttgagaatataaaaaaagaggtaaactcaaaagataaatcaaaatttgatactagaaaagaattgattgataaaataaatcagtttatttcttgattagaaaatcagttatgagactggaagaatatgtatgaaagatatgatttaatatctgtaaataaagattattataaaatacttgcaagaaaagcaaaatttgatgcttttaagaaagataaaaaatgagttaaacaaccacaagctaatcaaattaagttgtcatcattaagatataataaagaattaataataaattattggtgaaatatcatttcaagaagtgattatttaataaatgtttttaaaccaaaattagaacaatatctaaatgctgttaataatccaaataatagttctcatacaaaacctgatttaatagattttagaaaagtatttttacaactgttaaaaataagtgaagaatatttacaacctttatttaataaatctatacaatttgaaacatgaaaaaaagaaaattcttgagatattaaaagagtgaatgatttttcttgaaatgaaaataataaagaaattaatgatttgcttgatttatgaaaagaaattagagaatactttgaagcaaattgaagtcaagttccttattgaaaagttagtttaaactattatacagcagttcaaaaaccaaataattttgataaagaaatcaaagaatgaattaaagatttatgaataatagagtttttaaagaaaagcgaagaggatataaaaaattatttaaaacaagattcaaaagaaaaaatatatttattaaataattcaaaaaatccttactctattgagttaatacaactttttaaaccaaaaacaattcctttttcggtaaaatataatttatctaaatatttagagaagaattataatttaaaatatgaagatattttaaataaatttgatttactttgaaaatctgttgatatttgaaaagattatcttgaatgcaaagataaagaaaaattttcacttgaaaaatatcctattaaatcagcttttgattattcttgggaaaatttagcaagaagtctaaaaagagatgttgattttccaaaaaatgtttgtgaaaaatacttaaatgataattttaatataaatgtttgaaattcaagttttaatttatatgcaaatttactttttattgctgaaaatttagcaacaatagagtattgaaaaccaaataatgaaaaagaaattattgatagtattaaagaaactttcttagaattatcagatgaaatagaaaaaaataataaaaaaaatgaagttgaaaatattataaaatacttaaatttaaacaccgatgaaagaaaaaatattaaagacttacaaaaaaagtattttaaaaatttagatactaaagaacaaaatattctaaatatatttgatagttttacaaaatcaaagcaatctttatgacttctaagatgacaacaaaaaaataaaattgataaatatagaaatttaacacaaaagttagttgataaaaaggattctcatatttgaatagcaagttttatttgaagaactttggcttcaataagggaatgattaaaagaagaaaatgaactaaataaaattactgactattgaataataattgaagataaaaatcaagacaaatatattttaactctaaaacttaacggaaaagatacaagagaaaaaataaaaaataatttatgaaattgagaatataaagtttttgaaataaattcttttacatcaaaagcactcaataaatttataaaaaatcctttatgagaagattcaaagaaatttcattgatattttcaatataaacatagagaagtttcaatatatgatgaaaatgaaaaatgggtttgatataaagaagagtttttgaaacatttgaaacattctttaataaattctcaaattgcagtagaacaaaattggaaagatttttgatggaattttgataattgtgatacttatgaaaagattgaaaaagaagttgataaaaaatgatataaattaatagaaacctctatttcaaaagaaaatttagagaatttaatacataaagaagattgtttattatttccattgataaatcaagatatttctagcaaaaaagaggaaaataaaaatgactttacaaaaaattttgaaaaagtatttttatgagattgatatagaatacatccagagtttagtatattttatagacaaccaaatgaagaaaatttaaaaccaaacaaatcttgaattataaatcgtttttgaagattacaattacttgcaaatatttgagttgagtatattccacaaaacaatgattacacaacaagaaaagaacaaaataaaatttcaatagatcaaacaaaacaaaatgaatcagttcaaaaatttaacaaagaaaaagtaaatccatattttgatagtttagaagattattatattttttgaattgatagatgaattaaacaacttgcaactttgtgtattacaaataaaaaatgagttattcaaaactttgatatttatacaaaacattttaatgataattctaaaaattgggaatataaaaataatagaacagaatgaattttggatttaacaaatttaaaagttgagtcagacaaagaatgaaataaatatttagttgatttatctttatttgaagcaaaagatgaaaattgaaatctaacttgaacgaataagcaaaatgtaaaattaaagcagttagcttatattagaaaacttcaatatcaaatgtcttccaatgaagaatgagttttaagttttttaaataaatataaaacaaaagaagaaagacaaaataatataaaagagttaataacaccatataaagagtgacatcattttgaagacttaccgatgaatatttttgaagaaatgtttgaaaattatgaaaaattgaaaaataataaaactttatcagaatgagaaaaacaaaatttaatgaaactaacaactgaacttgatgcaagtgaagatttgaaaaaatgagttgttgcaaatataatttgagtaatagttcatttaatgaaagaatatgattataaagtaaaaattgcaattgaagatttatcaaatgcttggtatttttcaaaagattgattatcttgagattcaatactaaattccaaaattgatgaagaaatggatttaaaaaaacaagataatttggctttagcttgagtttgaacttaccatttttttgaaatgcagttatttaaaaaattatttaaaatttctgttgaaaaatgaattttacatttagttccaagtttttgaaatgtaagaaattatacagatttattgaaagaaaaatacaaataccaatatcaacaattttgagttatttattttataagcccaaagtttacaagttcaaagtgtcctatctgctgaaaatgatgaaaaaaacatattaagagagaaaacaatgtaataacttgtaaagaatgctgatttgtttcttgaaaagataattcaataaatattaagaacaataaaaaagaatgactaaatttagatttaattaaaaattgagatgataatggatcttataatatttgatgaaaaattaagtaa

SEQ ID NO: 102 - DNA, 24 bp, Oryza sativa
  source 1..24: mol_type = genomic DNA
tggagcaacacctgaaggaaggct

SEQ ID NO: 103 - DNA, 24 bp, Caenorhabditis elegans
  source 1..24: mol_type = genomic DNA
cggaatatgttccccacttcatcg

SEQ ID NO: 104 - DNA, 24 bp, Caenorhabditis elegans
  source 1..24: mol_type = genomic DNA
gccgaaattattcaactgtctgaa

SEQ ID NO: 105 - DNA, 24 bp, Caenorhabditis elegans
  source 1..24: mol_type = genomic DNA
tccagtgagtatcctagacaatga

SEQ ID NO: 106 - DNA, 24 bp, Caenorhabditis elegans
  source 1..24: mol_type = genomic DNA
cattaattcctcttgtggatttgc

SEQ ID NO: 107 - DNA, 24 bp, Caenorhabditis elegans
  source 1..24: mol_type = genomic DNA
gctgctgattctaaaagaattttt

SEQ ID NO: 108 - DNA, 24 bp, Caenorhabditis elegans
  source 1..24: mol_type = genomic DNA
gcagcttcgctggtgtcagcaata

SEQ ID NO: 109 - DNA, 24 bp, Homo sapiens
  source 1..24: mol_type = genomic DNA
gtagtaaacattctactaggattc

SEQ ID NO: 110 - DNA, 24 bp, Homo sapiens
  source 1..24: mol_type = genomic DNA
aaaagggttgaaaaagccgaaggt

SEQ ID NO: 111 - DNA, 24 bp, Homo sapiens
  source 1..24: mol_type = genomic DNA
tggttattaatgtagcctcacgga

SEQ ID NO: 112 - DNA, 24 bp, Pseudomonas syringae
  source 1..24: mol_type = genomic DNA
atgcgtctgcgcgcccgagctgct

SEQ ID NO: 113 - DNA, 24 bp, Pseudomonas syringae
  source 1..24: mol_type = genomic DNA
atcactcggtcgtcgaggccttcg

SEQ ID NO: 114 - DNA, 24 bp, Pseudomonas syringae
  source 1..24: mol_type = genomic DNA
tccttcaaaccgagcactaatgca

SEQ ID NO: 115 - DNA, 24 bp, Pseudomonas syringae
  source 1..24: mol_type = genomic DNA
accgcgggatcggttgtcagcgcg

SEQ ID NO: 116 - protein, 13 aa, synthetic construct
GRRRRRRRRRPPQ

SEQ ID NO: 117 - protein, 27 aa, synthetic construct
GWTLNSAGYLLGKINLKALAALAKKIL

SEQ ID NO: 118 - protein, 30 aa, synthetic construct
MGFVLFSQLPSFLLVSTLLLFLVISHSCRA

SEQ ID NO: 119 - protein, 7 aa, synthetic construct
PKKKRKV

SEQ ID NO: 120 - protein, 16 aa, synthetic construct
KRPAATKKAGQAKKKK

SEQ ID NO: 121 - protein, 20 aa, synthetic construct
FSLDNYPIKVAFDYAWEMCA

SEQ ID NO: 122 - protein, 20 aa, synthetic construct
FSLDKYPIKVAFDYAWERCA

SEQ ID NO: 123 - protein, 20 aa, synthetic construct
FDINHYPLKVAFDFAWESLA

SEQ ID NO: 124 - protein, 20 aa, synthetic construct
FNIEKYPLKVAFNFAWEGLA

SEQ ID NO: 125 - protein, 20 aa, synthetic construct
FNIEAYPLKVAFDFAWESLA

SEQ ID NO: 126 - protein, 20 aa, synthetic construct
FDLDAYPLKVAFDFAWENLA

SEQ ID NO: 127 - protein, 20 aa, synthetic construct
FSLEKYPIKSAFDYSWENLA

SEQ ID NO: 128 - protein, 20 aa, synthetic construct
FNLANYPLKVAFDYAWENCA

SEQ ID NO: 129 - protein, 20 aa, synthetic construct
FDLNHYPIKVAFDYAWEQLA

SEQ ID NO: 130 - protein, 20 aa, synthetic construct
FDLRKYPLKVAFDYAWETVA

SEQ ID NO: 131 - protein, 20 aa, synthetic construct
FNLYKYPLKVAFDYAWESLA

SEQ ID NO: 132 - protein, 20 aa, synthetic construct
FDLFQYPLKPAFDYAWENVA

SEQ ID NO: 133 - protein, 25 aa, synthetic construct
RDILDLSYLRVEKDENGESRLVDLS

SEQ ID NO: 134 - protein, 25 aa, synthetic construct
RTILDLSNLRVETTIDGKQVLVDLS

SEQ ID NO: 135 - protein, 31 aa, synthetic construct
RHILDLSNLRVETTIVIDGKPDVRKVLVDLS

SEQ ID NO: 136 - protein, 31 aa, synthetic construct
RHILDLSNLRVETTVFIDGKPEKTKVLVDLS

SEQ ID NO: 137 - protein, 31 aa, synthetic construct
RHILDLSNLRVETTASIDGKAEKKKVLVDLS

SEQ ID NO: 138 - protein, 31 aa, synthetic construct
RVILDLSNLRVETTIVIDGKPEKKKVLVDLS

SEQ ID NO: 139 - protein, 25 aa, synthetic construct
EGILDLTNLKIESDKDGNKFLVDLS

SEQ ID NO: 140 - protein, 25 aa, synthetic construct
EGILDLTNLKVESDKEGNKYLVDLS

SEQ ID NO: 141 - protein, 25 aa, synthetic construct
RNILDLSNLRVETTIKNEKVLVDLA

SEQ ID NO: 142 - protein, 25 aa, synthetic construct
RNILDLSNLRVETTIDGNKVLVDLA

SEQ ID NO: 143 - protein, 25 aa, synthetic construct
RHILDLSNLRVETTIEGKKVLVDQS

SEQ ID NO: 144 - protein, 25 aa, synthetic construct
RNILDLTNLRAETTIDGKKVLVDLS

SEQ ID NO: 145 - protein, 25 aa, synthetic construct
RNILDLSDLRVETTVEGKKVLVDLS

SEQ ID NO: 146 - protein, 25 aa, synthetic construct
RAILDLSNLRVETTVNGDKVLVDLA

SEQ ID NO: 147 - protein, 22 aa, synthetic construct
QLDASEYLKKGVVANMIGVVVY

SEQ ID NO: 148 - protein, 22 aa, synthetic construct
QLDATESLKKGVVANMIGVVVY

SEQ ID NO: 149 - protein, 22 aa, synthetic construct
QLEPVDNLKAGVVANMVGVIAH

SEQ ID NO: 150 - protein, 22 aa, synthetic construct
QLEPVDNLKNGVVANMVGVIAF

SEQ ID NO: 151 - protein, 22 aa, synthetic construct
QLEPVDNLKNGVVANMVGVIAY

SEQ ID NO: 152 - protein, 22 aa, synthetic construct
ELDSSEDLKKGVIANMIGVIVY

SEQ ID NO: 153 - protein, 22 aa, synthetic construct
ELDASEDLKKGVVANIIGVIVH

SEQ ID NO: 154 - protein, 22 aa, synthetic construct
ELDPTDSLKSGIVANIVGVIAF

SEQ ID NO: 155 - protein, 22 aa, synthetic construct
ELDPAQDLKSGIVANMIGVVAF

SEQ ID NO: 156 - protein, 22 aa, synthetic construct
ELDAADNLKQGIVANMIGIVNY

SEQ ID NO: 157 - protein, 22 aa, synthetic construct
ELDAADNLKGGIVANMVGVIAH

SEQ ID NO: 158 - protein, 22 aa, synthetic construct
ELDAVDDFKTGAVANMIGVIAY

SEQ ID NO: 159 - protein, 22 aa, synthetic construct
ELDSADDLKTGVVANMVGVIAF

SEQ ID NO: 160 - protein, 16 aa, synthetic construct
RQIKIWFQNRRMKWKK

SEQ ID NO: 161 - protein, 12 aa, synthetic construct
GGSGGSGGSGGS

SEQ ID NO: 162 - DNA, 24 bp, Pseudomonas sp.
  source 1..24: mol_type = genomic DNA
agatagaaaacgcccgctcttcgt

SEQ ID NO: 163 - DNA, 24 bp, Pseudomonas sp.
  source 1..24: mol_type = genomic DNA
ctgccgcctccttttgagcattca

SEQ ID NO: 164 - DNA, 24 bp, Leptinotarsa decemlineata
  source 1..24: mol_type = genomic DNA
ttgtgaatagcgttttgaccttcc

SEQ ID NO: 165 - DNA, 24 bp, Leptinotarsa decemlineata
  source 1..24: mol_type = genomic DNA
gcatcgtattggcacggggtatct

SEQ ID NO: 166 - protein, 1256 aa, synthetic construct
MNQEVQGKQYQFSKTLRFGLTSTNQNLYSEETMRLLKVSQEKIEKQVKKENNNTDKTNQLRNCLVQIKEYLKTWDNTYPQIDFLAITKDYYKVISRKARFDFDKGNGSEIKLSSLQSMYNNKKRYQYITDFWKENLHKTENLYRKSDDLLRIFEEAEKQNREDKKLNKVELRKTFLSLFNLVNESLKPLIEGNLFIVNDEKIDEQNPKHNYVSDFILKAEARKPLYNCIGNLQNYFKDNGGYVPFGRVTLNKWTALQKSNNRDTKINRIIKELKINSFLIKNINYKYNEFTSNFKEKKDKKGKIVKNKDGDIVWELEPNDKSVIELCQFFKYKKIPINACLNLAKRLIKENKLEKEKENTFLSELGVSKSPALDYKKDQSNFSLTNYPLKVAFDYAWENCAKAKYEDIPFPKKQCEKYLRDVFDLDIETNADFAKYALLLRFKILIGRIKVEETTRIENIATIKEFFNDVKSNLTKEKDKTVAEINNWLTFKENQTDKKAKYSNQDEFSEAMKTIGEERGGLKSKISRYKALTDMFKVCSSKFGKQFADLRDYFNEAYEVDKIKYRAWIIEDDKKNRFVLLADKGKEVGLTSGNGDLYFYEVKSLTSKSLVKFIKNKGAYPDFHNKKSEDGFCQIYLNSENKENKDRFIDDVKIHWSTYKNDQEFLKKLKECLKNSKMAIEQNWNEFNFDFSECDNYEKLEKEIDRKGYKFERKAISLTDITDLVENKECLLLPIVNHDINKEKQTENQSQFTKDWFAIFKNKKHLHPEFNIFYRFQTKDYLKTKFKNGTEKTKRYSRFQMLAHFGCEVIPQGDYLSKKEQIAIFNDDEKQKKEVENFKENISSDFDYVIGIDRGIKQLATLCVLDKKGVIQGDFQIFTRKFNDITKKWEHKELEKRNILDLSNLRVETTIAGEKVLVDLASIKTKKGENQQKIKLKELAYIRELQYAMQTRKDELLDFANKINSADDITEDSIKNFISPYKEGTRYADLPKSEFFNRLTEWKNADDKGKLKVAELDSADNLKSGIVANMIGVIAFLCEKYKYKVRISLEDLTRAYGIQKDALSGTAIYQNDEDFKEQENRRLAGVGTMQFFEMQLLRKLFKIQIDEKLCLIPSFRSVANYEKIVRRDRKSSGDKFVNYPFGIVCFVDPSYTSQKCPYCDNKHKKNDKETGKKAFYRDKGENKNSLLCKQCGVSTIKGQEKPSNKNDSKKQFNIHFITNGDENGAYHIAKKTLNNLIPNNKNNKNQPSDFPIGTCT

SEQ ID NO: 167 - DNA, 3771 bp, synthetic construct
  source 1..3771: mol_type = other DNA
atgaatcaagaagtacagggaaaacaatatcagttttcgaagaccctgcgcttcggtctgacctctaccaaccaaaacctttatagcgaggaaaccatgcgtctgttaaaggtgagccaagagaagatcgagaagcaggttaaaaaggagaataacaacaccgataaaaccaatcaacttcgcaattgcctggttcagattaaagaatatttgaaaacttgggataacacctatccgcagattgatttcttggcgattaccaaggactactataaagtgatttcccgcaaagcgcgttttgactttgacaaaggtaatggcagcgaaatcaaactcagcagcttgcaaagcatgtataataataaaaaacgttaccaatatattaccgacttctggaaagagaacctgcacaaaacggaaaatttataccgtaagtccgatgatctgctacgtattttcgaagaagcggagaagcaaaatcgcgaagataaaaagctgaataaggtagagctccgtaagacctttctttccctgttcaacctggttaacgagagccttaaaccgctgatcgagggcaatctcttcatcgtcaacgatgagaaaatcgacgaacaaaacccgaaacacaattacgttagcgactttatactgaaagcggaagcgcgtaaaccgttgtacaactgcattggtaacctgcagaactacttcaaggacaacggcggttatgttccgtttggcagagtcaccctgaataagtggaccgcgctgcagaaatctaacaaccgtgacaccaagatcaaccgcatcattaaggaactgaagatcaactccttcctgatcaagaacattaattacaaatacaacgaattcacatccaactttaaggaaaaaaaggacaagaagggtaaaatcgtaaagaacaaagacggcgacattgtttgggaactggagccgaatgacaagtccgtgatcgaattgtgccagttcttcaaatacaagaaaattccgattaacgcttgcctgaacttggccaaacgcctgatcaaagagaacaagttagagaaggagaaggagaatacgttcctgtcggagcttggtgtgagcaaaagcccagcgctggattacaaaaaggaccagagtaatttcagcctgacgaattaccctctgaaagtggctttcgactacgcgtgggaaaactgcgccaaggcaaagtatgaagacatcccgttcccgaaaaaacaatgtgaaaaatacctgcgcgatgtgtttgacctcgatatcgaaaccaacgcggacttcgccaaatacgcgctgctgctgcgttttaagattctgatcggcagaatcaaagttgaggaaactacacgcatcgaaaacatagctaccatcaaggagttcttcaacgatgtgaagtctaatcttaccaaagagaaagacaagaccgtggccgagatcaacaactggctgacgtttaaagaaaatcaaaccgacaagaaggcaaaatacagcaaccaggatgagttctccgaagctatgaaaacgatcggcgaagagcgtggtggtctgaaatccaagattagccgttacaaggcgctcactgatatgtttaaagtgtgtagctcgaagtttggcaaacaattcgcggatctgcgtgattatttcaacgaagcgtatgaagttgataaaattaaataccgtgcgtggattattgaggatgataagaaaaaccgtttcgtcctgttggctgataagggcaaggaggtgggtctgaccagcggtaacggcgatttgtatttttatgaagtgaaatcattgacctctaaatcccttgttaaattcataaaaaacaaaggcgcgtatccggattttcataataaaaagtccgaagatggtttttgtcagatctatctgaactctgaaaacaaggaaaacaaggaccgttttattgatgacgttaaaatccattggagcacgtacaagaacgaccaggagtttctgaaaaagctgaaagaatgtttgaaaaacagcaaaatggctatcgaacagaactggaatgagttcaattttgacttcagcgaatgcgataattacgagaaactggaaaaggagattgaccgcaaaggctacaagtttgaacgtaaggcaattagcctgacggacattaccgatctggttgagaacaaggagtgcctgcttctgccgatcgtgaatcatgatattaacaaagagaagcaaaccgaaaatcaatcgcagttcaccaaggactggtttgcaattttcaaaaacaaaaagcacctgcacccggaatttaacatcttctatcgttttcagaccaaggactatctgaagaccaagttcaaaaacggtaccgagaagaccaagcgttattctcggttccagatgctggcgcactttggctgcgaagttattccgcagggcgactacctgagcaaaaaggagcagatagccattttcaacgatgatgagaaacaaaaaaaggaagttgaaaatttcaaggaaaacatcagtagcgacttcgactacgtgatcggtatcgaccgcggtattaagcaactagcaaccctgtgcgtgctggacaaaaaaggggtcatccaaggtgatttccagatctttacccgcaagttcaatgatatcaccaaaaagtgggagcacaaagaattagaaaaacgtaatattttggatctgagcaatctgcgggtcgagacgaccattgctggtgaaaaagtcttggtggatttggcgagcatcaaaacgaaaaagggtgaaaatcaacagaaaatcaagctaaaagagttggcctacattcgtgaactgcaatatgctatgcagacccggaaagatgagctgttggacttcgcgaacaaaatcaacagcgccgatgatatcactgaggactccattaagaacttcatcagcccgtacaaagagggtacgcgttatgcggacttaccgaaatctgagttcttcaaccgtctgaccgagtggaaaaatgctgacgataagggtaagctgaaagtagcggaactagattctgcagacaacttgaagtccggcattgttgcgaacatgattggcgtgatcgcttttctgtgcgaaaagtataaatacaaggtgcgtatcagcctggaggacctgactcgtgcgtacggcattcagaaggacgcactctccggtaccgcaatctaccagaacgacgaagactttaaggagcaagaaaaccgccgtttggcgggtgttggcactatgcaatttttcgagatgcagctcttgcgaaagctgtttaagatccaaatcgacgagaagttatgcctgatcccgtcctttcgtagcgttgcgaattacgaaaaaatcgtgcgccgagatcgtaagagtagcggtgacaaatttgtcaactacccgtttggcatcgtttgttttgtggatccgagctatacgtcccagaagtgcccatattgcgacaacaaacataaaaaaaacgacaaagaaacgggtaaaaaggcgttttatcgtgacaagggcgagaataaaaactctctgctgtgcaaacagtgtggtgttagcaccatcaaaggccaggagaagccaagcaataagaatgactctaagaagcaattcaatatccacttcatcactaacggtgacgagaacggagcctatcatattgctaaaaaaaccttgaacaatctgattccgaataacaagaacaacaaaaaccaaccgagcgattttccgattggtacctgtacctga

SEQ ID NO: 168 - protein, 4 aa, synthetic construct
GGGS

SEQ ID NO: 169 - protein, 5 aa, synthetic construct
GGGGS

SEQ ID NO: 170 - protein, 5 aa, synthetic construct
GSGGS

SEQ ID NO: 171 - protein, 20 aa, synthetic construct
FSLTNYPLKVAFDYAWENCA

SEQ ID NO: 172 - protein, 25 aa, synthetic construct
RNILDLSNLRVETTIAGEKVLVDLA

SEQ ID NO: 173 - protein, 22 aa, synthetic construct
ELDSADNLKSGIVANMIGVIAF

SEQ ID NO: 174 - RNA, 24 nt, synthetic construct
  source 1..24: mol_type = other RNA
tggagcaacacctgaaggaaggct

SEQ ID NO: 175 - RNA, 24 nt, synthetic construct
  source 1..24: mol_type = other RNA
cggaatatgttccccacttcatcg

SEQ ID NO: 176 - RNA, 24 nt, synthetic construct
  source 1..24: mol_type = other RNA
gccgaaattattcaactgtctgaa

SEQ ID NO: 177 - RNA, 24 nt, synthetic construct
  source 1..24: mol_type = other RNA
tccagtgagtatcctagacaatga

SEQ ID NO: 178 - RNA, 24 nt, synthetic construct
  source 1..24: mol_type = other RNA
cattaattcctcttgtggatttgc

SEQ ID NO: 179 - RNA, 24 nt, synthetic construct
  source 1..24: mol_type = other RNA
gctgctgattctaaaagaattttt

SEQ ID NO: 180 - RNA, 24 nt, synthetic construct
  source 1..24: mol_type = other RNA
gcagcttcgctggtgtcagcaata

SEQ ID NO: 181 - RNA, 24 nt, synthetic construct
  source 1..24: mol_type = other RNA
gtagtaaacattctactaggattc

SEQ ID NO: 182 - RNA, 24 nt, synthetic construct
  source 1..24: mol_type = other RNA
aaaagggttgaaaaagccgaaggt

SEQ ID NO: 183 - RNA, 24 nt, synthetic construct
  source 1..24: mol_type = other RNA
tggttattaatgtagcctcacgga

SEQ ID NO: 184 - RNA, 24 nt, synthetic construct
  source 1..24: mol_type = other RNA
atgcgtctgcgcgcccgagctgct

SEQ ID NO: 185 - RNA, 24 nt, synthetic construct
  source 1..24: mol_type = other RNA
atcactcggtcgtcgaggccttcg

SEQ ID NO: 186 - RNA, 24 nt, synthetic construct
  source 1..24: mol_type = other RNA
tccttcaaaccgagcactaatgca

SEQ ID NO: 187 - RNA, 24 nt, synthetic construct
  source 1..24: mol_type = other RNA
accgcgggatcggttgtcagcgcg

SEQ ID NO: 188 - RNA, 24 nt, synthetic construct
  source 1..24: mol_type = other RNA
agatagaaaacgcccgctcttcgt

SEQ ID NO: 189 - RNA, 24 nt, synthetic construct
  source 1..24: mol_type = other RNA
ctgccgcctccttttgagcattca

SEQ ID NO: 190 - RNA, 24 nt, synthetic construct
  source 1..24: mol_type = other RNA
ttgtgaatagcgttttgaccttcc

SEQ ID NO: 191 - RNA, 24 nt, synthetic construct
  source 1..24: mol_type = other RNA
gcatcgtattggcacggggtatct

SEQ ID NO: 192 - RNA, 36 nt, synthetic construct
  source 1..36: mol_type = other RNA
gtctaaaccgacccaataatttctactgttgtagat